(19) United States
(12) Patent Application Publication
(10) Pub. No.: US 2024/0327889 A1
(43) Pub. Date: Oct. 3, 2024

(54) REGULATION OF CELLS AND ORGANISMS

(71) Applicants: Victor TETS, New York, NY (US); Georgy TETS, New York, NY (US)

(72) Inventors: Victor TETS, New York, NY (US); Georgy TETS, New York, NY (US)

(21) Appl. No.: 18/285,643

(22) PCT Filed: Apr. 4, 2022
(86) PCT No.: PCT/IB2022/053171

§ 371 Date: Oct. 4, 2023

Related U.S. Application Data
(60) Provisional application No. 63/170,885, filed on Apr. 5, 2021.

(51) Int. Cl. C12P 21/00; C12N 9/22

(52) U.S. Cl. CPC C12P 21/00; C12N 9/22; C12Y 301/21; C12Y 301/26

(57) ABSTRACT

The invention relates to medicine, biology, veterinary, pharmacology diagnostics, agriculture, ecology, meteorology, seismology, construction, biotechnology, biomanufacturing and provided herein are products and methods for managing cells behavior, memory of cells and erasure of cell memory. The present invention describes products and methods that, unlike the known ones, make it possible to control the properties of cells and organisms without the use of mutagens and/or the special introduction of genes and/or use of specific gene editing tools and/or changing its environmental conditions.

### FIELD OF THE INVENTION

The invention relates to medicine, biology, veterinary, pharmacology diagnostics, agriculture, ecology, meteorology, seismology, construction, biotechnology, biomanufacturing and provided herein are products and methods for managing cells behavior, memory of cells and erasure of cell memory.

### BACKGROUND OF THE INVENTION

A known method of introducing new genes. In this case, genes are introduced into the cell by various ways: transformation, transduction, etc. (Chen et al., 1987, Naldini et al., 1996). The introduced genes either carry new information or turn off the existing genes.

There is a known method for changing the properties of a cell by editing the genome, when molecules are introduced into the cell that can artificially change the structure of the genome, cutting out and sewing in the genes (Spicer et al 2018).

The present invention describes products and methods that, unlike the known ones, make it possible to control the properties of cells and organisms without the use of mutagens and/or the special introduction of genes and/or use of specific gene editing tools and/or changing its environmental conditions.

### Definitions

Inactivation—destruction; inactivation; cleavage; decrease of the number; inhibition of activity; that are done in vitro, in vivo and/or ex vivo and in any materials.

Alteration—modification; alteration of activity; alteration of structure; alteration of conformation; alteration of nucleic acid components; alteration of binding or association with other molecules i.e. metals, protein, lipid and other nucleic/non-nucleic acids components; qualitative and/or quantitative alterations; alteration of signal generation, reception, transduction, modification; increase of the number; disposition; alteration of activity; restoration after alteration; incomplete restoration after alteration; alteration of production; alteration of their secretion outside the cells; magnetization; that are done in vitro, in vivo and/or ex vivo and in any materials.

Cut-D cells One-time treatment with DNA inactivating product.

Cut-R cells—One-time treatment with RNA inactivating product.

Cut-DR cells or “Drunk cells” One-time treatment with DNA+RNA inactivating products.

Zero-D cells after 2 and more cycles with DNA inactivating products with placing of cells between treatments with DNA inactivating products to the minimal growth conditions (ZD).

Zero-R cells after 2 and more cycles with RNA inactivating products with placing of cells between treatments with RNA inactivating products to the minimal growth conditions (ZR).

Zero-DR Cells after 2 and more cycles with DNA and RNA inactivating products with placing of cells between treatments with DNA and RNA inactivating products to the minimal growth conditions (Z0).

Y-D cells—2 and more cycles with DNA inactivating products with placing of cells between treatments with DNA inactivating products to the same and/or nutritional rich growth conditions.

Y-R—2 and more cycles with RNA inactivating products with placing of cells between treatments with RNA inactivating products to the same and/or nutritional rich growth conditions.

Y-DR—2 and more cycles with DNA and RNA inactivating products with placing of cells between treatments with DNA and RNA inactivating products to the same and/or nutritional rich growth conditions.

NAMACS and NAMACS-ANA—nucleic acid molecule(s) associated with cell surfaces and/or other nucleic acids associated with these surface-associated nucleic acids.

TEZR is a nucleic acid molecule(s) associated with cell surfaces and/or other nucleic acids associated with these surface-associated nucleic acids, capable of recognizing biological, chemical, mechanical and physical factors and generating cell responses.

TEZR can be specific to different cell types and have a length from 2 to 1,000,000 nucleotides.

Microorganisms: bacteria, archaea, fungi, protists, unicellular eukaryotes, unicellular algae, viruses.

Managing: control, regulation, sensing, modulation, alteration, manipulation, management, adjustment.

### SUMMARY OF THE INVENTION

In some embodiments products can destroy and/or inactivate NAMACS and NAMACS-ANA, reverse transcriptase inhibitors, recombinase inhibitors including, protease inhibitors, integrase inhibitors, recombinases as well as cells, organoids, tissues formed following the treatment with these products.

In one embodiment products to be used in medicine, veterinary, ecology, meteorology, seismology agriculture, construction, biotechnology, biomanufacturing for managing functions of procaryotes, eukaryotes including mammalians, plants, fungi, animals, organoids, tissues, embryos, organs, single-cellular, and multicellular organisms.

In some embodiments the products are used for managing relationship to physical, chemical, mechanical and biological factors.

In some embodiments the products can violate signal generation and/or transmission in inside cells and/or outside cells.

In some embodiments the products are used for the diagnosis, treatment and prevention of diseases caused by protozoa, bacteria, fungi and viruses

In some embodiments products are used for managing the recombination of DNA and/or switching on and/or off of the genes.

In some embodiments products are used for managing the formation of spores of bacteria and fungi.

In some embodiments products are used for managing the synthesis of DNA and/or RNA and/or proteins.

In some embodiments products are used for managing post-synthetic modification of nucleic acids and/or proteins; DNA methylation.

In some embodiments products are used for managing the spread of cells; and the resettlement of bacterial biofilms.

In some embodiments products are used for managing the spread of metastases.

In some embodiments products are used for managing of cell properties by turning cells to “Cut” (including “Drunk cell”), “Zero”, “Y” states.

In some embodiments regulation of cells properties is by the inactivation TEZRs

In one embodiment of any of the methods of the invention, the subject is human.

In some embodiments products are used for managing single-strain DNA, double-strain DNA, single-strain RNA, double-strain RNA, DNA-RNA hybrid, Doble-helical DNA, Pauling triplex, G-quadruplex.

In some embodiments products are used for managing organoids including mitochondria and plastids.

In some embodiments TEZRs are on the surface or within membrane vesicles.

In some embodiments products are used for managing process that at least partially regulated by type IV secretion.

In some embodiments s, formation of TEZRs is done by management of type IV secretion In some embodiments products are used for managing the participation of reverse transcription, RNA dependent RNA synthesis, and the formation of nucleic acid molecule(s) associated with the surface of cells and/or associated with them that can trigger formation of the isoforms of proteins and nucleic acids with altered properties.

In some embodiments qualitative and/or quantitative alterations of TEZRs is done within extracellular vesicles.

In some embodiments products are used for managing the work of cell surface receptors with a non-limiting examples of protein receptors.

In some embodiments NAMACS and/or NAMACS-ANA and/or TEZRs are artificial.

In some embodiments products are used for managing the work of cell protein kinase.

In some embodiments products are used for managing signal transduction in mammals and microbial communities.

In some embodiments products are used for managing gene transfer by viruses in mammals and microbial communities.

In some embodiments products are used for managing cells activity within any of the component of microbiota-gut-brain axis.

In some embodiments products are used for managing bacterial colonization and migration

In some embodiments products are used for managing mutagenesis and/or cell adhesion to the substrate and/or rate of cells division, and/or limit of cell divisions.

In some embodiments In some embodiments products are used for managing of DNA recombination

In some embodiments products are used for managing interaction cells and extracellular molecules proteins and/or DNA and/or RNA with prion-like domains of proteins.

In some embodiments products are used for managing process that are associated with reverse transcriptase, of retroelements, group II introns, CRISPR-Cas systems, diversity-generating retroelements, Abi-related RTs, retrons, multicopy single-stranded DNA (msDNA), splicing process.

In some embodiments In some embodiments In some embodiments In some embodiments NAMACS and/or NAMACS-ANA and/or TEZRs are linked to the receptors with proteomic structure.

In some embodiments In some embodiments products are used for managing microbial dormancy and persistence.

In some embodiments products are used for the increase of cell survival at conditions when untreated cells will die.

In some embodiments products are used for managing the resurrection

In some embodiments products are used for managing the arrest or increase of apoptosis and/or necrosis and/or necroptosis and/or other types of cell deaths.

In some embodiments products are used for managing in cell to cell transport of different genes that can be coded in DNA or RNA molecules and activity of cell reverse transcriptase(s) by which RNA molecules can be transformed in DNA

In some embodiments products are used for managing targeted cell delivery.

In some embodiments products are used for managing nlrp3 inflammasome, caspase 1 work and pathway, NF-kB pathway.

In some embodiments products are used for managing of prokaryote-prokaryote prokaryote-eukaryote and eukaryote-eukaryote interactions.

In some embodiments In some embodiments negative impact of the outer environment is ameliorated by wearing clothing that modulates the effects of geomagnetic filed on NAMACS and/or NAMACS-ANA and/or TEZRs In some embodiments products are used for managing weather-dependence In some embodiments products as vaccina against cells NAMACS and/or NAMACS-ANA and/or TEZRs and/or DNase and/or RNase are used for the treatment of diseases and life prolongation

In some embodiments nucleoside and non-nucleoside inhibitors of reverse transcriptase are used alone or in combination with nucleases and/or antibiotics to treat bacterial infections.

In some embodiments qualitative and/or quantitative alterations of NAMACS and/or NAMACS-ANA and/or TEZRs are used for managing functions of procaryotes, eukaryotes including mammalians, plants, fungi, animals, cells, organoids, tissues, embryos, organs, single-cellular, and multicellular organisms with antibodies, mini antibodies, single-domain antibodies (nanobodies), antibodies with nuclease activity (abzymes), antibodies conjugated with nucleases, and other antibody variants, and/or nucleases endonucleases and/or restrictases, and/or exonuclease, with a non-limiting examples of DNase I, DNase X, DNase γ, DNase1L1, DNase1L2, DNase 1L3, DNase II (e.g., DNase IIα, DNase IIβ), caspase-activated DNase (CAD), endonuclease G (ENDOG), AbaSI, AccI, Acc65I, AciI, AclI, AcuI, AfeI, AflII, AflIII, AgeI, AhdI, AleI-v2, AluI, AlwI, AlwNI, ApaI, ApaLI, ApoI, AscI, AseI, AsiSI, Aval, AvaII, AvrII, BaeGI, BaeI, BamHI, BanI, BanII, BbsI, BbvCI, BbvI, BccI, BceAI, BcgI, BciVI, BclI BfaI BglI BglII BlpI, BmgBI, BmrI, BmtI, BpmI, BpuEI, Bpu10I, BsaAI, BsaBI, BsaHI, BsaI-HF, BsaJI, BsaWI, BsaXI, BseRI, BseYI, BsgI, BsiEI, BsiHKAI, BsiWI, BslI, BsmAI, BcoDI, BsmBI-v2, BsmFI, BsmI, BspCNI, BspEI, BspHI, Bsp12861, BspMI BfuAI, BsrBI, BsrDI, BsrFI-v2, BsrGI, BsrI, BssHII, BssSI-v2, BstAPI, BstBI, BstEII, BstNI, BstUI, BstXI, BstYI, BstZ17I, Bsu36I, BtgI, BtgZI, BtsCI, BtsIMutI, BtsI-v2, Cac8I, ClaI BspDI, CspCI, CviAII, CviKI-1, CviQI, DdeI, DpnI, DraI, DraII, DrdI, EaeI, EagI, EarI, EciI, Eco53kI, EcoNI, EcoO109I, EcoP15I, EcoRI, EcoRV, Esp3I, FatI, FauI, Fnu4HI, FokI, FseI, FspEI, FspI, HaeII, HaeIII, HgaI, HhaI, HincII, HindIII, HinfI, HinP1I, HpaI, HphI, HpyAV, HpyCH4III, HpyCH4IV, HpyCH4V, Hpy188I, Hpy99I, Hpy166II, Hpy188III, I-CeuI, I-SceI, KasI, KpnI, LpnPI, MboJ, MboII, MfeI, MluCI, MlyI, MmeI, MnlI, MscI, MseI, MslI, MspA1I, MspI HpaII, MspII, MwoI, NaeI, NarI, Nb.BbvCI, Nb.BsmI Nb.BsrDI, Nb.BssSI, Nb.BtsI, NciI, NcoI NcoI-HF, NdeI, NgoMIV, NheI NheI-HF, NlaIII, NlaIV, NmeAIII, NotI NotI-HF, NruI NruI-HF, NsiI NsiI-HF, NspI, Nt.AlwI, Nt.BbvCI, Nt.BsmAI, Nt.BspQI, Nt.BstNBI, Nt.CviPII, Pacd, PaqCI, PciI, PflMI, PI-PspI, PI-SceO, PleI, PluTI, PmeI, PmlI, PpuMI, PshAI, PsiI-v2, PspGI, PspOMI, PspXI, PstI PstI-HF, PvuI PvuI-HF, PvuII PvuJI-HF, RsaI, RsrII, Sac SacI-HF, SacII, SalI SalI-HF, SapI BspQI, Sau96I, SbfI SbfI-HF, ScaI-HF, ScrFI, SexAI, SfaNI, SfcI, SfiI, SfoI, SgrAI, SmaI, SmlI, SnaBI, SpeI SpeI-HF, SphI SphI-HF, SrfI, SspI SspI-HF, StuI, StyD4I, StyI-HF, SwaT, TaqI-v2, TfiI, TseI ApeKI, Tsp45I, TspRI, Tth111I PflFI, XbaI, XcmI, XhoI PaeR7I, XmaI TspMI, XmnI, ZraI, granzyme B (GZMB), Exonuclease I, Exonuclease V, Exonuclease VII, Exonuclease III, RNaself, RNase III, RNase H1, Exonuclease I, lambda exonuclease, REC BCD nuclease, REC J nuclease, T6 gene exonuclease, combination of thereof, and mutants or derivatives thereof], phosphodiesterase I, lactoferrin, acetylcholinesterase, engineered nucleases, transferases (i.e. methylase), intercalators, different molecules as adapters, mitomycin C, bleomycin, metals, oligonucleotides, polysaccharides, aptomers, protector from nucleases, reverse transcriptase inhibitors and/or salts of orotic acid, and/or ribavirin and/or acyclovir, and/or compound VTL and/or recombinases, protease inhibitors and/or integrase inhibitors, ultrasound and other wave-methods, viruses and their components.

In some embodiments alteration of NAMACS and/or NAMACS-ANA and/or TEZRs include destruction; inactivation; alteration of activity; alteration of structure; alteration of conformation; alteration of nucleic acid components; alteration of binding or association with other molecules i.e. metals, protein, lipid and other nucleic/non-nucleic acids components; qualitative and/or quantitative alterations; alteration of signal generation, reception, transduction, modification; increase or decrease of the number; disposition; restoration after alteration; alteration of production; alteration of their secretion outside the cells; magnetization; that are done in vitro, in vivo and/or ex vivo and in any materials.

In some embodiments products for managing functioning of cells, tissues, organs, organisms, plants and/or plant seeds can be used prior, together and/or after with reverse transcriptase inhibitors and/or recombinase inhibitors, and/or protease inhibitors and/or integrase inhibitors and/or proteases and/or salts of orotic acid, and/or ribavirin and/or acyclovir, antibodies and/or compound VTL.

In some embodiments for managing of plants characteristics treatment with integrase inhibitors prior, together or following the treatment by products are used during the soak.

In some embodiments water, soil, films that contact with seeds or plants or their parts contain and/or are impregnated with nucleases, transferases (i.e. methylase), intercalators, and/or different molecules binding to them of adapters, mitomycin C, bleomycin, metals, reverse transcriptase inhibitors of nucleoside and/or non-nucleoside reverse transcriptase inhibitors and/or salts of orotic acid, and/or ribavirin and/or acyclovir, recombinases and protease inhibitors and/or integrase inhibitors.

In some embodiments cells in “cut”, “Zero”, “Y” states are used as an antigen

In some embodiments treatment of cells and/or their components) with products alter TLRs activity.

In some embodiments treatment of cells and/or their components with products modulate MyD88-STAT3 or MyD88-NF-KB pathways.

In some embodiments, TezRs are restored with aptamers.

In some embodiments, wherein labware (tips, pipettes, dishes, plates, tubes), disposables, liquids, (i.e. PBS, water), nutrient media, contain products to generate cells with new characteristics.

In one embodiment microbial or eukaryotic cells in “Cut” including “Drunk cell”, “Zero”, “Y” states are transplanted to the individual including the same individual from whom these cells were collected with non-altered and/or reprogrammed and/or erased memory.

In one embodiment wherein, eukaryotic cells (i.e. stem cells, hematopoietic stem cell, fibroblasts, endothelial cells, renal cells, immune cells, blood cells) are treated by products to be turned to “Cut” including “drunk cell”, “Zero”, “Y” states prior of being transplanted to the recipient.

In one embodiment the cells in the states as “Cut” including “drunk cell”, “Zero”, “Y” states are used to transfer cells to/from a pluripotent state are used for the reparation and/or regeneration of tissues, organs, part of the body of animals, plants.

In some embodiments, treatment of prevention of diseases is caused by the destruction of TezRs outside or inside the cells.

In some embodiments wherein procaryotic and/or eucaryotic cells, that produce factors that inactivate DNA and/or RNA including representatives of Bacillaceae (i.e. Bacillus spp), Enterobacteriaceae (i.e. E. coli, Salmonella spp., Klebsiella spp.), Pseudomonadaceae, Lactococcoceae, Clostridiaceae families and fungi Aspergillus spp. are added to the soil or water for irrigation.

In some embodiments the products as enzymes which have a nuclease activity is DNase I, various mutants weakening actin-binding may be used. Specific non-limiting examples of residues in wild-type recombinant human DNase I that can be mutated include, e.g., Gln-9, Glu-13, Thr-14, His-44, Asp-53, Tyr-65, Val-66, Val-67, Glu-69, Asn-74, and Ala-114. In various embodiments, the Ala-114 mutation is used. For example, in human DNase I hyperactive mutant comprising the sequence of the Ala-114 residue is mutated. Complementary residues in other DNases may also be mutated. Specific non-limiting examples of mutations in wild-type human recombinant DNAse I include H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P70S, S68N:P70T, S94N:Y96S, S94N:Y96T. Various DNase mutants for increasing DNase activity may be used. Specific non-limiting examples of mutations in wild-type human recombinant DNAse I include, e.g., Gln-9, Glu-13, Thr-14, His-44, Asp-53, Tyr-65, Val-66, Val-67, Glu-69, Asn-74, and Ala-114. Specific non-limiting examples of mutations for increasing the activity of wild-type human recombinant DNase I include Q9R, E13R, E13K, T14R, T14K, H44R, H44K, N74K, and A114F. For example, a combination of the Q9R, E13R, N74K and A114F mutations may be used.

In some embodiments for cells managing for diagnosis, treatment and prevention of diseases and antibiotics resistance development as well as antibiotics resistance overcoming reverse transcriptase inhibitors and substances of the pyrimidine series, namely 2-chloro-5-phenyl-5H-pyrimido[5′,4′:5,6]pyrano[2,3-d]pyrimidine-4-ol derivatives are used.

In some embodiments products can be used in combination with drugs, formulations, procedures, medical interventions with a non-limiting examples of anticancer (with a non-limiting examples of chemotherapy, immunotherapy [PD-1, PD-L1, OX-40, CTLA-4 inhibitors], gene therapy, CAR-T, radiotherapy, antimicrobial, antiviral, antipain, antistress, antiaging, regenerative, hormones, stimulators, antibodies, antipyretics used to the prophylactic and treatment of the diseases and conditions of digestive; cardiovascular, central nervous, musculoskeletal, trauamas otolaryngology, ophthalmology, respiratory, endocrine, reproductive, urinary, obstetrician and gynecological, skin systems; immune and autoimmune diseases, immunosuppressive drugs (with a non-limiting examples of TNF blockers), antibiotic therapy, antipain medicine, siRNA, siDNA, oncolytic viruses, surgery, nutrition, pre-neoplastic and/or neoplastic processes.

In some embodiments, for prokaryote or eukaryote managing antibodies are used

In some embodiments turning cells to “Cut”, “Zero”, “Y” states may lead to the dysfunction of receptors with a non-limiting examples of tyrosin-kinase-based receptors such as EGFR, Tumor necrosis factor related apoptosis-inducing ligand, TLRs, Serotonin receptors, CTLA-4, PD-1, and PD-L1, PD-L2, B7 family, VISTA, Tim-3 and LAG-3, TCR, MHC, Gal-9, MHCII, HHLA2, LSECtin, CD80/86, CD5, CD7, CD4, CD3, CD28, TIL, estrogen receptor, progesterone receptor, human epidermal growth factor receptor, VEGF, VEGFR, RYK, GDNF, RET, ERBB, INSR, IGF-1R, IRR, PDGFR, CSF-1R, KIT/SCFR, FLK2/FLT3, FGFR, CCK4, TRKS, TRKB, TRKC, MEN, RON, EPHA, AXL, MER, TYRO, TIE, TEK, DDR, ROS, LTK, ALK, ROR, MUSK, AATYK, RTK INSR group, FGFR group, EGFR group, EPH group, ROR group; and that affect signaling pathway with a non-limiting examples of those associated with WNT, SRC, PI3K, PTEN, AKT, mTOR, PARP, CHK1/2, WEE, and can be used alone or in combination with other drugs targeting such a receptors with a non-limiting examples of monoclonal antibodies (mAbs) that target the extracellular domain and/or receptor catalytic domains, and that affect aberrant protein phosphorylation.

In some embodiments the use of information, which is recorded in NAMACS and/or NAMACS-ANA and/or TEZRs can be used for the diagnosis, treatment and prevention of neurodegenerative diseases; pain; cardiovascular diseases; diseases of the gastrointestinal tract; diseases of the urinary system; diseases of the musculoskeletal system; injuries; traumas, cancer; blood diseases; migraine and weather-dependent conditions; negative health conditions associated with air travel; conditions associated with poisoning of various nature; receiving doses of radiation; conditions associated with UV exposure; conditions associated with overheating; conditions associated with hypothermia; directions of repair processes for injuries and surgical interventions.

In some embodiments routes of administration of the invention include, e.g., intracerebral, intracerebroventricular, intraparenchymal injections, intrastriatal, intraspinal, parenteral (including subcutaneous, intramuscular, intravenous, intraarterial, inhalation, intradermal, intrathecal, intracisterna magna, epidural and infusion), subarachnoid injection, enteral (e.g., oral), intramuscular, intraperitoneal, transdermal, rectal, nasal, local (including buccal or sublingual), vaginal, intraperitoneal, a local, topical including transdermal, etc.

In some embodiments, DNase and/or RNase delivery to the cells is done by using Lipid Nanoparticle Delivery, Gold nanoconjugated particles, and/or loaded poly (D, L lactide-co-caprolactone) nanocapsules and/or other Nanoparticles and/or, Biohybrid microrobots, microorganisms are used to target the specific cells in mammalians.

In some embodiments, a method for the treatment and prevention of human diseases, by the therapeutic and prophylactic vaccines against NAMACS and/or NAMACS-ANA and/or TEZRs.

In some embodiments the specificity to deliver products is achieved with the delivery of armed antibodies of humanized or chimeric antibodies, antibody fragment targeting the antigen, targeted nanomedicines, peptides, antibody-drug conjugates against TezRs or their components.

In some embodiments the products are used for the treatment of bacterial/HIV-1 co-infection with non-limiting example to be used in patients administering reverse transcriptase inhibitors.

In some embodiments regulation or production, activation, work of NAMACS and/or NAMACS-ANA and/or TEZRs are regulated by genes that are related to retrons with a non-limiting examples of genes: msr, msd and RT (msr-msd-RT).

In some embodiments cells behavior is regulated by products or their mix with aminoglycosides, annamycin, beta-lactams, carbapenem, cephalosporins, carbapenems, chloramphenicol, fluoroquinolones, glycopeptides, lincosamides, lipopeptides, macrolides, monobactams, nitrofurans, oxazolidinones, penicillin, polypeptides, peptide antimicrobial agents, quinolones, sulfonamides, tetracyclines, streptogramins, rifamicin, myxopyronin, azoles, polyenes, 5-fluorocytosin, echinocandins, trimethoprim sulfamethoxazole, nitrofurantoin, urinary anti-infective, lipopeptides, sulfonamides, annamycin's, nitrofurantoin, nitroimidazole, triterpenoids, azoles, echinocandin, nitroimidazole, polyene antibiotics, triterpenoids, peptide antimicrobial agents, bacteriophages, as well as antiseptics and disinfectants (i.e. alcohols, aldehydes, anilids, biguanides, phenols, diamidines, halogen releasing agents, metal derivatives, peroxygens, quaternary ammonium compounds, vapor phase.

In some embodiments In some embodiments inactivation and/or alteration increase and/or decrease and/or modification activity of tumor cells or tumor microenvironment is done with the use cells that migrate to the tumors and/or metastasis (or having a tropism for tumor or tumor environment or capable of engulfing the solid tumors) carrying the genes for synthesis and/or excretion of nucleases with a non-limiting examples of DNase, RNase and their combinations that are delivered straight to tumors and that are administered by different ways with a non-limiting example of p.o, i.v. i.p., intra-tumor etc.

In some embodiments nuclease producing cells are in “Cut”, “Zero”, “Y” states and are used in combination with surgery, local or systemic chemotherapy, immunotherapy, radiotherapy and other targeted therapies.

In some embodiments Bacillaceae (Bacillus spp), Enterobacteriaceae (with a non-limiting examples of E. coli, Salmonella spp., Klebsiella spp.) Pseudomonadaceae, Bifidobacteriaceae, Clostridiaceae are used.

In some embodiments to increase the release of nucleases within the tumor, lytic phages are used against these bacteria or activation of prophages within bacteria after which bacterial subpopulation producing nucleases die with the release of nucleases.

In some embodiments typing of NAMACS and NAMACS-ANA, TEZRs can be used for the identification of the cells.

In some embodiments determination of the characteristics of NAMACS and/or NAMACS-ANA of bacteria and fungi are used to modulate efficacy of sterilization including pasteurization, estimating and/or predicting of the efficacy of sterilization.

In some embodiments products can manage activity of eukaryotic cells, tissues, organs for the modulation of microorganisms' and/or eukaryotic cells' of the immune cells and/or viruses (including oncolytic) migration towards these cells, tissues and organs with a non-limiting examples with the ability to boost immune response and or kill these cells

In some embodiments a qualitative or quantitative analysis of NAMACS and/or NAMACS-ANA and/or TEZRs on prokaryotes and eukaryotes can be used as a biomarkers for the drug therapy efficacy

In some embodiments analysis of the presence of NAMACS and NAMACS-ANA, and/or TEZRs and/or DNase and RNase genes, their expression, level and activity of microbial nucleases in cells, tissues, biofluids are used to analyze, predict and modulate bacterial and cellular growth, interactions and sensitivity to antibiotics, immunotherapy, chemotherapy.

In some embodiments therapeutic effect is achieved by colonization of macroorganism by nuclease-producing microorganisms and eukaryotes.

In some embodiments prophylactic and/or treatment of diseases is achieved by the decrease of DNase and/or RNase activity of cells, human tissues, extracellular space, biofluids of nervous tissue, brain, cerebrospinal fluid, including alterations of ion channels, membrane polarization, electrophysiological parameters, neuronal excitability and synaptic plasticity.

In some embodiments In some embodiments In some embodiments products are used to regulate the activity of nervous cells, formation and maintaining of memory

In some embodiments products can modulate mammalian memory

In some embodiments products are used for the modulation of the memory of “physiological conditions” it a non-limiting examples of pH, temperature, magnetic field, memory, cell memory, taxis, synergism and antagonism, nutrients, oxygen consumption, gas content.

In some embodiments, products are used for regulation memory of antibody-forming cells.

In some embodiments, products can disrupt sense, form and/or transmits and/or transfer signals between molecules, generate a response between cells, group of cells, tissues, organs, organisms.

In some embodiments products usage with/or without of plating cells to a new environment some part of which has to be remembered by the cells leads to the formation of a new and/or altered memory.

In some embodiments plating cells to “Cut”, “Zero”, “Y” state with plating cells to a new conditions results in cells reprogramming and will provide cells with the new properties.

In some embodiments products are used to boost immune cell memory to improve vaccines

In some embodiments analysis of NAMACS and/or NAMACS-ANA and/or TEZRs including those having non-coding genetic information, is used for diagnostics of age, cell health and disease, origin of cells.

In some embodiments, products make cell more susceptible to reprogramming and, consequently, makes the process of reprogramming quicker and more efficient.

In some embodiments, products for reprogramming of cells can be done together with the alterations and modifications of other chaperons, with a non-limiting example of CAF-1 histone chaperone.

In some embodiments products canto modulate adaptation, chemotaxis, taxis, reflexes of eukaryotes or prokaryotes.

In some embodiments products can enhance cells cognition and spatial memory.

In some embodiments treatment of cells with products and NAMACS and/or NAMACS-ANA and/or TEZRs can increase the efficacy of neurotechnology, computers interface, brain-machine interface, intelligence algorithms, can be used to connect computers to organisms, used for neuronets development.

In some embodiments products are used to regulate fertilization, speed and characteristics of the development of the embryo of fish, birds, other animals, humans.

In some embodiments products are used regulate remote sensing.

In some embodiments products are used for managing epigenetic memory

In some embodiments products are used for prokaryotic or eukaryotic cells forgetting

In some embodiments products are used to regulate memorization and/or speed of memorization, and/or long-term and/or short memory formation

In some embodiments products usage can alter methylation within the promoter regions of tumor suppressor genes causes their silencing, and methylation within the gene itself can induce mutational events.

In some embodiments In some embodiments products usage can modulate bacterial metabolism including metabolism of drugs such as hormones, corticosteroids, anticancer drugs, drugs used for the treatment of infectious diseases, drugs used for the treatment of neurodegenerative disorders.

In some embodiments human diseases are the result of inactivation and/or alteration of TEZRs and/or increase and/or decrease and/or modification their activity of prokaryotic and/or eukaryotic cells.

In some embodiments process of cells malignization and/or oncogene activation and/or prometastatic genes activation, turning normal cells to malignant, epithelial-mesenchymal transition can be regulated by the alteration of NAMACS and/or NAMACS-ANA and/or TEZRs.

In some embodiments products can make antibiotic resistant bacteria susceptible to antibiotics.

In some embodiments products can be used to modulate NAMACS and/or NAMACS-ANA disease-associated susceptibility genes, include, but are not limited to, ADAR1, MDA5 (IFIH1), RNase H subunits, SamHD1, TREX, TBK1, Optineurin, P62 (sequestosome 1), Progranulin, FUS, VCP, CHMP2B, Profilin-1, Amyloid-β, Tau, α-synuclein, PINK, Parkin, LRRK2, DJ-1, GBA, ATPA13A2, EXOSCIII, TSEN2, TBC1D23, Risk-factor alleles, PLCG2, TREM2, APOE, TOMM40, IL-33, Glucocerebrosidase, Ataxin2, C9orf72, SOD1, and FUS, ABL1 (ABL), ABL2(ABLL, ARG), AKAP13 (HT31, LBC. BRX), ARAF1, ARHGEF5 (TIM), ATFI, AXL, BCL2, BRAF (BRAF1, RAFB1), BRCA1, BRCA2(FANCD1), BRIP1, CBL (CBL2), CSF1R (CSF-1, FMS, MCSF), DAPK1 (DAPK), DEK (D6S231E), DUSP6(MKP3, PYST1), EGF, EGFR (ERBB, ERBB1), ERBB3 (HER3), ERG, ETS1, ETS2, EWSR1 (EWS, ES, PNE), FES (FPS), FGF4 (HSTF1, KFGF), FGFR1, FGFR10P (FOP), FLCN, FOS (c-fos), FRAP1, FUS (TLS), HRAS, GLI1, GLI2, GPC3, HER2 (ERBB2, TKR1, NEU), HGF (SF), IRF4 (LSIRF, MUM1), JUNB, KIT(SCFR), KRAS2 (RASK2), LCK, LCO, MAP3K8(TPL2, COT, EST), MCF2 (DBL), MDM2, MET(HGFR, RCCP2), MLH type genes, MMD, MOS (MSV), MRAS (RRAS3), MSH type genes, MYB (AMV), MYC, MYCLI (LMYC), MYCN, NCOA4 (ELE1, ARA70, PTC3), NF1 type genes, NMYC, NRAS, NTRK1 (TRK, TRKA), NUP214 (CAN, D9S46E), OVC, TP53 (P53), PALB2, PAX3 (HUP2), STAT1, PDGFB (SIS), PIM genes, PML (MYL), PMS (PMSL) genes, PPM1D (WIP1), PTEN (MMAC1), PVT1, RAF1 (CRAF), RB1 (RB), RET, RRAS2 (TC21), ROS1 (ROS, MCF3), SMAD type genes, SMARCB1(SNF5, INI1), SMURF1, SRC (AVS), STAT1, STAT3, STAT5, TDGF1 (CRGF), TGFBR2, THRA (ERBA, EAR7 etc.), TFG (TRKT3), TIF1 (TRIM24, TIF1A), TNC (TN, HXB), TRK, TUSC3, USP6 (TRE2), WNT1 (INTI), WT1, CCDC26, CDKN2BAS, RTEL1, TERT, ERCC1, ERCC2, ERCC5, BRCA2, IDH1/2, NF1, NF2, TSC1, TSC2, PTEN, CASP-9, CAMKK2, P2RX7, MSH6, PDTM25, KDR, VTI1A, ETFA, TMEM127, GSTT1, CHAFIA, RCC1, XRCC1, EME1, ATM, GLTSCR1, XRCC4, GLM2, PTEN, CDKN2A, CDKN2B, p14/ARF, XRCC3, MGMT, XRCC4, MMR, IDH1, ERBB2, CDKN2A, CCDC26, SUFU, NPAS2, CCDKN2A, PTCH2, CTNNB1, P21, RIC8A, CASP8, XRCC1, WRN, BRIP1, SMARCE1, MN1, PDGFB, VHL.

In some embodiments, diseases are caused by the interaction of NAMACS and/or NAMACS-ANA and/or TEZR of intracellular bacteria with host's cell cytosol.

In some embodiments products are done for the regulation of the interactions of microorganisms in mixed microbial communities, microbial antagonism, including biofilms, including obtaining stable mixtures of microorganisms.

In some embodiments products are done for changing the properties of the cell in order to prevent complications during air/space flights, staying at other planets, therapies and medical intervention, of transplantation (engraftment, rejection, transplant against the host), cancer therapy (chemo-radio-immunotherapy, cytokine release syndrome and other CAR-T therapy side effects)

In some embodiments products are done for changing the properties of the cell modification their activity of immune cells and/or cells targeted by the components of immune system are used to regulate immune response.

In some embodiments products are done for changing the properties of the cell on fecal microbiome transplantation and non-fecal microbiome transplantation (comprised of at least one microorganism species selected from the group consisting of Actinomycetales, Bifidobacteriales, Bacteroidales, Flavobacteriales, Bacillales, Lactobacillales, Firmicutes, Proteobacteria Spirochaetes, Bacteroidetes, Clostridiales, Erysipelotrichales, Selenomonadales, Fusobacteriales, Neisseriales, Campylobacterales, Pasteurellales) aimed to increase the efficacy of such a microbiome transplant for the therapy of human diseases with a non-limiting examples of IBD, Crohn's disease, ulcerative colitis, weight, Chronic Clostridium difficile Infection, colitis, Chronic constipation, Chronic Fatigue Syndrome (CFS), Collagenous Colitis, Colonic Polyps, Constipation Predominant FBD, Crohn's Disease, Functional Bowel Disease, Irritable bowel syndrome, constipation-predominant, IBS diarrhea/constipation alternating, IBS diarrhea-predominant, IBS pain-predominant, Indeterminate Colitis, Microscopic Colitis, Mucous Colitis, Non-ulcer Dyspepsia, Norwalk Viral Gastroenteritis, Pain Predominant FBD, Primary Clostridium difficile Infection, Primary Sclerosing Cholangitis, Pseudomembranous Colitis, Small Bowel Bacterial Overgrowth, NASH, fibrosis, Ulcerative Colitis, and Upper Abdominal FBD, Autoimmune disorders, neurodegenerative disorders with a non-limiting examples of Alzheimer's disease, Parkinson's disease, amyotrophic lateral sclerosis, Multiple Sclerosis, autism, cancers.

In some embodiments products are done in combination with antibiotics to regulate the formation of the spores of spore-forming bacteria

In some embodiments the treatment and prevention of human diseases, by products usage for managing activity within representatives of microbiota including skin, gut, brain, lung, vaginal, tumor microbiotas.

In some embodiments products are done for changing recipients' or/and donors' tissues for the improved efficacy of tissue and organs transplantation.

In some embodiments products are done for changing the properties of the recipient cells to increase the efficacy of CRISPR, TALEN, ZFN and other gene editing technologies.

In some embodiments products are done for the prevention of NAMACS and/or NAMACS-ANA interaction with proteins.

In some embodiments products are used to produce or modulate: ion channels, brain stimulation, cell signaling within nervous system, e.g. neurons, microglia, modification of responses to cortical stimulation, cell signaling between nervous cells and microglia with a non-limiting example of synaptic transmission, synaptic connectivity between neurons, neuronal excitability and synaptic plasticity, brain ageing, age-related deficits in learning and memory, cognitive decline, brain development, neurotoxicity, excitotoxicity, neurodegeneration, neourodevelopment, sleep disorders, epilepsy.

In some embodiments increase or decrease of DNase and/or RNase activity in human tissues, extracellular space, biofluids (with a non-limiting examples of nervous tissue, brain, cerebrospinal fluid) is used to prevent and treat human diseases.

In some embodiments products can be used to modulate the work of Ca, Na, K, channels.

In some embodiments products are used to modulate electrical properties, polarization, depolarization and extrapolarization of cell's membranes potential.

In some embodiments the decrease of RNase activity in human tissues, extracellular space, biofluids are used to modulate electrical properties and depolarization potential of the cells, polarization, depolarization and extrapolarization of membranes potential with a non-limiting examples of neurons.

In some embodiments products are used for managing activity within axons and/or dendrites and/or synapses.

In some embodiments In some embodiments products are done for managing process of viral and/or capsid surface of various delivery vehicles, including, without limitation, viral vectors (e.g., adeno-associated virus vectors, adenovirus vectors, retrovirus vectors [e.g., lentivirus vectors]) is used to increase the specificity of gene therapy.

In some embodiments In some embodiments In some embodiments In some embodiments products are used for regulation of miRNA, protein expression.

In some embodiments products are done for eukaryotic and prokaryotic cells to alter evolution process.

In some embodiments products are done for control activity within eukaryotic and prokaryotic cells to modulate increased intestinal permeability.

In some embodiments In some embodiments In some embodiments products are done for managing of normal lysosomal function, autophagy, control of protein export from neurons, anti-amyloid therapies (including active immunotherapy), drugs aimed targeting protein aggregation and other methods aimed prevents accumulation of misfolded proteins along or together with drugs having synergistic effects on these processes.

In some embodiments products are done within eukaryotic or prokaryotic cells to restore neuron injury and regeneration of neurons and neurological damage

In some embodiments alteration of NAMACS and/or NAMACS-ANA and/or TEZRs including the use of artificial ones and/or are done for formation of system for signal transferring and cellular cooperation and as an analogue of nervous system bringing signals between cells, cell groups, tissues, organs and their qualitative or quantitative change of can be used for the modification of such a signaling.

In some embodiments In some embodiments analysis of NAMACS and/or NAMACS-ANA and/or TEZRs are used to assess the effectiveness drugs in clinical trials.

In some embodiments In some embodiments products are done for managing of stem cells differentiation.

In some embodiments products are done for managing of embryo cells affect the embryogenesis.

In some embodiments products can be used to modulate the efficacy of transmitters formation, release and effects of glutamate, aspartate, D-serine, γ-aminobutyric acid (GABA), glycine, nitric oxide, carbon monoxide, hydrogensulfide, dopamine, norepinephrine (noradrenaline), epinephrine (adrenaline), histamine, serotonin, phenethylamine, N-methylphenethylamine, tyramine, 3-iodothyronamine, octopamine, tryptamine, oxytocin, somatostatin, substance P, cocaine and amphetamine regulated transcript, opioid peptides, adenosine triphosphate (ATP), adenosine, dopamine, acetylcholine, anandamide, etc.

In some embodiments products can be used to regulate work of nocioreceptors and/or opioid receptors and/or mechanoreceptors and/or magnetoreceptors and/or chemoreceptors is associated with.

In some embodiments products manage the release or effects of neutrophil extracellular traps.

In some embodiments products manage surgical outcomes, and/or can be used in vivo or ex vivo for pretransplant organ reconditioning

In some embodiments In some embodiments products are used to treat drug overdose including opioids, drug abuse, prophylactic and treatment of morphine and other drugs overdose, respiratory depression, neuropathic pain, gastrointestinal disfunction, addictions and substance use disorders.

In some embodiments products are used to regulate interferon-dependent cell protection.

In some embodiments products are used to regulate hormones levels, cells sensitivity to hormones with a non-limiting examples of insulin.

In some embodiments products are done for increase and/or decrease and/or modification cells activity with the use of skin products (cream, tonic, etc).

In some embodiments In some embodiments In some embodiments

In some embodiments products are done for mammalian cells affect longevity assurance mechanisms resulting in delay of DNA damage-driven aging

In some embodiments In some embodiments products affect longevity by alteration of mechanisms resulting in delay of DNA damage-driven aging activity is used to regulate DNA repair, DNA recombination, regulation of intragenomic rearrangements, the behavior of prophages, plasmids, transposons and other mobile genetic elements, regulation of protein synthesis in cells.

In some embodiments products usage can lead to the dysfunction of receptors with a non-limiting examples of tyrosin-kinase-based receptors such as EGFR, Tumor necrosis factor related apoptosis-inducing ligand, TLRs, Serotonin receptors, CTLA-4, PD-1, and PD-L1, PD-L2, B7 family, VISTA, Tim-3 and LAG-3, TCR, MHC, Gal-9, MHCII, HHLA2, LSECtin, CD80/86, CD4, CD3, CD28, TIL, estrogen receptor, progesterone receptor, human epidermal growth factor receptor, VEGF, VEGFR, RYK, GDNF, RET, ERBB, INSR, IGF-1R, IRR, PDGFR, CSF-1R, KIT/SCFR, FLK2/FLT3, FGFR, CCK4, TRKS, TRKB, TRKC, MEN, RON, EPHA, AXL, MER, TYRO, TIE, TEK, DDR, ROS, LTK, ALK, ROR, MUSK, AATYK, RTK, FLT3, JAK3, FAK, BCR, TCR, INSR group, FGFR group, EGFR group, EPH group, ROR group; and that affect signaling pathway with a non-limiting examples of those associated with WNT, SRC, PI3K, PTEN, AKT, mTOR, PARP, CHK1/2, WEE, insulin, opioid, and can be used alone or in combination with other drugs targeting such a receptors with a non-limiting examples of monoclonal antibodies (mAbs) that target the extracellular domain and/or receptor catalytic domains, and/or can be used to overcome drug-resistance mutations of such a receptors, with a non-limiting example to affect aberrant protein phosphorylation.

In some embodiments In one embodiment, alterations of cellular memory by products is inherited to the next generation of cells

In some embodiments

In one embodiment, the addition of cells in “Cut”, “Zero”, “Y” states to the organism can cause cascade alterations of other cells, leading to a health beneficial effects including rejuvenation within 24 h post their administration, from 1 day to 1 week, in a month, in a 6 month, in a year, during the time to 5 years, during the time to 10 years, during the time to 20 years, during the time to 50, during the time to 80 years, during the time to 120 years.

In one embodiment, NAMACS and/or NAMACS-ANA and/or TezRs of one cell and/or tissue and/or organism interact with the TezRs of another cell and/or tissue and/or organism

In one embodiment, NAMACS and/or NAMACS-ANA and/or TezRs regulate electrostatic interactions, hydrophobic interactions of cellular components.

In one embodiments, NAMACS and/or NAMACS-ANA and/or TEZRs are used to regulate biological rhythms including circadian rhythms

In some embodiments In some embodiments NAMACS and/or NAMACS-ANA and/or TEZRs can make cells immortal or increase maximum number of cell divisions.

In some embodiments In some embodiments products are used to generate naïve state of the cells more sensitive or resistant for physical, chemical, mechanical, biological factors.

In some embodiments In some embodiments products can be used to increase production of cells or/and their metabolites used in biotechnological applications.

In some embodiments including to control the synthesis and/or synthesis and/or secretion of DNA and/or RNA and/or proteins.

In some embodiments NAMACS and/or NAMACS-ANA and/or TEZRs are used to regulate work of cell receptors including their interactions with ligands.

In some embodiments products are used to increase production of energy by cells.

In some embodiments products are used to control regeneration

In some embodiments products are used control differentiation of cells for the prevention and treatment of diseases and creation of organisms with new characteristics.

In some embodiments products are used to obtain altered immune system cells and/or stem cells and/or mammalian and/or plant cells suitable for embriogenesis and to prevent the development of congenital defects, and can be used for artificial insemination.

In some embodiments products treatment of seeds, plants, are used for plant breeding and/or selection processes and/or regulation of plant productivity

In some embodiments eukaryotes and prokaryotes are treated with products to modulate and control food and beverages fermentation.

In some embodiments products are used for increase productivity of eukaryotic and prokaryotic cells, master cell line containing the gene that makes the desired proteins in biotechnology (e.g. associated with recombinant DNA and RNA; Amino acids; Biopharmaceuticals; Cytokines; Fusion proteins; Growth factors; Clotting and coagulation factors; TNF inhibitors; Interferons, Antibodies; Recombinant Antibodies; Recombinant proteins; AAVs, viruses, Antibodies; Vaccines, Vectors, Receptors, Hormones).

In some embodiments In some embodiments

In some embodiments In some embodiments In some embodiments products are used to change activity of plants and/or plant seeds before and/or after planting of agricultural plants.

In some embodiments products can be used for the production of bioenergy.

In some embodiments products are used for managing the energetic, glycemic, oxidation state of the cells, tissues, organs.

In some embodiments products can be used to increase transport of external molecules to the cell or secretion and excretion from the cells.

In some embodiments products are used to can be used to modulate bacterial, fungal, mammalian, or plant metabolism

In some embodiments products are used to can be used to modulate energy state of the cells (e.g. ATP content in cells) or prevention of recurrent formation ATP content in cells

In some embodiments products can modulate anaerobic survival metabolisms in aerobes (both prokaryotes and eucaryotes) with a non-limiting example of regulation of microbial colonization of the gut, site of anaerobic infections, outer space, places with a poorly vascularization.

In some embodiments products can modulate anaerobic cellular respiration and/or fermentation generate ATP under aerobic and anaerobic environments, and/or effects on NADH and FADH2 metabolism and/or ion channels and ionic passage.

In some embodiments products can be used to modulate somatic mosaicism

In some embodiments products are used for the development of artificial organs and organisms

In some embodiments In some embodiments products are used for the treatment of human diseases, including migraine, meteo-dependence, headaches.

In some embodiments products are used for the treatment of human diseases, including migraine, weather-dependence, headaches are replaced by other microorganisms without TEZRs.

In some embodiments products can be used to target pathways include KRAS/ERK/MEK, PI3K/AKT/mTOR, JAK-STAT, and FAK/SRC, WNT signaling, heat shock regulation, glycogen synthase kinase 3 (GSK-3), and transforming growth factor beta (TGFβ).

### BRIEF DESCRIPTION OF THE DRAWINGS

FIG. 1. shows the effect of tested compounds on swarming motility, biofilm formation and biofilm size.

FIG. 2. (A) shows the effects of products on managing swimming motility, chemotaxis and bacterial growth; (B) shows the use of 2,8-dichloro-5-(4-nitrophenyl)-5,9-dihydro-4H-pyrimido[5′,4′:5,6]pyrano[2,3-d]pyrimidine-4,6(1H)-dione (compound VTL) to mediate cell migration; (C) shows the use of raltegravir added to the media together with RNase A to mediate cell growth.

FIG. 3. shows the absence of RNase A internalization in B. pumilus.

FIG. 4. shows the control of the cell sizes with tested compounds.

FIG. 5. shows the effect of products on microbial growth (A) gram-positive bacteria and (B) gram-negative bacteria.

FIG. 6. shows the effects of used products on potentiation of bacterial growth (A) Control Bacillus VT 1200 24 h growth 37C, (B) Bacillus grown on the media supplemented with DNase I 24 h growth 37 C.

FIG. 7. shows the effects of used products on potentiation of bacterial virulence.

FIG. 8. shows the effects of used products on bacterial-phages interaction.

FIG. 9. shows values that represent the average of three independed experiments. (A) Heat map summarizing the effect of nucleases on survival after heating of a S. aureus culture at different temperatures for 10 min. The color intensity represents the average log 10 CFU/mL, from white (minimal) to blue (maximum). Values represent the average of three independed experiments.

FIG. 10. shows effects of tested compounds on sporulation.

FIG. 11. shows the role of TezRs in magnetoreception

FIG. 12. shows effects of different compounds to the adaptation of cells to gas composition

FIG. 13 shows effects of tested compounds on bacterial chemotaxis and substrate recognition

FIG. 14 shows effect of tested compounds on cell memory and forgetting

FIG. 15 shows effects of tested compounds (DNase and RNase) on generation of cells with a novel biochemical characteristics. The biochemical characteristics of (A) B. pulilus and (b) C. albicans following the use of the tested products were studied using a Vitek-2 system. Test reaction data are shown as “positive,” marked with a blue color or “negative”, marked with white color. Data are representative of three independent experiments.

FIG. 16 shows effect of treatment by reverse transcriptase inhibitors. Heat map representation of growth by control S. aureus or S. aureus following the treatment with nucleases and treatment with Reverse transcriptase inhibitors (RTIs). OD600 is labeled by a color scale, from white (minimal) to red (maximum). Values show representative results of three independent experiments.

FIG. 17 shows effects of tested compounds on signal trafficking. Heat map showing the effect of recombinases on signal transduction in relation to temperature tolerance. CFU are labeled by a color scale, from white (minimum) to blue (maximum). Values show representative results of three independent experiments

FIG. 18 shows transcriptome analysis of S. aureus following the treatment with tested products

FIG. 19 shows the morphology of cells following the use of DNase and RNase compounds (×40 microscopy)

FIG. 20 shows the role of surface-bound nucleic acids in survival of tumor cells

FIG. 21 shows effect of product on survival of non-tumor cells

FIG. 22 shows effect of tested products on cell cycle

FIG. 23 shows quantitative analysis of the distribution or proportion of cells in each phase

FIG. 24 shows the effect of tested products on plant growth

FIG. 25 shows the role of tested products on plants growth. Probes: 1-3 control, 4-6 raltegravir; 7-9 DNase; 10-12 raltegravir with DNase.

FIG. 26 shows the role of RNase in regulation of plants and seeds growth

FIG. 27 shows the effect of “Cut”, “Zero” and “Y” states on germination.

FIG. 28 shows the effect of “Cut”, “Zero” and “Y” states on plant characteristics

FIG. 29 shows the role of tested products in the regulation of different stages of virus-host interactions. The morphological changes indicated a reduction in the cytopathic effect (CPE) in Vero cells following the use of tested products captured at 48 h.p.i. (magnification, ×10). The scale bars represent 100 μm.

FIG. 30 shows tested products can ameliorate viral infection. The virus in the supernatant was harvested at 48 h.p.i. and subjected to titration. Data are expressed as the mean±SD (n=3). * p<0.05 as compared to the control Vero cells infected with HSV-1.

FIG. 31 shows the heatmap—of amyloid production. The color gradient is used, with high amyloid production marked with dark blue and absence of amyloid production with white

FIG. 32 shows regulation of the remote signal distribution with tested compounds

FIG. 33 shows bacterial motility

FIG. 34 shows regulation of signal generation and spread and intergenerational memory formation

FIG. 35 shows the use of tested products to mediate directional cell migration (sector 4 is supplemented with RNase A 100 μg/mL)

FIG. 36 shows the effect of products on protein-based insulin receptors

FIG. 37 shows the effect of tested products on protein-based insulin receptors

FIG. 38 shows the effects of tested products on neuronal excitability

FIG. 39 shows the effects of different products on cell's response to the light

FIG. 40 shows the effects of different products on cell's response to blue light

FIG. 41 shows the effects of different products on managing cell's response to visible light of mammalian cells

FIG. 42 shows the use of tested products for of cell's response to electric stimuli

FIG. 43 shows the effects of TezRs on modulation of microbial growth in different geomagnetic conditions.

FIG. 44 shows the use of μ-metal test systems to modulate cell activity

FIG. 45 shows the microbial response of healthy individual and subjects with weather dependence are shown

FIG. 46 shows the increase of RNase activity by isolated bacteria depending on geomagnetic conditions.

FIG. 47 Y190, wherein n is 1-3; m is 4-14; z is 1-6; and X is an acid.

FIG. 48 shows the effect tested compounds-inducted cell memory loss on modulation of proinflammatory cytokines production by immune cells

FIG. 49 shows the effect of product at cells' response for their stimulation with proinflammatory factors

FIG. 50 shows the effect of tested products on telomere shortening

FIG. 51 shows the effect of products on cell responses

FIG. 52 shows the effects of surface nucleic acids destruction on mRNA of E-cadherin in different cell types

FIG. 53 shows the protection of cell-surface nucleic acids from nucleases.

FIG. 54 shows the alteration of immune memory in cells

FIG. 55 shows the role of TezRs' inactivation in wound healing cellular model

FIG. 56 shows the effect of tested products on tramadol sensitivity in cells

FIG. 57 shows the effect of tested products on opioid receptors

FIG. 58 shows the effects of use of tested products in cell resistance to UV exposure

FIG. 59 shows the specificity of antibodies against NAMACS and/or NAMACS-ANA and/or TEZRs

FIG. 60 shows the alteration of fish gender with products.

FIG. 61 shows the effect of products on blood

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Products and Methods of Compounds Synthesis for Managing Cells' and Organisms' Behavior

To reduce the penetration of low-molecular compounds (proteins) into cells, the following methods of their modification were used

1. Quaternized (quaternary) aminoalkyl derivatives. The modification was carried out by introducing highly basic ionogenic groups into the molecule, such as quaternary amino groups or guanidine groups. The aminoalkyl group was introduced using aminomethylation reactions at the aromatic nucleus of the substrate (1), or aminoalkylation at oxygen, nitrogen atoms, or other nucleophilic centers (2), as well as reductive amination of carbonyl groups (3).

ArH→Ar-CH2NMe2→Ar-CH2N+Me3 (1)

X—OH→X—OCH2CH2NMe2→X—OCH2CH2N+Me3 (2)

X—C═O→X—CH—NHR→X—CH—N+Me2R (3)

2. Guanidino derivatives. To obtain them, a nitrile group was introduced into the substrate followed by amination (4), or an aminoalkyl group with subsequent replacement of the amino group by a guanidine group (5).

X—OH or X-Hal→X—CN→X—CH2NH2→X—CH2-N═C(NH2)2 (4)

ArH→Ar-CH2NH2→Ar-CH2-N═C(NH2)2 (5)

2. To reduce the penetration of high-molecular compounds (proteins) into cells, the following methods of their modification were used.

Modification of the protein with hydrophobic residues at the sulfur atoms of cysteine fragments. The modification is carried out by alkylation, with the introduction of such residues as alkyl groups with a number of carbon atoms from 6 and higher (6), aryl ketone groups (7), perfluoroalkyl groups (8), etc.

A-SH+CH3(CH2)10Hal→A-S—(CH2)10CH3 (6)

A-SH+PhCOCH2-Hal→A-S—CH2COPh (7)

A-SH+C6F5CH2Cl→A-S—CH2C6F5 (8)

2. Modification of terminal amino groups or OH groups by:
combination of protein with aldehydes and subsequent reduction of alkylimines to alkylamines (9),
acylation of protein with acid anhydrides (10),
thiocarbamoylation of protein with alkyl isothiocyanates (11)

A-NH2+C6H13CH═O→A-NH—CH2C6H13 (9)

A-NH2+(C6H13CO)2O→A-NH—COC6H13 (10)

A-NH2+C6H5N═C═S→A-NH—CSNH2 (11)

2. Modification of terminal amino groups or OH groups by:

To prevent the penetration of an organic compound into the cell, it is advisable to obtain its associate with an amino acid (preferably asparagine, glutamine, lysine). In addition, a carbohydrate fragment or its structural analog can be introduced into the substance molecule.

### Example 2: Vaccine and Antibodies Development for Managing Cells Activity

Prepared NAMACS and NAMACS-ANA were isolated from bacteria or eukaryotic cells with QIAamp DNA Mini Kit according to manufacturer's instructions. For some vaccines mouse DNase I or RNase were used with methylated bovine serum albumin (Sigma). Used mixtures consisting of 0.5 volume of full Freund's adjuvant and 0.5 volume of antigen solution. To obtain antibodies, animals (white rabbits, 4 months) were immunized iv using a mixture consisting of 0.5 volume of complete Freund's adjuvant and 0.5 volume of antigen solution. Two re-immunizations were carried out with a mixture of Freund's incomplete adjuvant after 21 and 28 days. The resulting antibodies interacted with the DNA used for immunization. In the follow-up experiments each vaccination includes from 1 to 3 doses of nucleic acid or proteins from 1.0 μg/dose to 1.0 g/dose and adjuvants (e.g. Freund's adjuvant) and are administrated by enteral, topical, intramuscular or intravenous or subcutaneous injections.

### Example 3: Products and Method for Managing Microbial Swarming Motility, Biofilm Formation and Biofilm Sizes

To study the effects of compounds on management of swarming motility bacterial biofilms, we prepared glass Petri dishes containing Columbia and Nutrient agar media mixt supplemented or not with tested compounds.

We used different compounds taken at various concentrations from 0.1 to 1000 μg/mL, some of them were used directly (table 1) and some were modified as described in the example 1 to avoid any penetration inside the cells (table 2). Then, 25 μL of a suspension containing 5.5 log 10 cells was inoculated in the center of the agar and the dishes were incubated at 37° C. for different times. The biofilms were photographed with a digital camera (Canon 6; Canon, Tokyo, Japan) and analyzed with Fiji/ImageJ software. The effects of tested compounds was analyzed by the alteration of swarming motility which was confirmed by the formation of a larger colonies on the agar with the irregular swarming pattern. All tested products have similar effect on bacteria (FIG. 1, Tables 1-2).

For data in FIG. 1, bacteria were harvested by centrifugation at 4000 rpm for 15 min (Microfuge 20R; Beckman Coulter, La Brea, CA, USA), the pellet was washed twice in phosphate-buffered saline (PBS, pH 7.2) (Sigma-Aldrich) or nutrient medium to an optical density at 600 nm (OD600) of 0.003 to 0.5. Bacteria were treated for 30 min at 37° C. with nuclease (DNase I), if not stated otherwise, washed three times in PBS or broth with centrifugation at 4000×g for 15 min after each wash, and resuspended in PBS or broth.

TABLE 1
Products tested and their effects on swarming motility and biofilm size

| | Potentiation | | Potentiation | | Potentiation |
|---|---|---|---|---|---|
| | of swarming | | of swarming | | of swarming |
| | motility and | | motility and | | motility and |
| | increased | | increased | | increasing |
| | bacterial | | bacterial | | bacterial |
| Tested product | growth | Tested product | growth | Tested product | growth |
| Alkylating agents | Yes | Anthraquinones | Yes | Anthraquinones | Yes |
| (Busulfan) | | (physcion) | | (1,8-dihydroxy | |
| | | | | anthraquinone) | |
| Piperazines | Yes | Polymerase (Tag) | Yes | RAPI family (lign) | Yes |
| (Pipobroman) | | | | | |
| Antineoplastic | Yes | T4 Polynucleotide | Yes | Prd paired domain | Yes |
| (Mitotane) | | Kinase | | family(1pdn) | |
| Antineoplastic | Yes | DNA | Yes | Tc3 transposase | Yes |
| (Bleomycin) | | Methyltransferases | | family: 1tc3 | |
| | | (DNMT1) | | | |
| Anthraquinones | Yes | HIV-1 reverse | Yes | Trp repressor family: | Yes |
| (chrysophanol) | | transcriptase | | 1trr | |
| Antineoplastics | Yes | M-MLV reverse | Yes | Diptheria Tox | Yes |
| (Methotrexate) | | transcriptase | | repressor family: 1ddn | |
| Porphyrins | Yes | AMV reverse | Yes | Transcription factor | Yes |
| | | transcriptase | | IIB: 1d3u | |
| Histone H1 | Yes | Telomerase | Yes | Interferon regulatory | Yes |
| | | | | factor: 1if1 | |
| Histone H2A | Yes | Lexitropsin | Yes | Catabolite | Yes |
| | | | | gene | |
| | | | | activator protein | |
| | | | | family: 2cgp | |
| Histone H2B | Yes | M-MuLV Reverse | Yes | Transcription factor | Yes |
| | | Transcriptase | | family: 3hts | |
| Histone H3 | Yes | Cro and Repressor | Yes | Ets domain family: | Yes |
| | | family (1lmb) | | 1bc8 | |
| Histone H4 | Yes | Homeodomain | Yes | ββα-zinc finger | Yes |
| | | family (1fjl) | | family: Zif268 zinc | |
| | | | | finger | |
| Histone H5 | Yes | LacI repressor | Yes | ββα-zinc finger | Yes |
| | | family (1 wet) | | family: Tramtrack | |
| | | | | protein | |
| Polymerase (Taq) | Yes | Endonuclease FokI | Yes | Hormone-nuclear | Yes |
| | | family (Ifok) | | receptor family: 2nll | |
| Polymerase (T4) | Yes | γδ-resolvase family | Yes | Loop-sheet-helix | Yes |
| | | (1gdt) | | family: 1tsr | |
| Polymerase (Pfu) | Yes | Hin recombinase | Yes | GAL4-type family: 1zme | Yes |
| | | family (1hcr) | | | |
| Leucine zipper | Yes | MetJ repressor | Yes | Skn-1 transcription | Yes |
| family: 2dgc | | protein: 1cma | | factor: 1skn | |
| Helix-loop-helix | Yes | Tus replication | Yes | Viral factors | Yes |
| family: 1am9 | | terminator family: | | (EBNA1 nuclear | |
| | | 1ecr | | protein family: 1b3t) | |
| Histone family: 1 aoi | Yes | Integration host | Yes | Cre recombinase | Yes |
| | | factor family: 1ihf | | family: 1crx | |
| EBNA1 nuclear | Yes | DNA polymerase T7 | Yes | TATA box-binding | Yes |
| protein family: 1b3t | | | | family: 1ytb | |
| Rel homology region | Yes | Transcription factor | Yes | Viral factors (HIV | Yes |
| family: 1a3q | | T-domain: 1xbr | | reverse | |
| | | | | transcriptase: 1hmi) | |
| Stat protein family: | Yes | Hyperthermophile | Yes | Cationic molecules | Yes |
| 1bf5 | | DNA-BP: 1azp | | with r benzimidazole- | |
| | | | | biphenyl core | |
| | | | | (tetrahydropyrimidinium) | |
| Methyltransferase | Yes | Uracil-DNA | Yes | netropsin | Yes |
| family: 6mht | | glycosylase | | | |
| Endonuclease PvuII | Yes | 3-Methyladenine | Yes | distamycin A | Yes |
| family: 1pvi | | DNA glycosylase | | | |
| Endonuclease V | Yes | Homing | Yes | pyrrole-imidazole- | Yes |
| family | | endonuclease | | pyrrole oligomer | |
| DNA mismatch | Yes | Topoisomerase I | Yes | imidazole pyrrole | Yes |
| endonuclease | | | | pyrrole oligomer | |
| DNA polymerase-β | Yes | Molecules with r | Yes | N-methyl-3- | Yes |
| family | | benzimidazole- | | hydroxypyrrole | |
| | | biphenyl core | | | |
| | | (Amidinium) | | | |
| DNA polymerase-β | Yes | Cationic molecules | Yes | pyrrole-imidazole- | Yes |
| family: 9icf | | with r benzimidazole- | | pyrrole oligomer | |
| | | biphenyl core | | | |
| | | (Amidinium) | | | |
| imidazole pyrrole | Yes | Psoralens | Yes | pyrrolo[2,1- | Yes |
| pyrrole oligomer | | | | c][1,4]benzodiazepine- | |
| | | | | benzimidazole hybrid | |
| N-methyl-3- | Yes | 4′-(Hydroxymethyl)- | Yes | pyrrolo[2,1- | Yes |
| hydroxypyrrole | | 4,5′,8- | | c][1,4]benzodiazepine- | |
| | | trimethylpsoralen | | naphthalimide | |
| Hairpin polyamide | Yes | N4C-ethyl-N4C | Yes | Adriamycin | Yes |
| N-methyl-3- | Yes | N2G-trimethylene- | Yes | daunomycin | Yes |
| hydroxypyrrole- | | N2G | | | |
| pyrrole | | | | | |
| Pyrrole-N-methyl- | Yes | neomycin-grove | Yes | poly(trimethylene | Yes |
| 3-hydroxypyrrole | | binder | | carbonate) | |
| Pyrrole-imidazole | Yes | nogalamycin | Yes | Platinum | Yes |
| polyamides | | | | | |
| pyrrole-imidazole | Yes | neocarzinostatin | Yes | Nucleic acids | Yes |
| derivatives | | | | binding domains of | |
| | | | | TLR9 | |
| bis(distamycin)fumaramide | Yes | ditercalinium | Yes | cryptolepine | Yes |
| Nuclear | Yes | Nuclear | Yes | Benzimidazole | Yes |
| ribonucleoproteins | | ribonucleoproteins | | | |
| BRCA1 | | p53 | | | |
| benzimidazol-2-yl- | Yes | 1,4-Bis{[1-(((5-(5- | Yes | 1,4-Bis{[1-(((5-(5- | Yes |
| fur-5-yl-(1,2,3)- | | N-isopropyl- | | imidazolin-2- | |
| triazolyl dimeric | | amidino)benzimidazol- | | yl)benzimidazol-2- | |
| derivative | | 2-y1) furan-2- | | yl)furan2- | |
| | | yl)methylene)-1H- | | yl)methylene)-1H- | |
| | | 1,2,3-triazole-4- | | 1,2,3-triazole-4- | |
| | | yl]methylene- | | yl]methyleneoxy}benzene | |
| | | oxy}benzene | | hydrochlorid | |
| | | hydrochloride | | | |
| 1,4-Bis{[1-(((5-(5- | Yes | Bis{1-[((5-(5-N- | Yes | 1,3-Bis{1-[((5-(5- | Yes |
| amidino)benzimidazol- | | isopropylamidino)b | | imidazolin-2- | |
| 2-y1)furan-2-y1) | | enzimidazol-2- | | yl)benzimidazol-2- | |
| methylene)-1H- | | yl)furan2- | | yl)furan2- | |
| 1,2,3-triazole-4- | | yl)methylene]-1H- | | yl)methylene]-1H- | |
| yl]methylene- | | 1,2,3-triazole-4- | | 1,2,3-triazole-4- | |
| oxy}benzene | | yl}dimethylene | | yl}propane | |
| hydrochloride | | ether hydrochloride | | hydrochloride | |
| 1,3-Bis{1-[((5-(5- | Yes | 1,3-Bis{1-[((5-(5- | Yes | Phenazine | Yes |
| N-isopropylami- | | amidino)benzimidazol- | | | |
| dino)benzimidazol- | | 2-yl)furan-2-yl) | | | |
| 2-yl) furan-2- | | methylene]-1H- | | | |
| yl)methylene]-1H- | | 1,2,3-triazole-4- | | | |
| 1,2,3-triazole-4- | | yl} propane | | | |
| yl} propane | | hydrochloride | | | |
| hydrochloride | | | | | |
| TATA-binding | Yes | Transcription factor | Yes | Transcription | Yes |
| protein | | TFIIA | | factor TF_{II}D | |
| transcriptional | Yes | transcriptional | Yes | C2H2-zinc finger | Yes |
| activator protein | | activator protein | | | |
| (transcription | | (transcription | | | |
| factor) PU.1 | | factor) GATA-1 | | | |
| Homeodomain | Yes | Basic helix-loop- | Yes | Basic leucine | Yes |
| | | helix | | zipper | |
| Nuclear hormone | Yes | NF-kappaB | Yes | AP-1 member c-FOS | Yes |
| receptor | | | | | |
| AP-1 member | Yes | Myb_DNA-binding | Yes | transcriptional | Yes |
| ATF-2 | | | | repressor protein | |
| | | | | Lambda repressor | |
| transcriptional | Yes | transcriptional | Yes | transcriptional | Yes |
| repressor TetR | | repressor MarR | | repressor MerR | |
| transcriptional | Yes | Transcriptional | Yes | Transcriptional | Yes |
| repressor CprB | | repressor CTCF | | repressor QacR | |
| replication protein | Yes | uracil-DNA | Yes | transcription | Yes |
| A | | glycosylase | | activator-like | |
| | | | | effector nucleases | |
| Leucine zipper | Yes | Cas9 | Yes | Primers specificity | Yes |
| mithramycin | Yes | Nucleophosmin | Yes | locked nucleic | Yes |
| | | | | acids | |
| Actinomycin | Yes | Nogalamycin | Yes | | Yes |
| DNase I, | Yes | DNase X | | DNase γ | Yes |
| DNase1L1 | Yes | DNase 1L2 | Yes | DNase 1L3 | Yes |
| DNase II | Yes | endonuclease G | Yes | caspase-activated | Yes |
| | | | | DNase | |
| ApoI | Yes | BamHI | Yes | EcoRI | Yes |
| EcoR | Yes | RsaI | Yes | granzyme B | Yes |
| Exonuclease I, | Yes | Exonuclease V | Yes | Exonuclease VII | Yes |
| Exonuclease III | Yes | ApaI | Yes | BanII | Yes |
| BclI-HF | Yes | EcoNI | Yes | EcoRV | Yes |
| PluTI | Yes | SfoI | Yes | XmnI | Yes |
| Antibodies against | Yes | XhoI | Yes | AciI | Yes |
| TezR_D | | | | | |
| S7 | Yes | BsaJI | Yes | CviKI-1 | Yes |
| lambda | Yes | REC BCD nuclease | Yes | T6 gene | Yes |
| exonuclease | | | | exonuclease | |
| Phosphodiesterase | Yes | | | | |

We also used compounds modified as previously described in order to prevent their penetration inside the cells.

TABLE 2
The effects of modified products on managing swarming motility

| | Potentiation | | Potentiation | | Potentiation |
|---|---|---|---|---|---|
| | of swarming | | of swarming | | of swarming |
| | motility and | | motility and | | motility and |
| | increased | | increased | | increased |
| Tested | bacterial | Tested | bacterial | Tested | bacterial |
| product | growth | product | growth | product | growth |
| Modified | Yes | Modified | Yes | Modified | Yes |
| DNase I | | Bleomycin | | Distamycin A | |
| Modified | Yes | Modified | Yes | Modified | Yes |
| Histone H1 | | Histone H2A | | Polyamide | |
| Modified | Yes | Modified | Yes | Modified | Yes |
| Polymerase | | pyrrole- | | nogalamycin | |
| (Taq) | | imidazole- | | | |
| | | pyrrole | | | |
| | | oligomer | | | |
| Modified | Yes | Modified | Yes | Modified | Yes |
| Benzimidazole | | TATA- | | transcriptional | |
| | | binding | | activator protein | |
| | | protein | | GATA-1 | |
| Modified | Yes | Modified | Yes | Modified | Yes |
| Leucine zipper | | Cas9 | | Phenazine | |

The results clearly show that the tested compounds can be used for the control of bacterial growth, biofilm formation and bacterial swarming motility and that happens due to the adding of the tested products to the medium.

Interestingly, the combined one-time treatment of cells with tested products along their adding to the medium led to a striking difference in swarming motility compared to the large biofilms formed by B. pumilus with tested products (nucleases) added only to the medium. The biofilms of B. pumilus pretreated with DNase I along with cultivated on the agar with DNase I were characterized by a lack of swarming motility. These data clearly show that the treatment of cells with tested products results in different biological effects comparing with the addition of testing nucleases to the media.

### Example 4: Products and Methods for Managing Bacterial Dispersal and Chemotaxis

To study effects of a tested products/compounds on bacterial dispersal and chemotaxis, assay plates containing Columbia agar (supplemented with tested compounds), were prepared by adding 250 μL fresh human plasma to a sector comprising ⅙ of the plate. We used different compounds taken at various concentrations from 0.1 to 1000 μg/mL, some of them were used directly (table 3) and some were modified as described in the example 1 to avoid any penetration inside the cells (table 4). The plasma was filtered through a 0.22-μm pore-size filter (Millipore Corp., Bedford, MA, USA) immediately prior to use. Written informed consent was obtained from all patients to use their blood samples for research purposes, and the study was approved by the institutional review board of the Human Microbiology Institute (#VB-021420).

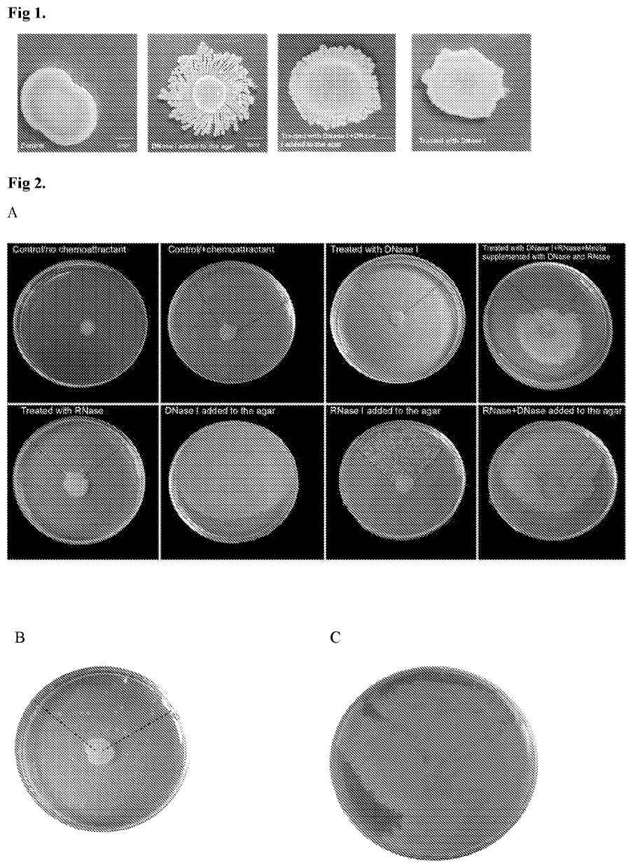

An aliquot containing 5.5 log 10 B. pumilus VT1200 in 25 μL was placed in the center of the plates, which were then incubated at 37° C. for 24 h and photographed with a Canon 6 digital camera. Swimming motility and chemotaxis was evaluated by measuring the migration of the central colony towards the plate sector containing plasma. Colony dispersal was assessed based on the appearance of small colonies on the agar surface. Data are presented in FIGS. 2a-b, 3, Tables 3 and 4.

We also controlled the internalization of RNase A in cells. For that B. pumilus (5.5 log 10 cells/ml) in PBS were incubated with fluorescein isothiocyanate (FITC) labeled RNase A at 37 C for 15 or 60 minutes. Bacteria were washed three times with PBS to remove any unbound protein. After washing the bacteria is cultivated for 2 h in LB broth, washed to remove residual media components, and placed on a microscope slide for visualization. Fluorescence was monitored using a fluorescence microscope (Axio Imager Z1, Carl Zeiss, Germany). To visualize the internalization of RNase A, the biofilms of B. pumilus incubated with 100 μg/mL fluorescein-labeled RNase A were obtained as described earlier. After 24 h of growth at 37 C, bacteria were washed three times with PBS to remove unbound proteins, and placed on a microscope to monitor the fluorescence using a fluorescence microscope (Axio Imager Z1, Carl Zeiss, Germany).

Control B. pumilus grew on the agar surface as round biofilms; however, addition of human plasma as a chemoattractant, triggered swimming motility and directional migration towards the plasma. Visual examination of biofilms revealed that use of compounds that inactivate or destroy cell-surface bound DNA results in the lost their chemotaxis and swimming ability. The use RNase for the one-time treatment of cells or the addition of RNase to the nutrient medium triggered swimming motility and biofilm dispersal towards the chemoattractant and was accompanied by the formation of multiple separate colonies in the agar zone where plasma was added (FIG. 2 a-b). Combined use of nucleases that were used to treat the cells and were added to the medium stimulated active sporulation in the center of colonies and negative chemotaxis.

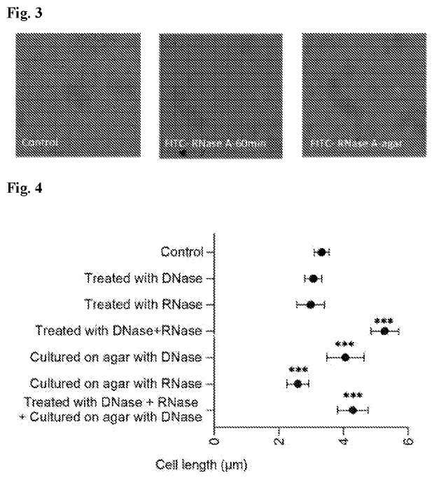

We also additionally tested could the compound 2,8-dichloro-5-(4-nitrophenyl)-5,9-dihydro-4H-pyrimido[5′,4′:5,6]pyrano[2,3-d]pyrimidine-4,6(1H)-dione added to the agar trigger cell migration towards chemoattractant (FIG. 2c). Under the used conditions, RNase A was not internalization by B. pumilus (FIG. 3).

TABLE 3
Effects of tested products on managing swimming motility, biofilm dispersal, and chemotaxis

| | Potentiation | | Potentiation | | Potentiation |
|---|---|---|---|---|---|
| | of swimming | | of swimming | | of swimming |
| | activity | | activity, | | activity, |
| | biofilm | | biofilm | | biofilm |
| | dispersal, and | | dispersal, and | | dispersal, and |
| Tested product | chemotaxis | Tested product | chemotaxis | Tested product | chemotaxis |
| TLR3 | Yes | Ribosomal | Yes | Ribosomal | Yes |
| | | protein bL34 | | protein L22 | |
| T7 RNA | Yes | Ribosomal | Yes | Ribosomal | Yes |
| polymerases | | protein L11 | | protein S19 | |
| Ribosomal | Yes | Ribosomal | Yes | Ribosomal | Yes |
| protein L11 238 | | protein eS31 | | protein L3 | |
| Ribosomal | Yes | Ribosomal | Yes | Ribosomal | Yes |
| protein eS1 | | protein eL43 | | protein L26 | |
| Ribosomal | Yes | Ribosomal | Yes | Ribosomal | Yes |
| protein L25-5S | | protein S14 | | protein S28 | |
| Ribosomal | Yes | Ribosomal | Yes | Ribosomal | Yes |
| protein S15a | | protein S21 | | protein uS7 | |
| Ribosomal | Yes | Ribosomal | Yes | Ribosomal | Yes |
| protein S18 | | protein S40 | | protein eS7 | |
| Ribosomal | Yes | Ribosomal | Yes | Ribosomal | Yes |
| protein S1 | | protein S60 | | protein bL12 | |
| RNase polymerase | Yes | Amikacin | Yes | Tobramycin | Yes |
| III | | | | | |
| Paromomycin | Yes | Pteridines | Yes | AC1MMYR2 | Yes |
| | | (tetrahydrobiopterin) | | | |
| Nuclear | Yes | Cold shock | Yes | RBD with a α1- | Yes |
| ribonucleoproteins | | protein | | L1-β1-L2-β2-L3- | |
| HER2 | | | | β3-LA-α topology | |
| ADENOSINE | Yes | Staufen is | Yes | Dicer- | Yes |
| DEAMINASES | | a protein | | like proteins | |
| ADAR1 | Yes | disco-interacting | Yes | ILF3 | Yes |
| | | protein | | | |
| RNA helicase | Yes | RNA recognition | Yes | K homology domain | Yes |
| | | motif | | RNA-binding | |
| | | | | protein | |
| RNA recognition | Yes | La motif | Yes | Argonaute protein | Yes |
| motif | | | | | |
| Piwi proteins | Yes | Pentatricopeptide | Yes | Pseudouridine | Yes |
| | | repeat protein | | synthase | |
| Pumillo-like | Yes | thiouridine | Yes | pseudouridine | Yes |
| repeat | | synthases | | synthases | |
| Ribosomal S1-like | Yes | RNA | Yes | linezolid | Yes |
| | | methylases and | | | |
| Sm RNA binding | Yes | YT521-B | Yes | ribocil | Yes |
| domain | | homology | | | |
| risdiplam | Yes | branaplam | Yes | riboflavin | Yes |
| artificial | Yes | Naphthalene- | Yes | miR-210 | Yes |
| cationic oligosacc | | based diimide | | | |
| haride (β-(1→4)- | | conjugated bis- | | | |
| Linked-2,6- | | aminoglycoside | | | |
| diamino-2,6- | | | | | |
| dideoxy-d- | | | | | |
| galactopyranose | | | | | |
| oligomers) | | | | | |
| short | Yes | Ribocil-D | Yes | CCCH zinc finger | Yes |
| hairpin RNA | | | | protein | |
| Pyrithiamine | Yes | 1-aminoethylcysteine | Yes | Netilmicin | Yes |
| Neomycin | Yes | 2-aminobenzimidazole | Yes | pentamidine | Yes |
| | | derivatives | | | |
| groove-binding | Yes | Myricetin | Yes | Branaplam | Yes |
| ligands | | | | | |
| streptavidin- | Yes | bis-benzimidazole | Yes | miRNA | Yes |
| binding | | | | | |
| RNA aptamer | | | | | |
| hnRNP C | Yes | small nuclear RNPs | Yes | vault cytoplasmic | Yes |
| | | | | ribonucleoprotein | |
| Nucleophosmin | Yes | locked nucleic acids | Yes | RNA-recognition | Yes |
| | | | | motif, RNP1 | |
| CCL2 | Yes | RNaseIf | Yes | RNase III | Yes |
| RNase A | Yes | RNase T1 | Yes | Antibodies | Yes |
| | | | | against cell | |
| | | | | surface associated | |
| | | | | RNA, RNA | |
| | | | | NAMACS and | |
| | | | | NAMACS-ANA | |
| RNaseIf | Yes | REC J nuclease | Yes | RNase U2 | Yes |
| RNase H1 | Yes | RNase PH | Yes | RNase V1 | Yes |
| RNase I | Yes | RNase II | Yes | Polynucleotide | Yes |
| | | | | phosphorylase | |
| exoribonuclease | Yes | oligoribonuclease | Yes | RNase P | Yes |

The results clearly show that the tested compounds manage swimming motility and chemotaxis. Moreover, different products that either inactivate or inhibit RNA molecules in these settings can contribute to identical biological effects.

TABLE 4
Effects of modified products on managing swimming
motility, biofilm dispersal, and chemotaxis

| | Potentiation | | Potentiation | | Potentiation |
|---|---|---|---|---|---|
| | of swimming | | of swimming | | of swimming |
| | activity, | | activity, | | activity, |
| | biofilm | | biofilm | | biofilm |
| Tested | dispersal, and | Tested | dispersal, and | Tested | dispersal, and |
| products | chemotaxis | product | chemotaxis | product | chemotaxis |
| RNase | Yes | Modified | Yes | Modified | Yes |
| | | Ribosomal | | RNase | |
| | | protein S1 | | polymerase | |
| | | | | III | |
| Modified | Yes | Modified RNA | Yes | Modified | Yes |
| Tobramycin | | helicase | | RNA | |
| | | | | recognition | |
| | | | | motif | |
| Modified | Yes | Modified ribocil | Yes | Modified | Yes |
| linezolid | | | | pentamidine | |
| Modified | Yes | Modified | Yes | Modified | Yes |
| RNA | | Argonaute protein | | T7 RNA | |
| helicase | | | | polymerases | |

The results summarized in Tables 3-4 clearly show that the tested modified compounds manage swimming motility and chemotaxis.

### Example 5: Products and Methods for Managing Cell Morphology

We treated B. pumilus 1200 with nucleases as described previously or cultivated on TGV agar with added nucleases and analyzed cell size 24 h after (FIG. 4) (Tetz et al., 2018). Cells treated with DNase and RNase resulted in increased cell sizes (p<0.001) and the same trend for the increased cell sizes was noticed for cells cultured on media with DNase or all treated with DNase+RNase and cultured on media with DNase+RNase, while cultured on media with RNase resulted in significant decrease of cell size (P<0.05). Our findings point out that cell morphology can also be modified and regulated with compounds tested.

### Example 6: Products and Method for Managing Cell Characteristics

We studied the effect of tested products on various cell lines characteristics growth and/development activity. Cells were separated from the extracellular matrix and left either untreated or treated with tested compounds. We studied the alteration of the monolayer formation in the wells of 96-well plate with the appropriate nutrient media and supplementary additives. Cell monolayers were analyzed at 6-12-24 and 48 hours, and the difference in the character of growth or cell behavior was monitored.

We analyzed the following parameters (1) size of the cell (2) cell morphology (3) presence of multinucleated cells (4) speed of monolayer formation. Control cultures had 1 point for each of these parameters, thus written as “++++”. Any alterations in any of these parameters excluded “+” Data are presented in table 5.

TABLE 5
Effect of tested products on cell characteristics

| | | | | | | | cultivated |
|---|---|---|---|---|---|---|---|
| | | | | Treated | cultivated | cultivated | in the |
| | | Treated | Treated | with | in the | in the | presence |
| | | with | with | DNase + | presence | presence | of DNase + |
| Cell type | Control | DNase | RNase | RNase | of DNase | of RNase | RNase |
| stem cells | ++++ | +++ | +++ | ++ | ++++ | +++ | ++ |
| leucocytes | ++++ | ++++ | +++ | +++ | ++ | + | +++ |
| lymphocytes | ++++ | +++ | ++ | ++ | ++ | ++ | ++ |
| neutrophils | ++++ | +++ | +++ | + | ++ | + | +++ |
| eosinophils | ++++ | +++ | +++ | +++ | +++ | +++ | +++ |
| macrophages | ++++ | +++ | +++ | ++ | ++++ | +++ | + |
| cortical | ++++ | ++ | ++ | ++ | +++ | ++++ | +++ |
| neuron | | | | | | | |
| astrocytes | ++++ | ++ | ++++ | +++ | +++ | +++ | +++ |
| microglial | ++++ | +++ | +++ | +++ | ++ | +++ | ++ |
| cells | | | | | | | |
| epithelial cells | ++++ | +++ | +++ | +++ | +++ | ++ | +++ |
| fibroblasts | ++++ | +++ | +++ | ++ | +++ | +++ | ++ |
| muscle cells | ++++ | + | +++ | + | +++ | ++++ | +++ |
| chondrocytes | ++++ | ++ | ++++ | +++ | +++ | +++ | +++ |
| osteoblast | ++++ | ++ | +++ | | + | ++++ | |
| endothelial | ++++ | +++ | +++ | ++ | +++ | +++ | +++ |
| cells | | | | | | | |
| adipose tissue | ++++ | + | +++ | ++ | ++ | +++ | + |
| retinal | ++++ | ++ | +++ | ++ | ++ | ++ | +++ |
| pigment | | | | | | | |
| epithelial cells | | | | | | | |
| kidney cells | ++++ | ++ | +++ | +++ | +++ | ++ | ++ |
| placenta cells | ++++ | +++ | +++ | +++ | ++++ | +++ | ++ |
| spermatozoids | ++++ | + | +++ | ++ | +++ | ++++ | +++ |
| tumor cells | ++++ | + | +++ | + | +++ | +++ | |
| (Patient- | | | | | | | |
| derived | | | | | | | |
| xenografts) | | | | | | | |
| cancer | ++++ | +++ | ++ | + | ++ | +++ | +++ |
| associated | | | | | | | |
| fibroblasts | | | | | | | |
| neuroendocrine | ++++ | +++ | +++ | +++ | +++ | +++ | ++ |
| cells | | | | | | | |
| (intestinal | | | | | | | |
| neuroendocrine | | | | | | | |
| tumor cells) | | | | | | | |
| pancreatic | ++++ | +++ | +++ | ++++ | +++ | +++ | +++ |
| cells | | | | | | | |

These data clearly shows that tested products can be used for managing cell characteristics and growth.

### Example 7: Products and Method for Managing Proteins Associated with Neurodegenerative and Autoimmune Diseases Development

Products for managing proteins associated with neurodegenerative and autoimmune disease formation where tested. The inventors examined whether prion-misfolding and aggregated fibril formation could be inhibited by tested products taken at 10 μg/mL.

For these studies as an examples of prion-like proteins full-length Tau, beta-amyloid, α-synuclein, SOD1, TDP-43, IAPP (proteins associated with Alzheimer's disease, Parkinson's disease, amyotrophic lateral sclerosis, diabetes) were used to prepare aggregated tau for seeding experiments. For that they were used at monomeric aggregate-free condition with a concentration of 10-100 μM, containing/not containing 25 μM heparin in a buffer and were incubated for different time periods at 37° C. The protein aggregation was followed by protein misfolding cyclic amplification (PMCA) method by monitoring the levels of Thioflavin T (ThT) fluorescence overtime from samples taken from replicate tubes and subjected to cyclic agitation. At various time points, ThT fluorescence was measured in the plates using a plate spectrofluorometer.

We used leucocytes and Escherichia coli VT27 cells either untreated or treated with tested compounds. Data are shown in tables 6, 7, 8.

TABLE 6
The products used to eliminate certain
types of nucleic acids on cell surface

| Product | Target on the cell surface |
|---|---|
| Exonuclease VII | ssDNA |
| Exonuclease III + Exonuclease VII | dsDNA |
| RNaseIf | ssRNA |
| RNase H1 | ssRNA |
| RNase H1 + Exonuclease VII | ssRNA and DNA |
| SMAD4 | dsDNA, ssDNA, dsRNA, ssRNA |

TABLE 7
Products used to detect the prion protein misfolding

| Number | Probe |
|---|---|
| 1 | Control |
| 2 | Prion protein + Prion seeds |
| 3 | E. coli treated with Exonuclease VII |
| 4 | E. coli treated with Exonuclease III + Exonuclease VII |
| 5 | E. coli treated with Notl |
| 6 | E. coli treated with RNase E |
| 7 | E. coli treated with RNase H1 |
| 8 | E. coli treated with DNase I |
| 9 | E. coli treated with RNaseIf |
| 10 | E. coli treated with RNase H1 + Exonuclease VII |
| 11 | E. coli treated with SMAD4 |
| 12 | Lymphocytes treated with Exonuclease VII |
| 13 | Lymphocytes treated with Exonuclease III + Exonuclease VII |
| 14 | Lymphocytes treated with EcoNI |
| 15 | Lymphocytes treated withRNase A |
| 16 | Lymphocytes treated with RNase H1 |
| 17 | Lymphocytes treated with DNase I |
| 18 | Lymphocytes treated with DNase 1L2 |
| 19 | Lymphocytes treated with RNase H1 + Exonuclease VII |

The results for the acceleration of protein misfolding vs untreated controls and positive controls having all cell surface DNA and/or RNA molecules are listed in the table 8.

TABLE 8
Effect of products usage in acceleration of protein misfolding.

| | % Faster than untreated control to reach the lag phase | | | | | |
|---|---|---|---|---|---|---|
| | | Beta- | Alpha- | | | |
| Probe | Tau | amyloid | synuclein | SOD1 | TDP43 | IAPP |
| 1 | NA | NA | NA | NA | NA | NA |
| 2 | 272 | 218 | 224 | 259 | 186 | 151 |
| 3 | 12* | 24* | 17* | 16* | 26* | 15* |
| 4 | 173* | 204 | 146* | 150* | 148 | 221 |
| 5 | 32* | 45* | 23* | 29* | 36* | 19* |
| 6 | 44* | 56* | 75* | 72* | 55* | 42* |
| 7 | 25* | 42* | 26* | 12* | 19* | 13* |
| 8 | 98* | 99* | 84* | 101* | 123* | 83* |
| 9 | 101* | 122* | 109* | 148* | 122* | 107* |
| 10 | 74* | 119* | 73* | 90* | 84* | 115* |
| 11 | 9* | 38 | 22 | 11 | 7 | 44 |
| 12 | 25* | 19* | 11* | 14* | 24* | 18* |
| 13 | 168 | 152* | 144 | 76* | 36* | 96* |
| 14 | 22* | 29* | 11* | 10* | 24* | 34* |
| 15 | 28* | 40* | 23* | 18* | 34* | 39* |
| 16 | 21* | 18* | 12* | 27* | 36* | 8* |
| 17 | 53* | 64* | 78* | 54* | 27* | 62* |
| 18 | 54* | 67* | 83* | 69* | 21* | 75* |
| 19 | 40* | 41* | 55* | 47* | 17* | 30* |

*p < 0.05

We unexpectedly found that the tested products significantly inhibit protein misfolding. The destruction of cell-surface bound DNA and RNA led to a significantly inhibition of protein misfolding.

### Example 8: Products and Methods for Managing Microbial Growth

S. aureus and E. coli were treated with different compounds as described earlier, after what compounds were washed away and bacteria were plated to LB broth. Growth curves are presented as OD600 values and as bacterial counts as a function of time in FIG. 5a,b respectively. Treatment of cells with tested products resulted in an altered bacterial growth of both Gram-positive and Gram-negative bacteria. Surprisingly, these data point out that different products can affect both synthetic activity (decreased OD600) as well as CFU number.

### Example 9: Products and Method for Managing of Microbial Growth Acceleration

Bacillus VT1200 were cultivated on the TGV agar supplemented with DNase I (1 g/ml), histone 5 (100 μg/ml), or TATA box-binding family (5 μg/ml). Control bacteria were cultivated on a regular agar.

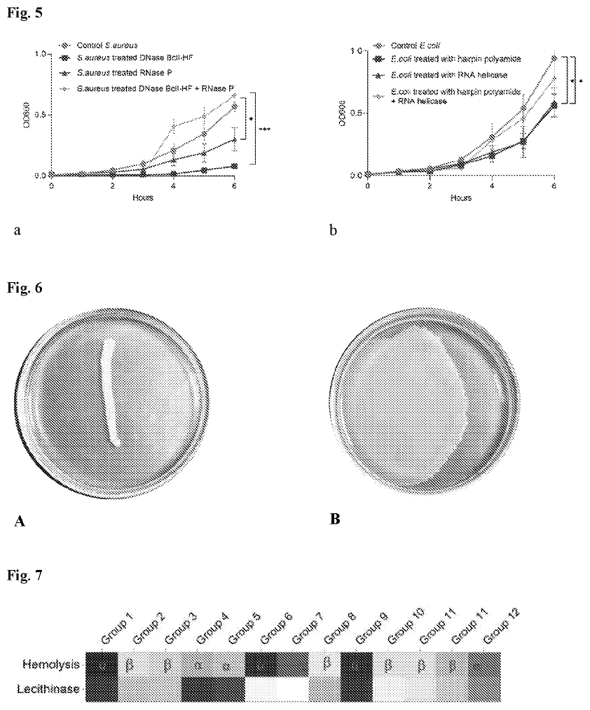

100 μL of broncho alveolar lavage (BAL) from the patient with pneumonia was dissolved in 200 μL of sterile water, separated on 2 parts one of which was treated with DNase I (Sigma, 2000 Kunitz units/mL) up to 100 μg/mL from 1.0 min up to 120 minutes, while the second part was supplemented with the equal amount of buffer. After that bacteria and BAL were washed from tested products with PBS with the following centrifugation 5 minutes 4000×g (Microfuge® 20R, Beckman Coulter), and resuspended in PBS (for bacteria the final concentration was 6 log 10 cell/mL). 20 μL of bacterial or BAL suspensions were added to the center of 24 well plate with LB agar. Plates were incubated 370C and the presence of bacterial growth were monitored hourly. Data are presented in table 9 and FIG. 6.

TABLE 9
The effect of tested products on acceleration of bacterial growth.

| | Size of bacterial growth zone to control at 24 h (%) | | | |
|---|---|---|---|---|
| | | | | BAL grown on |
| | | Bacillus grown | | the media |
| | | on the media | | supplemented |
| | Control | supplemented | Control | with TATA box- |
| Hours | Bacillus | with histone 5 | BAL | binding protein |
| 1 | 0 | 0 | 0 | 0 |
| 2 | 0 | 20 | 0 | 30 |
| 3 | 0 | 50 | 10 | 80 |
| 4 | 10 | 90 | 10 | 110 |
| 24 | 100 | 460 | 100 | 380 |

As it seen that Products used led to a significant acceleration of microbial growth. Different products may be used for the acceleration of microbial growth and early detection of bacterial growth that is important for the diagnosis, antibiotic selection, antimicrobial susceptibility testing, biomanufacturing.

### Example 10: Products and Method for Managing Microbial Virulence

To obtain oligonucleotides, the mix of gram-positive and gram negative bacteria were lysed and DNA was isolated according to the standard method or standard eukaryotic DNA was used (Salmon Sperm DNA, Thermofisher). 5 μl of 1 M CaCl2) and 1 M MgCl2 solutions were added to the resulting 10 mg DNA in 10 ml sterile water. 2.5 mg of DNase were added to the reaction mass and left overnight at room temperature (8-12 hours) or at 37° C. for 5 hours. To inactivate DNase at the end of the DNA depolymerization reaction, the reaction mass is placed for 5-10 minutes in a boiling water bath until the liquid in the test tube boils. After the enzyme inactivation, the reaction mass is poured into Millipore centrifuge concentrators with a 10 kDa membrane and centrifuged at 3000 rpm for the time necessary to completely separate the low molecular weight and concentrate the high molecular weight fractions. The low molecular weight fraction was collected and its optical density was measured against water at λ=260 nm.

S. aureus SA58-1 group 1 were left untreated (control), treated with DNase 1L3 1 pg/mL (group 2) or Histone H5 1 μg/mL (group 3), pseudouridine synthase (0.1 μg/mL) (group 4), RNase II (1 pg/mL) (group 5), group 6 treated with DNase 1L3+RNase II, group 7 treated with Histone H5+Pseudouridine synthase, group 8 DNase 1L3 added to the agar, group 9 RNase II added to the agar, group 10 DNase 1L3 and RNase II added to the agar, group 11 treated with DNase 1L3 and RNase II and additionally DNase 1L3 and RNase II added to the agar, group 12 cells treated with oligonucleotides obtained from bacterial DNA, group 13 were treated with oligonucleotides obtained from eucaryotic DNA.

The hemolytic test was performed as previously described with minor modifications (Manukumar et al., 2017). Briefly, 15 μl of 5×10e5 bacterial cells were plated in the center of Columbia agar plates supplemented with 5% sheep red blood cells and incubated at 37° C. for 24 h. A greenish zone around the colony denoted α-hemolysin activity; whereas β-hemolysin (positive) and γ-hemolysin (negative) activities were indicated by the presence or absence of a clear zone around the colonies. The size of the hemolysis zone (in mm) was measured (FIG. 7).

Lecithinase activity was determined by plating cells on egg-yolk agar and incubation at 37° C. for 48 h. The presence of the precipitation zone and its diameter were evaluated (Bennett et al., 2003).

S. aureus SA58-1 were obtained as previously described and were grown on the agar additionally supplemented with reverse transcription inhibitors, acyclovir, ribavirin, potassium orotate, lithium orotate, taken at concentrations from 0.1 to 1000 μg/mL on the Columbia agar supplemented with 5% erythrocytes.

Hemolytic activity of control cells or treated with tested products and grown on the media supplemented without reverse transcription inhibitors, acyclovir, ribavirin, potassium was used as an individual control, taken at 100% (table 10)

TABLE 10
Effect of products on microbial toxicity.

| | Hemolysis | | | | | | |
|---|---|---|---|---|---|---|---|
| | | | | | | | DNase + |
| | | | | DNase | RNase | Treated | RNase |
| | | Treated | Treated | added to | added to | with | added to |
| | | with | with | the | the | DNase + | the |
| | Control | DNase | RNase | medium | medium | RNase | medium |
| Control | 100% | 100% | 100% | 100% | 100% | 100% | 100% |
| Etraverin | 40% | 0% | 90% | 0% | 100% | 100% | 10% |
| Nevirapine | 50% | 0% | 70% | 0% | 90% | 80% | 10% |
| Lamivudine | 100% | 20% | 50% | 60% | 130% | 30% | 30% |
| Azidothymidine | 90% | 100% | 70% | 70% | 90% | 20% | 30% |
| Aciclovir | 90% | 90% | 10% | 90% | 150% | 90% | 90% |
| Ribavirin | 80% | 20% | 100% | 30% | 90% | 20% | 10% |
| Potassium | 80% | 20% | 80% | 30% | 90% | 20% | 10% |
| orotate | | | | | | | |
| Lithium orotate | 70% | 40% | 30% | 30% | 60% | 20% | 0% |

This result suggests that tested products can be used to regulate bacterial virulence.

### Example 11. Products and Method for Managing Cell Differentiation

We tested the effects of different products on cell differentiation and persisters formation. Stationary-phase cultures E. coli were separated from the extracellular matrix and left either untreated (control) or following pretreatment for 15 minutes with tested products. Probes were normalized by the CFU, diluted in LB broth supplemented with ampicillin (150 μg/ml) and incubated for 6 h. Samples were taken before the addition of ampicillin and after 6 h of ampicillin treatment by plating on LB agar without antibiotics to determine the number of colony forming units. The frequency of persisters was calculated as the ratio of the number of persisters in a sample to the initial number of total cells before antibiotic treatment in each probe (Table 11).

TABLE 11
Frequency of persister cells formation following the treatment with tested compounds.

| | | | | T4 | | Ribosomal | | |
|---|---|---|---|---|---|---|---|---|
| | DNase | DNase | Granzyme | Polynucleotide | | S1-like | | |
| | I | I | B | Kinase | RNase A | 1000 | Ribocil-D | DNase + |
| Control | 1 μg/mL | 1 pg/mL | 1 μg/mL | 100 μg/mL | 5 μg/mL | μg/mL | 100 μg/mL | RNase |
| 0.0007 | 0.011* | 0.013* | 0.02* | 0.016* | 0.0102* | 0.0095* | 0.0088* | 0.00049* |

*p < 0.05

As expected, in the control E. coli 1/1304 of original cells being ampicillin tolerant. However, the number of persisters was significantly increased following the use of tested products. Thus, tested products can be used to modulate persister formation and can be used for healing, prevention the spread of infections, and industry.

### Example 12. Products and Method for Managing of Mutagenesis

Next, we examined how different tested products could manage the rate of spontaneous mutagenesis. In these experiments, we measured spontaneous mutation frequency to rifampicin in E. coli ATCC 25922 by counting viable RifR mutants after cultivation on rifampicin-supplemented agar plates (Table 12). Spontaneous mutagenesis was inhibited by the products that inactivate surface-bound DNA molecules (DNase I, Cas9), meaning that they blocked the occurrence of replication errors. Surprisingly, the use of products that affected both surface-bound DNA- and RNA-molecules (DNase I+RNase; Cas9+ILF3) triggered spontaneous mutagenesis and led to significantly higher number of RifR mutants.

TABLE 12
Effects of tested products on mutagenesis.

| | RifR mutants per 9 | |
|---|---|---|
| | log10 E. coli cells | |
| Probe | (mean ± SD)a | P value |
| Control E. coli | 27 ± 6 | |
| E. coli treated with DNase I | 0 ± 0 | 0.015 |
| E. coli treated with Cas9 | 0 ± 0 | 0.015 |
| E. coli treated with RNase | 34 ± 8 | 0.297 |
| E. coli treated with ILF3 | 24 ± 5 | 0.543 |
| E. coli treated with DNase I and RNase | 1050 ± 258 | 0.021 |
| E. coli treated with DNase I + RNase + | 0 ± 0 | 0.015 |
| antibodies against DNase and RNase | | |
| E. coli treated with Cas9 and ILF3 | 867 ± 139 | 0.009 |

Values represent the mean from at least three independent experiments.

Data received clearly show that products used can manage mutagenesis.

### Example 13: Product and Method for Managing of DNA Recombination

To determine the role of studied products in bacterial recombination, we incubated control E. coli LE392 with λ phage (bearing Ampr and Kanr genes) for a time sufficient to cause phage adsorption and DNA injection. This was followed by treatment of the cells with nucleases (10 μg/mL), or propidium iodine (1 μg/mL) or the combination between modified short hairpin RNA (250 μg/mL) and modified T6 gene exonuclease (0.1 μg/mL).

Control E. coli LE392 were incubated with λ phage, but were not treated with nucleases. Treatment of cells with any tested compounds increased recombination frequency, as indicated by the increased rate at which phages lysogenized sensitive bacteria and, consequently, the higher number of antibiotic-resistant mutants (FIG. 8). The highest increase was observed in bacteria treated with compounds that inactivate both DNA and RNA. Taken together, these findings show that the tested compounds can be used to control regulate recombination frequency.

We also studied the effects of potassium orotate, Ribavirin, Acyclovir, Azidothymidine, Lamividine, Tenofovir, Nevirapine, Etravirine (all added to 50 μg/mL) and grown at 37 C for 24 h. Data are shown in table 13.

TABLE 13
Induction of prophages and inhibition of bacterial growth.

| | Type of microbial growth | | | | |
|---|---|---|---|---|---|
| | | | | Treated | Treated |
| | | Treated | Treated | with | with |
| | | with | with | DNase + | propidium |
| Group | Control | DNase | RNase | RNase | iodine |
| Control | Lawn | Lawn | Single | Lawn | Lawn |
| | | | colonies, | | |
| | | | lysis | | |
| Potassium | Single | Single | Lawn | Lawn | Lawn |
| orotate | colonies, | colonies, | | | |
| | lysis | lysis | | | |
| Ribavirin | Lawn | Lawn | Growth | Single | Single |
| | | | inhibition | colonies, | colonies, |
| | | | | lysis | lysis |
| Acyclovir | Single | Lawn | Lawn | Single | Single |
| | colonies, | | | colonies, | colonies, |
| | lysis | | | lysis | lysis |
| Azidothy- | Growth | Growth | No | Growth | Growth |
| midine | inhibition, | inhibition, | growth | inhibition, | inhibition, |
| | single | single | | single | single |
| | colonies | colonies | | colonies | colonies |
| Lamividine | Lawn | Single | Lawn | Lawn | Lawn |
| | | colonies, | | | |
| | | lysis | | | |
| Tenofovir | Growth | Lawn | Single | Single | Single |
| | inhibition | | colonies, | colonies, | colonies, |
| | | | lysis | lysis | lysis |
| Nevirapine | Lawn | Growth | Growth | Lawn | Lawn |
| | | inhibition | inhibition | | |
| Etravirine | Lawn | Single | Lawn | Lawn | Lawn |
| | | colonies, | | | |
| | | lysis | | | |

Results indicate on the possibility to manage DNA recombination with tested compounds.

### Example 14: Product and Method for Managing Host-Viral Interactions

Products for managing host-viral interactions where tested on the overnight cultures of Staphylococcus aureus ATCC 29213, Pseudomonas aeruginosa VT-16-20B. Bacteriophages used: Staphylococcal phage VTSA-29213, Pseudomonas aeruginosa VTPA-20B phage. Bacteria were separated from the extracellular matrix and were pretreated with nucleases (10 μg/mL) for 15 minutes as previously shown and o with Histone H2B (1000 μg/ml) and Ribosomal protein L22 and Cold shock protein A (100 μg/ml) action were plated with phages by agar layer method on the media and the number of negative colonies was determined after 48 h of incubation at 27 C. Results are presented in Table 14.

TABLE 14
Effect of tested products on cell-virus interaction

Phage titer

| | Pseudomonas | S. aureus |
|---|---|---|
| Products | aeruginosa VT-16-20B | ATCC 29213 |
| Control | 9 × 10e9 | 2 × 10e10 |
| DNase I | 2 × 10e11 | 3 × 10e11 |
| RNase | 1 × 10e11 | 1 × 10e11 |
| DNAse + RNase | 7 × 10e7 | 5 × 10e8 |
| Cold shock protein A | 8 × 10e7 | 9 × 10e8 |
| Histone H2B + | 6 × 10e8 | 3 × 10e6 |
| Ribosomal protein L22 | | |

The data obtained indicate that products acting to control the interaction of viruses with cells, including increasing viral output. Moreover, it is possible to regulate different steps of pathogen-host interaction including virus-host integration, blocking cell recognition by virus, viral reproduction.

### Example 15. Products and Method for Managing Cells Temperature Sensitivity

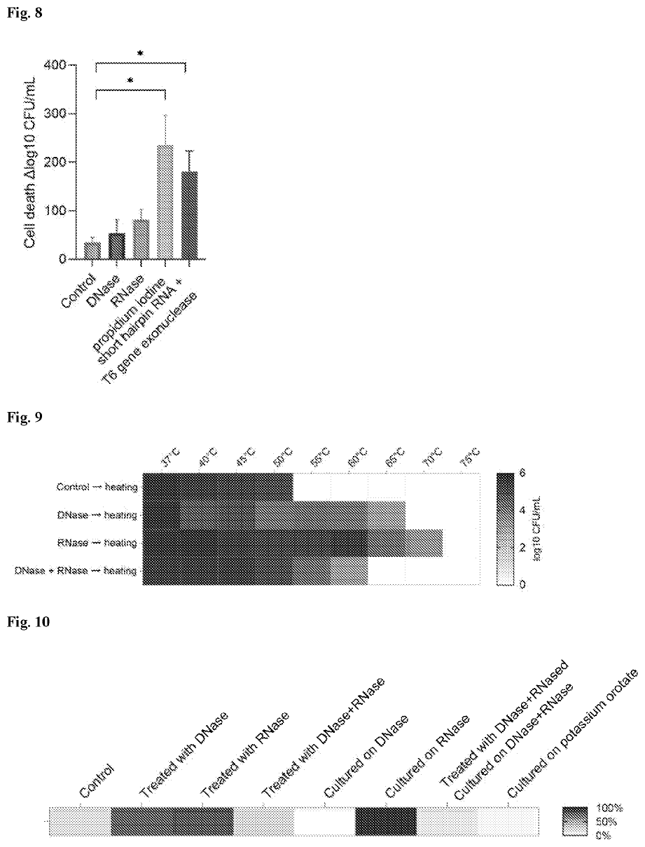

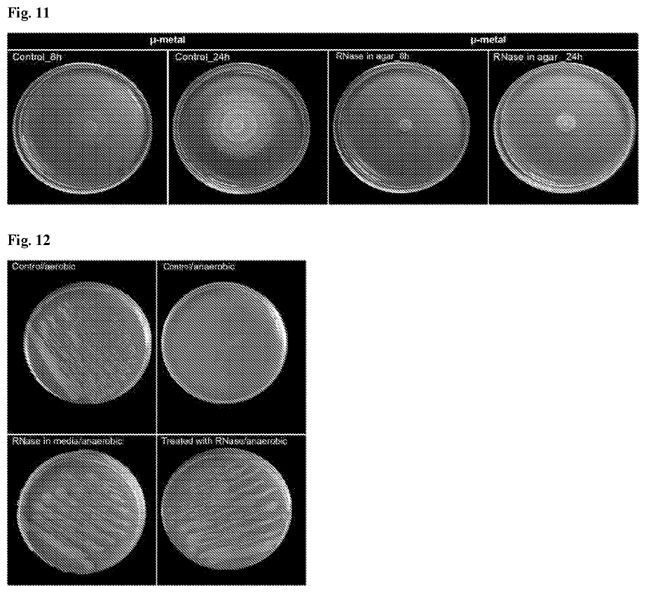

Assessment of whether tested products could modulate bacterial thermotolerance revealed that control S. aureus VT209 exhibited maximum tolerance at up to 50° C., whereas S. aureus following the use of studied products could survive at higher temperatures (FIG. 9, table 15). Overnight S. aureus VT209 cultured in LB broth was separated from the extracellular matrix by washing in PBS and then diluted with PBS to OD600 of 0.5. Bacteria were separated from the extracellular matrix and were left untreated or treated with nucleases (10 μg/mL), or treated with proteins listed in table 14 for 5 minutes and 5.5 log 10 CFU/mL were placed in 2-mL microcentrifuge tubes (Axygen Scientific Inc., Union City, CA, USA). Each tube was heated to 37, 40, 45, 50, 55, 60, 65, 70 or 75° C. in a dry bath (LSE™ Digital Dry Bath; Corning, Corning, NY, USA) for 15 min. After heating, control S. aureus were immediately treated with nucleases to delete primary TezRs, washed three times to remove nucleases, serially diluted, plated on LB agar, and the number of CFU was determined within 24 h.

TABLE 15
Effect of tested compounds on maximum tolerance of S. aureus

| | Concentration | Maximum | |
|---|---|---|---|
| Tested compound | μg/mL | tolerance (C.) | P value |
| Control | — | 50 | |
| DNase I | 0.001 | 60 | <0.05 |
| RNase A | 0.001 | 65 | <0.05 |
| HIV-1 reverse transcriptase | 10 | 65 | <0.05 |
| Trp repressor family: 1trr | 1 | 60 | <0.05 |
| M-MLV reverse | 1000 | 60 | <0.05 |
| transcriptase | | | |
| Ribosomal protein L11 | 100 | 70 | <0.05 |
| Ribosomal protein L3 | 100 | 75 | <0.05 |
| ADAR1 | 10 | 65 | <0.05 |
| Artificial | 10 | 65 | <0.05 |
| cationic oligosaccharide (β- | | | |
| (1→4)-Linked-2,6- | | | |
| diamino-2,6-dideoxy-d- | | | |
| galactopyranose oligomers) | | | |
| Vault cytoplasmic | 1 | 70 | <0.05 |
| ribonucleoprotein | | | |

Data received clearly show that tested products can be used for the regulation of the responses to temperatures, thermosensitivity and heat resistance.

### Example 16: Products and Method for Managing Sporulation and Treatment of Diseases Associated with Spore Forming Bacteria

We first checked whether tested products regulate sporulation using B. pumilus VT1200. For the analysis of sporulation B. pumilus were separated from the extracellular matrix and left either untreated (control) or incubated for 60 minutes with tested products (10 μg/mL). 5.5 log 10 and 100 μl bacterial culture were plated to the Columbia agar media as a loan and the number of spores was assessed in 24 hours under the microscope by counting cells and spores in 20 microscope fields and three replicates. For each image, we calculated the number of spores and the number of cells. Then, we plotted the ratio of spores to the combined number of cells and spores in each bin (FIG. 10). Data received indicated that tested products can be used to control sporulation and treatment disease associated with the spore forming bacteria

### Example 17: Products and Method for Managing Sensitivity of Cells to Environmental Factors

Products for managing cell sensitivity to pH were studied using a model of E. coli VT-267 cultivated at different levels of pH. For that E. coli VT-267 were separated from the extracellular matrix and were pretreated with tested compounds for 30 minutes and plated to LB broth (Oxoid) with pH value adjusted from 3 to 9 (Table 16).

TABLE 16
Effect of different products in cells sensitivity to pH

| | Growth/no growth of E. coli | | | | | | |
|---|---|---|---|---|---|---|---|
| Product/effect | pH 9 | pH 8 | pH 7 | pH 6 | pH 5 | pH 4 | pH 3 |
| Control | No | Growth | Growth | Growth | Growth | Growth | No |
| DNase 10 μg/mL | Growth | Growth | Growth | Growth | Growth | Growth | Growth |
| RNase 1 μg/mL | Growth | Growth | Growth | Growth | Growth | Growth | Growth |
| Transcription factor | Growth | Growth | Growth | Growth | Growth | Growth | Growth |
| 10 μg/mL IIB | | | | | | | |
| ZNF3 1000 μg/mL | Growth | Growth | Growth | Growth | Growth | Growth | Growth |
| ZNF239 1 μg/mL | Growth | Growth | Growth | Growth | Growth | Growth | Growth |

Data received clearly show that tested products can be used for managing of the responses to environmental conditions.

### Example 18: Products and Method for Managing Magnetosensitivity

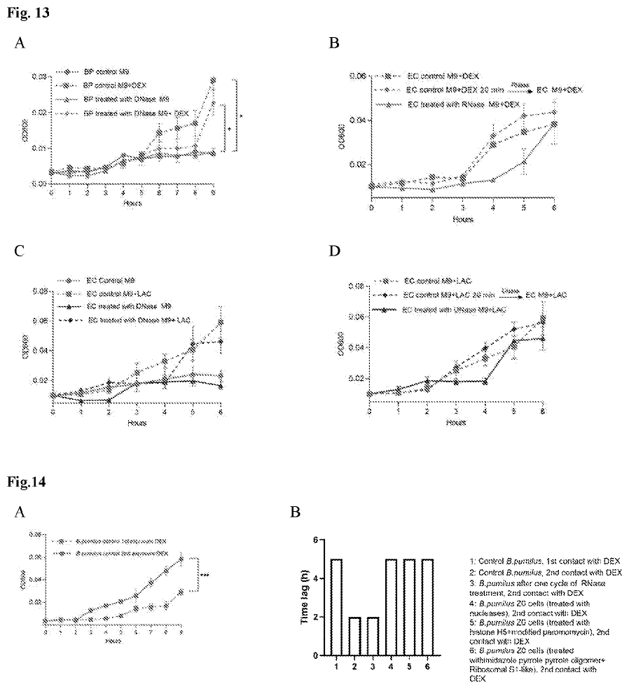

The effect of the tested compounds on magnetosensitivity was done using a model of B. pumilus VT1200 growth when exposed to regular magnetic and shielded geomagnetic fields. B. pumilus treated with tested products were obtained as previously discussed. Final inoculum of 5.5 log 10 CFU/mL in 25 μL were dropped in the center of agar-filled Petri dishes. Magnetic exposure conditions were modulated by placing the Petri dish in a custom-made box made of from two to five layers of 10-μm-thick μ metal (to shield geomagnetic field) at 37° C. for 24 h (Table 17). In a second experimental, control B. pumilus were separated from the extracellular matrix and treated with RNase and were exposed to regular magnetic conditions or a shielded geomagnetic field as described above in, and colony morphology was analyzed after 8 and 24 h. Images of the plates were acquired using a Canon 6 digital camera (FIG. 12).

TABLE 17
Effects of tested products on magnetosensitivity

| | | | Inhibition of |
|---|---|---|---|
| | Concen- | | magnetosen- |
| Product | tration | | sitivity |
| Recombinant Human RNA | 10 | μg/mL | Yes |
| binding protein fox-1 homolog 2 | | | |
| RNase III | 100 | μg/mL | Yes |
| RNase III | 10 | μg/mL | Yes |
| Antibodies against RNA NAMACS | 1 | μg/mL | Yes |
| Cold Inducible RNA Binding | 100 | μg/mL | Yes |

Protein Recombinant Protein-

Data received clearly show that tested products can be used for managing of magnetosensitivity.

### Example 19: Products and Method for Managing Growth in Different Gas Compositions

We analyzed could the tested products modulate response of cells to a changing gas composition. P. putida were separated from the extracellular matrix and were left either untreated (control) or treated with tested compounds for 15 minutes were placed on agar and cultivated under anoxic conditions. While control P. putida could not grow under anaerobic conditions, treatment with RNase and other tested compounds allowed for anaerobic growth of P. putida (FIG. 12, table 18). Collectively, the findings point to that the tested compounds can be used for the adaptation to variations in gas composition.

TABLE 18
Effects of tested products on cell
growth in different gas environment

| | Growth of P. putida | |
|---|---|---|
| Probe | Aerobic | Anaerobic |
| Control | + | + |
| RNase T1 | + | + |
| Nucleophosmin | + | + |
| riboflavin | + | + |
| Argonaute protein | + | + |
| Antibodies against cell-surface-bound RNA | + | + |
| Ribosomal protein | + | + |

There results also show that products used can manage cell responses to gas composition.

### Example 20: Products and Methods for Managing Chemosensing and Utilization of Nutrients and Xenobiotics

To investigate the role of tested compounds in xenobiotics utilization, B. pumilus and E. coli were separated from the extracellular matrix and were left either untreated (control) or pretreated with tested compounds and inoculated in M9 minimal medium supplemented with the xenobiotic dexamethasone (100 μg/mL) or lactose (100 μg/mL) as the sole source of carbon and energy. We compared the effects of the tested compounds on the lag phase, which comprises the time required for sensing and starting the utilization of these nutrients.

The time lag following the treatment with tested compounds (FIG. 13a) was delayed by 3 and 2 h compared with that of control bacteria (p<0.05), indicating a delay in the uptake and consumption of dexamethasone (Table 18).

We hypothesized that the prolonged time required by bacteria after the treatment with the tested products to start using dexamethasone resulted from disruption of sensing and alteration of control nutrient consumption, rather than an alteration of transcriptional activity. To verify this hypothesis, we conducted an experiment when E. coli pretreated with dexamethasone followed by treatment with tested products and cultivation in M9 supplemented with dexamethasone would have the same time lag as control E. coli in the same M9 medium. In other words, the presence of cell-surface bound nucleic acids is a prerequisite for cell to sense and utilize nutrients and once control cells sensed dexamethasone, they would continue utilizing to it even if they were subsequently treated with tested products.

In agreement with this hypothesis, control E. coli exposed to dexamethasone for at least 20 min with subsequent treatment with tested products and inoculation in dexamethasone-supplemented M9 exhibited similar growth and time lag as control E. coli (FIG. 13B).

We evaluated the universal effects of tested products on regulation of cells interaction with exogenous nutrients, by cultivating the lac-positive strain E. coli in M9 medium supplemented with lactose as the sole source of carbon and energy. Treatment of cells with the tested compounds increased the time lag by 2 h compared with control E. coli, indicating that these tested products could control utilization of lactose (FIG. 13C, table 18). As with dexamethasone, when control E. coli were pre-exposed to lactose for 20 min, followed by treatment with tested products and subsequent cultivation on M9 medium supplemented with lactose, their behavior and time lag was similar to that of control E. coli (FIG. 13D). This finding further confirmed the supervised role of tested products being able to regulate lactose metabolism over lac-operon and the efficacy of tested compounds for managing these processes.

TABLE 19
Effect of tested compounds on substrate recognition

| | Delay | | Delay |
|---|---|---|---|
| | lag phase | | lag phase |
| Compound | B. pumilus (h) | Compound | E. coli (h) |
| Ribosomal S1-like | 3 | S7 | 4 |
| Pumillo-like repeat | 3 | Polymerase | 3 |
| RNA helicase | 2 | Gal4 | 3 |
| RNase polymerase III | 4 | Histone H1 | 2 |
| Nuclear | 3 | DNA | 3 |
| ribonucleoproteins | | Methyltrans- | |
| HER2 | | ferases | |
| | | (DNMT1) | |

### Example 21: Products and Methods for Managing Cell Memory and Forgetting

We studied could we by tested products modulate cell memory formation and verified this possibility using an ‘adaptive’ memory experiment. We found that control B. pumilus “remembered” the first exposure to dexamethasone, as indicated by shortening of the lag phase from 5 h upon first exposure to 2 h upon second exposure for B. pumilus (FIG. 14A). Dexamethasone-sentient B. pumilus following treatment by products and subsequent restoration maintained a time lag below 2 h (FIG. 14B), Several repeated rounds of treatment with tested products taken in concentrations from 10 μg/mL to 1000 μg/mL and restoration led to “forgetting” of any previous exposure to dexamethasone and the behavior of the corresponding B. pumilus became similar (5-h lag phase) to that of control B. pumilus upon first exposure to dexamethasone.

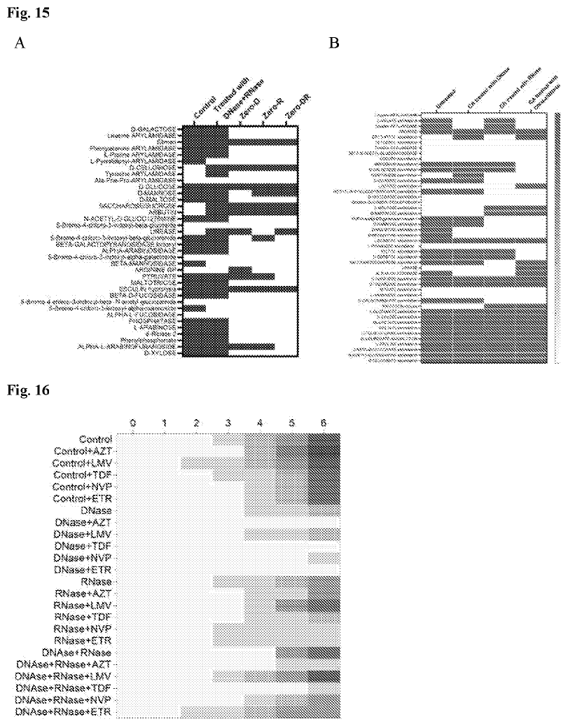

We found that after one or two-time treatment with tested products, cells continued to react faster to the substrate than at the very first contact (FIG. 15B). However, B. pumilus after three-time cycles inactivation with tested products required the same contact time as naïve cells to sense and trigger substrate utilization. We reasoned that multiple cycles of cells' treatment with tested results in destruction retained a type of “memory” (a reduced time required to launch substrate utilization) which is capable of maintaining and losing past histories of interactions.

### Example 22: Products and Methods for Managing Generation of Cells with Novel Properties

We next studied, how the tested compounds could be used for the development of the cells with a unique properties. We used products to generate Zero cells as previously described (several cycles of treatment with 10 μg/mL-100 μg/mL and analyzed biochemical properties of the resulted zero cells). Biochemical tests were carried out using the colorimetric reagent cards GN (gram-negative) and BCL (gram-positive spore-forming bacilli) of the VITEK® 2 Compact 30 system (BioMérieux, Marcy l'Étoile, France) according to the manufacturer's instructions. The generated data were analyzed using VITEK® 2 software version 7.01, according to the manufacturer's instructions. We also used eucaryotic Candida cells to generate cells with the unique properties by a single time use of tested products. The results clearly show that by putting cells to “zero state” or a single time treatment with tested products we were able to generate cells with the unique biochemical properties, being able to metabolize and degrade products that can't be metabolized by control cells (FIG. 15A,B).

### Example 23: Products and Methods to Managing Cell Growth Characteristics

We used reverse transcriptase inhibitors (taken at concentrations more than 2-100 fold lower than their MICs) against control S. aureus and S. aureus following the treatment with different nucleases (10 μg/mL). Zidovudine (AZT), Tenofovir (TNF), Nevirapine (NVP) and etravirine (ETR) at 5 μg/mL were added to the broth and OD600 was monitored hourly for 6 h at 37° C. Data (FIG. 16) shows the unique characteristics of combined use of nucleases and reverse transcriptase inhibitors on cell characteristics.

### Example 24. Products and Methods for Managing Signal Trafficking Inside Cells

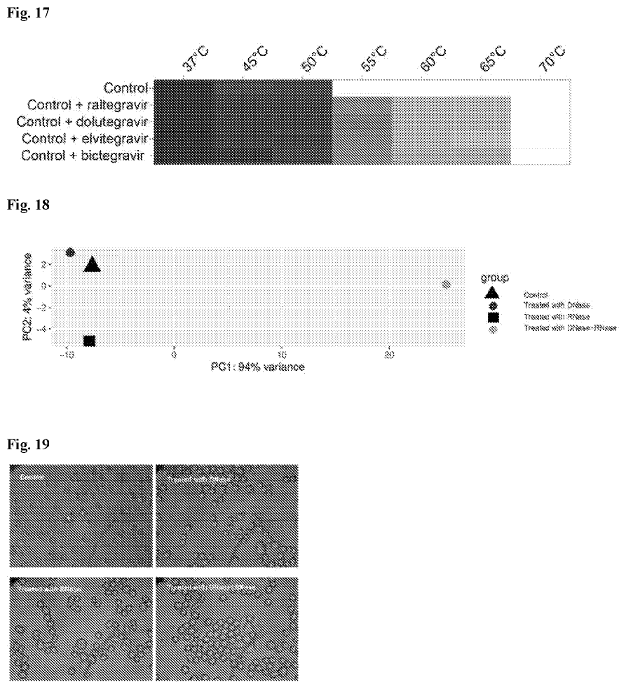

Next, we found that the onset of a signal transduction cascade following the interaction between cell and ligands depend on recombinases (HIV integrase inhibitors). Given that we previously showed that the treatment with tested products enhanced survival at higher temperatures, we hypothesized that raltegravir might block signal transduction and lead to higher heat tolerance even in control bacteria not treated with nucleases. S. aureus treated or not treated with raltegravir, dolutegravir, elvitegravir, bictegravir taken in non-toxic concentrations from 0.01 to 10 μg/mL was gradually heated up to 65° C. and the presence of viable bacteria was analyzed. S. aureus treated with recombinases could survive at temperatures over 15° C. higher than those of cells not treated with them (FIG. 17). There results clearly show that recombinases block signal transduction from the ligand to cell.

### Example 25: Products and Method for Managing Bacterial Sensitivity to Antibiotics

The standard NCCLS disk diffusion test was performed on isolate using supplemented mixed Columbia and Pepted Meat agar and standard ampicillin 10 ug, Gentamicin 10 ug, Azithromycin 15 ug, Clindamycin 10 ug, co-trimoxazole 25 μg test disks (Hardy diagnostics) were used. S. aureus VT 213 either separated or not separated from the extracellular matrix and treated with tested products (from 2 to 180 minutes). Following incubation for 24 h at 37° C., zone diameters were measured in the usual manner; significant ingrowth within a zone up to the edge of the disk was considered constitutive resistance. Data are shown in table 20.

TABLE 20
Inhibition zone diameter

| | Inhibition zone diameter (mm) | | | |
|---|---|---|---|---|
| | | DNase + RNase | DNase + RNase | NONO protein |
| | | (each 10 ug/mL, | (each 10 ug/mL, | (100 ug/mL, |
| Antibiotic | Control | 2 minutes) | 60 minutes) | 180 minutes) |

Extracellular matrix removed

| | | | | |
|---|---|---|---|---|
| Ampicillin | 14 | 21* | 24* | 24* |
| Gentamicin | 26 | 32* | 38* | 36* |
| Azithromycin | 20 | 28* | 27* | 25* |
| Clindamycin | 26 | 32* | 34* | 28 |

Extracellular matrix not removed

| | | | | |
|---|---|---|---|---|
| Ampicillin | 14 | 13 | 17 | 14 |
| Gentamicin | 26 | 28 | 21 | 25 |
| Azithromycin | 20 | 22 | 28* | 23 |
| Clindamycin | 26 | 24 | 31* | 27 |

*p < 0.05

We also studied effects of protease or integrase inhibitors (taken at concentration below their MIC) on cells treated with tested products. Data are presented in table 21 and 22

TABLE 21
Effect tested compounds on sensitivity to antibiotics

| | Inhibition | Sensi- |
|---|---|---|
| | zone diameter (mm) | tivity |
| Treatment | Co-trimoxazole | |
| Control | 10 | R |
| Control + Lopinavir/Ritonavir | 24 | S |
| DNase II + Lopinavir/Ritonavir | 23 | S |
| RNase + Lopinavir/Ritonavir | 25 | S |
| DNase + RNase + Lopinavir/Ritonavir | 26 | S |
| Control + raltegravir | 10 | R |
| RNase V1 + raltegravir | 27 | S |
| Ribosomal protein L3 + raltegravir | 22 | S |
| DNase+ RNase V1 + raltegravir | 10 | R |

The use of tested products alone or together with integrase inhibitors or protease inhibitors allows to modulate microbial sensitivity of bacteria to antibiotics.

TABLE 22
Effect of products on sensitivity of bacteria to antibiotics

| | Inhibition zone (mm) | | | | | |
|---|---|---|---|---|---|---|
| | | | | | Major vault | |
| | | | DNase + RNase | | protein + BamHI | |
| | | | (each 10 ug/mL, | | (each 10 ug/mL, | |
| | Control cells | | 2 minutes) | | 30 minutes) | |
| | | Potassium | | Potassium | | Potassium |
| Antibiotic | PBS | orotate | PBS | orotate | PBS | orotate |
| Ampicillin | 14 | 34 | 21 | 44 | 17 | 45 |
| Gentamycin | 26 | 33 | 32 | 41 | 34 | 39 |
| Azithromycin | 20 | 34 | 28 | 39 | 27 | 35 |

The use of tested products alone or together with integrase inhibitors or protease inhibitors allows to modulate microbial sensitivity of bacteria to antibiotics and that their effects on cells lacking extracellular matrix was more pronounced.

These data clearly shows that products potassium orotate increased bacterial sensitivity to antibiotics. Antibacterial effect of potassium orotate was more pronounced in cells with following the treatment with tested products.

### Example 26: Products and Method for Managing Gene Activity and Epigenetic Processes in Prokaryotes

We next studied how tested products could be used for the regulation of gene expression. To isolate RNA, the cell suspension obtained 2.5 h post-nuclease treatment were washed thrice in PBS, pH 7.2 (Sigma) and centrifuged each time at 4000×g for 15 min (Microfuge 20R, Beckman Coulter) followed by resuspension in PBS. RNA was purified using RNeasy Mini Kit (Qiagen) according to the manufacturer's protocol. The quantity and quality of RNA was spectrophotometrically evaluated by measuring the UV absorbance at 230/260/280 nm with the NanoDrop OneC spectrophotometer (ThermoFisher Scientific). Transcriptome sequencing (RNA-Seq) libraries were prepared using an Illumina TruSeq Stranded Total RNA Library Prep kit. RNA was ribodepleted using the EpicenterRibo-Zero magnetic gold kit (catalog no. RZE1224) according to the manufacturer's guidelines. The libraries were pooled equimolarly and sequenced in an Illumina NextSeq 500 (Illumona, San Diego CA) platform with paired 150-nucleotide reads (130 MM reads max).

Cells were separated from the extracellular matrix and treated with the tested products. It resulted in significant alteration of bacterial gene expression with a large number of differentially expressed proteins (|log 2-fold change|>0.5 and p-value <0.05) (FIG. 18, tables 23-25). There were major shifts in the regulation of genes responsible for ATP production, secretion systems, virulence factors, efflux pumps, synthetic activity.

TABLE 23
The list of selected differentially expressed genes that are differentially
expressed following primary the treatment of cells with DNase (log2
fold > 0.5 change plotted against the −log10 P-value).

| | | log2Fold | | | log2Fold |
|---|---|---|---|---|---|
| gene | protein | Change | gene | protein | Change |
| RsaA | non-coding RNAs | −0.52464 | hisH | Imidazole glycerol | 0.729793 |
| | | | | phosphate synthase | |
| | | | | subunit HisH | |
| RsaH | non-coding RNAs | −0.50462 | hrcA | Heat-inducible | −0.74132 |
| | | | | transcription repressor | |
| | | | | HrcA | |
| SA0205 | Lysostaphin | −0.80622 | hutI | Imidazolonepropionase | −0.64431 |
| SA0235 | EIIA | −1.05719 | hutU | Urocanate hydratase | −0.61493 |
| SA0271 | Type VII secretion | 0.665833 | ilvA | L-threonine | −0.94255 |
| | system extracellular | | | dehydratase | |
| | protein A | | | | |
| SA0272 | Type VII secretion | 0.56005 | ilvB | Acetolactate synthase | −0.74303 |
| | system accessory | | | | |
| | factor EsaA | | | | |
| SA0273 | ESAT-6 secretion | 0.680721 | ilvC | Ketol-acid | −0.75075 |
| | machinery protein EssA | | | reductoisomerase | |
| | | | | (NADP(+)) | |
| SA0275 | Type VII secretion | 0.606766 | leuA | 2-isopropylmalate | −0.84772 |
| | system protein EssB | | | synthase | |
| SA0276 | Type VII secretion | 0.516419 | leuB | 3-isopropylmalate | −0.81808 |
| | system protein EssC | | | dehydrogenase | |
| SA0308 | Gate domain-containing | −0.69438 | leuC | 3-isopropylmalate | −0.69053 |
| | protein | | | dehydratase large | |
| | | | | subunit | |
| SA0337 | SA0337 protein | 1.449767 | leuD | 3-isopropylmalate | −0.95587 |
| | | | | dehydratase small | |
| | | | | subunit | |
| SA0417 | Transporter | 0.710908 | msmX | Multiple sugar-binding | 0.74039 |
| | | | | transporter ATP- | |
| | | | | binding protein | |
| SA0482 | Protein-arginine kinase | −0.70815 | mtlA | PTS system mannitol- | −0.62255 |
| | | | | specific EIICB | |
| | | | | component | |
| SA0607 | Phosphoenolpyruvate -- | −0.41632 | mtlD | Mannitol-1-phosphate | −0.68691 |
| | glycerone | | | 5-dehydrogenase | |
| | phosphotransferase | | | | |
| SA1220 | Phosphate transport | −0.57372 | mtlF | EIICB-Mtl | −0.79557 |
| | system permease protein | | | | |
| SA1221 | Phosphate-binding | −0.53047 | nanA | N-acetylneuraminate | 0.671233 |
| | protein PstS | | | lyase | |
| SA1252 | N-acetyltransferase | 0.555391 | narI | Respiratory nitrate | −0.40226 |
| | domain-containing | | | reductase gamma chain | |
| | protein | | | | |
| SA1674 | Glutamate ABC | 1.046379 | orfX | Ribosomal RNA large | 0.633996 |
| | transporter ATP-binding | | | subunit | |
| | protein | | | methyltransferase H | |
| SA1898 | Probable | −1.11634 | pckA | Phosphoenolpyruvate | −0.44136 |
| | transglycosylase SceD | | | carboxykinase (ATP) | |
| SA1961 | EIIA | −0.74812 | pmi | Mannose-6-phosphate | −0.75769 |
| | | | | isomerase | |
| SA2179 | Oxygen regulatory | −0.4308 | rpmG | 50S ribosomal protein | 0.411525 |
| | protein NreC | | | L33 | |
| SA2180 | Oxygen sensor histidine | −0.44857 | rpsF | 30S ribosomal protein | 0.561903 |
| | kinase NreB | | | S6 | |
| SA2424 | HTH-type transcriptional | −0.46303 | rpsP | 30S ribosomal protein | 0.474323 |
| | regulator ArcR | | | S16 | |
| SA2448 | Flavin Reduct domain- | −0.93721 | rpsR | 30S ribosomal protein | 0.487543 |
| | containing protein | | | S18 | |
| SA2466 | | 1.034729 | sgtB | Monofunctional | −0.44849 |
| | | | | glycosyltransferase | |
| SA2469 | Putative pyridoxal | 0.65013 | ssb | Single-stranded DNA- | 0.591299 |
| | phosphate-dependent | | | binding protein | |
| | acyltransferase | | | | |
| SAS016 | Protein VraX | −1.1722 | tnp | IS6 family transposase | −0.4557 |
| aldA | Putative aldehyde | −0.71756 | trmD | tRNA (guanine-N(1)-)- | 0.429238 |
| | dehydrogenase AldA | | | methyltransferase | |
| argG | Argininosuccinate | 0.773082 | yent2 | Enterotoxin YENT2 | 0.92754 |
| | synthase | | | | |
| argH | Argininosuccinate lyase | 0.983391 | glmS | Glutamine--fructose- | −0.6199 |
| | | | | 6-phosphate | |
| | | | | aminotransferase | |
| clpB | Chaperone protein ClpB | −0.55285 | glpT | Glycerol-3-phosphate | 0.553561 |
| | | | | transporter | |
| ctsR | Transcriptional regulator | −0.47307 | gltB | Glutamate synthase | −0.6479 |
| | CtsR | | | large subunit | |
| dltA | D-alanine--D-alanyl | −0.56997 | gltC | LysR family | −0.74566 |
| | carrier protein ligase | | | transcriptional | |
| | | | | regulator | |
| dltB | Teichoic acid D- | −0.54777 | grpE | Protein GrpE | −0.76193 |
| | alanyltransferase | | | | |
| dltC | D-alanine--D-alanyl | −0.45714 | hisF | Imidazole glycerol | 0.851572 |
| | carrier protein ligase | | | phosphate synthase | |
| | | | | subunit HisF | |
| dltD | Protein DltD | −0.52729 | fhuC | ABC transporter | −0.58117 |
| dnaK | Chaperone protein DnaK | −0.86706 | gatC | Aspartyl/glutamyl- | −0.7494 |
| | | | | tRNA | |
| | | | | amidotransferase | |
| | | | | subunit C | |

TABLE 24
The list of selected differentially expressed genes that are differentially
expressed following the treatment of cells with RNase (log₂ fold >0.5
change plotted against the −log₁₀ P-value).

| row | log2FoldChange | row | log2FoldChange | row | log2FoldChange |
|---|---|---|---|---|---|
| RsaC | −2.02794 | SA1822 | 2.542264 | purC | 2.045844 |
| RsaH | −1.79759 | SA1982 | 3.575119 | purK | 2.128814 |
| SA0123 | 3.760189 | SA1983 | 1.978193 | ribA | 2.183933 |
| SA0124 | 4.139603 | SA2006 | 2.396529 | ribB | 2.112468 |
| SA0125 | 3.695716 | SA2009 | 4.001197 | ribD | 2.003981 |
| SA0126 | 2.568299 | SA2092 | 2.55528 | sak | −1.73932 |
| SA0164 | 1.984914 | SA2113 | 2.603624 | set15 | 4.068311 |
| SA0166 | 2.197103 | SA2174 | −1.74635 | sgtB | 4.62973 |
| SA0221 | 2.19545 | SA2177 | 2.870936 | sin | 1.994151 |
| SA0223 | 2.347172 | SA2220 | 2.227582 | ssaA | −1.70907 |
| SA0224 | 2.32358 | SA2221 | 2.479655 | ssp | 2.910317 |
| SA0225 | 2.057345 | SA2223 | 2.398185 | tnp | 2.309609 |
| SA0295 | −1.77593 | SA2297 | 1.735465 | truncated(radC) | 4.222394 |
| SA0378 | 2.036711 | SA2307 | 2.083224 | veg | 1.741464 |
| SA0407 | 1.861112 | SA2308 | 1.985593 | vraA | 4.962177 |
| SA0410 | 2.949748 | SA2315 | 2.78528 | vraB | 2.519028 |
| SA0453 | 2.997239 | SA2331 | −1.60243 | vraC | 3.348577 |
| SA0530 | 3.846535 | SA2343 | 4.232018 | vraD | 2.120242 |
| SA0532 | 1.513542 | SA2346 | 2.622487 | vraE | 2.569824 |
| SA0536 | 2.389639 | SA2347 | 2.485071 | vraR | 2.376511 |
| SA0550 | 1.738466 | SA2434 | −1.93625 | vraS | 2.778257 |
| SA0552 | 2.41206 | SA2454 | 3.384785 | SA1717 | 2.017368 |
| SA0553 | 1.831124 | SA2455 | 3.232942 | SA1821 | 2.61163 |
| SA0574 | 1.88142 | SA2457 | 3.794996 | SA1416 | 2.942128 |
| SA0578 | 1.869041 | SA2474 | 5.615799 | SA1418 | 3.543656 |
| SA0587 | −1.99921 | SA2481 | 1.987261 | SA1448 | 1.958864 |
| SA0591 | 1.889129 | SA2488 | 2.072205 | SA1474 | 1.662022 |
| SA0611 | 2.1997 | SAP023 | 2.191451 | SA1475 | 2.411525 |
| SA0634 | 1.890991 | SAP024 | 2.280428 | SA1476 | 3.433591 |
| SA0675 | 1.459974 | SAP025 | 2.057867 | SA1477 | 3.012576 |
| SA0705 | 2.484581 | SAS011 | 3.452245 | SA1486 | 5.914374 |
| SA0743 | 4.097884 | SAS014 | 4.16834 | SA1514 | 1.891583 |
| SA0745 | 2.711459 | SAS034 | 4.441121 | SA1534 | 1.578071 |
| SA0750 | 2.559779 | ahrC | 1.84051 | SA1685 | 3.103981 |
| SA0782 | 1.600356 | binL | 3.162673 | SA1688 | 2.881662 |
| SA0836 | 3.607095 | cadD | 2.46788 | SA1689 | 3.318298 |
| SA0840 | 3.274762 | copA | 2.74274 | SA1690 | 3.869985 |
| SA0858 | 3.464639 | fmtA | 4.024518 | SA1702 | 2.970515 |
| SA0914 | 1.492051 | fnb | 2.325192 | SA1703 | 3.121467 |
| SA0916 | 1.922044 | fnbB | 2.667835 | SA1706 | 3.509333 |
| SA1037 | 1.610453 | gapR | 1.889 | SA1712 | 1.705331 |
| SA1172 | 1.759181 | kdpA | 3.314208 | SA1371 | 4.145084 |
| SA1180 | 2.192593 | kdpB | 2.269185 | SA1372 | 3.589273 |
| SA1196 | 2.738235 | lpl7 | 2.600261 | SA1373 | 3.236583 |
| SA1219 | 2.085466 | lpl9 | 2.240395 | SA1374 | 2.563379 |
| SA1220 | 1.943926 | lrgA | −2.22396 | SA1389 | 1.643966 |
| SA1221 | 2.628793 | lrgB | −2.7117 | SA1415 | 1.973882 |
| SA1270 | −1.58399 | mscL | 3.508171 | proP | 2.220232 |
| SA1275 | 2.099097 | pmi | −1.9295 | prsA | 2.446794 |
| SA1369 | 4.136056 | SA1370 | 4.497155 | pstB | 2.188066 |

TABLE 25
The list of selected differentially expressed genes that are
differentially expressed following the treatment of cells with DNase +
RNase (log₂ fold >0.5 change plotted against the −log₁₀ P-value).

| row | log2FoldChange | row | log2FoldChange |
|---|---|---|---|
| Bacteria_large_SRP | 1.739106 | SA2009 | 2.001824 |
| LSU_rRNA_bacteria | 4.461256 | SA2321 | −1.11136 |
| SA0276 | 1.005593 | SA2490 | −1.69218 |
| SA0530 | −1.92041 | SAP007 | 1.612565 |
| SA0970 | −1.01401 | SAS059 | 1.550807 |
| SA1021 | −1.20292 | SARNA14 | 2.114453 |
| SA1281 | −1.01242 | ilvA | −1.04354 |
| SA1486 | 1.266669 | leuA | −1.05624 |
| SA1665 | −1.19627 | leuC | −1.07209 |
| SA1767 | 2.245527 | rpsT | −1.34622 |
| SA1791 | 1.117999 | veg | −1.23421 |
| SA1798 | 2.208869 | SA1898 | −1.31808 |

The use of tested products is possible to manage gene activity and epigenetic processes in prokaryotes.

### Example 27: Products and Method for Managing Gene Activity and Epigenetic Processes in Eukaryotes

We next found that the use of the tested products has a global impact on gene expression on eukaryotic organisms using Vero cells. RNA extraction and transcriptome sequencing were conducted as previously discussed. Treatment of cells following the separation form the extracellular matrix with products resulted in significant alteration of multiple critical gene expression with a large number of differentially expressed proteins (|log 2-fold change|>0.5 and p-value <0.05) (tables 26-28). There were major shifts in the regulation of genes responsible for ATP production, secretion systems, virulence factors, efflux pumps, synthetic activity.

TABLE 26
The list of selected differentially expressed genes that are differentially expressed following
the treatment with DNase (log2 fold >0.5 change plotted against the −log10 P-value).

| row | log2FoldChange | row | log2FoldChange | row | log2FoldChange |
|---|---|---|---|---|---|
| ABAT | −1.54537 | CSMD2 | −1.30241 | IL36G | −4.73932 |
| ADAMTS18 | −1.64956 | CSRNP3 | 1.014335 | ING1 | −1.29533 |
| ADGRG3 | 1.208517 | CYP4F11 | Inf | INPP5D | 1.477427 |
| AFF3 | −3.04744 | CYTB | 2.008872 | INSIG1 | −1.28895 |
| ANK1 | −2.22147 | DACH1 | −1.8889 | ITGAX | −1.127 |
| ANKRD1 | 1.100457 | DAPK2 | 2.711892 | KCNJ5 | −1.59854 |
| ANKRD37 | −1.425 | DES | 1.652998 | KCNMB4 | −1.09514 |
| APBA1 | 3.460353 | DNAAF11 | 2.035468 | KLF2 | 1.444161 |
| AQP5 | Disappearance | DNAJB13 | 1.460353 | LARGE2 | −1.21207 |
| ARL4C | −1.03367 | DUOX1 | 2.03151 | LCP1 | 1.285266 |
| ATP6 | 1.807284 | DUSP9 | −1.37224 | LGI3 | −1.46831 |
| AXIN2 | −1.4813 | EBF4 | −1.72551 | LRFN5 | −1.22031 |
| BACH2 | −1.06321 | EDA | 1.926017 | LRRC15 | 1.017896 |
| BARHL1 | 1.398425 | EFCAB6 | 1.023836 | LRRC17 | −1.68768 |
| BEAN1 | −1.83639 | EFEMP1 | −1.39901 | LRRN4CL | −1.03596 |
| BGN | −1.14496 | EFNB3 | −1.06559 | LURAP1L | 1.520895 |
| BLNK | −1.58801 | EFR3B | −1.37254 | MAP1LC3C | −1.56939 |
| BMPER | −1.37675 | EMG1 | 1.551119 | MAP3K5 | −1.06925 |
| C11orf1 | 1.004471 | ENC1 | −2.04793 | MBP | −1.1223 |
| C17orf97 | 1.723388 | F2RL1 | −1.38958 | MCHR1 | −1.18735 |
| C1R | −1.07613 | FAM107A | 1.170456 | MEX3B | −1.47376 |
| CACNA1C | −1.23334 | FAM131B | −2.93196 | MFAP2 | −1.40145 |
| CACNG8 | Disappearance | FCER2 | 2.353438 | MIR106B | −2.46248 |
| CADM1 | −1.12361 | FGF21 | 1.17846 | MIR199A1 | 3.17656 |
| CALCB | −1.23597 | FOXN3 | 2.652998 | MIR3074 | −2.347 |
| CBLIF | −1.66893 | FSTL5 | −1.09199 | MMP1 | −1.80866 |
| CCL2 | −2.20813 | FUT1 | 1.455059 | MMP10 | −2.13777 |
| CCN2 | 1.272064 | FZD4 | −1.32456 | MMP17 | −1.42396 |
| CD34 | Inf | GAB3 | Inf | MMP23B | 1.619831 |
| CD82 | −1.15215 | GADD45A | 1.136782 | MMP3 | −1.72551 |
| CDC42EP5 | −1.95844 | GALNT16 | −1.55345 | MN1 | 1.058584 |
| CEP126 | 1.217783 | GAS1 | −1.38562 | MRC2 | −1.12903 |
| CFAP43 | 2.194892 | GBP1 | −2.195 | MT-ND2 | 2.325916 |
| CLDN5 | 2.652998 | GFI1B | Inf | MT-ND4 | 1.92231 |
| CLEC2L | −3.66893 | GIMAP6 | 1.389964 | MTMR7 | −1.41412 |
| CNN1 | −1.67157 | GPR146 | −1.09907 | MYB | Inf |
| COL13A1 | −1.49995 | GRAP2 | 1.237961 | MYL3 | −1.74555 |
| COL21A1 | −1.30518 | GRIN3B | −1.35699 | MYLIP | −2.65116 |
| COX2 | 1.304372 | GRIP2 | −1.56939 | NAGS | −1.15893 |
| COX3 | 1.172228 | HEPACAM | Disappearance | NCKAP5 | −1.03627 |
| CRYBA1 | −1.01769 | HEPACAM2 | Inf | ND1 | 2.201368 |
| CSF2 | −1.44654 | HHIP | −2.83243 | ND4L | 1.918924 |
| ND6 | 1.923558 | HS3ST5 | −1.66893 | ND5 | 1.769032 |
| NEFL | −2.56939 | SCD | −1.00039 | SNORD83 | −1.18946 |
| NHSL2 | −1.09116 | SERPINA10 | 1.652998 | SOD2 | −1.0509 |
| NKD1 | −1.8889 | SGCG | 1.822923 | SPDEF | −1.49139 |
| NTN1 | −1.10754 | SLC16A2 | −1.80739 | SPDYC | 3.353438 |
| OR10P1 | 1.974926 | SLC1A1 | −1.20084 | SPEF1 | 2.32285 |
| OTOF | −2.195 | SLC25A47 | 2.03151 | SPTBN5 | −1.04031 |
| PAPLN | −1.29381 | WDR93 | 1.890037 | SSTR5 | Inf |
| PCSK9 | −2.10781 | SLC29A4 | −1.62002 | ST3GAL6 | −1.18142 |
| PDE1A | Disappearance | SLC6A15 | 3.652998 | ST8SIA4 | −1.93196 |
| PGM5 | Disappearance | SLCO2A1 | −1.05964 | STAC2 | −1.59829 |
| PKD1L3 | −2.11254 | SLIT2 | −1.16709 | STC1 | −1.36526 |
| PLEK | −1.2256 | SNAI1 | 2.407886 | TAMALIN | −1.07304 |
| PLG | −2.25389 | SNORA40B | 1.578998 | TAS2R42 | −2.56939 |
| PLN | Inf | SNORA68 | −1.03767 | TEX26 | −1.66893 |
| PLXNC1 | −1.88542 | SNORD126 | −3.80643 | TGM2 | −1.39442 |
| PMEPA1 | −1.31362 | SNORD17 | −1.08072 | TLCD3B | Inf |
| PNPLA4 | −1.15436 | SNORD49A | −1.0114 | TNC | −1.71002 |
| PPM1J | −1.05002 | SNORD82 | −1.93196 | TRAJ18 | −2.80643 |
| PTH1R | −1.281 | WIF1 | −1.79307 | TRMT9B | 2.193567 |
| PTPDC1 | 2.974926 | WNT11 | −1.43107 | TRPM8 | Disappearance |
| QPCT | −1.22917 | WNT3A | −2.46248 | TTYH1 | −1.13876 |
| RAB20 | −1.42025 | ZACN | −2.347 | UGT1A5 | 1.430606 |
| RET | −1.26983 | ZBTB7C | −1.5214 | UNC80 | −2.59493 |
| RUNDC3A | 1.434358 | ZFP36 | 1.040804 | VASH1 | −1.14593 |
| SCARNA10 | −1.99251 | SCARNA6 | −1.57519 | VNN2 | 1.672898 |

TABLE 27
The list of selected differentially expressed genes that are differentially expressed following
the treatment with RNase (log₂ fold >0.5 change plotted against the −log₁₀ P-value).

| row | log2FoldChange | row | log2FoldChange | row | log2FoldChange |
|---|---|---|---|---|---|
| AATK | −2.98744 | MBP | −1.00784 | RPRML | Disappearance |
| ADH4 | Disappearance | MC1R | Inf | RSPH6A | −1.8879 |
| ANO4 | −1.51795 | MIR132 | −2.0399 | SCARNA18 | −2.30294 |
| AQP5 | Disappearance | MIR148B | −3.62487 | SCARNA21 | 1.048632 |
| AQP9 | Disappearance | MIR219A2 | Disappearance | SLC10A1 | −2.05852 |
| ASB11 | 2.706049 | MIR29C | −2.55448 | SLC10A5 | −1.9395 |
| ASB16 | −1.07467 | MIR3074 | −1.4252 | SLC25A34 | −1.88164 |
| ASPN | −1.4907 | MIR3610 | 1.020057 | SLC6A15 | 3.430415 |
| ATP6 | 2.612707 | MIR554 | −1.19938 | SLC7A10 | −3.62487 |
| B3GALT2 | −1.09623 | MIR624 | −3.23255 | SNAI1 | 2.226882 |
| BFSP1 | 1.532985 | MORN1 | −1.37005 | SNORA20 | 2.159767 |
| C15orf62 | −1.70204 | MORN5 | −3.81751 | SNORA47 | 2.226882 |
| C17orf97 | 1.641919 | MT-ND2 | 3.106659 | SNORA53 | 1.247443 |
| C6orf132 | −1.35185 | MT-ND4 | 2.706288 | SNORA80B | −1.83589 |
| CBLN2 | −2.30294 | MT1B | 1.002383 | SNORD114-31 | −1.2623 |
| CCDC103 | 1.198789 | MTMR7 | −1.81751 | SNORD115 | −3.90498 |
| CCL19 | Disappearance | MYB | Inf | SNORD12B | −1.1572 |
| CEP295NL | −1.13399 | NAGS | −1.26331 | SNORD70B | −2.06544 |
| CHCHD5 | 1.103359 | ND1 | 2.451467 | SNORD82 | −2.18008 |
| CLCN1 | −3.62487 | ND3 | 1.537819 | SPATC1L | −2.69198 |
| COX2 | 1.888932 | ND4L | 2.495928 | TEAD2 | 1.27441 |
| COX3 | 1.873649 | ND5 | 2.353517 | TMEM150A | 1.528262 |
| CXCR3 | −2.93299 | ND6 | 2.332151 | TPD52L3 | Disappearance |
| CXCR4 | −4.51795 | NDUFS6 | 1.010523 | TSSK1B | Disappearance |
| CYTB | 2.291074 | NECAB1 | −1.81751 | VGLL2 | 1.737076 |
| DCAF4L1 | −1.11029 | NR4A2 | −1.2275 | WNT3A | −2.19602 |
| EDNRB | Disappearance | P2RX2 | −1.13944 | ZNF610 | 1.08519 |
| EGFL6 | −1.90498 | PEAK3 | −2.40247 | ZNF83 | −2.40247 |
| EGR1 | 1.040539 | PLG | −2.40247 | GHRH | −3.40247 |
| EMG1 | 1.352413 | PPDPF | 1.128204 | GNAT1 | −2.27694 |
| EVA1B | Disappearance | PTPRZ1 | −2.40247 | H2AC7 | 1.275198 |
| FAM189A1 | 3.26995 | PYCARD | −1.23001 | FXYD4 | −3.81751 |
| FAM71A | −2.19056 | RNF113B | −1.43222 | FXYD5 | 1.125698 |
| FITM1 | −3.62487 | RNU4ATAC18P | −1.90498 | GABPB2 | 2.070013 |
| FLRT1 | −1.08403 | FOXN3 | 2.76745 | MAMDC4 | −1.04725 |
| HCRTR1 | −1.41791 | LAG3 | −1.90498 | MATN2 | −1.47648 |
| KLHDC1 | −1.40247 | LHX4 | −1.28958 | KRT79 | 1.020057 |
| LINGO3 | −3.13944 | | | | |

TABLE 28
The list of selected differentially expressed genes that are differentially expressed following
the treatment with DNase + RNase (log2 fold >0.5 change plotted against the −log10 P-value).

| row | log2FoldChange | row | log2FoldChange | row | log2FoldChange |
|---|---|---|---|---|---|
| ADAMTS8 | 1.2654 | FOXN3 | 2.998885 | PPARA | 2.271903 |
| ADGRG3 | 1.369528 | FUT5 | Inf | PPARGC1A | 2.413922 |
| AKAP12 | 1.030693 | GADD45A | 1.031093 | PRODH2 | −1.74434 |
| APOH | 2.413922 | GCA | −1.20079 | PROX1 | 3.501385 |
| ATOH8 | −1.05019 | GNAT1 | −2.72358 | PYROXD2 | 1.035411 |
| ATP6 | 2.260088 | H1-6 | −3.04551 | RASAL1 | 1.035411 |
| AXIN2 | −1.24504 | H1-8 | −3.58608 | RUNX3 | Disappearance |
| BCAM | 1.318087 | HDC | Disappearance | SAMSN1 | Disappearance |
| BMP3 | −4.04551 | HHIPL1 | −1.23517 | SCARNA10 | −2.13445 |
| C12orf56 | −3.04551 | HLF | 1.328192 | SCARNA6 | −1.55462 |
| C17orf97 | 2.395775 | HPD | −3.756 | SCN9A | 1.650962 |
| C9orf116 | 1.145274 | IL17F | Inf | SERPING1 | 1.635403 |
| CACNG4 | Disappearance | IL1RL2 | Disappearance | SHOX2 | −1.12922 |
| CCDC89 | 1.73585 | IQCF1 | Inf | SLC10A1 | −1.70155 |
| CCL2 | −2.42906 | KCNH3 | 1.034954 | SLC10A5 | −1.40321 |
| CCL20 | −3.58608 | KLHL24 | −1.04466 | SLC6A3 | 1.73585 |
| CDKL4 | Inf | LARGE2 | −1.17104 | SLCO2B1 | −1.80847 |
| CEP126 | 1.150888 | LRRC25 | −2.35161 | SNORA21B | −1.43407 |
| CFAP251 | 1.094463 | MAFA | −2.22351 | SNORA23 | −1.45281 |
| CFAP43 | 1.896315 | MAGEA11 | Inf | SNORA28 | −1.73808 |
| CFAP61 | 1.280171 | MBP | −1.06181 | SNORA52 | −1.15723 |
| CHCHD5 | 1.171809 | MC3R | 2.271903 | SNORA63 | −1.14067 |
| CHRNA10 | −1.65021 | MEX3B | −1.57266 | SNORA67 | −1.41099 |
| CISH | 1.086348 | MFAP2 | −1.14457 | SNORA73 | −1.00196 |
| CLDN4 | −1.0354 | MIR1296 | 3.73585 | SNORA80B | −1.18942 |
| COX2 | 1.652697 | MIR132 | −2.80847 | SNORD10 | −1.10876 |
| COX3 | 1.794653 | MIR148B | Disappearance | SNORD111B | −2.58608 |
| CREB5 | 1.003025 | MIR29C | −2.90801 | SNORD114-20 | −1.2535 |
| CXCL8 | −1.34654 | MIR454 | −2.70155 | SNORD17 | −1.77316 |
| CXCR4 | −3.28652 | MMP10 | −1.81125 | SNORD46 | −1.50362 |
| CYP26C1 | −4.04551 | MMP9 | −1.18332 | SNORD49A | −1.42381 |
| CYTB | 1.856835 | MN1 | 1.757224 | SNORD82 | −2.17104 |
| DMRT1 | −1.83401 | MT-ND2 | 2.79108 | SNORD97 | −1.24446 |
| DNAAF11 | 2.032832 | MT-ND4 | 2.279754 | SPA17 | 1.046191 |
| DNAJB13 | 1.449546 | MTMR7 | −1.34507 | SPEF1 | 2.612702 |
| DPF3 | 2.66185 | NAGS | −1.05697 | SPEF2 | 1.55688 |
| EDNRB | −3.90801 | NANOS3 | −2.70155 | SPOCK3 | −1.03086 |
| EFCAB6 | 1.047794 | ND1 | 2.067813 | SPRY1 | 1.444949 |
| EMG1 | 1.446344 | ND3 | 1.215696 | STRA6 | −1.53361 |
| FAM189A1 | 3.413922 | ND4L | 2.28454 | TAMALIN | −1.41554 |
| FAM71A | −1.0825 | ND5 | 2.216039 | TCF7L1 | 1.064687 |
| FLT4 | −1.29977 | ND6 | 2.078527 | TMEM150A | 1.472816 |
| FOS | −1.17503 | NEDD9 | 1.152261 | TMEM200A | 1.692728 |
| NR4A2 | −1.07912 | NMRK1 | 1.011163 | TMEM249 | −3.756 |
| OR6B1 | −2.58608 | VWA5A | 1.967176 | TPD52L1 | 1.104818 |
| PCSK9 | −1.19489 | WNT3A | −4.28652 | TRAF1 | −1.13482 |
| PDE7B | −1.01701 | WNT7B | 1.131225 | TSPAN11 | −1.4736 |
| PECAM1 | 1.039527 | ZACN | −2.58608 | TSPAN13 | −1.01952 |
| PER2 | 1.042796 | ZNF423 | 1.348827 | TXNDC8 | Inf |
| ZNF704 | 1.203999 | VSIG2 | −2.00112 | | |

Collectively with the by the product treatment it is possible to modulate different cellular processes and pathways. Some of them are listed in table 29.

TABLE 29
The list of selected pathways

| | | |
|---|---|---|
| acrosome reaction | | calcium ion transport |
| postsynapse to nucleus | modulation of chemical | calcium-independent cell- |
| signaling pathway | synaptic transmission | cell adhesion |
| adenylate cyclase activity | monocyte chemotaxis | calcium-mediated signaling |
| angiogenesis | mucociliary clearance | mRNA processing |
| apoptotic process | myelin maintenance | canonical Wnt signaling |
| | | pathway |
| apoptotic signaling pathway | myeloid dendritic cell | carbohydrate metabolic |
| | chemotaxis | process |
| ATP biosynthetic process | myosin light chain binding | carbon dioxide transport |
| ATPase activity | NAD biosynthetic process | cardiac muscle contraction |
| Notch signaling pathway | nervous system development | cardiac muscle tissue |
| | | development |
| B cell proliferation | neural crest cell migration | cation homeostasis |
| blood coagulation | neurogenesis | C-C chemokine binding |
| brown fat cell differentiation | neuron differentiation | C-C chemokine receptor |
| | | activity |
| calcium ion binding | neuropeptide hormone activity | CCR chemokine receptor |
| | | binding |
| calcium ion transport | neutrophil chemotaxis | CCR10 chemokine |
| | | receptor binding |
| pancreatic cell proliferation | oxidoreductase activity | CCR7 chemokine receptor |
| | | binding |
| Wnt signaling pathway | nuclear receptor binding | cell adhesion |
| cardiac muscle cell | nuclear receptor coactivator | cell chemotaxis |
| differentiation | activity | |
| catalytic activity | osteoblast differentiation | cell differentiation |
| cell cycle | ovulation | cell division |
| cell migration | paracrine signaling | cell fate commitment |
| cell migration involved in | axon extension involved in | paraxial mesodermal cell |
| sprouting angiogenesis | axon guidance | fate commitment |
| cell motility | cell maturation | cell migration |
| cell population proliferation | peptide hormone binding | cell motility |
| cell population proliferation | peroxidase activity | cell population |
| | | proliferation |
| cell-cell adhesion mediated | peripheral nervous system | mitotic G2 DNA damage |
| by cadherin | development | checkpoint signaling |
| cell-substrate adhesion | peroxisome proliferator | cell proliferation in |
| | activated receptor binding | midbrain |
| cellular protein metabolic | phospholipase C-activating G | cell surface receptor |
| process | protein-coupled receptor | signaling pathway |
| | signaling pathway | |
| cellular respiration | phospholipid biosynthetic | cell-cell signaling |
| | process | |
| cellular response to heat | platelet aggregation | cell-matrix adhesion |
| cellular response to hypoxia | post-anal tail morphogenesis | cellular calcium ion |
| | | homeostasis |
| adaptive immune response | cell proliferation in forebrain | cellular glucose |
| | | homeostasis |
| chemotaxis | | cellular protein |
| | | localization |
| cold-induced thermogenesis | collagen biosynthetic process | cellular respiration |
| potassium ion transmembrane | presynapse assembly Source: | collateral sprouting in |
| transport | ParkinsonsUK-UCL | absence of injury |
| cellular response to ATP | proline catabolic process | cellular response to |
| | | caffeine |
| cysteine-type endopeptidase | promoter-specific chromatin | cellular response to |
| activity involved in apoptosis | binding | calcium ion |
| cytokine production | prostaglandin biosynthetic | cellular response to |
| | process | cytokine |
| cytosolic calcium ion | prostaglandin-endoperoxide | cellular response to drug |
| concentration | synthase activity | |
| dendrite extension | protein arginylation | cellular response to |
| | | estradiol |
| dendritic cell antigen | protein domain specific | cellular response to fluid |
| processing and presentation | binding | shear stress |
| DNA-binding transcription | posterior midgut development | cellular response to |
| factor activity | | follicle-stimulating |
| | | hormone |
| dendritic cell dendrite | cellular response to glucose | protein-containing complex |
| assembly | stimulus | assembly |
| dermatome development | protein stabilization | protein phosphorylation |
| DNA recombination | cellular response to fructose | cellular response to heat |
| dendritic cell apoptosis | receptor ligand activity | cellular response to |
| | | hypoxia |
| dopaminergic neuron | regulation of ATPase-coupled | cellular response to |
| differentiation | calcium transmembrane | hydrogen peroxide |
| | transporter activity | |
| endocytosis | regulation of blood pressure | cellular response to |
| | | hypoxia |
| endothelial cell proliferation | regulation of calcium ion | ERK1 and ERK2 cascade |
| | transport | |
| cellular response to increased | regulation of cardiac muscle | cellular response to |
| oxygen level | cell contraction | interferon-gamma |
| fat cell differentiation | protein homodimerization | response to interleukins |
| | activity | |
| fatty acid oxidation | fever generation | cellular response to nitrite |
| cellular response to | regulation of cell | cellular response to |
| lipopolysaccharide | morphogenesis Source: ARUK- | mechanical stimulus |
| | UCL | |
| fibroblast growth factor | regulation of cell projection | regulation of epithelial cell |
| production | assembly | proliferation |
| G1/S transition of mitotic cell | cellular response to oxidative | cellular response to |
| cycle | stress | potassium ion |
| gene expression | regulation of circadian rhythm | regulation of chemotaxis |
| cellular response to thyroid | regulation of cell membrane | cellular response to non- |
| hormone stimulus | potential | ionic osmotic stress |
| gene expression | gluconeogenesis | cellular response to |
| | | resveratrol |
| glial cell proliferation | regulation of fever generation | regulation of gene |
| | | expression |
| glial cell-derived | cellular response to | glomerular visceral |
| neurotrophic factor | transforming growth factor beta | epithelial cell apoptotic |
| production | stimulus | process |
| cerebellum development | acid regulation of heart | cellular response to retinoic |
| | contraction | |
| regulation of NMDA receptor | regulation of inflammatory | cellular response to tumor |
| activity | response | necrosis factor |
| glycolytic process | regulation of membrane | cellular response to UV |
| | potential | |
| glycoprotein biosynthetic | regulation of synapse | cellular response to virus |
| process | organization | |
| granulocyte colony- | regulation of | heart induction by |
| stimulating factor production | neuroinflammatory response | canonical Wnt signaling |
| | | pathway |
| GTPase activity | regulation of presynapse | cGMP catabolic process |
| | assembly | |
| heart rate | regulation of pH | cGMP-mediated signaling |
| hematopoietic progenitor cell | regulation of programmed cell | regulation of Na ion |
| differentiation | death | transporter activity |
| hepatocyte proliferation | chemokine activity | chemokine receptor |
| | | binding |
| hepatic stellate cell | regulation of protein binding | chemokine-mediated |
| activation | | signaling |
| regulation of cytosolic Ca | regulation of relaxation of | regulation of the force of |
| concentration | cardiac muscle | heart contraction by |
| | | cardiac conduction |
| histone acetylation | regulation of ryanodine- | chemotaxis |
| | sensitive calcium-release | |
| | channel activity | |
| I-kappaB kinase/NF-kappaB | regulation of the force of heart | regulation of microtubule |
| signaling | contraction | cytoskeleton organization |
| interleukin-1 beta production | histone H3-K9 methylation | chromatin DNA binding |
| interleukin-10 production | chloride transport -KW | chromatin remodeling |
| interleukin-12 production | respiratory electron transport | cilium assembly |
| | chain | |
| regulation of transcription, | regulation of transcription by | circadian regulation of |
| DNA-templated | RNA polymerase II | gene expression |
| intrinsic apoptotic signaling | regulation of transcription, | macrophage migration |
| pathway in response to | DNA-templated | inhibitory factor signaling |
| osmotic stress | | pathway |
| JNK cascade | interleukin-2 production | cobalamin binding |
| JUN kinase activity | regulation of viral process | collagen catabolic process |
| circadian rhythm | relaxation of cardiac muscle | COP9 signalosome |
| | | assembly |
| mitochondrion organization | renal absorption | coreceptor activity |
| mesodermal cell fate | release of sequestered calcium | regulation of sensory |
| specification | ion into cytosol | perception of pain |
| metallopeptidase activity | chloride transmembrane | neural precursor cell |
| | transport | proliferation |
| mitochondrial DNA | CXCL12-activated CXCR4 | C-X-C motif chemokine 12 |
| metabolic process | signaling pathway | receptor activity |
| mitochondrial fission | response to cadmium ion | response to angiotensin |
| response to cold | response to calcium ion | cyclooxygenase pathway |
| muscle tissue development | response to cobalamin | cytokine activity |
| co-receptor binding | mesenchymal stem cell | C-X-C chemokine receptor |
| | migration | activity |
| neurogenesis | response to copper ion | decidualization |
| neuron apoptotic process | response to dietary excess | neuron maturation |
| neuron death | response to drug | dendritic cell chemotaxis |
| defense response to | platelet-derived growth factor | detection of chemical |
| bacterium -KW | production | stimulus involved in |
| | | sensory perception |
| maintenance of blood-brain | oligodendrocyte differentiation | DNA-binding transcription |
| barrier | | factor binding |
| neutrophil chemotaxis | response to epinephrine | detection of temperature |
| NIK/NF-kappaB signaling | response to estradiol | digestion |
| response to fructose | response to ethanol | DNA binding |
| odontogenesis | response to fatty acid | DNA repair |
| response to electrical | nitric oxide biosynthetic | response to endothelin |
| stimulus | process | |
| penile erection | response to glucagon | response to glucocorticoid |
| peptidyl-serine | dorsal/ventral neural tube | electron transport coupled |
| phosphorylation | patterning | proton transport |
| phosphatidylinositol 3-kinase | detection of stimulus involved | electron transport coupled |
| activity | in sensory perception of pain | proton transport |
| response to mercury ion | response to heat | embryo implantation |
| progesterone biosynthetic | enteric nervous system | prostaglandin biosynthetic |
| process | development | process |
| endothelial cell | endothelial tube morphogenesis | endothelial cell |
| differentiation | | proliferation |
| protein binding | extracellular exosome | response to hypoxia |
| | assembly | |
| protein catabolic process | response to ischemia | endothelin receptor |
| | | activity |
| protein import into nucleus | response to leucine | response to insulin |
| protein kinase A signaling | response to lipopolysaccharide | endothelium development |
| protein kinase activity | response to lithium ion | energy homeostasis |
| protein kinase B signaling | response to manganese ion | response to methionine |
| protein localization to plasma | glomerular endothelium | enteric smooth muscle cell |
| membrane | development | differentiation |
| protein phosphorylation | response to metformin | enzyme binding |
| renal sodium excretion | receptor-mediated endocytosis | smooth muscle cell |
| | | migration |
| receptor internalization | response to morphine | epithelial cell development |
| enzyme inhibitor activity | response to muscle activity | epithelial fluid transport |
| protein tyrosine kinase | endothelin receptor signaling | smooth muscle cell |
| activity | | proliferation |
| signaling receptor activity | response to norepinephrine | establishment of skin |
| | | barrier |
| response to nitric oxide | fatty acid metabolic process | establishment of T cell |
| | | polarity |
| response to progesterone | response to oxidative stress | estrogen receptor binding |
| RNA splicing | response to oxidative stress | muscle tissue development |
| smooth muscle contraction | response to pain | extracellular matrix |
| | | binding |
| T-helper 1 cell | response to prostaglandin E | extracellular matrix |
| differentiation | | organization |
| transcription by RNA pol. II | synaptic transmission | response to cyclic |
| | | compound |
| synaptic plasticity | response to reactive oxygen | fatty acid oxidation |
| | species | |
| T cell migration | response to starvation | fatty acid transport |
| T cell proliferation | response to testosterone | female pregnancy |
| testosterone secretion | response to toxic substance | flavone metabolic process |
| response to virus | response to tumor necrosis | forebrain development |
| | factor | |
| T-helper cell differentiation | response to ultrasound | frizzled binding |
| G protein-coupled receptor | synaptic transmission, | G protein-coupled receptor |
| signaling pathway, coupled to | dopaminergic | signaling pathway |
| cyclic nucleotide second | | |
| messenger | | |
| transcription | response to zinc ion | response to vitamin D |
| transcription by RNA pol. II | epithelial to mesenchymal | neuron development |
| | transition | |
| transcription, DNA- | RNA binding | G2/M transition of mitotic |
| templated | | cell cycle |
| hair cycle | tumor necrosis factor | galactose metabolic |
| | production | process |
| transforming growth factor | Wnt signaling involved in | gamma-aminobutyric acid |
| beta production | forebrain neuroblast division | biosynthetic process |
| scavenger receptor activity | secondary palate development | retinoic acid catabolic |
| | | process |
| urine volume | sensory perception of pain | glomerular filtration |
| hemopoiesis | sequence-specific DNA | gluconeogenesis |
| | binding | |
| vascular wound healing | signal transduction | glucose metabolic process |
| vasculogenesis | signaling receptor binding | growth factor activity -KW |
| vasoconstriction | signaling receptor binding | GTPase activity |
| wound healing | signaling receptor binding | stem cell proliferation |
| actin binding | skeletal muscle atrophy | heart looping |
| activation of MAPK activity | skeletal muscle cell | hippocampus development |
| | differentiation | |
| adaptive thermogenesis | somatic stem cell division | heme binding |
| spinal cord association neuron | sodium ion transmembrane | vascular endothelial |
| differentiation | transport | growth factor production |
| adenylate cyclase-inhibiting G | transcription initiation from | transmembrane receptor |
| receptor signaling pathway | RNA polymerase II promoter | protein tyrosine kinase |
| | | adaptor activity |
| synaptic vesicle recycling | immunological synapse | hyperosmotic salinity |
| | formation | response |
| aging | stem cell proliferation | temperature homeostasis |
| alcohol metabolic process | T cell costimulation | immune response |
| alpha-tubulin binding | T cell migration | inflammatory response |
| amino acid transport | telencephalon cell migration | in utero embryonic |
| | | development |
| adipose tissue development | killing of cells of other | intracellular signal |
| | organism | transduction |
| angiogenesis | thymocyte migration | inner ear morphogenesis |
| animal organ regeneration | tissue homeostasis | integral component of |
| | | membrane |
| androgen metabolic process | transcription coactivator | integral component of |
| | activity | membrane |
| apoptotic process | transcription coregulator | intracellular protein |
| | activity | transport |
| apoptotic process | ATPase binding | transdifferentiation |
| mitochondrial electron | leukocyte migration | |
| transport | | |
| ATPase inhibitor activity | tRNA modification | learning |
| autophagy of mitochondrion | bicellular tight junction | axis elongation |
| | assembly | |
| axon guidance | type 1 angiotensin receptor | lipid homeostasis |
| | binding | |
| vascular wound healing | ubiquitin binding | lymphocyte chemotaxis |
| BMP receptor binding | ubiquitin protein ligase binding | macrophage chemotaxis |
| blood circulation | ubiquitin protein ligase binding | macrophage differentiation |
| water channel activity | UV-damage excision repair | Cell diffrentiation |
| mature conventional dendritic | antimicrobial humoral immune | maintenance of epithelial |
| cell differentiation | response | cell apical/basal polarity |
| bone mineralization | vascular wound healing | vasodilation |
| brain development | vasoconstriction | melanocyte differentiation |
| brown fat cell differentiation | metal ion bindin | ion channel regulator |
| | | activity |
| virus receptor activity -KW | vein smooth muscle | memory |
| | contraction | |
| calcium ion transmembrane | mitochondrial electron | metanephric glomerular |
| transport | transport, NADH to ubiquinone | mesangial cell |
| | | differentiation |
| adenylate cyclase-activating | blood vessel endothelial cell | Wnt signaling involved in |
| adrenergic receptor signaling | proliferation involved in | midbrain dopaminergic |
| pathway | angiogenesis | neuron differentiation |
| response to heat | mammary gland development | mesangial cell-matrix |
| | | adhesion |
| mitochondrion organization | mitotic cell cycle | vesicle-mediated transport |

These data clearly show that by the products used it is possible to manage genes activity without of any multiple cell processes including KRAS/BRAF/MEK pathway.

### Example 28: Products and Method for Managing Eukaryotic Cell Behavior

In this study we used Ehrlich Ascites Carcinoma cells as a tumor cell culture and mouse fibroblasts as a non-tumor. Cells were cultured in RPMI 1640 medium containing 10% heat inactivated fetal bovine serum (FBS) (Sigma), 100 g/mL streptomycin and 100 U/mL penicillin G in a humidified atmosphere of 5% CO2 in air at 37 C (all Sigma). Prior to use of the tested compounds, DMEM was removed from cell monolayers, and cells were treated with tested products at 37° C. for 15-60 min in fresh DMEM without FBS. Then, cell monolayers were washed three times with PBS to eliminate remaining tested products. Negative control—H202.

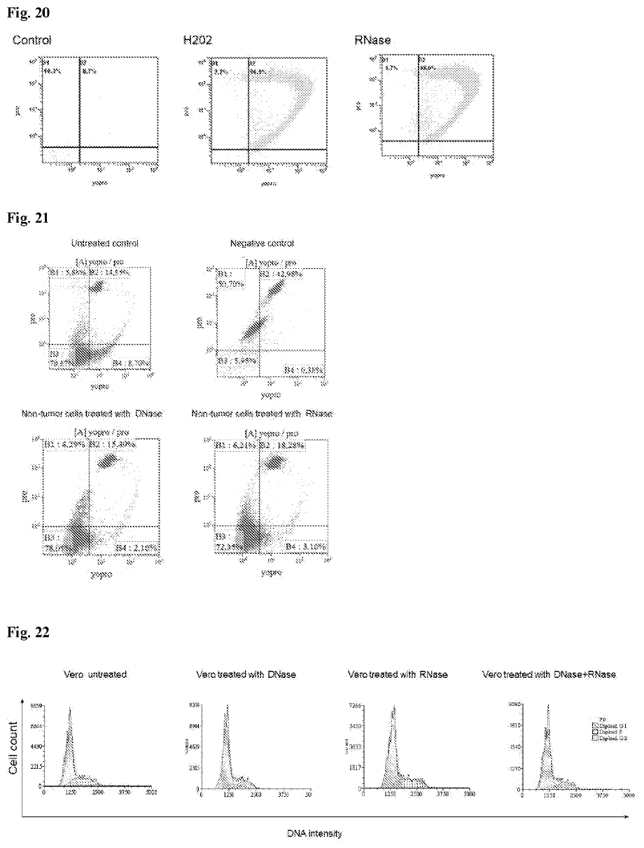

For flow cytometric subconfluent cell cultures were collected, washed twice with DMEM without FBS, and resuspended in DMEM supplemented with FBS. Products were added at a final concentration of from 1.0 to 100 μg/ml for 1.0 to 120 min as previously described. Cells were washed from nucleases and incubated for another 2.5 h in fresh DMEM with FBS at 37° C. as previously described. Cells were suspended in PBS containing 0.2 μM YO-PRO-1 (Invitrogen, Y3603) and 1.5 μM PI (Invitrogen, P3566). In total, 10,000 cells were analyzed for each measurement. The percentage of apoptotic cells was determined by flow cytometry using a CytoFLEX flow cytometer (Beckman Coulter, Brea, CA, USA). Cells undergoing apoptosis were stained with YO-PRO-1 but were impermeable to PI. Dead cells and cells in late apoptosis were permeable to both dyes. The results were expressed as the percentage of permeabilized cells. The experiment was performed in triplicate. Data were analyzed using FlowJo 10 software (Treestar Inc., Ashland, US). Data are presented in table 30, FIGS. 19-21.

TABLE 30
Effect of products on tumor cells and non-tumor cells

| | Tumor cells | | | Non-tumor cells | | |
|---|---|---|---|---|---|---|
| | | Early | Late | | Early | Late |
| | Alive | apoptosis | apoptosis | Alive | apoptosis | apoptosis |
| Treatemnt | (%) | (%) | (%) | (%) | (%) | (%) |
| Intact control | 69.1 | 9.5 | 21.4 | 70.87 | 8.70 | 20.43 |
| Negative control | 7.1 | 0 | 92.9 | 5.95 | 0.38 | 9.67 |
| Modified DNase 10 μg/mL | 27.09 | 3.1 | 69.81 | 78.05 | 2.14 | 19.81 |
| 15 min | | | | | | |
| Modified RNase 10 μg/mL | 0 | 7.1 | 92.9 | 72.35 | 3.16 | 24.49 |
| 15 min | | | | | | |
| DNase 10 μg/mL 15 min | 64.78 | 9.7 | 25.52 | 83.72 | 7.55 | 8.73 |
| RNase 10 μg/mL 15 min | 3.82 | 7.42 | 88.76 | 82.35 | 3.16 | 14.49 |
| EcoRV 10 μg/mL 15 min | 2.87 | 6.52 | 90.61 | 74.55 | 6.51 | 18.94 |
| RNA aptamer 1 μg/mL | 5.88 | 1.98 | 92.14 | 80.21 | 4.49 | 15.30 |
| 60 min | | | | | | |
| 2-Aminobenzimidazole | 7.18 | 10.09 | 82.73 | 74.75 | 10.69 | 14.56 |
| derivative 100 μg/mL 30 min | | | | | | |

Data clearly show that surprisingly tested products increase viability of non-tumor cells while in tumor cells, have the opposite effect, reducing their viability.

### Example 29: Products and Method for Managing Eukaryotic Cell Cycle

The effects of tested products on cell cycle phases were analyzed using flow cytometry in Vero cells (FIG. 22). Quantitative analysis of the distribution or proportion of cells in each phase was performed from at least 10,000 cells per sample. Each bar represents the mean±SD of the data obtained from three independent experiments. ****p<0.0001

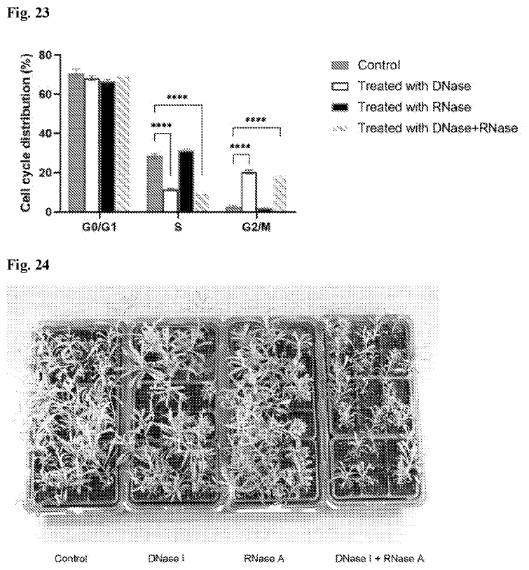

Quantitative data revealed that Vero cells following separation from extracellular matrix and treatment with products. accelerated S phase progression (FIG. 23). Thus, Vero cells treated with DNase 25 μg/mL alone or in combination with RNase 25 μg/mL DNase had an approximate 2.4-fold reduced distribution in S phase and 7.6-fold increased distribution in G2 phase (p<0.0001). Similarity, Vero cells treated with DNase and RNase showed a 2.7-fold lesser proportion in S phase with a 7.2-fold increase in G2 phase (p<0.0001). In both cases, the number of cells in the G1 phase continued to remain the same as that seen in controls. These results show that the products control S-phase length and modulate DNA replication, p53-DREAM pathway and S-phase checkpoint kinases.

### Example 30: Products and Method for Managing Cancer Therapy and Increase of Chemotherapy Efficacy

We studied the products for brain tumors' treatment. Acute growth inhibition/cytotoxicity assays was found following the exposure of the U87-MG human glioblastoma cells seeded at 3.0×10e4 cells/well in 24-well plates (Corning), separated from the extracellular matrix and treated with the tested products (taken at concentrations from 0.01 to 250 g/mL for the 5-60 minutes treatment in the presence or absence of temozolomide (200 mM) for 72 h. Cells were counted using a Z2 coulter particle count and size analyzer (Beckman Coulter).

U87-MG human glioblastoma cells were maintained in DMEM media supplemented with 10%0 FBS (Sigma), L-glutamine and antibiotics (all Sigma).

A significant difference was observed between temozolomide treated tumors and tumors after products and temozolomide. Data are shown in table 31.

TABLE 31
Effect of tested products for anticancer therapy

| | Normalized cell | | | Normalized cell | |
|---|---|---|---|---|---|
| | number | | | number | |
| Group | DMSO | Temozolomide | Group | DMSO | Temozolomide |
| Control | 1 | 0.65 | pyrrole-imidazole- | 0.30* | 0.1* |
| | | | pyrrole oligomer | | |
| | | | 100 μg/ml | | |
| DNase I | 0.55* | 0.2* | Sm protein 100 μg/ml | 0.60* | 0.35* |
| 10 μg/ml | | | | | |
| DNase I 1 pg/ml | 0.7* | 0.4* | RNase I 1 pg/ml | 0.55* | 0.3* |
| RNase | 0.45* | 0.15* | Linezolid 0.01 μg/ml | 0.80* | 0.45* |
| A10 μg/ml | | | | | |
| DNaseA + RNase | 0.35* | 0* | Propidium iodine 1 | 0.45* | 0.3* |
| A (each | | | μg/ml | | |
| 10 μg/ml) | | | | | |
| Loop-sheet- | 0.65* | 0.35* | Polymerase (T4) + | 0.35* | 0.2* |
| helix family: | | | Sm protein (10 μg/ml | | |
| 1tsr 250 μg/ml | | | each) | | |
| Polymerase | 0.75* | 0.5* | Antibody against | 0.25* | 0* |
| (T4) 50 μg/ml | | | surface-bound DNA + | | |
| | | | Antibody against | | |
| | | | surface-bound RNA | | |
| | | | (10 μg/ml each) | | |

*p < 0.05

Data present indicate that tested products, unexpectedly affected the viability of glioma cells and potentiated the efficacy of chemotherapy.

### Example 31: Products and Method for Managing of Eukaryotic Cells in Chemotherapy Resistance

Cells A549 (wild-type EGFR/mutant K-Ras) maintained in RPMI-1640 medium (Sigma, USA), supplemented with 10% heat-inactivated fetal bovine serum (Thermo Fisher), penicillin (100 U/ml), streptomycin (100 μg/ml) and L-glutamine (2 mM) at 37° C. in a 5% C02 atmosphere, and then harvested with trypsin-EDTA when the cells reached exponential growth. Cells were cultured in 96-well plates, in which the number of A549 was 6,000 per well. Prior to the treatment with tested products some cells were separated from the extracellular matrix. After that cells were exposed to Gemcitabine at different concentrations for 72 h in 96-well plates to determine the IC50. IC50 values of gemcitabine were determined by MTT (MTT solution was added to each well). The optical density (OD) of each well was measured at 490 nm following incubation for 4 h. The percentage of cell growth inhibition resulting from v was calculated as: [(OD 490 control cells−OD 490 treated cells)/OD 490 control cells]×100.

Table IC50, concentration resulting in inhibition of 50% of the maximal cell growth based on the type of product used. Data are shown in table 32.

TABLE 32
Effect of different products on sensitivity to anticancer therapy

| | IC50 of | | IC50 of |
|---|---|---|---|
| | Gemcitabine | | Gemcitabine |
| Compound | (nM) | Compound | (nM) |
| Control | 14.87 ± 1.26 | DNase 10 μg/ml | 10.20 ± 0.74* |
| | | added to medium | |
| DNase 10 μg/ml | 4.23 ± 0.38* | DNase 10 μg/ml | 8.62 ± 0.73* |
| | | added to medium | |
| RNase 10 μg/ml | 2.17 ± 0.51* | DNase 10 μg/ml | 8.30 ± 0.94* |
| | | added to medium | |
| DNase + RNase both 10 | 1.92 ± 0.16* | Antibody against | 2.72 ± 0.30* |
| μg/ml | | surface-bound DNA + | |
| | | Antibody against | |
| | | surface-bound RNA | |
| | | (10 μg/ml each) | |
| Ribocil* 10 μg/ml | 3.74 ± 0.45* | Antibody against | 9.30 ± 1.14* |
| | | surface-bound DNA + | |
| | | Antibody against | |
| | | surface-bound RNA | |
| | | (1 μg/ml each) | |
| Modified mitomycin | 2.24 ± 0.62* | Ribosomal | 4.8 ± 0.57* |
| C* 0.1 μg/ml | | protein S21* 100 | |
| | | μg/ml | |
| Modified tobramycin* | 4.86 ± 0.82* | Pentamidine* 10 | 6.25 ± 0.51* |
| 1 μg/ml | | μg/ml | |
| Histone H2 100 μg/ml | 7.53 ± 0.59* | Imidazole pyrrole | 8.62 ± 0.88* |
| | | pyrrole oligomer 50 | |
| | | μg/ml | |
| EcoNI 1 μg/ml | 4.39 ± 0.75* | REC J nuclease 1 | 7.49 ± 0.54* |
| | | μg/ml | |

*p < 0.05

The results obtained show that different products change cells sensitivity to chemotherapeutic agents. This effect is more pronounced when the extracellular matrix is removed and cell surface bound nucleic acids are affected.

### Example 32: Products and Method for Managing Neoplasm Transformation

We evaluated the role of tested products to prevent neoplastic transformation. For that, serum-supplemented medium of RWPE-1 cells was removed and the cell monolayer was washed once with PBS and once serum-free medium. After that the cells were treated with the tested compounds and exposed to phorbol 12 myristate (PMA) 50 ng/mL and the expression of MMVP9 was monitored. Data are presented in Table 33.

TABLE 33
Effect of products on antitumor response

| | MMP9 fold |
|---|---|
| Group | change |
| Control | 1 |
| Control + PMA | 2.9 ± 0.2 |
| RsaI 0.1 μg/ml | 1.3 ± 0.1* |
| Modified Bleomycin 1 μg/ml | 1.3 ± 0.1* |
| Histone H1 10 μg/ml | 1.4 ± 0.1* |
| Modified Histone H1 1000 μg/ml | 1.5 ± 0.1* |
| imidazole pyrrole pyrrole oligomer 10 μg/ml | 2.0 ± 0.3* |
| Modified imidazole pyrrole pyrrole oligomer 100 μg/ml | 1.4 ± 0.1* |
| Dnmt1 DNA-(cytosine-C5)-methyltransferase 50 μg/ml | 1.3 ± 0.1* |
| RNase H1 0.1 μg/ml | 1.3 ± 0.1* |
| Ribosomal protein S1 10 μg/ml | 1.5 ± 0.3* |
| Modified Ribosomal protein S1 1 μg/ml | 1.6 ± 0.1* |
| T7 RNA polymerases 1 μg/ml | 1.7 ± 0.1* |
| Neomycin 10 μg/ml | 1.5 ± 0.3* |
| RNA methylase 100 μg/ml | 1.3 ± 0.1* |
| DNA Methyltransferases 10 mg/ml | 1.2 ± 0.1* |
| Propidium iodine 10 μg/ml | 1.4 ± 0.1* |
| DNase I 10 μg/ml | 2.1 ± 0.2* |
| RNase A 10 μg/ml | 2.0 ± 0.3* |
| DNase I + RNase A (each 5 μg/ml) | 0.9 ± 0.3* |

*p < 0.05

It is clearly seen that the tested products inhibited cancer transformation and modulates anticancer response.

### Example 33: Products and Method for Managing the Growth of Eukaryotic Cells

In this study we used Ehrlich Ascites Carcinoma cells as a tumor cell culture and mouse fibroblasts as a non-tumor. Cells were cultured in RPMI 1640 medium containing 10% heat inactivated fetal bovine serum (FBS) (Sigma), 100 g/mL streptomycin and 100 U/mL penicillin G in a humidified atmosphere of 5% CO2 in air at 37 C (all Sigma).

Cells were treated with Ribavirin, Abacavir, Azidothymidine, Tenofovir, Etravirine, Lamividine, potassium orotate all taken in concentration from 0.1 ag/ml up to 100 μg/ml. Optical density OD600 (microtiter plate reader (Epoch 2—BioTek) every hour at 37 C. Data are shown in table 34.

TABLE 34
Effect of products on tumor growth

| | OD600 nm | | Cells | Cell |
|---|---|---|---|---|
| Drug | 24 h | 48 h | square | perimeter |
| Control | 0.095 | 0.205 | 24 014 | 658 |
| Ribavirin | 0.054* | 0.102* | 36 880* | 619 |
| Abacavir | 0.022* | — | 32 861 | 649 |
| Azidothymidine | 0.099 | 0.065* | 31 279 | 661 |
| Tenofovir | 0.016* | 0.106* | 37 945* | 743* |
| Etravirine | 0.050* | — | 37 958 | 705 |
| Lamividine | 0.075* | 0.120* | 28 042 | 664 |
| Potassium orotate | 0.009* | 0.072* | 44 858* | 782* |

*p < 0.05

The data obtained unexpectedly indicate that the use of tested products allows to change the properties of cancer cells. Potassium Orotate and Tenofovir also changed cell size.

### Example 34. Products and Method for Managing of Eukaryotic Cells' Memory

We studied the effects of tested products on eucaryotic cells memory formation using an ‘adaptive’ memory experiment. We used 10 different Candida albicans strains of clinical isolates. C. albicans were cultivated for 24 h on a Sabouraud media, washed from the extracellular matrix and either left untreated (control) or treated with the tested products as previously described. The time required for the cells to begin utilize maltose was expressed as a duration of lag phase during first and second exposure to this xenobiotic. To modulate the secondary maltose exposure, we collected control cells grown for 18 h on M9 broth supplemented with maltose 50 g/mL (that corresponds to the first exposure), treated them or not treated with tested products, adjusted OD600 and then again plated to the M9 broth supplemented with maltose for the second maltose exposure. Data are presented in Table 35.

TABLE 35
Products for managing cell memory of eukaryotes

| | Mean log phase (hours) | | |
|---|---|---|---|
| | across 10 strains | | |
| | of C. albicans | | |
| | Primary | Secondary | |
| | Maltose | Maltose | |
| Products | exposure | exposure | p |
| Control | 3 ± 1 | 1 ± 1 | |
| DNase I 10 μg/mL | 4 ± 1 | 4 ± 1 | <0.05 |
| DNase 0.001 μg/mL | 4 ± 1 | 2 ± 1 | <0.05 |
| Histone 3 100 μg/mL | 4 ± 1 | 4 ± 1 | <0.05 |
| Histone-3 0.1 μg/mL | 4 ± 1 | 4 ± 1 | <0.05 |
| RNase I 10 μg/mL | 6 ± 3 | 3 ± 1 | <0.05 |
| RNase 0.001 μg/mL | 6 ± 1 | 7 ± 1 | <0.05 |
| RNAsubunit-30 1000 μg/mL | 6 ± 1 | 3 ± 0 | <0.05 |
| RNAsubunit-30 0.1 μg/mL | 6 ± 1 | 3 ± 0 | <0.05 |
| DNase I + RNase 100 μg/mL | 7 ± 2 | 6 ± 1 | <0.05 |
| DNase I + RNase 0.001 μg/mL | 7 ± 1 | 4 ± 1 | <0.05 |
| Branaplam 100 μg/mL | 7 ± 2 | 6 ± 1 | <0.05 |
| Ribosomal protein S18 1 μg/mL | 7 ± 0 | 3 ± 1 | <0.05 |
| HindIII 10 μg/mL | 4 ± 1 | 4 ± 1 | <0.05 |
| Ribosomal protein eS31 10 | 6 ± 1 | 6 ± 0 | <0.05 |
| μg/mL | | | |
| DNA polymerase T7 0.1 | 5 ± 1 | 5 ± 1 | <0.05 |
| μg/mL | | | |
| Imidazole pyrrole pyrrole | 6 ± 1 | 6 ± 1 | <0.05 |
| oligomer 100 μg/mL | | | |
| TATA protein 10 μg/mL | 6 ± 1 | 7 ± 1 | <0.05 |
| EBNA1 100 μg/mL | 3 ± 1 | 3 ± 1 | <0.05 |
| Helix-loop-helix family protein | 4 ± 1 | 4 ± 0 | <0.05 |
| 100 μg/mL | | | |

It is clearly seen that control C. albicans could “remember” the first exposure and the second exposure to maltose shortened the lag phase by 3 h, meaning that bacteria could “remember” the first exposure and start utilize maltose faster. We found the tested products were able to prevent memorization by cells, thus cells were unable to recognize second exposure to maltose.

### Example 35: Products and Methods for Cells Memory Managing

We used an ‘adaptive’ memory experiment to generate C. albicans with the “memory” for maltose as described above. In this study we used 10 different strains of C. albicans, cultivated as previously described. Next, we exposed “maltose-sentient” C. albicans treated with tested products in a range of concentrations from 1 μg/ml up to 10 mg/ml for 30 sec-24 h. Cells were treated, either with tested products once or had multiple rounds of treatment followed by a wash-out period in minimal media without nutrients (i.e. M9 media without maltose). As depicted in tables 36 and 37, one cycles of cell's treatment and restoration for 24 h did not affect the memory of maltose-sentient cells, and the time lag of such cells during the second maltose exposure was shortened, compared with that in maltose-naïve cells. However, for some cells conducting over two rounds, and for all cells conducting over three rounds of treatment with nucleases and other tested products with formation of a so-called “zero” state led to the forgetting of the previous exposure to maltose. Thus, the behavior of C. albicans at “zero state” at the second contact with maltose was similar to that of control C. albicans at the first maltose exposure, with a minimal time of contact to trigger maltose utilization of 3 h.

Data received that the use of the tested products and putting the cells to a “zero state” can be used to modulate cell memory and forgetting.

TABLE 36
Effect of number of treatment cycles
with nucleases on cell memory

| | Number of strains |
|---|---|
| | with erased memory |
| Treatment regimen | to maltose |
| One-time treatment DNase I 1 μg/mL, 10 min | 0/10 |
| One-time treatment DNase I 1000 μg/mL, 6 h | 0/10 |
| One-time treatment RNase 1 pg/mL, 10 min | 0/10 |
| One-time treatment RNase 1000 μg/mL, 6 h | 0/10 |
| One-time treatment DNase + RNase 1 pg/mL, | 0/10 |
| 10 min | |
| One-time treatment DNase + RNase 1000 | 0/10 |
| μg/mL, 6 h | |
| Two-time treatment DNase I 1 pg/mL, 10 min | 0/10 |
| Two-time treatment DNase I 1000 μg/mL, 6 h | 0/10 |
| Two-time treatment RNase 1 pg/mL, 10 min | 2/10 |
| Two-time treatment RNase 1000 μg/mL, 6 h | 3/10 |
| Two-time treatment DNase + RNase 1 pg/mL, | 2/10 |
| 10 min | |
| Two-time treatment DNase + RNase 1000 | 2/10 |
| μg/mL, 6 h | |
| Three-time treatment DNase I 1 pg/mL, 10 min | 3/10 |
| Three-time treatment DNase I 1000 μg/mL, 6 h | 3/10 |
| Three-time treatment RNase 1 pg/mL, 10 min | 10/10 |
| Three-time treatment RNase 1000 μg/mL, 6 h | 10/10 |
| Three-time treatment DNase + RNase 1 pg/mL, | 7/10 |
| 10 min | |
| Three-time treatment DNase + RNase 1000 | 6/10 |
| μg/mL, 6 h | |

TABLE 97
Effect of tested compounds to erase cell memory

| | Treatment, | minimal time of | |
|---|---|---|---|
| | concentration, | contact to trigger | |
| | treatment time | maltose utilization | |
| maltose- | Control | 3 | h |
| naïve | | | |
| maltose- | Control | 1 | h |
| sentient | | | |
| | DNase I 1 pg/mL, 10 min | 1 | h |
| | Zero-D cells | 3 | h |
| | (DNase I 1 pg/mL, 10 min) | | |
| | DNase I 10 mg/mL, 24 h | 1 | h |
| | Zero-D cells | 3 | h |
| | (DNase I 10 mg/mL, 60 min) | | |
| | RNase A 1 pg/mL, 30 sec | 1 | h |
| | Zero-R cells | 3 | h |
| | (RNase A 1 pg/mL, 30 sec) | | |
| | RNase A 100 μg/mL, 24 h | 1 | h |
| maltose- | 1-time DNase I + RNase | 0.5 | h |
| sentient | A, each 100 μg/mL, 30 min | | |
| | Zero-DR cells | 2 | h |
| | (DNase I + RNase A, each | | |
| | 100 μg/mL, 30 min) | | |
| | Histone H2B + T7 RNA | 1 | h |
| | polymerases, each 100 | | |
| | μg/mL, 240 min | | |
| | Zero-DR cells | 3 | h |
| | (Histone H2B + T7 RNA | | |
| | polymerases, each 100 | | |
| | μg/mL, 240 min) | | |
| | Zero-R cells | 3 | h |
| | (RNase A 100 μg/mL, 2 h) | | |
| | Zero-DR cells | 3 | h |
| | (5-times TATA box- | | |
| | binding + Ribosomal | | |
| | protein S40 each 10 | | |
| | μg/mL, 60 min) | | |
| | Zero-DR cells | 3 | h |
| | (modified Bleomycin + | | |
| | modified tobramycin, each | | |
| | 1 μg/mL, 10 min) | | |
| | Zero-DR cells | 3 | h |
| | (propidium iodine, 10 | | |
| | μg/mL, 30 min) | | |
| | Zero-DR cells | 3 | h |
| | (ApoI + RNase P, each 10 | | |
| | μg/mL, 30 min) | | |

### Example 36: Products and Methods for the Managing of Cells' Forgetting

Given a broad range of cell memories that are able to be managed and erased with tested compounds, we next decided to trigger cell forgetting of its resistance to certain therapies. For that human breast cancer cells MCF-7 resistant to adriamycin (ADR) (MCF-7/ADR) were cultivated in RPMI 1640 medium supplemented with 10% FBS, 0.1 mg/mL streptomycin and 100 units/mL penicillin at 37° C. and 5% CO2.

Cells were either washed from the extracellular matrix or were not separated from the matrix and were treated with tested products taken at 25 μg/mL for 15 minutes. Some cells were treated with tested products to generate “zero-cells” as previously described. After that, tested compounds were washed out and cells were seeded in 96-well plates (8000 cells/well) and then treated with different concentrations of ADR. The ability of cells to forget was determined as the cells that were able to withstand therapy which was determined using an MTT assay as described above. Data are shown in table 38.

TABLE 38
Effects of tested compounds on cell's memories

| | | % of cells that forgot resistance to ADR | |
|---|---|---|---|
| | | (% survival) | |
| | | Tested | Tested compounds + |
| Group | | compounds | ADR 25 μM |
| Extracellular | Control | 100% | 81% |
| matrix | Cut-D (DNase I) | 87% | 53%* |
| removed | Cut-R (RNase A) | 92% | 47%* |
| | Cut-DR (DNase I + RNase) | 85% | 44%* |
| | Zero-D cells (DNase) | 95% | 28%* |
| | Zero-R cells (RNase) | 92% | 3%* |
| | Zero-DR cells (DNase + RNase) | 109% | 0%* |
| | Three-time treatment netropsin + | 87% | 14%* |
| | RNA helicase (Zero-DR) | | |
| | Three-time treatment Histone H3 + | 114% | 9%* |
| | Ribosomal protein L3 (Zero-DR) | | |
| | Modified amikacin | 89% | 62%* |
| | ADAR1 | 91% | 73%* |
| | T7 RNA polymerase | 98% | 66%* |
| | Histone H2A | 106% | 54%* |
| Extracellular | Control | 100% | 84% |
| matrix | Cut-D (DNase I ) | 91% | 64%*^{,}** |
| not | Cut-R (RNase A) | 88% | 71%*^{,}** |
| removed | Cut-DR (DNase I + RNase) | 88% | 62%*^{,}** |
| | Zero-D cells (DNase) | 110% | 49%*^{,}** |
| | Zero-R cells (RNase) | 104% | 41%*^{,}** |
| | Zero-DR cells (DNase + RNase) | 87% | 37%*^{,}** |
| | Three-time treatment netropsin + | 85% | 50%*^{,}** |
| | RNA helicase (Zero-DR) | | |
| | Three-time treatment Histone H3 + | 83% | 42%*^{,}** |
| | Ribosomal protein L3 (Zero-DR) | | |
| | Modified amikacin | 106% | 87%*^{,}** |
| | ADAR1 | 98% | 84%*^{,}** |
| | T7 RNA polymerase | 103% | 72%*^{,}** |
| | Histone H2A | 96% | 67%*^{,}** |

*p < 0.05 comparing with control;
**p < 0.05 between probes in which extracellular matrix was removed vs extracellular matrix was left

Data received clearly show that cells after the treatment with tested compounds were able to forget the pattern of ADR resistance and to become sensitive for it.

### Example 37: Products and Method for the Treatment of Tumors by Product—Antibody Conjugates

Human adenocarcinomic alveolar epithelial cell line A549 cell line was grown in DMEM medium (Sigma), supplemented with 10% fetal bovine serum (Gibco) and 1% streptomycin (Sigma).

A549 cells were seeded at a density of 5×10e5 cells per well into 6-well plates (Coring) for 24 h at 37 C. Next the culture medium was replaced with fresh medium and washed from the extracellular matrix with extracellular TezRs and next placed to the fresh media supplemented or not containing monoclonal antibody Cetuximab (IMC-C225) a recombinant, chimeric monoclonal antibody that binds to the extracellular domain of the epidermal growth factor receptor.

Products were conjugated with cysteamine hydrochloride (7.0 ng, 60 pmol in 2.2 μL PBS, pH 8.8) for 1 h at room temperature. The reaction solution was transferred to a tube with p-SCN-Bz-DOTA (35 μg, 49.0 nmol) and reacted for 1 h at room temperature. The reaction mixture was centrifuged at 1000×g for 40 min and pellet was resuspended with deionized. The C225 (1.0 mg, 6.58 nmol, 2 mg mL-1) was modified with N-succinimidyl S-acetylthioacetate (15.5 μg, 66. nmol) for 1 h at room temperature and applied to a Sephadex G50 superfine column. DNA-abzymes were obtained in HMI lab (know-how of prof. V.Tets).

SATA-modified C225 (1 mL, 400 μg mL-1) was treated with hydroxylamine (200 μL, 0.5 M) at room temperature for 2 h and applied to a Sephadex G50 superfine column. C225-SH (10 μg mL-1) was conjugated with DOTA-DNase, DOTA-RNase, suspension (10×1010 particles mL-1).

Probes: Probes were incubated for 24 h, media was replaced and cells were counted on the next day with a cell counter after the cells were removed from the plates pre-made trypsin-EDTA solution (Sigma).

Cells, grown on the coverslips were stained with propidium iodide (Sigma) according to the protocol: 50 μl of 15 μM propidium iodide was added per well before incubating additional 15 minutes on the orbital shaker in the dark and measuring fluorescence intensity with the same filter sets. Presence of a particular receptor was determined by measuring fluorescence intensity with microplate reader (Synergy Neo2, BioTek, VT, USA) using a 488/20 nm excitation filter and 645/40 nm emission filter. Data are shown in table 39 and 40.

TABLE 39
Difference in the fluorescence of propidium iodide
following the destruction of certain cell-surface
bound nucleic with antibody-nucleases conjugates.

| Probe | Target | Fluorescence |
|---|---|---|
| C225 (6 × 10⁹ particles mL−1) | NA | 100% |
| C225-DNase (6 × 10⁹ | cell-surface bound | 42% |
| particles mL−1) | DNA | |
| C225-RNase (6 × 10⁹ | cell-surface bound | 58% |
| particles mL−1) | RNA | |
| C225-DNase + C225-RNase (6 × 10⁹ | cell-surface bound | 0% |
| particles mL−1) | DNA + RNA | |

TABLE 40
Effect of products on cell proliferation

| | Cell |
|---|---|
| | number |
| | (% of |
| Probe | control) |
| Control | 100 |
| C225 (6 × 10⁹ particles mL−1) | 86 |
| C225-DNase (6 × 10⁹ particles mL−1) | 54* |
| C225-RNase (6 × 10⁹ particles mL−1) | 43* |
| C225-DNase + C225-RNase (6 × 10⁹ particles mL−1) | 18* |
| C225 (6 × 10⁹ particles mL−1) + gemcitabine 1 ug/mL | 56* |
| C225-DNase (6 × 10⁹ particles mL−1) + gemcitabine 1 ug/mL | 5* |
| C225-RNase (6 × 10⁹ particles mL−1) + gemcitabine 1 ug/mL | 9* |
| C225-DNase + C225-RNase (6 × 10⁹ particles | 0* |
| mL−1) + gemcitabine 1 ug/mL | |
| DNA-abzyme | 41* |
| DNA-abzyme + gemcitabine 1 ug/mL | 17* |

*p < 0.05

As it is seen, the delivery of products that destroy cell-surface bound nucleic acids led to a significant antitumor effect alone and in combination with targeted antitumor therapy.

### Example 38: Products and Method for Managing of Different Side Effects of Therapy

We used SCTD-beige mice 4-6 weeks. Raji tumor cells (ATCC® CCL-86) were injected intraperitoneally and were allowed to grow for 21 days. 7.3 log 10 human 1928z CAR T cells were used to target B leukemia cells and trigger cytokine release syndrome.

### Groups:

1. Control—untreated
2. Antibodies against P. aeruginosa DNA from 1 μg/mL one time in 7 days
3. Antibodies against P. aeruginosa DNA from 1000 μg/mL two times a day
4. Antibodies against P. aeruginosa DNA 1 μg/mL one time every three days+Nevirapine 7.5 mg/kg once daily
5. Antibodies against P. aeruginosa DNA 1000 μg/mL two times a day+Nevirapine 7.5 mg/kg once daily
6. Antibodies against E. coli RNA from 1 μg/mL one time every five days
7. Antibodies against E. coli RNA from 1000 μg/mL two times a day
8. AntiD8 conjugated antibodies with DNase I two times a day
9. Antibodies against P. aeruginosa DNA from 1 μg/mL two times a day+lamivudine 10 μg/mL
10. Antibodies against P. aeruginosa DNA from 1 μg/mL two times a day+tenofovir 10 μg/mL
11. Antibodies against P. aeruginosa DNA from 1 μg/mL two times a day+etravirine 10 μg/mL
12. Antibodies against P. aeruginosa DNA from 1 μg/mL two times a day+abacavir 10 μg/mL

The survival data are presented in Table 41, below.

TABLE 41
Effect of products on the regulation of CAR-T therapy side effects

Dead/alive

| Group | Day 0 | Day 5 |
|---|---|---|
| Group 1 | 0/10 | 5/5 |
| Group 2 | 0/10 | 3/7 |
| Group 3 | 0/10 | 0/10 |
| Group 4 | 0/10 | 3/7 |
| Group 5 | 0/10 | 2/8 |
| Group 6 | 0/10 | 3/7 |
| Group 7 | 0/10 | 2/8 |
| Group 8 | 0/10 | 2/8 |
| Group 9 | 0/10 | 0/10 |
| Group 10 | 0/10 | 0/10 |
| Group 11 | 0/10 | 0/10 |
| Group 12 | 0/10 | 0/10 |

Data received show that the products alone and in combination with nucleoside inhibitors led to a significant amelioration of the cytokine release syndrome and other CAR-T therapy side effects

### Example 39: Products and Method of Managing of Disease-Associate Receptors Activity

Panc-1 cancer cells were grown in DMEM medium (Sigma), supplemented with 1000 fetal bovine serum (Gibco) and 10 streptomycin (Sigma) at 37° C. in a humidified atmosphere containing 50 CO2.

Analyzed migration of Panc-1 cells through the BD-Matrigel Invasion Chamber (24-transwell, 8 μm pore size). Cells were treated with tested products at concentrations varying from 1 to 1000 μg/mL as previously discussed, some cells were additionally treated with recombinant human-EGF 20 ng/ml (Sigma-Aldrich) washed in PBS, resuspended in DMEM (serum-free) and added to the upper compartment of the Invasion Chamber (1×10e5 cells/well). Into the lower compartment of the chamber, conditioned medium was placed. After 24 h of incubation at 37 C, the cells on the upper surface were completely removed by wiping with a cotton swab,

After incubation, cells remained in upper surface of the membrane were removed by wiping with a cotton swab. Cells that had migrated from the upper to the lower side of the filter were fixed with methanol, stained with crystal violet solution and counted with a light microscope (40 fields/filter) (table 42).

TABLE 42
Effect of tested products on disease-associate pathways

| Group | Relative invasion (%) |
|---|---|
| Control | 100 |
| Untreated, EGF stimulated | 297 ± 34 |
| DNase I, EGF stimulated | 213 ± 28* |
| RNase I, EGF stimulated | 157 ± 33* |
| DNase I + RNase, EGF stimulated | 192 ± 37* |
| Zidovudine (AZT), Tenofovir (TNF), | 246 ± 31 |
| Nevirapine (NVP) and etravirine (ETR) at | |
| 5 μg/mL, EGF stimulated | |
| DNase I + Zidovudine (AZT), Tenofovir | 115 ± 20* |
| (TNF), Nevirapine (NVP) and etravirine | |
| (ETR) at 5 μg/mL, EGF stimulated | |
| RNase + Zidovudine (AZT), Tenofovir | 102 ± 18* |
| (TNF), Nevirapine (NVP) and etravirine | |
| (ETR) at 5 μg/mL, EGF stimulated | |
| Zero-D cells, EGF stimulated | 123 ± 35* |
| Zero-R cells, EGF stimulated | 107 ± 22* |
| Zero-DR cells, EGF stimulated | 101 ± 8* |
| Antibodies against cell-surface bound | 204 ± 41* |
| DNA, EGF stimulated | |
| Antibodies against cell-surface bound | 188 ± 23* |
| RNA, EGF stimulated | |
| Antibodies against cell-surface bound | 164 ± 30* |
| DNA + RNA, EGF stimulated | |
| Histone H2A, EGF stimulated | 197 ± 38* |
| Ribosomal protein, EGF stimulated | 174 ± 12* |
| Histone H2A + Ribosomal protein, | 145 ± 31* |
| EGF stimulated | |
| TLR9, EGF stimulated | 153 ± 19* |

*p < 0.05 compared to stimulated cells

These data clearly show that the use of tested compounds including the formation of zero cells can be used to inhibit disease-associated reception, including EGFR phosphorylation and inactivation EGFR signaling pathway

### Example 40: Products and Method for Managing Fungal Sensitivity to Antifungal Drugs

Nystatin resistant strain of Candida albicans F4 were isolated from the extracellular matrix and treated with testing products as previously discussed. The resulting fungi were plated to Sabouraud dextrose agar supplemented with nystatin (Sigma) 5 μg/mL and incubated 24 h at 37° C. and the number of colony-forming units was accessed (table 43).

TABLE 43
C. albicans antifungal drug sensitivity

| | C. albicans | | C. albicans |
|---|---|---|---|
| Product | CFU(log10)/mL | Product | CFU(log10)/mL |
| Control | 14.1 ± 0.2 | Ribosomal protein | 10.1 ± 0.2* |
| | | L26 | |
| DNase | 8.5 ± 0.3* | Small Nuclear | 7.3 ± 0.3* |
| | | Ribonucleoprotein | |
| RNase | 6.5 ± 0.8* | ββα-zinc finger | 6.4 ± 0.4* |
| | | family: Tramtrack | |
| | | protein | |
| DNase + | 4.2 ± 0.2* | NF-kappaB | 5.9 ± 0.2* |
| RNase | | | |

*p < 0.05

Tested products can increase sensitivity of fungi to antifungal antibiotics and allow to overcome antibiotic resistance.

### Example 41: Products and Methods for Disease Diagnosis

We studied the composition of cell-surface bound nucleic acids of normal and malignant cells. We used needle biopsy material of the colorectal cancer (Stage III) established from a biopsy specimen of a histologically confirmed adenocarcinoma or normal tumor tissues and PDX cells from BXPc3 (pancreatic cancer), BL0293 (bladder cancer), LG1049F (lung cancer), MC38 (colorectal cancer), BR1126F (breast cancer) and the PDX from patients with no malignancies.

The needle biopsy of the primary tumor/control was collected under sterile conditions into a specimen bottle containing RPMI 1640 medium supplemented with 5% penicillin-streptomycin-neomycin mixture (GIBCO). The specimen weighting 20 mg were put in each well of 12 well plate on shaker in fridge at 4° C. for 16-20 hr, supplemented with tripsin and then were carefully transferred or a fresh RPMI 1640 supplemented with 10% horse serum and penicillin-streptomycin to 1% of total solution. Then plates were put to warm water bath at 37° C. for 20 min and next transferred the tissue to a 20 ml vial containing Hanks' Balanced Salt Solution and gently shacked, then, 0.1% collagenase solution was added for 45 minutes at 37 C. After the tissue dissociation, probes were centrifuged at 100×g for 10 min at room temperature. Supernatant was removed and cell homogenate was resuspend in 2.5 ml RPMI 1640 media.

Cell-surface bound nucleic acids were visualized with DAPI, SYTOX green (Excitation: 504; Emission 523), Propidium Iodine (Excitation: 493; Emission 636) with Revolve microscope from ECHO (ECHO San Diego CA) and Synergy Neo2 Multi-Mode Microplate Reader (Biotek).

To isolate cell-surface bound DNA and/or RNA tumor and control cells were washed from the nutrient medium matrix in PBS with a subsequent centrifugation 3000 g×10 minutes. Next, cells were placed to a 0.9% NaCl supplemented with EchoR1 and HindIII nucleases, with added Mg buffer for 1 h at 37 C. Cells were separated by centrifugation 3000 g×10 minutes and supernatant was filtered through the 0.22 uM filter (Millipore). DNA was isolated from the supernatant with QIAamp DNA Mini Kit (Qiagen). The RNA was isolated with a Quick-RNA Kits (Zymo research).

Immune cells were obtained as described below with a Ficoll centrifugation.

The whole-genome sequence was obtained using the Illumina HiSeq 2500 sequencing platform (Illumina GAIIx, Illumina, San Diego, CA, USA). Library preparation, sequencing reactions, and runs were carried out according to the manufacturer's instructions. *Amount of cell-surface-bound nucleic acids of non-treaty control cells of each type was suggested as “norma”.

Also, some cells were stained with Sytox as described above and the alteration of the surface green fluorescence corresponds was analyzed as the sign of cell-surface-bound nucleic acids alterations. Data are shown in tables 44 and 45.

TABLE 44
Analysis of distribution of cell-surface-bound DNA and/or RNA on the surface of tumor vs normal cells

| | Type of cell* | | | | | | | | | | | |
|---|---|---|---|---|---|---|---|---|---|---|---|---|
| Type of | Biopsy | | | | | | | | | | | |
| cell- | derived | | | | | | | | | | | |
| surface- | tumor | | | | | | | | | | | |
| bound | cells | Normal | | Normal | | Normal | | Normal | | Normal | | Normal |
| nucleic | from | colon | | pancreatic | | bladder | | lung | | colon | | Breast |
| acid | CRC | cells | BXPc3 | cells | BL0293 | cells | LG1049F | cells | MC38 | cells | BR1126F | cells |
| DNA | Lower | Norma | Higher | Norma | Lower | Norma | Higher | Norma | Lower | Norma | Lower | Norma |
| R | Lower | Norma | Lower | Norma | Lower | Norma | Higher | Norma | Lower | Norma | Higher | Norma |
| D1/R1 | Lower | Norma | Higher | Norma | Lower | Norma | Lower | Norma | Lower | Norma | Higher | Norma |

TABLE 45
Sequence identity of cell-surface-bound DNA and/or RNA on the surface of tumor vs normal cells

| | Type of cell | | | | | | | | | | | |
|---|---|---|---|---|---|---|---|---|---|---|---|---|
| Type of | Biopsy | | | | | | | | | | | |
| cell- | derived | | | | | | | | | | | |
| surface- | tumor | | | | | | | | | | | |
| bound | cells | Normal | | Normal | | Normal | | Normal | | Normal | | Normal |
| nucleic | from | colon | | pancreatic | | bladder | | lung | | colon | | Breast |
| acid | CRC | cells | BXPc3 | cells | BL0293 | cells | LG1049F | cells | MC38 | cells | BR1126F | cells |
| DNA | SNPs, | Norma | SNPs, | Norma | SNPs, | Norma | SNPs, | Norma | SNPs, | Norma | SNPs, | Norma |
| | mutations | | mutations | | mutations | | mutations | | mutations | | mutations | |
| RNA | SNPs, | Norma | SNPs, | Norma | SNPs, | Norma | SNPs, | Norma | SNPs, | Norma | SNPs, | Norma |
| | mutations | | mutations | | mutations | | mutations | | mutations | | mutations | |
| DNA/ | SNPs, | Norma | SNPs, | Norma | SNPs, | Norma | SNPs, | Norma | SNPs, | Norma | SNPs, | Norma |
| RNA | mutations | | mutations | | mutations | | mutations | | mutations | | mutations | |

*Sequence of cell-surface-bound nucleic acids of control, untreated cells of each type was suggested as “normal”

Thus, in mammalian diseases, qualitatively-quantitative changes cell-surface-bound nucleic acids occur and can be used for diagnostic purposes of mammalian diseases.

### Example 42: Product and Method for Treatment Mental Illnesses

The experiment involved 25 volunteers from among people suffering from schizophrenia with severe agitation. For relief of exacerbation, volunteers received a drug given to them in conjunction with basic therapy. The efficacy was analyzed based on the Change in Total Positive and Negative Syndrome Scale (PANSS) Score within 2 weeks timeframe. Potassium orotate, Etinavir, Ribavirin, Abacavir, tobramycin, were given at regular doses; DNase, RNase were given orally 50 mg×BID. Data are shown in table 46.

TABLE 46
Change in Total PANSS Score From Baseline to
the End of the Double Blind Treatment Period

| | Total | | | Total | |
|---|---|---|---|---|---|
| | PANSS | | | PANSS | |
| | Base- | Week | | Base- | Week |
| Group | line | 2/3 | Group | line | 2/3 |
| Standard of care | 84.11 | 76.34 | Standard of care + | 82.55 | 64.73 |
| | | | DNase I | | |
| Standard of care + | 80.02 | 54.20 | Standard of care + | 81.16 | 67.14 |
| Potassium orotate | | | RNase | | |
| Standard of care + | 83.54 | 63.40 | Standard of care + | 80.41 | 55.30 |
| Etravirine | | | DNase + RNase | | |
| Standard of care + | 80.23 | 64.15 | Combined: | 82.58 | 44.34 |
| Abacavir | | | Potassium | | |
| | | | orotate + DNase | | |
| Standard of care + | 81.86 | 64.29 | Combined: | 81.17 | 46.24 |
| Ribavirin | | | Potassium | | |
| | | | orotate + RNase | | |
| Tobramycin | 82.58 | 67.60 | Combined: | 83.49 | 47.45 |
| | | | Potassium | | |
| | | | orotate + DNase + | | |
| | | | Etravirine | | |

Data received point out that the use of the tested products might be beneficial for mental and neurological disorders. Products can trigger auto-reprogramming and restoration of proper functions. We also found that the combined use of reverse inhibitors as products that inhibit cell-surface-bound nucleic acids formation and products that destroy them are highly effective for treatment of mental and psychiatric disorders.

### Example 43: Products and Method for Managing of Plant Growth

We measured the emergence of plants and the yield of the products on different plants including Arabidopsis spp. Dry, vernalized seeds were sterilized in microcentrifuge tubes with a 70% (v/v) ethanol wash followed by treatment in a solution of 50% (v/v) bleach and approximately 0.5% (v/v) Tween 20 for 10 min. The bleach solution was removed in a laminar flow hood with a sterile transfer pipette, and then the seeds were rinsed 8 to 10 times with sterile water. Seeds were incubated in the water solution containing different compounds that were previously shown to bind or inactivate cell-surface-bound nucleic acids taken at concentration from 0.01 μg/ml up to 1000 μg/ml.

Control seeds were put to the water with no tested compounds added. Next, seeds was sown at 5 cm depth in plowed, disked, and harrowed clay loam soil. The soil in some probes was supplemented with fertilizer according to the manufacture instruction. We measured the emergence, shoot length, root length and chlorophyll at day 5 or 7. The chlorophyll content of leaves was determined 7 d after seed placement. Fresh leaf material (50 mg) was homogenized in 10 ml of 95% ethanol. The homogenate was centrifuged at 1500×g for 20 min, and the supernatant was collected. was measured using a NanoDrop OneC spectrophotometer (ThermoFisher Scientific, Waltham, MA, USA) at 649 and 665 nm. The concentrations of chlorophyll-α, chlorophyll-β, and total chlorophyll (α+β) were calculated using the equations. The total chlorophyll content was determined using the following formula:

$$chlorophyll - α = 13.95 \times A 665 - 6.68 \times A 649 \qquad ( 1 )$$

$$chlorophyll - β = 24.96 \times A 649 - 7.32 \times A 649 \qquad ( 2 )$$

$$Total chlorophyll = ( chlorophyll - α + chlorophyll - β ) \times final volume of sample ( ml ) \times dilution fold / fresh weight of sample taken \qquad ( 3 )$$

The concentration was expressed as mg chlorophyll g-1 fresh weight by using the following equation:

$$Total chlorophyll \text{} ( mg g - 1 FW ) = [ 20.2 ( D 645 ) + 8.02 ( D 663 ) ] \times [ V / ( 1000 \times W ) ] ,$$

$$where V = volume of 80 \% aqueous acetone ( ml ) , \text{} W = weight of fresh leaf ( g ) , \text{} D 645 = absorbance at 645 nm wavelength , and D 663 = absorbance at 663 nm wavelength .$$

Products tested had a significant impact on seedling emergence and the germination percentages of plants.

As it can be seen, the use of the tested products, affected a variety of characteristics of plants and significantly increased the growth of the plants.

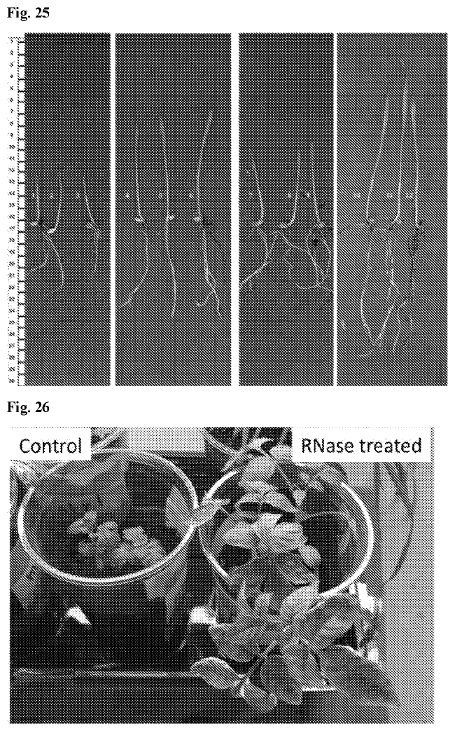

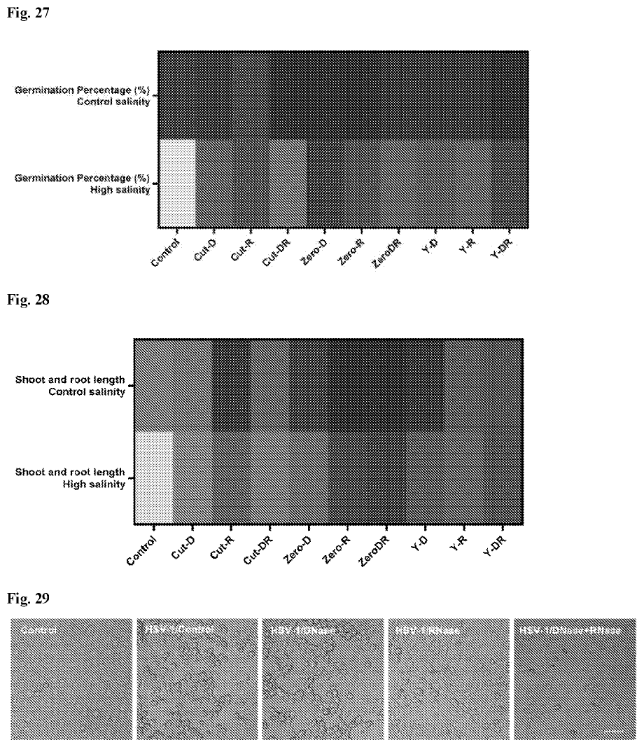

We also studied the effect of tested products on regulation of plants and seeds growth in optimal and stressful conditions (table 47, 48, 49 FIGS. 24, 25, 26).

TABLE 47
Effect of tested products on the time of seedling emergence
(50% of the seeds) and the germination percentages

| Product | Seedling emergence | | Germination percentages |
|---|---|---|---|
| Control | 16 | day | 33% |
| DNase I 10 μg/mL | 11 | day | 67% |
| RNase A 10 μg/mL | 11 | day | 50% |
| DNase + RNase 1 μg/mL | 7 | day | 81% |
| bZIP | 12 | day | 55% |
| Ribosomal protein eS1 | 11 | day | 59% |
| Netilmicin | 9 | day | 69% |
| Modified Netilmicin | 10 | day | 74% |
| Argonaute protein | 12 | day | 68% |
| bZIP + Netilmicin | 6 | day | 77% |

As it can be seen, the tomatoes grown following treatment with RNase exhibited much intense growth.

TABLE 48
Effect of tested products for managing of seeds germination and plants
(Arabidopsis spp) growth (in stressful temperature conditions)

| | Germination | Shoot | Root |
|---|---|---|---|
| | percentages | length (cm) | length (cm) |
| Product | at day 5 | at day 5 | at day 5 |
| Control | 33% | 3.4 | 2.8 |
| Etravirine | 75% | 6.1 | 7.0 |
| Raltegravir | 42% | 5.3 | 5.0 |
| Lopinavir + ritonavir | 80% | 5.7 | 6.4 |
| DNase | 75% | 5.4 | 4.6 |
| RNase | 46% | 4.1 | 4.4 |
| Tenofovir | 64% | 5.5 | 3.9 |
| Lamivudine | 55% | 4.7 | 4.7 |
| Abacavir | 63% | 5.1 | 6.8 |
| Azidothymidine | 76% | 5.6 | 5.2 |
| 2-chloro-5-phenyl-5H- | 40% | 5.05 | 4.7 |
| pyrimido[5′,4′:5,6]pyrano[2,3- | | | |
| d]pyrimidine-4-ol derivatives | | | |
| Etravirine and DNase | 67% | 6.9 | 7.6 |
| Etravirine and RNase | 54% | 10 | 9.2 |
| Raltegravir + DNase | 83% | 7.5 | 9.3 |
| RNase + Raltegravir | 79% | 7.2 | 8.0 |
| DNase and Lopinavir and | 71% | 5.6 | 6.6 |
| ritonavir | | | |
| RNase and Lopinavir and | 71% | 6.6 | 8.0 |
| ritonavir | | | |
| Trypsin | 50% | 7.6 | 9.0 |
| Proteinase K | 42% | 7.6 | 8.3 |

It can be clearly seen that tested products affect different plants characteristics.

TABLE 49
Effect of tested products on regulation of seeds germination and plants
(Arabidopsis spp) growth in optimal temperature conditions

| | Germination | Shoot | Root |
|---|---|---|---|
| | percentages | length (cm) | length (cm) |
| Product | at day 5 | at day 5 | at day 5 |
| Control | 45% | 6.25 | 4.4 |
| Nevirapine | 45% | 4.05 | 3.8 |
| Etravirine | 85% | 11.5 | 8.0 |
| Tenofovir | 75% | 10.75 | 7.8 |
| Lamivudine | 80% | 9.75 | 7.7 |
| Abacavir | 85% | 10.9 | 10.3 |
| Azidothymidine | 75% | 11.5 | 8.4 |
| 2-chloro-5-phenyl-5H- | 40% | 5.05 | 4.7 |
| pyrimido[5′,4′:5,6]pyrano[2,3-d]pyrimidine-4-ol | | | |
| derivatives | | | |
| Raltegravir | 70% | 8.6 | 6.8 |
| Lopinavir + ritonavir | 90% | 11.9 | 10.3 |
| DNase | 80% | 10.75 | 7.6 |
| RNase | 75% | 9.4 | 6.8 |
| DNase + RNase | 70% | 8 | 5.9 |
| Bleomycin | 77% | 9.8 | 6.6 |
| 1pdn | 71% | 8.3 | 7.5 |
| Histone H1 | 84% | 10.8 | 7.9 |
| 1d3u | 81% | 11.4 | 7.1 |
| Taq polymerase | 80% | 9.4 | 6.8 |
| Basic leucine zipper | 55% | 6.9 | 6.0 |
| Transcription factor TF_{II}A | 88% | 10.7 | 7.7 |
| netropsin | 76% | 10.4 | 6.8 |
| pyrrole-imidazole-pyrrole oligomer | 65% | 7.6 | 7.3 |
| 1,4-Bis{[1-(((5-(5-N- | 58% | 8.5 | 7.6 |
| isopropylamidino)benzimidazol-2-yl) furan-2- | | | |
| yl)methylene)-1H-1,2,3-triazole-4- | | | |
| yl]methyleneoxy}benzene hydrochloride | | | |
| NF-kappaB | 59% | 8.3 | 7.4 |
| T7 RNA polymerase | 63% | 9.2 | 8.3 |
| Ribosomal protein S1 | 73% | 8.7 | 6.4 |
| linezolid | 82% | 9.3 | 7.6 |
| riboflavin | 54% | 9.1 | 5.9 |
| Neomycin | 83% | 7.6 | 6.7 |
| pentamidine | 75% | 11.2 | 7.3 |
| Netilmicin | 63% | 8.2 | 7.3 |
| Propidium iodide | 83% | 9.7 | 6.5 |
| Tobramycin | 70% | 8.0 | 6.9 |
| Ribocil-D | 85% | 10.6 | 6.4 |
| Control | 43% | 6.1 | 4.3 |
| Modified Bleomycin | 89% | 10.4 | 7.4 |
| Modified 1pdn | 91% | 9.5 | 8.7 |
| Modified Histone H1 | 83% | 12.2 | 8.9 |
| Modified 1d3u | 90% | 14.7 | 7.5 |
| Modified Taq polymerase | 92% | 12.6 | 7.3 |
| Modified Basic leucine zipper | 67% | 9.0 | 7.2 |
| Modified Transcription factor TF_{II}A | 94% | 11.5 | 8.2 |
| Modified netropsin | 85% | 11.7 | 7.3 |
| Modified pyrrole-imidazole-pyrrole oligomer | 75% | 8.6 | 8.3 |
| Modified 1,4-Bis{[1-(((5-(5-N- | 69% | 9.3 | 8.5 |
| isopropylamidino)benzimidazol-2-yl) furan-2- | | | |
| yl)methylene)-1H-1,2,3-triazole-4- | | | |
| yl]methyleneoxy}benzene hydrochloride | | | |
| Modified NF-kappaB | 71% | 8.2 | 8.3 |
| Modified T7 RNA polymerase | 74% | 9.7 | 9.2 |
| Modified Ribosomal protein S1 | 85% | 6.3 | 7.5 |
| Modified linezolid | 90% | 9.9 | 8.9 |
| Modified riboflavin | 63% | 9.5 | 6.8 |
| Modified Neomycin | 82% | 8.7 | 7.3 |
| Modified pentamidine | 80% | 11.9 | 8.1 |
| Modified Netilmicin | 78% | 9.4 | 7.9 |
| Modified Propidium iodone | 89% | 10.5 | 7.3 |
| Modified Tobramycin | 88% | 8.4 | 7.6 |
| Modified Ribocil-D | 86% | 11.4 | 7.3 |

The effects tested products on plant characteristics was also assed in terms of chlorophyll amount (table 50, 51).

TABLE 50
Effect of tested products on chlorophyl content

| | chlorophyll | chlorophyll | Chlorophyll |
|---|---|---|---|
| | a | b | total |
| Product | mg/g | mg/g | mg/g |
| Control | 18.0 | 5.3 | 23.6 |
| Etravirine | 19.1 | 5.6 | 25.0 |
| Raltegravir | 19.5 | 6.2 | 25.9 |
| Lopinavir + ritonavir | 21.0 | 6.5 | 27.8 |
| DNase | 19.1 | 5.3 | 24.7 |
| RNase | 24.1 | 11.1 | 35.5 |
| Bleomycin | 22.6 | 7.9 | 26.3 |
| Histone H1 | 29.7 | 9.2 | 24.5 |
| NF-kappaB | 23.6 | 8.7 | 26.3 |
| Ribosomal protein S1 | 20.3 | 6.9 | 25.0 |
| Tobramycin | 22.7 | 8.5 | 28.4 |
| Modified Bleomycin | 23.8 | 7.9 | 27.7 |
| Modified Histone H1 | 30.7 | 10.1 | 26.7 |
| Modified NF-kappaB | 25.8 | 10.9 | 28.5 |
| Modified Ribosomal | 23.7 | 8.3 | 27.2 |
| protein S1 | | | |
| Modified Tobramycin | 24.8 | 9.2 | 29.3 |

It is clearly seen that tested products modulate chlorophyll content.

TABLE 51
Effect of tested products on product yield (soy) and plants
characteristics grown under stressful conditions

| | | |
|---|---|---|
| | Root length dark-induced | Number |
| | leaf senescence | of pods |
| Product | (15 days after germination) | from plant |

Without fertilizer

| | | |
|---|---|---|
| Control | 100% | 100% |
| DNase I | 187%* | 180%* |
| RNase I | 253%* | 171%* |
| DNase I + RNase I | 297%* | 209%* |
| DNA mismatch endonuclease | 202%* | 148%* |
| Benzimidazole | 160* | 177%* |
| Modified Benzimidazole + | 185%* | 213%* |
| Ribosomal S1-like | | |
| T6 gene exonuclease + | 248%* | 305%* |
| Ribosomal protein L25-5S | | |

With fertilizer (15 percent nitrogen, 30 percent
phosphorous, and 15 percent potassium)

| | | |
|---|---|---|
| Control | 100% | 100% |
| DNase I | 139%* | 156%* |
| RNase I | 192%* | 185%* |
| DNase I + RNase I | 215%* | 194%* |

*p < 0.05

Tested products have a significant impact on plants and product yield. Moreover, the use of these products allows to overcome stressful conditions for plants.

### Example 44: Products and Methods for Managing of Plant Growth

To study effects of nucleases use on plants tomato seeds were pretreated with DNase I o RNase A at concentrations from 10 to 10000 μg/mL for 60 minutes, washed from nucleases and sown in plastic trays and were transplanted with a single seedling in three liter capacity plastic pots filled with compost. The experiment was carried out in greenhouse with the medium temperature 22C and 34 humidity. Data are shown on table 52.

TABLE 52
Effect of tested products on plants characteristics

| | | | 30 days | | 30 days | Number | Number | | Number |
|---|---|---|---|---|---|---|---|---|---|
| | | | growth | 30 days | growth, | of | of | | of |
| | Germination | Seedling | Shoot | growth, | Seedling | flowers | fruits | | seeds |
| | percentage, | survival | length, | Root | length, | per | per | Fruit | per |
| Group | % (day 5) | percentage, % | cm | length | cm | plant | plant | weight | fruit |
| Control | 31.3 ± 1.411 | 49 ± 3.331 | 12 ± 1.25 | 6.5 ± 0.681 | 18.6 ± 1.667 | 23.3 ± 2.325 | 8.7 ± 1.923 | 2.2 ± 0.373 | 16.7 ± 1.411 |
| DNase I | 32.3 ± 2.823 | 58.7 ± 3.734 | 12.9 ± 1.154 | 4.8 ± 0.925 | 17.7 ± 1.321 | 30.3 ± 2.97 | 16.7 ± 2.325 | 3.4 ± 0.35 | 20 ± 1.848 |
| RNase I | 74 ± 4.234 | 82.7 ± 2.325 | 11.5 ± 0.832 | 7.3 ± 0.693 | 19.5 ± 4.016 | 31 ± 4.234 | 18 ± 1.848 | 2.9 ± 0.324 | 23.7 ± 2.823 |
| DNase I + | 35 ± 3.331 | 92.7 ± 1.923 | 14.5 ± 1.991 | 10.2 ± 0.971 | 25.1 ± 1.53 | 61.7 ± 1.4 | 38.3 ± 2.823 | 3.7 ± 0.437 | 39.7 ± 6.811 |
| RNase I | | | | | | | | | |

These data clearly show that tested compounds significantly improved plants characteristics

### Example 45: Products and Method for Managing Plants Characteristics

We measured the effect of different plant characteristics by different products using as a not-limiting examples of plants spring wheat, soy, tomato, rice, potato, barley, maize, oat, corn, cotton, cassava seeds were used. Dry, vernalized seeds were processed as described above and pretreated with tested compound. Data are presented in table 53.

TABLE 53
Performance of plants being treated with tested products.

| | Germination day 5 (% to control) | | | | | | |
|---|---|---|---|---|---|---|---|
| | | | | Treated | | | Treated |
| | | Treated | Treated | with | Treated | Treated | with |
| | | with | with | DNase I + | with | with | EcoRI + |
| Group | Control | DNase I | RNase I | RNase | EcoRI | Raltegravir | Raltegravir |
| wheat | 100 | 205* | 156* | 169* | 188* | 122* | 356* |
| soy | 100 | 134* | 98 | 155* | 140* | 83 | 295* |
| tomato | 100 | 207* | 192 | 279* | 155* | 163* | 351* |
| rice | 100 | 187* | 209* | 284* | 146* | 102* | 297* |
| potato | 100 | 172* | 105* | 133* | 162* | 94 | 190* |
| barley | 100 | 116* | 154* | 73* | 151* | 113 | 145* |
| maize | 100 | 162* | 172* | 179* | 141* | 109 | 232* |
| oat | 100 | 154* | 199* | 268* | 167* | 116 | 381* |
| corn | 100 | 187* | 105* | 224* | 253* | 122 | 443* |
| cassava | 100 | 207* | 283* | 150 | 264* | 194* | 372* |

It can be clearly seen that seeds treated with tested products, possess unique growth characteristics.

### Example 46: Products and Method for Seeds Treatment and Memory Management to be Passed Through Generations

Seeds of Dianthus amurensis were obtained after the one treatment with tested products (DNase I or/and RNase A) as described above. Seeds of the second generation were obtained from the plants that were grown following the treatment with tested products (without any additional nuclease treatment). Flower were cultivated according to recommendation of https://plantcaretoday.com/dianthus-care.html.

Seeds were transplanted into plastic nursery pot for plants (L×W×D of 3.25″×2.75″×2.75″) filled with a mixture of soil and peat moss (3:1, v/v) containing organic fertilizer. The temperature of the greenhouse was maintained at 25±2° C. and 10±2° C. during day and night, respectively. Each treatment consisted of three replicates and 1/100 plant were planted per plastic pot. At harvest, after treatment, plant growth parameters, including plant height, leaf area, flower weight, dry weight of leaf, stem and root, were determined (tables 54, 55). Plant height was determined by measuring the height from the stem base to first leaf. Leaf length was measured using ruler. After measuring the fresh weight, plant material was dried at 70° C. for 2 days to measure the corresponding dry weight (Kwon et al., 2019). The effect of treatment on chlorophyll stability was estimated by measuring the chlorophyll content following treatment. Chlorophyll was extracted from fresh leaf samples, from both treated and untreated plants as described above. The represented values were shown as mean±SE with a minimum of three independent replicates (n=3). Obtained results were considered statistically significant at p<0.05.

TABLE 54
Effect of Zero-state on plants characteristics (first generation)

| | | Plant | Leaf | Leaf | Stem | Root | | | Total |
|---|---|---|---|---|---|---|---|---|---|
| | | height | length | DW (g/ | DW (g/ | DW (g/ | Days | Flower | seed |
| | Germination | (cm), | (cm), | plant), | plant), | plant), | to | weight | weight, |
| Cells | (%) | 99 day | 99 day | 99 day | 99 day | 99 day | flower | (g/plant) | mg |
| Control | 91.7 ± 4.7 | 5.3 ± 2.8 | 2.4 ± 0.519 | 3.2 ± 0.2 | 4.5 ± 0.67 | 2.7 ± 0.85 | 210.25 ± 10.475 | 5.875 ± 0.395 | 152.7 ± 7.4 |
| | (±5.14%) | (±53.40%) | | | (±14.63%) | (±31.07%) | (±4.98%) | (±6.72%) | (±4.82%) |
| Zero-D | 93 ± 2.3 | 9 ± 2.23 | 3.3 ± 0.707 | 4.4 ± 0.3 | 4.5 ± 0.35 | 3.8 ± 0.51 | 185 ± 10.216 | 8.3 ± 0.788 | 114.7 ± 7.7 |
| seeds | (±3.22%) | (±25.15%) | (±21.41%) | (±6.80%) | (±7.63%) | (±13.55%) | (±5.52%) | (±9.49%) | (±6.72%) |
| Zero-R | 82.3 ± 1.8 | 6 ± 2.3 | 2.5 ± 0.5 | 4.1 ± 0.3 | 5.1 ± 0.23 | 2.5 ± 0.5 | 187.25 ± 13.799 | 8.625 ± 0.748 | 220.3 ± 14.3 |
| seeds | (±2.10%) | (±37.72%) | (±18.05%) | (±6.89%) | (±4.44%) | (±18.11%) | (±7.37%) | (±8.67%) | (±6.50%) |
| Zero-DR | 96.3 ± 1.7 | 3.7 ± 1.7 | 2.5 ± 0.3 | 5.0 ± 0.2 | 5.5 ± 0.3 | 4.7 ± 0.57 | 176.25 ± 9.845 | 6.45 ± 0.509 | 153.3 ± 9.6 |
| seeds | (±1.79%) | (±47.14%) | (±11.55%) | (±4.68%) | (±6.25%) | (±12.20%) | (±5.59%) | (±7.89%) | (±6.28%) |

TABLE 55
Characteristics of the second generation of plants grown from the seeds of plants which were tuned to “zero-state”

| | | Plant | Leaf | Stem | Root | | | Total |
|---|---|---|---|---|---|---|---|---|
| | | height | DW (g/ | DW (g/ | DW (g/ | Days | Flower | seed |
| | Germination | (cm), | plant), | plant), | plant), | to | weight | weight, |
| Cells | (%) | 99 day | 99 day | 99 day | 99 day | flower | (g/plant) | mg |
| Control | 81.75 ± 2.173 | 6.03 ± 0.864 | 3.7 ± 0.51 | 5.1 ± 0.3 | 3.7 ± 0.493 | 262.3 ± 3.5 | 6.7 ± 0.5 | 170.4 ± 4.538 |
| | (±2.66%) | (±14.33%) | (±13.92%) | (±5.62%) | (±13.33%) | (±1.32%) | (±7.00%) | (±2.66%) |
| Obtained | 82 ± 2.263 | 8 ± 0.408 | 4.6 ± 0.51 | 4.7 ± 0.5 | 5.4 ± 0.299 | 208.7 ± 3.9 | 9.5 ± 1.2 | 129 ± 4.249 |
| from plants | (±2.76%) | (±5.10%) | (±11.01%) | (±9.88%) | (±5.54%) | (±1.90%) | (±13.11%) | (±3.29%) |
| grown from | | | | | | | | |
| Zero-D seeds | | | | | | | | |
| Obtained | 63.5 ± 1.877 | 5.9 ± 0.24 | 3.9 ± 0.226 | 5.7 ± 0.599 | 3.5 ± 0.3 | 210.3 ± 7.5 | 9.9 ± 1.9 | 264.4 ± 11.589 |
| from plants | (±2.96%) | (±3.95%) | (±5.80%) | (±10.51%) | (±8.21%) | (±3.58%) | (±20.00%) | (±4.38%) |
| grown from | | | | | | | | |
| Zero-R seeds | | | | | | | | |
| Obtained | 90.5 ± 2.593 | 4.1 ± 0.3 | 5.2 ± 0.682 | 6.2 ± 0.77 | 6.4 ± 0.3 | 186.3 ± 4.6 | 6.4 ± 0.9 | 173.8 ± 6.303 |
| from plants | (±2.87%) | (±6.89%) | (±13.20%) | (±12.49%) | (±4.47%) | (±2.45%) | (±15.53%) | (±3.63%) |
| grown from | | | | | | | | |
| Zero-DR seeds | | | | | | | | |

Seeds treated with nucleases showed significant benefits over control plants especially in the speed of growth. Seeds harvested from plants of the first generation saved growth characteristics thus the second generation of plants that were grown from these seeds saved all characteristic as plants of first generation plants.

### Example 47: Products and Method Managing of Seeds Characteristics

Seed of Triricale were treated with nucleases (DNase and/or RNase) as previously discussed. Characteristics of plants from these seeds comparing with those grown from control untreated seeds are listed in table 56.

TABLE 56

| Parameter | Zero-D | Zero-R | Zero-DR | Cut-D | Cut-R | Cut-DR |
|---|---|---|---|---|---|---|
| Water uptake | increased | as control | as control | as control | increased | as control |
| percentage | | | | | | |
| (the actual | | | | | | |
| percentage of | | | | | | |
| total number of | | | | | | |
| seeds in the | | | | | | |
| sample that are | | | | | | |
| germinated in | | | | | | |
| an experiment) | | | | | | |
| Germination | decreased | increased | as control | increased | as control | increased |
| Percentage | | | | | | |
| (the sum of | | | | | | |
| germinated | | | | | | |
| seeds in certain | | | | | | |
| day divided by | | | | | | |
| the number of | | | | | | |
| germination | | | | | | |
| days | | | | | | |
| corresponding) | | | | | | |
| Mean | Radicle | | | | | |
| Germination | decreased | as control | as control | increased | as control | increased |
| Time (the | Hypocotyl | | | | | |
| average time a | decreased | increased | increased | increased | increased | increased |
| seed needs for | Radical + hypocotyl | | | | | |
| initiation and | as control | increased | increased | increased | as control | as control |
| ending of | | | | | | |
| germination | | | | | | |
| process) | | | | | | |
| Seed vigor (the | decreased | increased | as control | as control | increased | increased |
| indicator for | | | | | | |
| activity level | | | | | | |
| and | | | | | | |
| performance | | | | | | |
| of seed during | | | | | | |
| germination | | | | | | |
| and seedling | | | | | | |
| emergence; | | | | | | |
| ability to carry | | | | | | |
| out all | | | | | | |
| physiological | | | | | | |
| activities that | | | | | | |
| enable them to | | | | | | |
| perform) | | | | | | |
| Root\|Shoot | Shoot Weight | | | | | |
| Weight | decreased | increased | as control | increased | increased | as control |
| | Root Weight | | | | | |
| | decreased | increased | as control | as control | increased | as control |
| Seedling | Shoot Length | | | | | |
| height (root | decreased | increased | as control | increased | as control | as control |
| length and | Root Length | | | | | |
| shoots length, | as control | increased | as control | increased | as control | as control |
| cm) | | | | | | |

Seed treated with nucleases an turning seeds to of “Cut” and “Zero” states showed significant benefits over control plants in different aspects.

### Example 48: Products and Method for Plants and Seeds Growth in not Optimal Conditions

We studied how plating seeds to the state “Cut”, “Zero” and “Y” affected plant growth at higher soil salinity. For that seeds of Triticale (x Triticosecale Wittmack) were spread and allowed to grow on Potato dextrose agar with 0 (deionized water, as a control) and 250 mM salt (MgSO4) in a 9-cm-diam Petri dish. Seeds were pretreated with nucleases taken from 0.1 to 5000 μg/ml once, or three times to generate “Y” or “Zero” state. Nucleases were washed out and cells were placed in growth chamber at 25±1° C. with 12 h daylight. Daily observation and counting of the number of seeds which were sprouted and germinated were done up to 7 days. Sprouted seeds were referred to the seeds which have reached the ability to produce at least one noticeable plumule or radicle. Seeds were considered germinated with at least 2 mm radicle emergence from the seed coat. After seven days of treatment application, measurement of parameters was done and calculated.

Seeds were transplanted into plastic nursery pot for plants (L×W×D of 3.25″×2.75″×2.75″) filled with a mixture of soil and peat moss (3:1, v/v) containing organic fertilizer. The temperature of the greenhouse was maintained at 25±2° C. and 10±2° C. during day and night, respectively. Each treatment consisted of three replicates and 1/100 plant were planted per plastic pot. At harvest, after treatment, plant growth parameters, were measured. The represented values were shown as mean±SE with a minimum of three independent replicates (n=3). Data are presented in FIGS. 28 and 28.

Data obtained clearly show that the treatment of seeds with tested products and protects the growing plants from the negative effects of not optimal growth conditions.

### Example 49: Products and Method for Managing of Interaction Cells with DNA-Viruses

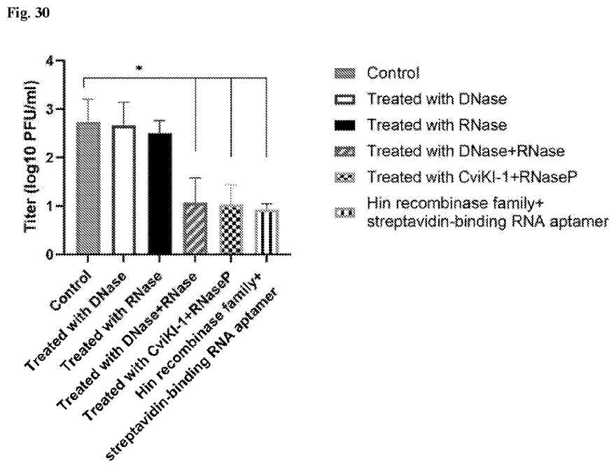

Vero cells were cultured in RPMI 1640 medium containing 10% heat inactivated fetal bovine serum (FBS) (Sigma), 100 g/mL streptomycin and 100 U/mL penicillin G in a humidified atmosphere of 5% CO2 in air at 37° C. (all Sigma) in 96 well plate (2×10e4 cells/well) for 22 hours. Media was replaced with the fresh one, supplemented with nucleases (0.01 μg/mL) or proteins that bind nucleic acids (100 mg/mL) or their combinations and incubated for 1 h at 37° C. Media was removed, cells were washed with PBS and HSV-1 was added, incubated at 1.5 h at 37° C. Next, media was replaced with the fresh one and cells were incubated for another 48 h. The virus titer in the cell medium was determined by standard plaque assays using 10-fold serial dilutions of cell supernatants of Vero cells incubated for 48 h, after which cells were fixed and stained to count the plaques. Data are shows in FIGS. 29 and 30.

It is clearly seen that cells treated with testing compounds exhibited less cytotoxic effect following the viral infection and can be used for managing of viral infections.

### Example 50: Products and Method Managing of Tumor Progression

Lewis carcinoma cells were separated from the extracellular matrix and left either untreated or treated for 30 min with tested products as discussed previously. After the treatment, cells were washed to avoid further contact of the tested products with cells and were subcutaneously injected to C57BL/6 mice weighing approximately 18 g (12 weeks old; 20 mice). Effect of tested products destruction in cancerogenesis is presented in table 57.

TABLE 57
Effect of tested products on tumor progression

| | Tumor description | | | | | |
|---|---|---|---|---|---|---|
| Group | 1 week | 2 week | 3 week | | 4 week | |
| Untreated cells | Fibrosis | Presence of tumor | 20 × 13 | mm | 37 × 20 | mm |
| Treated with DNase 10 μg/mL | Fibrosis | Fibrosis | 4 × 4 | mm | 7 × 3 | mm |
| Treated with RNase 10 μg/mL | Fibrosis | Presence of tumor | 17 × 10 | mm | 22 × 15 | mm |
| Treated with DNase + RNase each | Fibrosis | Fibrosis | 0 | mm | 0 | mm |
| 10 μg/mL | | | | | | |
| Propidium iodine 1 μg/mL | Fibrosis | Fibrosis | 0 | mm | 0 | mm |
| Antibodies against cell surface | Fibrosis | Fibrosis | 0 | mm | 0 | mm |
| bound DNA and RNA, 1000 | | | | | | |
| μg/mL | | | | | | |
| Recombinant Human RNA | Fibrosis | Fibrosis | 2 × 2 | mm | 3 × 4 | mm |
| binding protein fox-1 homolog 2 + | | | | | | |
| uracil-DNA glycosylase each 10 | | | | | | |
| μg/mL | | | | | | |
| Modified nucleophosmin + | Fibrosis | Fibrosis | 1 × 1 | mm | 2 × 2 | mm |
| Ribosomal protein S60 each 100 | | | | | | |
| μg/mL | | | | | | |

As can be seen from the presented data, the use of tested products leads to a decrease in their invasive activity and can be used as antitumor strategy.

### Example 51: Products and Method for Managing Metastasis

MC38 control cells or after being treated with tested products were studied for their potency to develop metastasis. To induce colorectal liver metastases 5×10e4 MC38 were injected through a 1 cm midline laparotomy into the spleen of 8-10 week old C57BL/6J WT mice using a 23 ga needle. Tumor cells were allowed to circulate for 30 minutes followed by splenectomy and closure (to prevent the formation of splenic tumor). Presence of hepatic metastases, calculated as metastatic rate (%) was calculated on day 21. Data are shown in table 58.

TABLE 58
Effect of the tested products on metastasis formation

| Metastasis rate (%) | Liver Metastasis rate (%) |
|---|---|
| Control | 100 |
| Treated with DNase 10 μg/mL | 50* |
| Treated with RNase 10 μg/mL | 50* |
| Treated with DNase + RNase each 10 μg/mL | 0* |
| Treated with DNase 10 μg/mL + Ribosomal | 0* |
| protein S14 10 μg/mL | |
| Treated with DNase 10 μg/mL + modified | 0* |
| amikacin 1 μg/mL | |
| Pyrrole-imidazole polyamide 1 μg/mL + | 0* |
| RNase U2 1 μg/mL | |
| Histone 1 + T7 RNA polymerase | 0* |

*p < 0.05

It is clearly shown that the use of tested products decreased metastatic activity of tumor cells.

### Example 52: Products and Method for the Treatment of Diabetes and Diabetic Retinopathy

Patients 15 people (5 males, 10 females) with type 1 and 2 diabetes with confirmed severe Nonproliferative Retinopathy/Proliferative diabetic retinopathy enrolled in the study.

Each patient was on individual insulin regimen for at least 3 years. Blood glucose level was measured by applying a drop of finger blood to a ‘test-strip’, which was next inserted into an electronic blood glucose meter.

Patients have administered Group 1—DNase I (bovine), Group 2—RNase (bovine) or Group 3—combination DNase+RNase (bovine) BID 200 mg in capsules. Group 4—administered riboflavin 800 mg×times a day. Each treatment group n=3. Two patients Group 5—modified bleomycin. Each patient signed a comprehensive consent form before administration of the drugs.

There was a significant improvement in normalization of blood glucose levels in all therapeutic groups of this study compared with pretreatment period (table 59).

TABLE 59
Effect of products on glucose level

| | | | Pretreatment | Pretreatment | Pretreatment |
|---|---|---|---|---|---|
| | Pretreatment | | Fasting | Fasting | Fasting |
| | Fasting | Day, | Glucose 7 days | Glucose 14 days | Glucose 28 days |
| | Glucose before | when the | after the initiating | after the initiating | after the initiating |
| | experimental | patient | of experimental | of experimental | of experimental |
| | therapy (mean | stopped | therapy (mean | therapy (mean | therapy (mean |
| | measurement for | using | measurement for | measurement for | measurement for |
| Group | the last 7 days) | insulin | the last 7 days) | the last 7 days) | the last 7 days) |
| 1 | 8.3 | 25 | 7.1 | 6.8 | 5.7 |
| 2 | 7.6 | 6 | 6.2 | 5.6 | 5.8 |
| 3 | 8.9 | 26 | 7.7 | 6.8 | 6.1 |
| 4 | 8.2 | 11 | 7.1 | 6.2 | 6.2 |
| 5 | 9.5 | 13 | 7.5 | 5.6 | 6.4 |

There was a significant improvement in the visual acuity of patients in all therapeutic groups of this study compared with pretreatment period. Data are shown in Table 60

TABLE 60
Effect of tested products on visual acuity

| | | Visual acuity | | Retinal detachment | |
|---|---|---|---|---|---|
| | | Before | 14 days | Before | 28 days |
| Group | Patient | therapy | after | therapy | after |
| 1 | 1 | 20/200 | 20/40 | − | − |
| | 2 | 20/300 | 20/100 | − | − |
| | 3 | 20/600 | 20/200 | − | − |
| 2 | 1 | 20/500 | 20/100 | + | − |
| | 2 | 20/300 | 20/100 | − | − |
| | 3 | 20/250 | 20/80 | − | − |
| 3 | 1 | 20/200 | 20/63 | + | − |
| | 2 | 20/125 | 20/63 | − | − |
| | 3 | 20/125 | 20/40 | − | − |
| 4 | 1 | 20/250 | 20/80 | + | − |
| | 2 | 20/500 | 20/125 | + | − |
| | 3 | 20/600 | 20/200 | + | + |
| 5 | 1 | 20/600 | 20/250 | + | − |
| | 2 | 20/200 | 20/63 | − | − |

As it is seen tested products significantly improved vision and retinal detachment. The use of the tested products also allowed to lower the glucose level including patient refractory to insulin. Moreover, patients were able to step out form the insulin therapy.

### Example 53: Products and Method for Prophylactic and Treatment of Diseases Associated with Protein Misfolding

E. coli 25922 after the treatment with tested products taken at concentrations from 0.1 μg/ml up to 100 mg/ml action were obtained as previously described and plated on the Columbia agar (Oxoid), supplemented or not supplemented with reverse transcriptase inhibitors (100 mg/ml).

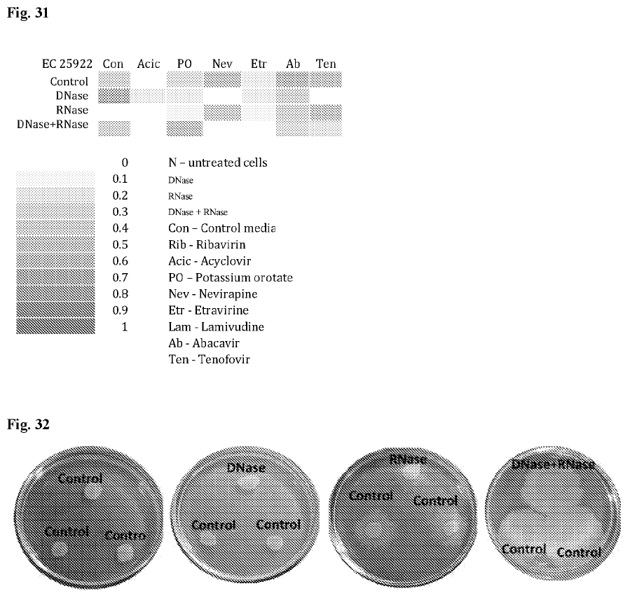

Next, bacteria were washed with PBS, supernatant was filtered with 0.2 uM filer and measured with OD500 using a microtiter plate reader (Epoch 2—BioTek). The amount of amyloid was recalculated total OD600. Data are shows in FIG. 31.

Tested products significantly decreased the amount of amyloid production by bacteria in biofilms.

Some of the reverse transcription inhibitors also decreased amyloid production and this alteration was dependent on the pretreatment of cells with nucleases. The decrease of amyloid production by cells can be used for its antibacterial potential, as well as for the prevention and/or treatment of infections and neurodegenerative diseases.

### Example 54: Products and Method for Managing of Cells Interaction

Bacillus VT1200 were washed out form the extracellular matrix and treated with nucleases as described earlier. 10 μL of 10e7 bacteria were plated on Columbia agar in different combinations. Analysis of microbial growth was evaluated in 24 h. Data are presented in FIG. 32 and table 61.

TABLE 61
Managing of remote signal distribution with tested compounds

| | Alteration | | Alteration |
|---|---|---|---|
| | of the | | of the |
| | growth of | | growth of |
| | remote | | remote |
| Product | colonies | Product | colonies |
| T7 RNA polymerase | Yes | RNase PH 100 mg/mL | Yes |
| 0.01 μg/mL | | | |
| Ribosomal protein S19 | Yes | Branaplam 1 μg/mL | Yes |
| 0.01 μg/mL | | | |
| MSI1 0.01 μg/mL | Yes | Pre-miR 100 μg/mL | Yes |
| pteridine-2,4-dione | Yes | Myricetin 1 μg/mL | Yes |
| 10 μg/mL | | | |
| Modified tedizolid | Yes | Pyrithiamine 1 mg/mL | Yes |
| 1 μg/mL | | | |

It is clearly seen that the tested products can lead to a remote alteration of other non-treated cells, meaning that treated cells can be used for the managing of cells interaction.

### Example 55: Products and Method for Managing of Cells Motility

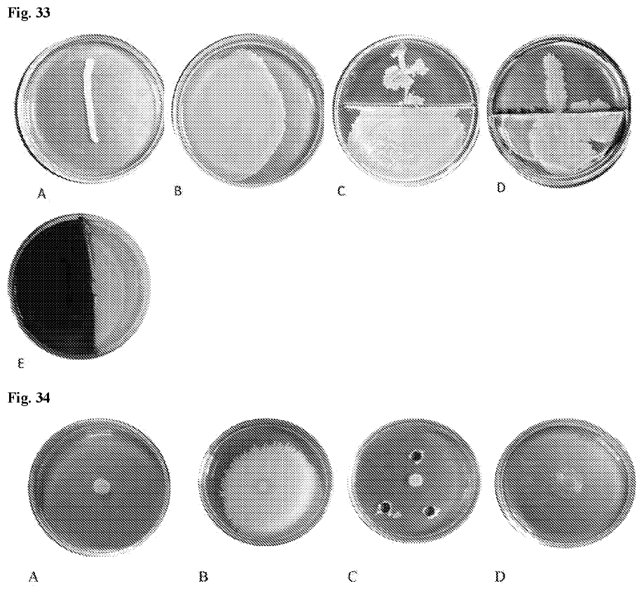

Bacillus VT1200 were grown overnight on Columbia agar (Oxoid). Cells were washed with PBS buffer and cells were separated from the extracellular matrix by 2 sets of centrifugation 5 minutes 4000×g (Microfuge® 20R, Beckman Coulter). Next, two 90 mm Petri dish, filled with Columbia agar (Oxoid), one of which was supplemented with tested products from 0.1 to 1000 μg/mL. Next, the agar was cut on 2 identical pieces and two halves of the agar (supplement and not supplemented with product) were put on a same Petri dish and separated with foil or plastic bridge. Then, washed bacteria were standardized up to 6 log 10 cells/ml and plated as a line through the “bridge” from the agar not supplemented with tested products to a part of agar supplemented with tested products. The same lines were made on two control Petri dishes: with the agar not supplemented with products that affect cells (FIG. 33A) and agar supplemented with tested products (DNase I) (FIG. 33B). Experimental dish with two halves of the agar without of any supplementation (upper part of FIG. 33C,D) and agar supplemented with tested products (lower part of FIG. 33C,D).

To control limitation of tested products penetration from the part that was supplemented to one that was not supplemented we made the identical composite plate with added blue dye (FIG. 33E)—there were no signs of paint penetration form one part of agar to another. FIG. 33 presents data for DNase I and table 62 summarizes the results obtained for other products.

TABLE 62
Regulation and acceleration the signal trafficking by tested products.

| | Acceleration the | | Acceleration the |
|---|---|---|---|
| Product | signal trafficking | Product | signal trafficking |
| Homeodomain 10 μg/mL | Yes | DNase1L 0.1 mg/mL | Yes |
| C2H2-zinc finger 1000 | Yes | granzyme B 1 μg/mL | Yes |
| μg/mL | | | |
| Myb_DNA-binding 1000 | Yes | Modified Actinomycin | Yes |
| μg/mL | | 10 μg/mL | |
| Exonuclease VII 10 μg/mL | Yes | Transcriptional | Yes |
| | | repressor QacR 100 | |
| | | μg/mL | |

As it is seen, tested products can trigger the formation of identical alterations at the very distant parts of the whole system, meaning that products can manage identical alterations triggering cells' migration.

### Example 56: Products and Method for Managing of Intergenerational Memory

Bacillus VT1200 were cultivated on the medium supplemented with tested products as described previously.

Control probes (FIG. 33A) revealed regular growth, while cells grown on the medium supplemented with DNase I (FIG. 33B) were grown on intact agar had revealed unusual expanded bacterial growth. Cells grown on the medium supplemented with DNase I were also cultivated on an agar with the defect on its surface (FIG. 33C) to modulate alteration of electric/magnetic field. When we took bacteria from the medium supplemented with DNase I (from 33B) and cultivated on the control agar with no products added (FIG. 33D), the biofilm still had an altered morphology, similar to alteration that were observed on the media with added products (Table 63).

TABLE 63
Effects of regulation of signal generation
and spread and intergenerational memory

| | Cells | | Cells |
|---|---|---|---|
| | retain | | retain |
| Product (added | the | Product (added | the |
| to the media) | memory | to the media) | memory |
| Exonuclease I 100 μg/mL | Yes | Phenazine 0.1 mg/mL | Yes |
| Exonuclease I 0.01 μg/mL | Yes | N4C-ethyl-N4C 10 | Yes |
| | | μg/mL | |
| XhoI_I 0.1 μg/mL | Yes | XhoI_10 μg/mL | Yes |
| Modified daunomycin 10 | Yes | Modified daunomycin | Yes |
| μg/mL | | 1000 μg/mL | |

Data received indicate that products can managing cell alterations that could be fixed in cell memory and these alterations can be passed to another generations. Moreover, data received show that the signaling depends on electrical and/or magnetic conditions which in turn can be regulated with tested compounds.

### Example 57: Products and Method for Managing Cell Directional Movement and Colonization

Bacillus VT1200 were grown overnight on 90 mm Petri dish, filled with Columbia agar (Oxoid) separated into 4 sectors each was processed as the following:
Sector #1—control
Sector #2—Agar was supplemented with products tested in a range of concentrations from 0.01 μg/ml up to 100 mg/ml
Sector #3—Agar was supplemented with human plasma form volunteer
Sector #4—Agar was supplemented with human plasma form volunteer pretreated with products RNase A 100 μg/mL.

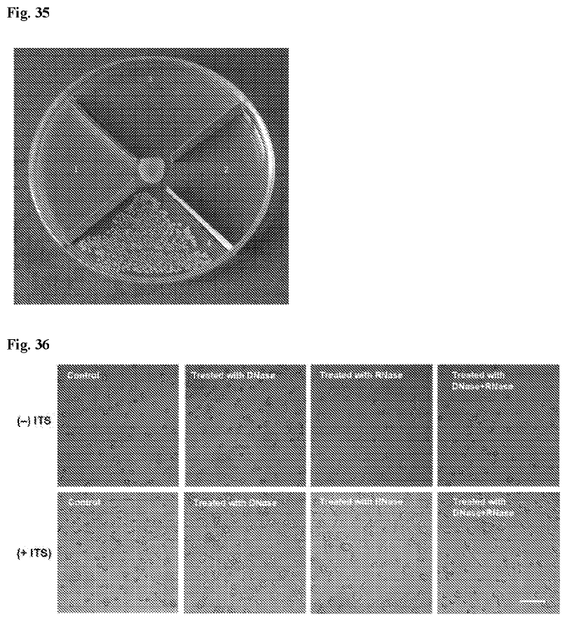

Data are presented in FIG. 35 and table 64.

It is clearly seen that the tested products (sector #4) triggered cell migration towards the chemoattractant (plasma); however, (sector #3 blood) with intact cells had no such a triggering effect.

TABLE 64
Effect of tested compounds on the control of directed cell migration and colonization

| | Modulation of | | Modulation of |
|---|---|---|---|
| | directed cell | | directed cell |
| | migration and | | migration and |
| Product | colonization | Product | colonization |
| β-(1→4)-Linked-2,6-diamino- | Yes | β-(1→4)-Linked-2,6- | Yes |
| 2,6-dideoxy-d-galactopyranose | | diamino-2,6-dideoxy-d- | |
| oligomers 1 μg/mL | | galactopyranose oligomers | |
| | | 1000 μg/mL | |
| Modified RNase II 1000 | Yes | RNA methylase 1000 | Yes |
| μg/mL | | μg/mL | |
| TLR3 1 μg/mL | | TLR3 1000 μg/mL | |
| RNA methylase 1 μg/mL | | RNA methylase 1000 | |
| | | μg/mL | |
| RNA-recognition motif, RNP1 | Yes | RNA-recognition motif, | Yes |
| 1000 μg/mL | | RNP1 10 μg/mL | |
| RNase II 0.01 μg/mL | Yes | RNase A 0.1 μg/mL | Yes |

This experiment demonstrates that tested products and method can be used to regulate cell migration, directed colonization, invasion as well as infectious process, dispersal, movement, directed taxis and can be utilized in biomanufacturing, infection treatment and microbiome transplantation.

### Example 58: Products and Method for the Prevention and Treatment of Autoimmune Conditions

Serum antibodies to DNA of P. aeruginosa, E. coli RNA, antibodies conjugated with DNase I were obtained as described earlier.

To model GVHD we used the MHC class I and II disparate model, C57BL/6 (H-2b) to BALB/c (H-2d). The recipient animals were females, 8 weeks of age. To prepare a cell suspensions from the euthanize donor mice we used CD8 purification kits (Miltenyi Biotec) according to the manufacturer instruction to isolate CD8 T cells from the spleen. The yield was 6.7 log 10 cells that were resuspend pellets in 1640 RPMI with 5% FBS (all Gibco). A suspensions of bone marrow cells and splenocytes were prepared in saline for injection.

Next, mice were irradiated by 2 equal doses 4.5 cGy each and then, mice were injected with 6.5 log 10 bone marrow cells and 7 log 10 splenocytes. Starting the same day as the BMT mie were randomized to the groups with the following treatment of the tested products in a range of concentrations from 1 μg/ml up to 1000 μg/ml

### Groups:

1. Control—untreated
2. Antibodies against DNA of P. aeruginosa 1 μg/ml two times a day
3. Antibodies against DNA of P. aeruginosa 100 μg/ml two times a day
4. Antibodies against DNA of P. aeruginosa 10 μg/ml two times a day+Nevirapine from 0.1-50.0 mg/kg once daily
5. Antibodies against RNA of E. coli 0.1 μg/mL once a day
6. Antibodies against RNA of E. coli 1000 μg/mL once a day
7. AntiD8 conjugated antibodies with DNase I two times a day
8. Cells prior to the injection were treated with DNase 0.1 μg/mL once every 48 h
9. Cells prior to the injection were treated with DNase 1000 μg/mL once every 48 h
10. Antibodies against surface-bound DNA once a day

The survival data are presented in Table 65, below:

TABLE 65
Effect of tested products managing of autoimmune conditions

Dead/alive

| Group | Day 0 | Day 7 | Day 14 | Day 21 |
|---|---|---|---|---|
| 1 | 0/10 | 6/4 | 8/2 | 9/1 |
| 2 | 0/10 | 3/7 | 3/7 | 4/6 |
| 3 | 0/10 | 2/8 | 2/8 | 3/7 |
| 4 | 0/10 | 1/9 | 3/7 | 3/7 |
| 5 | 0/10 | 2/8 | 5/5 | 5/5 |
| 6 | 0/10 | 2/8 | 3/7 | 3/7 |
| 7 | 0/10 | 2/8 | 3/7 | 3/7 |
| 8 | 0/10 | 3/7 | 3/7 | 3/7 |
| 9 | 0/10 | 0/10 | 1/9 | 1/9 |
| 10 | 0/10 | 1/9 | 2/8 | 2/8 |

Data received show that the tested products and methods led to a significant amelioration of the severity of autoimmune processes and GVHD symptoms and increased the survival rate.

### Example 59: Products and Methods for the Treatment of Cancers

Vaccines from intracellular DNA or DNA of NAMACS and NAMACS-ANA of P. aeruginosa or E. coli biofilms or from the mix of microorganisms isolated from the feces of mammal (mice) were obtained as described earlier. Mice (c57bl/6,8-week old, #6 per group) were subcutaneously injected with H59. Mice were divided into untreated, one time or two-times i.v. injected with vaccines.

Livers were excised from mice when the flank tumor size reached 2.5 cm3 and hepatic metastatic nodules were analyzed (table 66).

TABLE 66
Anticancer effects of vaccines

| | Mean number of |
|---|---|
| Type of vaccine | nodules per liver ± SD |
| Control | 12.4 ± 3.7 |
| Intracellular DNA of P. aeruginosa, 1 time injection | 2.5 ± 1.1* |
| Intracellular DNA of E. coli, 1 time injection | 7.2 ± 2.2* |
| Intracellular DNA of mix of microorganisms isolated from the | 6.1 ± 1.3* |
| feces, 1 time injection | |
| DNA of NAMACS and NAMACS-ANA of P. aeruginosa, 2 time injection | 1.4 ± 0.9* |
| DNA of NAMACS and NAMACS-ANA of E. coli, 2 time injection | 3.9 ± 0.4* |
| DNA of NAMACS and NAMACS-ANA of mix of microorganisms | 5.4 ± 1.0* |
| isolated from the feces, 2 time injection | |
| Intracellular DNA of P. aeruginosa, 3 time injection | 1.6 ± 0.2* |
| Intracellular DNA of E. coli, 3 time injection | 2.2 ± 0.4* |
| Intracellular DNA of mix of microorganisms isolated from the | 3.3 ± 0.5* |
| feces, 3 time injection | |

*p < 0.05

Data clearly show that vaccines having in their components bacterial DNA and NAMACS and NAMVACS-ANA possess high anticancer activity.

### Example 60: Products and Methods for Regulation of Protein-Based Receptors

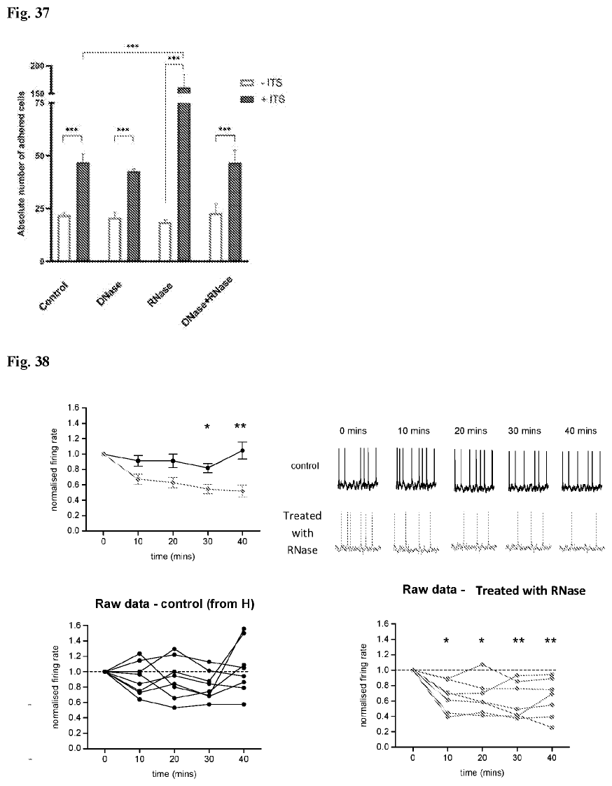

CHO cells were initially serum-starved for 24 h and plated at a density of 4.2 log cells/well in 48-well culture plates. Cells were separated from the extracellular matrix as previously described, treated with the PBS to generate C17 control, or with tested productsas previously described, and treated with ITS-complex (insulin, 5 μg; transferrin, 5 μg; selenium, 5 ng/ml) according to the manufacturer's instructions (Sigma-Aldrich) in DMEM. The number of attached cells was determined after 24 h of growth, according to previously established methods. Results are presented in FIGS. 36, 37 and table 67.

TABLE 67
Effect of tested products on the number of cells per sight

| | Mean number of | | Mean number of |
|---|---|---|---|
| | cells per sight | | cells per sight |
| | (mean from 10 | | (mean from 10 |
| Group | different sights) | Group | different sights) |
| Control | 64 | CCCH zinc finger | 176 |
| | | protein 50 μg/mL | |
| DNase I 0.1 μg/mL | 59 | Tobramycin 1 μg/mL | 215 |
| DNase I 250 μg/mL | 77 | Modified tobramycin | 248 |
| | | 1 μg/mL | |
| RNase I 0.1 μg/mL | 204 | Modified tobramycin | 239 |
| | | 100 μg/mL | |
| RNase I 250 μg/mL | 227 | Ribosomal | 148 |
| | | protein L22 100 | |
| | | μg/mL | |
| DNase I + RNase each | 86 | Ribosomal | 181 |
| 0.1 μg/mL | | protein L22 1000 | |
| | | μg/mL | |
| DNase I + RNase each | 82 | RNA recognition | 174 |
| 250 μg/mL | | motif 100 μg/mL | |
| RBPs CsrA 50 | 289 | Modified | 193 |
| μg/mL | | pentamidine 100 | |
| | | μg/mL | |
| Modified | 184 | Modified T7 RNA | 217 |
| Tobramycin 100 | | polymerases 100 | |
| μg/mL | | μg/mL | |
| Modified RNA | 155 | Modified | 175 |
| helicase 100 μg/mL | | linezolid100 μg/mL | |

As it is seen the use of tested products can supervise and govern the protein receptors.

### Example 61: Products and Method for Managing of Wound Healing

We studied the effects of tested products on management of stem cells to be used for on wound healing. Mouse embryonic stem cell (CGR8, Sigma) (MESC) were cultured on GMEM±2 mM Glutamine+0.05 mM 2-Mercaptoethanol (2ME)±1000 units/ml DIA/LIF+1000 Foetal Bovine Serum (FBS). MESC were treated with different testing products in a range of concentrations from 1 μg/ml up to 1000 μg/ml from 1.0 to 60.0 minutes prior to the application to the wound. 8-week-old C57BL/6 mice (n=30) with were anesthetized with ketamine and xylazine.

A full thickness 1 cm diameter skin defect was done for each animal on the neck region after removal of hair from the selected areas and surgical preparation with alcohol scrub. Full-thickness burn wounds were established under general anesthesia bilaterally on the dorsolateral trunk.

5×10e4 cells/mi ME SC were transferred to each the wound and covered with a sterile dressing. New cells were added every 4 days. Control animals were left untreated, but covered with the sterile dressing. Data are shown in table 68.

TABLE 68
Effect of products on wound size

| MESC | Wound size | | MESC | Wound size | |
|---|---|---|---|---|---|
| treated with | Day 0 | Day 8 | treated with | Day 0 | Day 8 |
| Untreated | 100% | 48% | NONO protein | 100% | 11%* |
| | | | 100 μg/mL, 60 | | |
| | | | min | | |
| RNase A 100 | 100% | 0%* | EcoR + | 100% | 12%* |
| μg/mL, 60 min | | | Ribosomal | | |
| | | | protein S1 | | |
| | | | each 1 μg/mL, | | |
| | | | 60 min | | |
| RNase A 1 | 100% | 14%* | EcoR + | 100% | 0%* |
| μg/mL, 1 min | | | Ribosomal | | |
| | | | protein S1 | | |
| | | | each 1000 | | |
| | | | μg/mL, 60 min | | |
| RNase + DNase, | 100% | 0%* | Thiouridine | 100% | 4%* |
| each 100 | | | synthase with | | |
| μg/mL, 60 min | | | N-terminal | | |
| | | | ferredoxin-like | | |
| | | | domain 12 | | |
| | | | mg/mL, 60 | | |
| | | | min | | |
| RNase + DNase, | 100% | 9%* | Thiouridine | 100% | 8%* |
| each 10 μg/mL, | | | synthase with | | |
| 1 min | | | N-terminal | | |
| | | | ferredoxin-like | | |
| | | | domain 1 | | |
| | | | μg/mL, 60 min | | |
| Riboflavin 10 | 100% | 12%* | Modified | 100% | 15%* |
| μg/mL, 60 min | | | Riboflavin 10 | | |
| | | | μg/mL, 3 h | | |

*p < 0.05 to Untreated Day 8;

As it is seen the products and method demonstrate significantly higher rate of wound healing.

### Example 62: Products and Method for Management of Memory and Cognitive Processes

Male BL6 mice (approximately three to four weeks and P12-P21 for paired-synaptic transmission

studies) were killed by cervical dislocation and decapitated. Parasagittal hippocampal and neocortical slices (350 mM) were cut with a Microm HM 650V microslicer in cold (2-4° C.) high Mg2, lowCa2 aCSF, composed of the following: 127 mM NaCl, 1.9 mM KCl, 8 mM MgCl2, 0.5 mM CaCl2), 1.2 mM KH2PO4, 26 mM NaHCO3, and 10 mM D-glucose (pH 7.4 when bubbled with 95% O2 and 5% CO2, 300 mOsm).

Neocortical slices were cut at an angle of 15°, such that the blade started cutting from the surface (layer 1) of the neocortex toward the caudal border of the neocortex [to ensure the integrity of Layer V pyramidal cell (Layer V PC) dendrites]. Slices were stored at 34° C. in standard aCSF (1 mM Mg2 and 2 mMCa2) for between 1 and 8 h. Statistical analysis was done conducted with 2-way ANOVA.

Control probes were left untreated, experimental treated with tested products as previously described. Results are shown in FIG. 38 and tables 69, 70, 71. It is clear that the use of tested products reduces neuronal excitability.

TABLE 69
Variation data

| Source of Variation | % of total variation | P value |
|---|---|---|
| Time × Probe interaction | 9.325 | 0.0021 |
| Time | 14.65 | 0.0010 |
| Probe | 23.20 | 0.0031 |

TABLE 70
Effect of RNase on studied parameters

| | Mean Diff. | 95.00% CI of diff. | P Value |
|---|---|---|---|
| 0 mins vs. 10 mins | 0.3264 | 0.05907 to 0.5937 | 0.0193 |
| 0 mins vs. 20 mins | 0.3700 | 0.08808 to 0.6519 | 0.0133 |
| 0 mins vs. 30 mins | 0.4541 | 0.2099 to 0.6983 | 0.0018 |
| 0 mins vs. 40 mins | 0.4824 | 0.1690 to 0.7957 | 0.0056 |

TABLE 71
Effect of tested products on neuronal excitability.

| | Inhibition | | Inhibition |
|---|---|---|---|
| | of neuronal | | of neuronal |
| Tested product | excitability | Tested product | excitability |
| RNase A 0.01 μg/mL | Yes | Antibodies against | Yes |
| | | TezR_R1 1 μg/mL | |
| Exoribonuclease 1 | Yes | Antibodies against | Yes |
| μg/mL | | TezR_R1 100 | |
| | | μg/mL | |
| T7 RNA polymerase | Yes | T7 RNA polymerase | Yes |
| 10 μg/mL | | 100 μg/mL | |
| Stem-loop binding | Yes | Stem-loop binding | Yes |
| protein 1 mg/mL | | protein 1 μg/mL | |

Data received clearly show the effect of tested products on neuronal excitability, that is a critical element of synaptic plasticity, learning and memory and is a component of aging, impairments of which are related to age-related deficits in learning and memory. Moreover tested products can be used to enhance the human brain's cognitive capabilities, restore the memory, speech and movement by managing of sending and/or receiving electrical signals through the brain from and to machines.

### Example 63: Products and Method for Managing Cell Reaction to Light

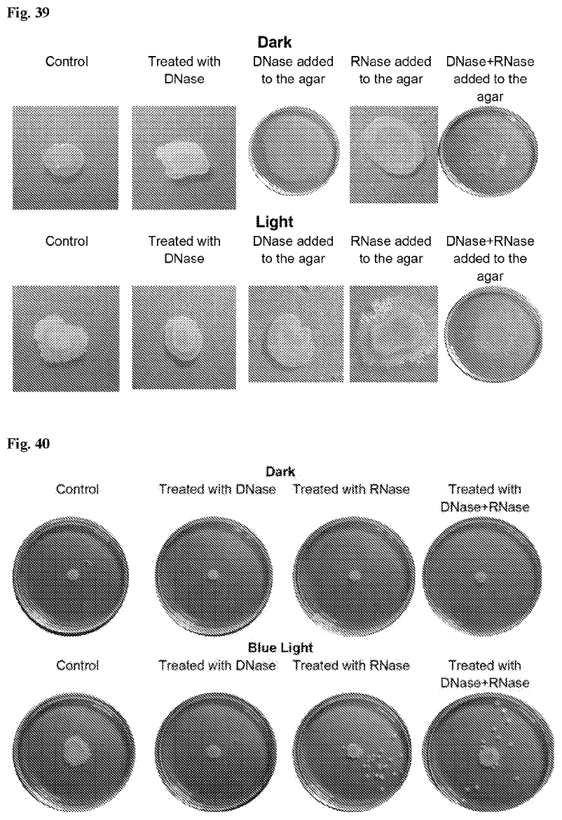

We studied the effects of tested products in a range of concentrations from 1 μg/ml up to 1000 μg/ml with the exposure from 1 to 60 minutes on managing of cell characteristics by light. Bacillus VT1200 were separated from the extracellular matrix and treated with tested products as previously discussed. Cells were cultivated on Columbia agar 24 h at 37 C in the incubator under (i) dark, or (ii) light (visible or blue). Data are presented in FIGS. 39 and 40, Table 72.

TABLE 72
Effect of different products on cell's response to visible light

| | Alteration | Inhibited | | Alteration | Inhibited |
|---|---|---|---|---|---|
| | of response | response | | of response | response |
| Tested product | to light | to light | Tested product | to light | to light |
| Treated with | Yes | | Endonuclease FokI | Yes | Yes |
| DNase 100 μg/mL | | | family added to | | |
| | | | agar 1 μg/mL | | |
| Treated with | Yes | Yes | Treated with | Yes | Yes |
| DNase 0.1 μg/mL | | | Histone H4 0.1 | | |
| | | | μg/mL | | |
| DNase added to | Yes | Yes | Treated with | Yes | Yes |
| agar 1 μg/mL | | | Modified Histone | | |
| | | | H4 1000 μg/mL | | |
| Treated with | Yes | | Pipobroman 0.1 | Yes | Yes |
| RNase A 10 μg/mL | | | μg/mL | | |
| RNase added to | Yes | | Busulfan added to | Yes | Yes |
| agar A 1 μg/mL | | | agar 0.1 μg/mL | | |
| Treated with | Yes | | Treated with | Yes | Yes |
| DNase + RNase A | | | Modified Busulfan | | |
| 10 μg/mL | | | 0.1 μg/mL | | |
| DNase + RNase | Yes | | Treated with T4 | Yes | |
| added to agar A 0.1 | | | Polynucleotide | | |
| μg/mL | | | Kinase 1 μg/mL | | |

It is clearly seen that the colonies of control bacteria grown under the light displayed altered morphology, while the morphology of the colonies formed after the treatment of tested products were almost not altered, meaning that after the treatment with tested products cells were unable to respond for the appearance of light and have different regulation towards physical factors.

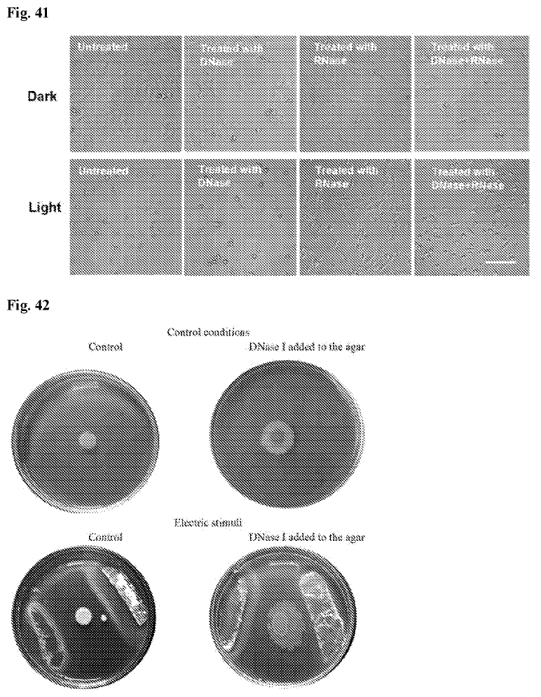

We also analyzed different tested products on the response to light of eukaryotic cells. For light irradiation, an aliquot of 4.0 log 10 Vero cells was placed in the wells of 24-well plates. Cells were allowed to attach for 3.5 h at 37° C. in DMEM with 10% FBS, the medium was replaced with DMEM, and cells were treated with nucleases as previously described. The medium was replaced for 30 min, cells were washed with DMEM, and fresh DMEM with FBS was added. The plates were exposed to visible light sources supplied with 150 W (840 lm) halogen lamps (Philips, Shanghai, China) for 24 h at 37° C. The cellular state was observed and photographed under a Zeiss Axiovert 40C microscope (10× magnification). Results are presented in FIG. 41. We observed that within 24 h after the 30 min exposure to RNase majority of cells had a triradiate morphotype, whereas all Vero control were unable to grow due to phototoxicity. Together, these results suggest that tested products can manage cell responses to light and plays an important role in photoprotection from light-induced cytotoxicity.

### Example 64: Products and Method for Managing Cell's Reaction to Electrical Stimuli

We studied the effects of tested products in a range of concentrations from 1 μg/ml up to 1000 μg/ml with the exposure from 1 to 60 minutes on managing cell characteristics to electrical stimuli. Bacillus VT1200 were separated from the extracellular matrix and left either untreated or treated with tested products as previously discussed and were cultivated on Columbia agar 10.0-48 h at 37° C. in the incubator under (i) dark, or (ii) electric stimulation 1 mA. Data are presented in FIG. 42 and table 73.

TABLE 73
Use of tested products for managing of
cell's response to electric stimuli

| | Alteration | | |
|---|---|---|---|
| | of response | | Alteration |
| | to electrical | | of response |
| Tested product | stimuli | Tested product | to light |
| DNase 0.1 μg/mL | Yes | 1,8-dihydroxy | Yes |
| | | anthraquinone 10 | |
| | | mg/mL | |
| DNase 10 mg/mL | Yes | Histone H5 0.1 μg/mL | Yes |
| Modified physcion | Yes | Histone H5 500 μg/mL | Yes |
| 1 μg/mL | | | |
| Modified physcion | Yes | Heat shock protein 1 | Yes |
| 1 mg/mL | | μg/mL | |
| Modified Bleomycin | Yes | Heat shock protein | Yes |
| 10 μg/mL | | 1000 μg/mL | |

It is clearly seen that the tested products manage the behavior of bacteria in response to electrical stimuli.

### Example 65: Products and Method to Monitor Environmental Conditions, Radiation and Ecology

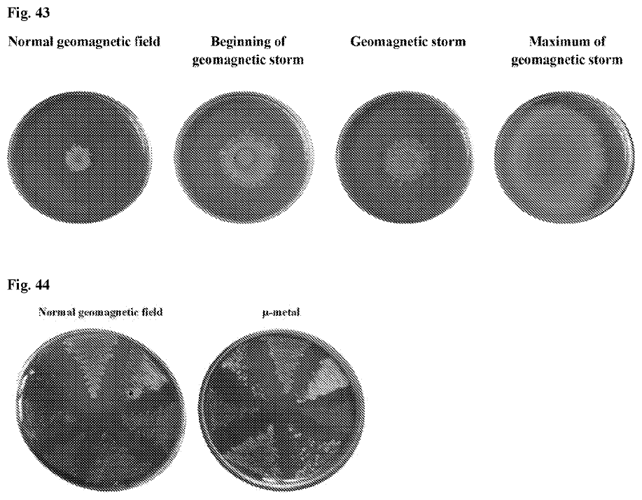

We used TezRs to monitor environmental, weather and geomagnetic conditions. For that, daily we plated B. pumilus VT 1200 separated from the extracellular matrix and treated with DNase as previously discussed on the surface of Columbia and Pepted Meat 90 mm Petri dishes, placed in Mu-metal boxes and cultivated for 24 h at 37 C. We analyzed alterations of biofilm morphology and aligned these alterations with the geomagnetic storms. Data are presented in FIG. 43.

As it seen, by cultivating microorganisms treated by products in Mu-metal enables to detect geomagnetic storms and other alterations and disturbance of the magnetosphere as well as other environmental factors such as geological exploration, water condition, radiation and magnetic conditions, sun exposure, flooding, earthquake.

### Example 66: Products and Methods for Managing Magneto-Dependent Cell Activity

We found certain bacteria within human microbiota react on the alteration of geomagnetic field. 1 ml saliva sample from an individual suffering from magneto-dependence was dissolved in PBS by 10,000 fold and plated on Columbia and Pepted Meat agar supplemented with 10% erythrocytes on 90 mm Petri dishes and cultivated from 10 up to 72 h at 37 C and pure bacterial cultures were obtained.

Next, these pure bacterial cultures were subcultivated in the normal or altered geomagnetic field (in μ-metal as described above). We found that the growth and activity of some bacteria was changed when grown in altered geomagnetic field (FIG. 44).

We suggested that since the growth and activity of this bacteria can be regulated with the alteration of geomagnetic field, we can use this phenomenon to switch on or off the activity of certain genes. One of such bacteria was B. pumilus VT1200 which naturally produces RNase I. DNA fragment was purified and ligated into the pET-15b vector (Novagen, Madison, WI) to construct the expression pETDNaseI plasmid. The plasmid was transformed into Bacillus pumilus VT 1200 (also shown as one with the high tropism to the tumor) for initial cloning. The Pst I and Sac I sites in the DNase I gene were used for selecting positive clones. Next, in B. pumilus VT1200 T7 RNA polymerase gene was added to turn on the pET-15b protein expression system for production of DNase I (SEQ No 1).

SEQ No 1
MRGMKLLGALLALAALLQGAVSLKIAAFNIRTFGRTKMSNATLVSY
IVQILSRYDIALVQEVRDSHLTAVGKLLDNLNQDAPDTYHYVVSEP
LGRKSYKERYLFVYRPDQVSAVDSYYYDDGCEPCGNDTFNREPFIV
RFFSRFTEVREFAIVPLHAAPGDAVAEIDALYDVYLDVQEKWGLED
VMLMGDFNAGCSYVRPSQWSSIRLWTSPTFQWLIPDSADTTATPTH
CAYDRIVVAGMLLRGAVVPDSALPFNFQAAYGLSDQLAQAISDHYB
VEVMLK

Colonies of B. pumilus VT1200 were cultured at 37° C. in Columbia agar, supplemented with ampicillin 50 mg/mL. DNase I activities was measured using the method described by Kunitz. Overnight colonies were washed with sterile PBS, bacteria were spun down (3000×g) and washed three times with sterile PBS (Ginco) before injection into 8-week-old BALB/C mice (N 8 per group). Intravenous (into a tail vein) injections of bacteria were performed at a concentration of 7.0 log 10 in 50 μl PBS.

We studied could the alteration of geomagnetic field cause the increase of B. pumilus VT 1200 activity. For that we placed animals in a four-layer μ-metal envelops for 120 minutes and measured DNase and RNase activity in the blood. DNase and RNase activity at each timepoint of B. pumilus in normal geomagnetic field was taken as 100% Data are shown in table 74.

TABLE 74
Modulation of cell activity with placing of the
macroorganism in altered geomagnetic field

| B. pumilus VT1200 | DNase activity | RNase activity |
|---|---|---|
| 24 h Normal | 100% | 100% |
| geomagnetic field | | |
| 28 h Normal | 100% | 100% |
| geomagnetic field | | |
| 36 h Normal | 100% | 100% |
| geomagnetic field | | |
| 24 h Normal | 249%* | 204% |
| geomagnetic field | | |
| after 2 h of altered | | |
| geomagnetic field | | |
| and another 4 h at | | |
| Normal geomagnetic | | |
| field | | |
| 24 h Normal | 256%* | 371% |
| geomagnetic field | | |
| after 2 h of altered | | |
| geomagnetic field | | |
| and another 10 h at | | |
| Normal geomagnetic | | |
| field | | |

*p < 0.05

Data presented show that we can switch on and off the activity of certain genes in macroorganism as well as in cells cultured ex vivo and injected to the macroorganism.

### Example 67: Method of Diagnostic and Treatment of Associated with Alterations of Geomagnetic Activity and Weather Dependency

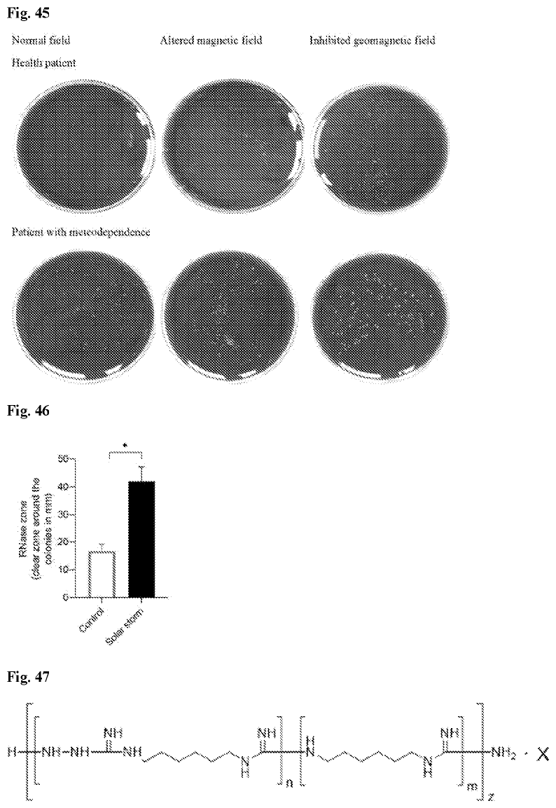

Saliva samples of 5 healthy individual and 5 subjects suffering from weather-dependence, head aches, migraines, airplane headaches were dissolved in PBS by 10,000 fold and plated to Columbia agar supplemented with erythrocytes (5%). Probes were cultivated in normal, altered or inhibited geomagnetic filed (μ-metal) for 24 h at 37 C. Representative image of control and probes of the patients are shown on FIG. 45.

It is clearly seen that some microorganisms from the oral cavity of the patient with weather dependency altered their growth after plating to an altered magnetic conditions. It can be used for the identification of bacterial strains with weather-dependent status, patient diagnose of weather dependence and underlying conditions and target for treatment intervention.

We isolated two bacterial strains that had an enhanced growth in inhibited geomagnetic field and using previously described method found that they produced a lot of RNase particularly in response to the altered geomagnetic condition. For that we maintained bacterial cultures at 37 C of these bacteria on agar plates supplemented with 10 μg/ml RNA as previously described and the RNase activity assessed as a clear zone around the colony was assessed. Daily, for 30 days 25 μl of bacterial culture (1×10e5 bacteria/ml) were plated on a center of 90 mm glass Petri dish and zone around the colony was analyzed. AT the end of observation period we compared RNase activity of bacteria between the days with normal sun activity and solar storms (FIG. 46).

After that we modified bacteria to develop Zero-D, Zero-R and Zero-DR cells as previously described to trigger cells to forget weather dependence and found that after that bacteria had a reduced expression of RNase (measured as previously described) triggered by solar storms comparing with untreated control and taken as 100%. Data is shown in table 75.

TABLE 75
Level of RNase expression by bacteria

| Cell | RNase activity | Cell | RNase activity |
|---|---|---|---|
| Control | 100% | Zero-R | 7%* |
| Zero-D | 11%* | Zero-DR | 0%* |

*p < 0.05

Next we enrolled 40 patients retrospectively suffering of >4 episodes per year of different types of weather dependence, such as: headaches, migraines, airplane headaches. These patients were treated with: (1) Oral rinse with Zero-D, Zero-R, Zero-DR bacteria from the same patient at 10e5/ml two times a week (2) Oral rinse with Zero-D, Zero-R, Zero-DR bacteria from the another patient patient at 10e5/ml two times a week (3) some patients received antibiotics (Penicillins, Tetracyclines, Macrolides at ½ recommended doses) to the airplane trips or during the aura before the onset the migraine, or (4) oral rinse with 0.0100 of compound Y190 (FIG. 47), or (5) Potassium orotate, Etinavir, Ribavirin, Abacavir were given at regular doses. All patients were monitored for another 12 months (table 76).

TABLE 76
Duration and the severity of the attack

| | Duration of the attack (hours)/Severity | | |
|---|---|---|---|
| | of the attack (in points) | | |
| | Weather dependent | | Airplane |
| Group | head aches | Migraines | headaches |
| Retrospectively | 6/3 | 18/3 | 7/3 |
| (before treatment) | | | |
| Treated with | 3/1* | 1/1* | 0/0* |
| Penicillins | | | |
| Treated with | 2/2* | 7/1* | 2/1* |
| Tetracyclines | | | |
| Treated with | 2/1* | 5/1* | 0/0* |
| Macrolides | | | |
| Oral rinse with Y190 | 1/1* | 3/1* | 0/0* |
| 0.01% | | | |
| Oral rinse with Zero- | 2/1* | 6/1* | 2/1* |
| D cells (from the | | | |
| same patient) | | | |
| Oral rinse with Zero- | 2/1* | 5/2* | 2/1* |
| R cells (from the | | | |
| same patient) | | | |
| Oral rinse with Zero- | 0/0* | 4/1* | 0/0* |
| DR cells (from the | | | |
| same patient) | | | |
| Oral rinse with Zero- | 2/1* | 5/1* | 2/1* |
| D cells (from another | | | |
| patient) | | | |
| Oral rinse with Zero- | 2/1* | 5/1* | 2/1* |
| R cells (from another | | | |
| patient) | | | |
| Oral rinse with Zero- | 1/1* | 3/1* | 1/1* |
| DR cells (from | | | |
| another patient) | | | |
| Potassium orotate | 4/2* | 8/2* | 1/1* |
| Etinavir | 2/2* | 2/3* | 4/1* |
| Ribavirin | 3/3* | 7/2* | 3/2* |
| Abacavir | 2/2* | 6/2* | 4/1* |
| DNase 100 mg | 3/1* | 3/1* | 2/1* |
| RNase 100 mg | 2/1* | 2/2* | 1/1* |
| DNase + RNase each | 0/0* | 0/0* | 0/0* |
| 100 mg | | | |

Thus, the use of tested products enables to decrease the development of weather dependence and migraines. Moreover, since previously we have shown that the higher expression of RNase is associated with the reduction of the lifespan the use or products and methods that inhibit RNase activity of microbiota can be used for the increase of the lifespan.

### Example 68: Products and Method for Management of Autoimmune Diseases by Management of Cells Memory

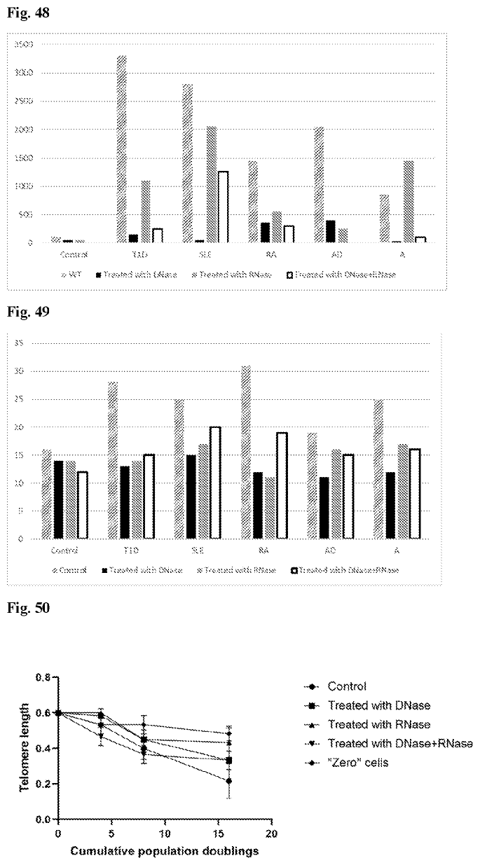

Peripheral venous blood was obtained from patients with type 1 diabetes (t1D), systemic lupus erythematosus (SLE), rheumatoid arthritis (RA), atopic dermatitis (AD), asthma (A) or healthy subjects (age matched). Monocytes were obtained using density centrifugation on Ficoll with the follow up negative selection using magnetic beads and further sorted with specific antibodies (keeping CD14+CD16− fraction). Monocytes at 5×10e5 cells/well were plated in 96 well plates containing HL-1 medium with 2 mM L-glutamine, 100 U/ml penicillin and streptomycin mix, nonessential amino acids and heat-inactivated serum. Cells were separated from the extracellular matrix and left either untreated or treated with products in a range of concentrations from 1 μg/ml up to 1000 μg/ml with the exposure from 1 to 60 minutes on managing as previously discussed. Number of IL-6 secreting cells were counted. Data are presented in FIG. 48.

It is clearly seen that patients with different autoimmune diseases have a higher number of IL-6− monocytes compared with controls. The use of tested products could regulate and inhibit IL production by cells and modulate autoimmune behavior of immune cells.

Studying the way, how different tested products can protect cells by being targeted by the components of immune system, we co-cultured memory T cells treated or not treated with tested products to alter their memory with monocytes from control, T1D, SLE, RA, AD and patients for 120 hours in the presence of anti-CD3. Memory T cells were then grown for additional 144 hours with the supplementation of IL-2. The number of IL-17 producing cells was counted. Data are presented in FIG. 48, 49 and table 77.

TABLE 77
Effects of tested compounds manage cell memory loss of
proinflammatory cytokines production by immune cells

| | Inhibition of | | Inhibition of |
|---|---|---|---|
| | proinflammatory | | proinflammatory |
| | cytokines | | cytokines |
| Tested product | production | Tested product | production |
| DNase 0.1 μg/mL | Yes | Propidium iodine 1 μg/mL | Yes |
| DNase 10 mg/mL | Yes | Histone H5 0.1 μg/mL | Yes |
| Antibodies againt primary | Yes | Histone H5 500 μg/mL | Yes |
| TezRs 1 μg/mL | | | |
| Antibodies againt primary | Yes | CytR protein1 μg/mL | Yes |
| TezRs 1 mg/mL | | | |
| Histone H5 10 μg/mL | Yes | Modified Mitotane 1000 | Yes |
| | | μg/mL | |
| Modified chrysophanol 1 | Yes | Modified Mitotane 1 | Yes |
| μg/mL | | μg/mL | |
| Modified chrysophanol 10 | Yes | Modified bleomycin 10 | Yes |
| mg/mL | | μg/mL | |

It is clearly seen that the erasure of the cell memory of T cells with the tested products inhibited their activation with IL-17 by monocytes from patients with autoimmune diseases; therefore, preventing these cells of being targeted by the components of immune system.

### Example 69: Products and Method Managing of Synthesis and Transportation of Products from Cells

We used rat INS-1 cell line that can produce and release hormone insulin release following glucose stimulation. Cells were maintained in RPMI 1640 serum-free culture medium supplemented with D-glucose supplemented and nutritional and antimicrobial factors as previously described in a humidified atmosphere. Cells were either untreated (control) or treated with tested products for different periods from 3 minutes to 24 h in a range of concentrations from 1 μg/ml up to 1000 μg/ml. The culture media was collected and stored at −80° C. until the use in insulin release assay. Insulin release was detected by using a rodent insulin ELIZA.

Comparison of insulin release and content between mBMDS and INS-1 cells. Data are presented in Table 78.

TABLE 78
Effect of tested products on synthesis,
transportation of products from cells

| | Insulin release | Insulin content |
|---|---|---|
| Probe | (ng/ml) | (ng/ml) |
| Control | 100% | 100% |
| Treated with DNase 10 μg/mL | 356% | 187% |
| Treated with RNase 1 μg/mL | 152% | 261% |
| Treated with DNase + RNase both 10 | 34% | 49% |
| μg/mL | | |
| Incubated with DNase 50 μg/mL | 269% | 202% |
| Incubated with RNase 50 μg/mL | 244% | 425% |
| Treated with Histone H3 1000 μg/mL | 190% | 253% |
| Treated with MetJ 1 μg/mL | 289% | 154% |
| Treated with HIV reverse transcriptase | 167% | 150% |
| 100 μg/mL | | |
| Treated with imidazole pyrrole pyrrole | 419% | 342% |
| oligomer 10 μg/mL | | |
| 4,5′,8-trimethylpsoralen 10 μg/mL | 265% | 176% |
| Modified 8-methoxypsoralen 50 μg/mL | 229% | 346% |
| 8-methoxypsoralen 50 μg/mL | 159% | 294% |

It is clearly seen that products can be used for managing production and secretion of different products by cells including hormones.

### Example 70. Products and Method for Managing of Eukaryotic Cells Memory for Generating Novel Sensors and Sensing Systems

We studied the use of tested products to reprogram cells in adaptive memory experiments. For that control C. albicans or following treatment with tested products were placed to M9 supplemented with dexamethasone and the beginning of growth was monitored. After each passage, cells were placed to Sabouraud broth for from 1.0 up to 72 h, then washed out, placed for M9 supplemented with dexamethasone for 4 h, after which, extracellular matrix was removed, cells were treated with tested products in a range of concentrations from 1 μg/ml up to 1000 μg/ml and fungi were again placed to M9 with dexamethasone for the next 20 h of growth. Data are shown in table 79.

TABLE 79
Use of products for managing of cells genome information.

| | OD600 (24 h of growth) | | | |
|---|---|---|---|---|
| | | | | Basic helix- |
| | | DNase | Nuclear | loop-helix + |
| | | I + RNase A | ribonucleoproteins | Ribosomal |
| | | each 100 | p53 + RNase If | protein each |
| | Control | μg/mL | each 100 μg/mL | 100 μg/mL |
| 1 passage | 0.012 | 0.015 | 0.014 | 0.01 |
| 2 passage | 0.01 | 0.011 | 0.01 | 0.014 |
| 3 passage | 0.015 | 0.009 | 0.01 | 0.012 |
| 4 passage | 0.009 | 0.165 | 0.059 | 0.096 |
| 5 passage | 0.007 | 0.188 | 0.092 | 0.134 |
| 15 passage | 0.012 | 0.230 | 0.169 | 0.207 |

As it can be seen the formation of cells with multiple cycles of the use of tested products each followed by a wash-out period enabled these cells to start sensing and fermenting novel products without of any artificial genome modifications. Such managing of cells genome information enables makes them recognize and inactivate xenobiotics; to form cells sensing novel factors, to inactivate, utilize and synthesis of programmed products with a non-limiting examples for the use of such organisms for the modulation of environmental pollution, waste management, construction, food preparation (i.e. fermenting products, serving as probiotics), biotechnology.

### Example 71: Products and Method Managing Stem Cells and Increase Longevity

To evaluate the effect of tested product on longevity and stem cells differentiation, we used umbilical cord-derived mesenchymal stromal cells treated or not treated with products in a range of concentrations from 1 μg/ml up to 1000 μg/ml with the exposure from 1 to 240 minutes on managing and evaluated the antioxidant and antiaging activity of mesenchymal stromal cell-conditioned medium (MSCM). Briefly, mesenchymal stromal cells were isolated from umbilical cord. Fibroblasts were isolated from human foreskin, incubated in collagenase for 90 minutes, and incubated in DMEM supplemented with 10% FBS and antibiotics as described before. Control probes were cultivated in normal glucose (6 mmol/L) level. To modulate stress, cells were placed to a high-glucose level of 30 mmol/L. To induce fibroblasts' differentiation cells were separated from the extracellular matrix and left either untreated or treated with tested products were further incubated with recombinant human TGF-β1 (5 ng/mL for 40 hours). Intracellular ROS were determined by DCFH-DA fluorescence. For that cells were incubated with 10 μmol/L DCFH-DA. The regulatory role of MSC-CM (treated or not treated with nucleases) was assessed by pretreating fibroblasts with 2.5% a of MSC-CM grown and plating to a high glucose environment (Table 80).

TABLE 80
Effects of tested products on regulation of mesenchymal stromal cells

| | DCHF positive cells (%) | | | | | |
|---|---|---|---|---|---|---|
| | | | | | | High glucose |
| | | | | High glucose | High glucose | level MSCM |
| | Normal | | High glucose | level MSCM | level MSCM | treated with |
| | glucose | High glucose | level MSCM | treated with | treated with | DNase and |
| | level | level | control | DNase | RNase | RNase |
| Oxidative | 5 + 0.3 | 67 + 5 | 38 + 2 | 23 + 3* | 140 + 2* | 8 + 1* |
| stress | | | | | | |
| Upregulation | + | ++++ | ++ | ++ | ++ | + |
| of p16 | | | | | | |
| Upregulation | + | ++++ | ++ | ++ | + | + |
| of p21 | | | | | | |

The results shown here are from on triplicate experiments. *P<0.05, for MSCM vs cells with altered by tested product

These results show that effect of tested products on cells including stem cells can managing oxidative stress that is related to cells' senescence. Moreover, we have demonstrated that effect of tested products can be used for managing the upregulation of genes associated with cellular aging, such as p16 and p21.

We also analyzed, how tested products can managing cell differentiation (tables 81, 82).

TABLE 81
Regulation of cell differentiation.

| | | % of fibroblasts |
|---|---|---|
| Glucose | | differentiated into |
| level | Probe | myofibroblasts |
| Normal | Control | 23 + 4 |
| High | Control | 62 ± 6 |
| | Treated with DNase | 0 |
| | Treated with RNase | 40 + 8 |
| | Treated with DNase + RNase | 68 + 12 |
| | Cultured in the presence of DNase | 29 + 8 |
| | Cultured in the presence of RNase | 55 + 9 |
| | Cultured in the presence of DNase + RNase | 44 + 6 |
| | Histone H1 | 0 |
| | pyrrole-imidazole-pyrrole oligomer | 0 |
| | Modified pyrrole-imidazole-pyrrole | 48 ± 8 |
| | oligomer | |
| | T7 RNA polymerases | 54 ± 12 |
| | Modified Ribosomal protein S1 | 45 ± 7 |
| | DNA Methyltransferases | 67 ± 9 |
| | Propidium iodine | 82 ± 14 |

TABLE 82
Effects of tested products in regulation of cell differentiation

| | Increase/decrease | | Inhibition of |
|---|---|---|---|
| | of fibroblasts | | proinflammatory |
| | differentiated into | | cytokines |
| Tested product | myofibroblasts | Tested product | production |
| Antibodies againt primary | Decrease | hnRNP C 1 μg/mL | Increase |
| DNA-based TezRs 1 μg/mL | | | |
| Antibodies againt primary | Increase | Modified 2- | Increase |
| RNA-based 1 μg/mL | | aminobenzimidazole | |
| | | derivative 1 μg/mL | |
| Modified mitomycin C 10 | Decrease | Histone H5 500 μg/mL | Increase |
| μg/mL | | | |
| Modified Mitotane 1000 | Increase | Naphthalene-based | Increase |
| μg/mL | | diimide conjugated bis- | |
| | | aminoglycoside 5 μg/mL | |
| Histone H5 10 μg/mL | Decrease | HuR 100 μg/mL | Increase |
| Transcriptional repressor | Increase | Dicer-like protein 10 | Increase |
| protein Lambda repressor | | μg/mL | |
| 1000 μg/mL | | | |
| Modified chrysophanol 10 | Yes | Tobramycin 1 μg/mL | Increase |
| μg mL | | | |

At normal glucose level, in the presence of TGF-β1, 62±6% of fibroblasts differentiate into myofibroblasts in 72 h. These data clearly show that treatment by products differentially affected cells' differentiation a behavior of pluripotent cells.

### Example 72: Products and Method of Ageing Managing

Normal human dermal fibroblasts were isolated from a juvenile foreskin and cultivated according to standard procedures throughout several passages. Cells were separated from the extracellular matrix and untreated or treated with tested products in a range of concentrations from 1 μg/ml up to 1000 μg/ml and incubated from 30 sec to 60 minutes. Some cells had multiple cycles of treatment with nucleases followed by wash-out period to generate “zero cells”. Average telomere length was measured from total genomic DNA. DNA was extracted with Qiagen DNA kit. We measured the mean telomere length by using the qPCR method previously described [Salpea K D, Nicaud V, Tiret L, Talmud P J, Humphries S E (2008) The association of telomere length with paternal history of premature myocardial infarction in the European Atherosclerosis Research Study II. J Mol Med 86: 815-824]. The relative telomere length which is known to correlate with chronological age was calculated as the ratio of telomere repeats to single-copy gene copies (T/S ratio) which were determined with quantitative PCR and adjusted for the cumulative population doublings. Cumulative population doublings was estimated as the number of population doubling (population doubling=[ln(number of cells harvested)−ln(number of cells seeded)]/ln2) with progressively adding the population doubling in each passage. The results are shown in FIG. 50.

As it can be seen, the use of tested products as well as transferring cells to a “zero” state inhibited telomere shortening (all p<0.05). This effect was the most pronounced in a “zero” state cells.

### Example 78: Products and Method for Managing Cells Characteristics Ex Vivo with Subsequent Allogeneic Transplantation

Female NOD SCID (CB17-Prkdcscid/NcrCrl) mice weighting 18 to 20 g were used. Subcutaneous tumors were established by injection of 7.0 log 10 Raji cells. CD8 T were collected. Some of CD8 T were treated with products in a range of concentrations from 1 μg/ml up to 1000 μg/ml and some transduced with lentiviral vector coding for CD19 CAR and after that treated with nucleases. Some cells had multiple cycles (from 2 up to 10) of treatment with tested products followed by a wash-out period to generate “zero cells”, or had a continuous treatment over 48 h. Some cells were also pretreated with combination of reverse transcriptase and integrase inhibitors (from 0.1 up to 1000.0 μg/mL). Cells were transplanted back to animals on day 8 post tumor implantation. Tumor volume was measured on day 60 post tumor implantation and rounded up to “5” (Table 83)

TABLE 83
Effect of tested products on modulation
of cells characteristics ex vivo.

| | Tumor size |
|---|---|
| Group | (mm3) |
| CD8 T control | 1480 + 160 |
| CD8 T treated with DNase 50 μg/ml | 970 + 115* |
| CD8 T treated with RNase 10 μg/ml | 1360 + 220* |
| CD8 T treated with Nuclease S1 50 μg/ml | 1150 + 325* |
| CD8 T treated with DNase + RNase each 10 μg/ml | 550 + 185* |
| CD8 T treated with multiple rounds of DNase + | 335 + 145* |
| RNase each at 10 μg/ml to generate “zero cells” | |
| CD8 T treated with with DNase + RNase each at | 205 + 45* |
| 10 μg/ml and mix of reverse transcriptase and integrases | |
| inhibitors (etravirine, tenofovir, raltegravir) | |
| CD19 CAR T control | 770 + 220* |
| CD19 CAR T treated with Histone 5 1 μg/ml | 415 + 170* |
| CD19 CAR T treated with ribosomal protein S15a 1000 | 565 + 135* |
| μg/ml | |
| CD19 CAR T cultivated in presence of DNase I and | 680 + 295* |
| RNase A each 50 μg/ml | |
| CD19 CAR T treated with XmnI + RNase P each 100 | 310 + 100* |
| μg/ml | |
| CD19 CAR T with multiple rounds of DNase 50 μg/ml | 115 + 20* |
| to generate “zero-D cells” | |
| CD19 CAR T with multiple rounds of RNase 10 μg/ml | 160 + 40* |
| to generate “zero-R cells” | |
| CD19 CAR T with multiple rounds of DNase + RNase | 30 + 150* |
| 100 μg/ml to generate “zero-DR cells” | |

*p < 0.05

These data clearly show that tested products can be used for managing gene information of cells to reprogram the cells and subsequent transplantation to the results in the altered functioning of these cells. Combination of products can potentiate this effect.

### Example 79: Product and Method for Managing Resistance of Tumors

ATCC cell line E0771 were maintained in DMEM supplemented with 1000 FBS and 100 penicillin/streptomycin, at 37° C. under 50% C02 atmosphere.

Control E0771, or treated with products in a range of concentrations from 1 μg/ml up to 10 mg/ml alone or as a combinations with reverse transcriptase and integrase inhibitors (from 0.1 up to 1000 μg/mL) were used. Stimulation of PD-L1 expression was done by treating cells with IFN-γ. The level of PD-L1 expression was assed with anti-PD-L1-antibody and rounded up to “1”. Data are presented in table 84.

TABLE 84
Effect of tested products on PD-L1 expression

| | Alteration | | Alteration |
|---|---|---|---|
| | of PD-L1 | | of PD-L1 |
| | mRNA | | mRNA |
| Group | expression | Group | expression |
| Control (stimulated | No | Exonuclease III 10 | Yes |
| with IFN) | | μg/ml | |
| | | Histone H2A 1 | Yes |
| | | mg/ml | |
| Treated with DNase | Yes | Uracil-DNA | Yes |
| 10 μg/ml | | glycosylase 100 | |
| | | μg/ml | |
| Treated with RNase | Yes | Topoisomerase I 1 | Yes |
| 1 μg/ml | | mg/ml | |
| Treated with DNase + | Yes | Pentatricopeptide | Yes |
| RNase “drunk cells” | | repeat protein 10 | |
| each at 100 μg/ml | | μg/ml | |
| Zero-DR cells | Yes | Modified amikacin | Yes |
| | | 10 μg/ml | |
| CD8 T treated with | Yes | N-methyl-3- | Yes |
| combination of | | hydroxypyrrole 1 | |
| DNase + RNase and | | μg/ml | |
| mix of reverse | | | |
| transcriptase and | | | |
| integrases inhibitors | | | |
| (etravirine, tenofovir, | | | |
| raltegravir) | | | |
| BsaJI 10 mg/ml | Yes | DNase1L2 1 μg/ml | Yes |

These data clearly shows that tested products can be used for the regulation of PD-L1 expression, proto-oncogene expression and crosstalk between cancer and immune cells.

### Example 80 Products and Method for Managing of Longevity

C. elegans (Carolina biosciences) were maintained using standard methods on nematode growth media. Synchronized samples were prepared by the egg-laying method by placing young adults for 4 h onto E. coli-seeded plates and subsequently removing them. Eggs (#100) were pretreated with products in a range of concentrations from 0.1 μg/ml up to 1 mg/ml. All lifespan analyses were carried out at 22° C. and rounded up to “0.1”. Viability was evaluated every 2 days, and death was considered when worms did not respond to a gentle touch with a sterilized wire. Some cells were also pretreated with combination of reverse transcriptase and integrase inhibitors (from 0.1 up to 1000.0 μg/mL). Data are presented in table 85.

TABLE 85
Effect of tested products in modulation of the mean lifespan

| | Mean |
|---|---|
| Group | lifespan |
| Control | 14.1 ± 1.2 |
| Treated with DNase 0.1 μg/ml | 19.6 ± 1.5* |
| Treated with DNase 1 mg/ml | 19.6 ± 1.5* |
| Treated with RNase 0.1 μg/ml | 18.3 ± 2.2* |
| Treated with DNase + RNase each taken at 100 μg/ml | 22.7 ± 0.9* |
| Treated with Histone 5 10 μg/ml | 18.9 ± 1.9* |
| Treated with T7 RNA polymerases 1 μg/ml | 21.1 ± 2.3* |
| Treated with imidazole pyrrole pyrrole oligomer 100 μg/ml | 23.2 ± 3.0* |
| Five cycles of treatment with DNase + RNase each taken | 28.9 ± 3.3* |
| at 10 μg/ml | |
| Treated with combination of DNase + RNase each taken at | 33.1 ± 4.0* |
| 10 μg/ml and mix of reverse transcriptase and integrases | |
| inhibitors (etravirine, tenofovir, raltegravir) | |

*p < 0.05

These data clearly demonstrate that the use of tested products can be used to increase longevity.

### Example 81. Products and Method for Managing Product Yield in Biomanufacturing

To study effect of tested products on insulin precursor (IP) production, we used pPIC9K expression vector construction that was used for the transformation of P. pastoris strain GS 115his-. After that cells were pretreated or not pretreated with in a range of concentrations from 1 μg/ml up to 1000 μg/ml. Some cells were also pretreated with combination of reverse transcriptase and integrase inhibitors (from 0.1 up to 1000.0 μg/mL). Transformants were plated to Mini Bioreactors 500 mL (in normal or altered geomagnetic condition by placing them in μ-tissue) filled with 250 mL of autoclaved growth media, adjusted to pH 5.0 with 25 NH4H. Stirrer speed was controlled between 200 to 800 rpm at 30° C. After the growth stage when glycerol was depleted, glycerol-enrichment stage was initiated with glycerol solution (50 glycerol (w/w), biotin and PTM1). After 6 h, production of IP was initiated by addition of 990 methanol, Biotin 0.2 and PTM1). TP quantification was done with HPLC. Data are presented in table 86.

TABLE 86
Effect of tested products in biomanufacturing

| | IP (g/L − 1) | |
|---|---|---|
| | Normal magnetic | Altered magnetic |
| Group | field | field |
| Control | 0.31 ± 0.12 | 1.87 ± 0.46 |
| Treated with DNase 10 μg/mL | 1.90 ± 0.18* | 3.77 ± 0.49* |
| Treated with RNase 10 μg/mL | 1.46 ± 0.33* | 4.35 ± 0.25* |
| Treated with DNase + RNase | 2.12 ± 0.47* | 5.32 ± 0.46* |
| each 100 μg/mL | | |
| Zero-DR cells | 3.76 ± 0.54* | 6.89 ± 1.72* |
| Treated with combination of | 3.0 ± 0.45* | 6.42 ± 0.93* |
| DNase + RNase each at 10 μg/mL | | |
| and mix of reverse transcriptase | | |
| and integrases inhibitors | | |
| (etravirine, tenofovir, raltegravir) | | |
| Ribosomal protein S40 10 | 2.54 ± 0.35* | 2.77 ± 0.34* |
| μg/mL | | |
| Modified Paromomycin 1000 | 1.97 ± 0.29* | 3.52 ± 0.41* |
| μg/mL | | |
| DNA polymerase- β family 1 | 0.94 ± 0.08* | 3.80 ± 0.56* |
| μg/mL | | |
| Modified Amidinium 10 μg/mL | 1.19 ± 0.20* | 5.53 ± 0.325* |

*p < 0.05

Data received point out that the use of tested products in normal and altered magnetic field can be used for managing of biomanufacturing including increase of the product yield.

### Example 82: Products and Method Cell Protection Against Products for the Managing Cells Behavior

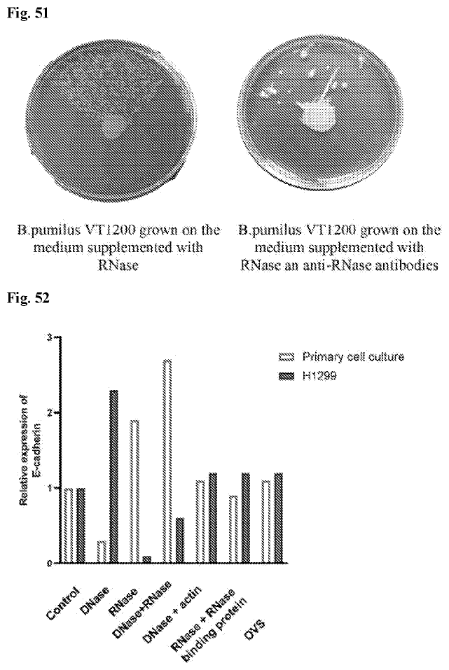

Antibodies against RNase at 10 μg/ml were added to the agar of 90 mm Petri dish filled the mix of Columbia and Pepted meat agar with ⅙ sector containing from 50 μL fresh human volunteer plasma filtered through 0.22 uM filter. Control plated had no antibodies. 25 uL of overnight B. pumilus VT1200 was placed on the center of the plates, and plates were incubated at 37° C. for 24 hours and photographed with Canon 6D (Canon, Japan). Data are presented in FIG. 51.

It is clearly seen that product as anti RNase antibody can be used for managing of cell responses.

### Example 83: Products for Managing Virulence of Eukaryotes and Prokaryotes and for Diagnostic of Diseases Associated with NAMACS and/or NAMACS-ANA Capable of Recognizing Biological, Chemical and Physical Factors

Surgical cells of patient with pancreatic cancer were trypsonized and were either left untreated or treated with tested products in a range of concentrations from 1 μg/ml up to 10 mg/ml.

Oral microbiota of healthy individual was either left untreated or treated with tested products in a range of concentrations from 1 μg/ml up to 10 mg/ml.

The pooled blood of health volunteers (n=5, mean age 43.4) was either left untreated, or treated with (i) isolated cancer cells from 10e2 to 10e8 cells/ml, or with (ii) oral microbiota from 10e2 to 10e9 bacteria/ml, and incubated for from 1.0 up to 360 minutes at 37° C. and subsequently heated up to 100° C. for from 10 sec up to 60 min. LC/MS was conducted. Table 87 below shows effect of products at formation of found in the plasma of a healthy volunteers and cancer patients.

TABLE 87
Effect of products to inhibit formation of disease associated heat-resistant proteins.

| | Probe | | | | |
|---|---|---|---|---|---|
| | | Eukaryotic cells | Eukaryotic | Microbiota | Microbiota |
| | | treated with | cells | treated with | cells |
| | Untreated | products | untreated | products | untreated |
| Colorectal cancer | No | No | Yes | No | Yes |
| (Reversion-inducing | | | | | |
| cysteine-rich protein with | | | | | |
| Kazal motifs) | | | | | |
| Ovarian cancer | No | No | Yes | No | Yes |
| (Eukaryotic translation | | | | | |
| initiation factor 5A-1) | | | | | |
| Ovarian cancer (Inter-a- | No | No | Yes | No | Yes |
| trypsin inhibitor heavy | | | | | |
| chain H4 fragment) | | | | | |
| Ovarian cancer (CD5L) | No | No | Yes | No | Yes |
| Pancreatic cancer | No | No | Yes | No | Yes |
| (Serotransferrin) | | | | | |
| Pancreatic cancer | No | No | Yes | No | Yes |
| (Complement factor H- | | | | | |
| related protein) | | | | | |
| Pancreatic cancer | No | No | Yes | No | Yes |
| (Immunoglobulin | | | | | |
| lambda constant 7) | | | | | |
| Hairy leukemia | No | No | Yes | No | Yes |
| (Immunoglobulin kappa | | | | | |
| variable) | | | | | |
| Lung Cancer (ITIH4) | No | No | Yes | No | Yes |
| Lung Cancer (Plasma | No | No | Yes | No | Yes |
| protease C1 inhibitor) | | | | | |
| Lung Cancer | No | No | Yes | No | Yes |
| (Immunoglobulin lambda | | | | | |
| constant 7) | | | | | |
| Melanoma (CD5 antigen- | No | No | Yes | No | Yes |
| like) | | | | | |
| Melanoma (Keratin) | No | No | Yes | No | Yes |
| Melanoma (Type I | No | No | Yes | No | Yes |
| cytoskeletal 9) | | | | | |
| Prostatic cancer | No | No | Yes | No | Yes |
| (Selenoprotein P) | | | | | |
| Prostatic cancer | No | No | Yes | No | Yes |
| (kallikrein 2) | | | | | |
| Prostatic cancer | No | No | Yes | No | Yes |
| (apolipoprotein A-II | | | | | |
| Proteins associated with | No | No | No | No | Yes |
| Congenital | | | | | |
| analbuminemia | | | | | |
| Proteins associated with | No | No | No | No | Yes |
| Hyperthyroxinemia | | | | | |
| Proteins associated with | No | No | No | No | Yes |
| Thyroid carcinoma | | | | | |
| Proteins associated with | No | No | No | No | Yes |
| Noonan syndrome | | | | | |
| Proteins associated with | No | No | No | No | Yes |
| Glioma | | | | | |
| Proteins associated with | No | No | No | No | Yes |
| Schizophrenia | | | | | |
| Proteins associated with | No | No | No | No | Yes |
| Retinal dystrophy | | | | | |
| Proteins associated with | No | No | No | No | Yes |
| Alzheimer disease | | | | | |
| Proteins associated with | No | No | No | No | Yes |
| Corneal dystrophy | | | | | |
| Proteins associated with | No | No | No | No | Yes |
| Dilated cardiomyopathy | | | | | |
| Proteins associated with | No | No | No | No | Yes |
| Congenital | | | | | |
| atransferrinemia | | | | | |
| Proteins associated with | No | No | No | No | Yes |
| Primary glomerular | | | | | |
| disease | | | | | |
| Proteins associated with | No | No | No | No | Yes |
| Primary glomerular | | | | | |
| disease | | | | | |
| Proteins associated with | No | No | No | No | Yes |
| Fibronectin | | | | | |
| glomerulopathy | | | | | |

Products may be used for prophylactic and treatment of disease associated with NAMACS and NAMACS-ANA of eukaryotic and microbiota cells and/or associated with them. These nucleic acids molecules as well as proteins formed in the test plasma of healthy people following their adding, can be used to diagnose various diseases.

### Example 84: Analysis of Cell-Surface Bound Nucleic Acids as a Sign of Health and Disease Together with Other Diagnostics Tests

12 patients suspected according to routine analysis (screening tests including colonoscopy, prostate specific antigen, mammography, cytology, circulating tumor DNA, biomarker detection,) were suspected to have certain malignancies (pancreatic cancer, lung cancer, colorectal cancer, prostate cancer, liver cancer, mesothelioma), but the diagnose was not established yet and required other confirmational analysis. We studied to the composition of cell-surface bound nucleic acids of cells needle biopsy material of the cancer or from sputum (for patient with the lung cancer). Cells from the same location were obtained from surgical material from non-oncological patients.

cell-surface bound nucleic acids were visualized with DAPI, SYTOX green (Excitation: 504; Emission 523), Propidium Iodine (Excitation: 493; Emission 636) with Revolve microscope from ECHO (ECHO San Diego CA) and Synergy Neo2 Multi-Mode Microplate Reader (Biotek).

To isolate cell-surface bound nucleic acids from tissues of patients suspected to have tumors or control, tissues were homogenated, collagenase was added. Cells were gently washed and filtered through 0.22 uM, to let debris and some intracellular nucleic acids that could be in the material to pass through. After that cells were placed to a 0.9% NaCl supplemented with BamHI and HindIII BbvCI, BgII, FokI, AcuI nucleases, with added Mg buffer for 1 h at 37 C. Cells were separated by centrifugation 3000 g×10 minutes and supernatant was filtered through the 0.22 uM filter (Millipore). DNA was isolated from the supernatant with QIAamp DNA Mini Kit (Qiagen). The RNA was isolated with a Quick-RNA Kits (Zymo research).

The whole-genome sequence was obtained using the Illumina HiSeq 2500 sequencing platform (Illumina GAIIx, Illumina, San Diego, CA, USA). Library preparation, sequencing reactions, and runs were carried out according to the manufacturer's instructions. *Amount of cell-surface bound nucleic acids of Non-altered cells of each type was suggested as “norma.” Also, some cells were stained with Sytox as described above and the alteration of the surface green fluorescence corresponds was analyzed as the sign of cell-surface bound nucleic acids alterations. Data are presented in table 88.

TABLE 88
Use of TezRs_D1/R1 to diagnose human
disease when accompanied with other methods

| | Presence of | | Presence of |
|---|---|---|---|
| | pathological | | pathological |
| | alterations of cell- | | alterations of cell- |
| | surface bound | | surface bound |
| Diagnose | nucleic acids | Diagnose | nucleic acids |
| Patient 1. | Yes | Patient 1. | Yes |
| Suspected to | | Suspected to | |
| colorectal cancer | | lung cancer | |
| Patient 2. | Yes | Patient 2. | Yes |
| Suspected to | | Suspected to | |
| colorectal cancer | | lung cancer | |
| Patient 1. | No | Patient 1. | No |
| Control for | | Control for | |
| colorectal cancer | | lung cancer | |
| Patient 2. | No | Patient 2. | No |
| Control for | | Control for | |
| colorectal cancer | | lung cancer | |
| Patient 1. | Yes | Patient 1. | Yes |
| Suspected to | | Suspected to | |
| pancreatic cancer | | prostate cancer | |
| Patient 2. | Yes | Patient 2. | Yes |
| Suspected to | | Suspected to | |
| pancreatic cancer | | prostate cancer | |
| Patient 1. | No | Patient 1. | No |
| Control for | | Control for | |
| pancreatic cancer | | prostate cancer | |
| Patient 2. | No | Patient 2. | No |
| Control for | | Control for | |
| pancreatic cancer | | prostate cancer | |
| Patient 1. | Yes | Patient 1. | Yes |
| Suspected to | | Suspected to | |
| liver cancer | | mesothelioma | |
| Patient 2. | Yes | Patient 2. | Yes |
| Suspected to | | Suspected to | |
| liver cancer | | mesothelioma | |
| | | cancer | |
| Patient 1. | No | Patient 1. | No |
| Control for | | Control for | |
| liver cancer | | mesothelioma | |
| | | cancer | |
| Patient 2. | No | Patient 2. | No |
| Control for | | Control for | |
| liver cancer | | mesothelioma | |
| | | cancer | |

*Sequence of cell-surface bound nucleic acids of non-altered cells of each type was suggested as “normal”.

These data clearly show that cell-surface bound nucleic acids can be used for the highly accurate diagnostic of mammalian diseases together with other diagnostic methods.

### Example 85: Products and Method for Managing of Product Yield in Biomanufacturing

To study the effect of tested products on the product yield in biomanufacturing, we used a cell line with insulin precursor (IP) production. For that E. coli expression vector construction was used to transform E. coli ATCC 25922 strain. After that, cells were pretreated or not pretreated with tested compounds. Transformants were plated to flask that model bioreactors 500 mL (in normal or altered geomagnetic condition by placing them in μ-tissue) filled with 250 mL of growth media.

The suspension CH0 cell line producing recombinant lgG treated or not treated with nucleases were seeded at 2×10e cells/mi in 30 ml of nutrient medium. Recombinant mouse lgG production yield was assayed 1 to 6 days after transfection using protein G biosensor (fortéBIO® octet RED96 system).

TP quantification was done with HPLC. The yield of lgG production was assayed by day 5. Data are presented in table 89.

TABLE 89
Effect of tested products on biomanufacturing

| | IP (% to WT in normal magnetic filed) | | | |
|---|---|---|---|---|
| | Normal magnetic | | Altered magnetic | |
| | field | | field | |
| Group | No shaker | Shaker | No shaker | Shaker |
| | E. coli | | | |
| Control | 100% | 178% | 225% | 364% |
| DNase I 100 μg/mL | 194%* | 289%* | 438%* | 513%* |
| DNase I 10 pg/mL | 165%* | 229%* | 384%* | 497%* |
| RNase A 100 μg/mL | 150%* | 203%* | 339%* | 542%* |
| RNase A 0.1 μg/mL | 146%* | 221%* | 343%* | 472%* |
| Modified RNase A 100 μg/mL | 168%* | 215%* | 351%* | 490%* |
| Treated with combination | 172% | 319% | 588% | 884% |
| DNase I + RNase A each 10 | | | | |
| μg/mL and mix of reverse | | | | |
| transcriptase and integrases | | | | |
| inhibitors (Etravirine, | | | | |
| tenofovir, raltegravir) | | | | |
| BanII 10 μg/mL | 148%* | 210%* | 385%* | 427%* |
| RNase polymerase III 100 | 189%* | 242%* | 323%* | 475%* |
| μg/mL | | | | |
| Ribosomal protein L11 10 | 187%* | 237%* | 389%* | 436%* |
| μg/mL | | | | |
| Histone H5 100 μg/mL | 204%* | 309%* | 512%* | 625%* |
| Modified Pyrrole-imidazole | 197%* | 311%* | 375%* | 430%* |
| polyamide1 μg/mL | | | | |
| benzimidazol-2-yl-fur-5-yl- | 158%* | 296%* | 336%* | 437%* |
| (1,2,3)-triazolyl dimeric | | | | |
| derivative 10 μg/mL | | | | |
| N-methyl-3-hydroxypyrrole- | 173%* | 317%* | 519%* | 606%* |
| pyrrol 1 μg/mL | | | | |
| | CHO cells | | | |
| Control | n/a | 100% | n/a | n/a |
| DNase I 100 μg/mL | n/a | 245%* | n/a | n/a |
| RNase A 100 μg/mL | n/a | 260%* | n/a | n/a |
| RBP ProQ 100 μg/mL | n/a | 315%* | n/a | n/a |
| Ribosomal protein L11 10 | n/a | 276%* | n/a | n/a |
| μg/mL | | | | |
| Modified Histone H5 100 | n/a | 212%* | n/a | n/a |
| μg/mL | | | | |

Data received point out that tested products manage the yield of products in both normal and altered magnetic field with or without of shaking and can be used for the management of biomanufacturing and increasing of the product yield.

### Example 86: Products and Method for Managing Neoplasm Transformation

We evaluated the effect of products on preventing of the neoplastic transformations. For that, serum-supplemented medium of RWPE-1 cells was removed and the cell monolayer was washed once with PBS and once serum-free medium. After that cells were separated from the extracellular matrix, treated with tested compounds in a range of concentrations from 1 μg/ml up to 10 mg/ml and exposed to phorbol 12 myristate (PMA) 50 ng/mL and the expression of MMVP9 as a signature of the neoplastic transformation was monitored. Data are presented in FIG. 90.

TABLE 90
Effect of tested products on cancerogenic transformation

| Group | MMP9 fold change |
|---|---|
| Control | 1 |
| Control + PMA | 2.9 |
| Treated with DNase 1 μg/ml + PMA | 1.2* |
| Treated with RNase 100 μg/ml + PMA | 1.3* |
| Treated with DNase + RNase each 100 μg/ml + | 1.1* |
| PMA | |
| Treated with ApaI 1 μg/ml + PMA | 1.2* |
| Treated with Ribosomal protein S28 + PMA | 1.6* |
| Treated with modified riboflavin 10 μg/ml + | 1.9* |
| PMA | |
| Treated with modified nogalamycin 10 μg/ml + | 1.4* |
| PMA | |

*p < 0.05

It is clearly seen that products that the tested products can inhibit cancer transformation.

### Example 87: Products and Method for the Control of Active Regulatory Substances Synthesis and Production by Cells

5 patients with obesity (group OB-CONTROL) collected intact their samples using a plastic stool collection container. Probes were frozen. Prior to the analysis these probes were thawed in an anaerobic chamber, extracellular matrix was removed, probes were dissolved with PBS, treated or not treated with products in a range of concentrations from 1 μg/ml up to 1000 μg/ml and left for 6 h at 37° C. and filtered through 0.33 mm pore-size filter. The liquid fraction was removed for analysis of the SCFA using Agilent 7890b gas chromatograph. Data are presented in table 91.

TABLE 91
Effect of tested products on cells' metabolites
production including short chain fatty acids

| | Total SCFA |
|---|---|
| Group | concentrating (mM) |
| OB-CONTROL | 24 ± 5 |
| Treated with DNase 10 μg/ml | 8 ± 4* |
| Treated with RNase 1 μg/ml | 6 ± 3* |
| Treated with DNase + RNase each 1000 μg/ml | 9 ± 4* |
| Treated with Hairpin polyamide 100 μg/ml | 8 ± 3* |
| 1,3-Bis{1-[((5-(5-amidino)benzimidazol-2- | 5 ± 2* |
| yl)furan-2-yl) methylene]-1H-1,2,3-triazole- | |
| 4-yl}propane hydrochloride 1 μg/ml | |
| Treated with Ribosomal protein L3 100 μg/ml | 12 ± 3* |
| Histone H1 10 μg/ml | 9 ± 4* |
| Modified Mitotane 1000 μg/ml | 15 ± 4* |

*p < 0.05

It is clearly seen that tested products can regulate metabolites production including short chain fatty acids.

### Example 88: Products and Method for Managing of Cells Responses

CAR-T and Mock T cells were obtained as previously described [7], separated from the extracellular matrix, treated with tested compounds in a range of concentrations from 1 μg/ml up to 10 mg/ml from 1 to 240 minutes and resuspended in RPMI+IL-2/RPMI. Some CAR- and Mock T cells were treated with multiple rounds of nucleases to generate Zero-D, Zero-R or Zero-DR cells as previously described. Raji and Jeko cells were also separated from the extracellular matrix, treated with tested compounds in a range of concentrations from 1 μg/ml up to 1 mg/ml from 1 to 240 minutes and resuspended in RPMI+IL-2/RPMI. The effects of tested products were assessed by monitoring specific lysis.

Results are shown in tables 92 and 93.

TABLE 92
Effect of treatment of tumor cells with tested products
on the antitumor activity of CAR-T and Mock T cells.

| | Specific lysis of Raji (%) | |
|---|---|---|
| Group of Raji cells | MockT | CAR19 |
| Control | 12 | 48 |
| Treated with DNase 1 μg/ml | 24* | 63* |
| Treated with RNase 1 μg/ml | 32* | 92* |
| Treated with DNase + RNase each | 39* | 71* |
| 1000 μg/ml | | |
| Treated with DNase 100 μg/ml | 29* | 87* |
| Treated with modified RNase 100 | 38* | 85* |
| μg/ml | | |
| Treated with DNase + modified RNase | 44* | 77* |
| each 1000 μg/ml | | |

These data point out that the used compounds in these settings increase sensitivity of cells for the anticancer cell therapies and immune cells.

TABLE 93
Effect tested compounds on immune
cells-induced antitumor activity.

| | Specific lysis of Jeko (%) | |
|---|---|---|
| Group | MockT | CAR19 |
| Control | 10 | 54 |
| Treated with DNase 10 μg/ml | 49* | 91* |
| Treated with RNase 1 μg/ml | 28* | 79* |
| Treated with DNase + RNase each 1000 | 30* | 88* |
| μg/ml | | |
| Treated with BclI-HF 10 μg/ml | 38* | 78* |
| Treated with Histone H5 10 mg/ml | 40* | 82* |
| Treated with TLR9 100 μg/ml | 42* | 69* |
| REC J nuclease | 19* | 83* |
| Treated with pyrrolo[2,1- | 23* | 75* |
| c][1,4]benzodiazepine-benzimidazole | | |
| hybrid 10 μg/ml | | |
| Zero-D | 69* | 97* |
| Zero-R | 46* | 86* |
| Zero-DR | 75% | 94* |

These data point out that the treatment of immune cells with tested compounds increase their antitumor activity.

### Example 89: Products and Method for Regulation of Pathological Disease when Administered Systemically or Locally

The goal was to show that the tested products can be used for the modulation of fibrosis and NASH formation when used systemically and locally.

We used the STAM mouse model of NASH. The STAM model is created by a combination of chemical treatment (streptozotocin 200 μg) and high fat diet (60% energy from fat) in C57BL/6 mice. NASH was developed at week 7-8, and is advanced to fibrosis in weeks 10-12. Animals were treated with i.v (two times a week) with nuclease inhibitors (mouse actin and recombinant murine RNase inhibitor). Some animals received 2 times intrahepatic injections of these products on week 2 and week 4 after the start of the experiment. Comparison of NAS from mouse liver specimens in 10 week old mice included steatosis and fibrosis.

Results are shown in tables 94 and 95.

TABLE 94
Effect of tested compounds on the development of steatosis.

| Group | Score of the steatosis |
|---|---|
| Control | 2.8 |
| Actin i.v. 1 mg | 2.3* |
| Actin i.v. 20 mg | 1.7* |
| Murine RNase inhibitor i.v. 40U | 2.1* |
| Murine RNase inhibitor i.v. 4000U | 1.6* |
| Actin + Murine RNase inhibitor i.v. | 0.6* |
| Actin, intrahepatic injection | 1.2* |
| Murine RNase inhibitor, | 0.7* |
| intrahepatic injection | |
| Actin + Murine RNase inhibitor, | 0.2* |
| intrahepatic injection | |

*p < 0.05

These data point out that the inhibition of nucleases can be used for the therapy of mammalian diseases including the control of steatosis.

TABLE 95
Effect tested compounds on the development of fibrosis.

| Group | Score of the fibrosis |
|---|---|
| Control | 1.8 |
| Actin i.v. 1 mg | 1.5* |
| Actin i.v. 20 mg | 1.3* |
| Murine RNase inhibitor i.v. 40U | 1.4* |
| Murine RNase inhibitor i.v. 4000U | 1.1* |
| Actin + Murine RNase inhibitor i.v. | 0.6* |
| Actin, intrahepatic injection | 0.7* |
| Murine RNase inhibitor, intrahepatic | 0.5* |
| injection | |
| Actin + Murine RNase inhibitor, | 0.2* |
| intrahepatic injection | |

*p < 0.05

It is clearly seen that the use of tested compounds reduces NAS and fibrosis.

### Example 90: Products and Method for Regulation of Pathological Disease when Administered Systemically or Locally

The goal was to show that the tested products can be used for the modulation of fibrosis and NASH formation when used systemically and locally.

We used the STAM mouse model of NASH. The STAM model is created by a combination of chemical treatment (streptozotocin 200 μg) and high fat diet (60% energy from fat) in C57BL/6 mice. NASH was developed at week 7-8, and is advanced to fibrosis in weeks 10-12. Animals were treated with i.v (two times a week) with nuclease inhibitors (mouse actin and recombinant murine RNase inhibitor). Some animals received 2 times intrahepatic injections of these products on week 2 and week 4 after the start of the experiment. Comparison of NAS from mouse liver specimens in 10 week old mice included steatosis and fibrosis.

Results are shown in tables 96 and 97.

TABLE 96
Effect of tested compounds on the development of steatosis.

| Group | Score of the steatosis |
|---|---|
| Control | 2.8 |
| Actin i.v. 1 mg | 2.3* |
| Actin i.v. 20 mg | 1.7* |
| Murine RNase inhibitor i.v. 40U | 2.1* |
| Murine RNase inhibitor i.v. 4000U | 1.6* |
| Actin + Murine RNase inhibitor i.v. | 0.6* |
| Actin, intrahepatic injection | 1.2* |
| Murine RNase inhibitor, | 0.7* |
| intrahepatic injection | |
| Actin + Murine RNase inhibitor, | 0.2* |
| intrahepatic injection | |

*p < 0.05

These data point out that the inhibition of nucleases can be used for the therapy of mammalian diseases including the control of steatosis.

TABLE 97
Effect tested compounds on the development of fibrosis.

| Group | Score of the fibrosis |
|---|---|
| Control | 1.8 |
| Actin i.v. 1 mg | 1.5* |
| Actin i.v. 20 mg | 1.3* |
| Murine RNase inhibitor i.v. 40U | 1.4* |
| Murine RNase inhibitor i.v. 4000U | 1.1* |
| Actin + Murine RNase inhibitor i.v. | 0.6* |
| Actin, intrahepatic injection | 0.7* |
| Murine RNase inhibitor, intrahepatic | 0.5* |
| injection | |
| Actin + Murine RNase inhibitor, | 0.2* |
| intrahepatic injection | |

*p < 0.05

It is clearly seen that the use of tested compounds reduces NAS and fibrosis.

### Example 91: Products and Methods for Managing of Cell Activity

We studied the effect of cell surface nucleic acids destruction and protection of cell-surface nucleic acids destruction as a therapeutic intervention to treat and prevent disease progression. We protected cell-surface nucleic acids by inhibiting DNase with actin and RNase with RNase binding protein as previously described or oligomers of vinylsulfonic acid (OVS) derivatives.

Primary lung cancer cells isolated as described previously (Zheng et al 2011) and maintained for over 25 passages and H1299 cells were used. To determine whether the loss of surface nucleic acids destruction can lead to a pro-disease state or vice-a versa we examined the expressions of E-cadherin which is known as a key factor of epithelial-to-mesenchymal transition after the loss of surface nucleic acids destruction (Loh et al 2019).

RNA extraction and transcriptomic analysis was carried out as described previously. As shown in FIG. 52, the destruction of different surface nucleic acids destruction had different effects in different cells on the up- and downregulation of E-cadherin expression.

These data point out that both the destruction/inactivation as well as protection of surface nucleic acids destruction in some cells has a therapeutic potential.

### Example 96: Developing of Products and Methods for the Protection of Primary Cell-Surface Nucleic Acids

We studied could protection of extracellular nucleic acids from nucleases be used to prevent cellular alterations typical for cells following the destruction of their cell-surface nucleic acids. We studied it using a dispersal model of B. pumilus VT1200. Control bacteria were treated with RNase to trigger their dispersal and experimental were either treated with RNase together with Ribonuclease Inhibitor or with anti RNase antibodies for 30 min at 37 C. Data are presented in FIG. 53.

As it can be seen the protection of cell-surface nucleic acids resulted in significant inhibition of alterations of cells' characteristics following the loss of cell-surface nucleic acids.

### Example 97: Modulation of RNase Expression in Organism to Control Health State and Longevity

Given the broad range of characteristics modulated by cell-surface bound nucleic acids we suggested that alteration of RNase expression might be associated with the development of different diseases and longevity.

A wild-type E. coli strain VT-9 and the isogenic mutant with RNase gene (E. coli VT-9 RNase+) were obtained through from the laboratory of Human Microbiology Institute. The RNase expression was also confirmed by RNA destruction in the media as previously described.

C. elegans were propagated in standard conditions on nematode growth medium. Pates seeded with E. coli VT-9 WT or E. coli VT-9 RNase+ or at 20° C. Some animals were left untreated and to some recombinant murine RNase inhibitor (40 U/ml) were added, Data are presented in table 98.

TABLE 98
The influence of RNase level on lifespan.

| E. coli strain | Mean Lifespan (days) |
|---|---|
| E. coli strain VT-9 WT | 19.7 |
| E. coli strain VT-9 WT on media | 20.4 |
| supplemented with recombinant murine | |
| RNase inhibitor | |
| E. coli VT-9 RNase + on control media | 12.5* |
| E. coli VT-9 RNase + on media supplemented | 18.7 |
| with recombinant murine RNase inhibitor | |

*p < 0.05

These data point out that the longevity and healthiness can be modulated by the alteration of RNase expression level.

We used pyrosequencing (454 platform; Roche) to identify genome-wide base-substitution mutations in C. elegans fed with control and RNase producing E. coli strains. We found that an average μbs for E. coli fed with E. coli strain VT-9 WT was 2.9×10e-9 per site per generation. An average μbs for E. coli fed with E. coli strain VT-9 RNase+ was 1.3×10e-8 per site per generation. However, the E. coli strain VT-9 RNase+ grown on the media supplemented with recombinant murine RNase inhibitor had μbs 9.7×10e-8 per site per generation.

These data, surprisingly point out that the level of RNase production in organism regulates the frequency of spontaneous mutation and genome stability and by the regulation of RNase activity it is possible to control these processes.

### Example 98: Developing of Products and Methods for Erasure of Autoimmune Memory

Given that for the first-time discovered here role of NAMACS and NAMACS-ANA and TEZRs

in memory formation we studied the use of different products to erase memory in immune cells. Blood was drawn from ten patients with type 1 diabetes positive for HLA-A2. Peripheral blood mononuclear cells were isolated using Ficoll density gradient centrifugation and CD8+ T cell were isolated using StemCell Isolation Kit (STEMCELL Technologies). CD8+ T cells were left either untreated or put to the “zero state” by multiple times destruction with DNase and RNase as described previously and were cultured in 96-well round-bottom plates at 3×10e3 cells per well in DMEM medium. Next, 5×10e5 CD8+ T cells were co-cultured with K562 cells transfected with HLA-A*0201 (2.5e10×5 cells) which were either untreated or treated for 4 h with the set of islet antigen peptides (IA-2 797-805 ZnT8186-194, IGRP228-236, PPI2-10, PPI34-42). After that the level of cytokines in supernatant was measured by Luminex. Results are shown in FIG. 54.

Data received clearly show that the proposed products and methods can be used to control cytokines production, regulation of FOXP3 pathway and to erase cell memory and immunological memory as well.

### Example 99: Use of Products and Method to Regulate Cell Migration and Metastasis

A549 cell line were grown as monolayers in RPMI-1640 medium supplemented with 10% fetal calf serum (FCS) and L-glutamine (2 mM). The cell lines were maintained in an incubator with a humidified atmosphere (5% CO2 in air at 37° C.).

A549 cells were seeded (10,000 cells/well) in 24-well plates and allowed to attach to the surface under standard incubation conditions (RPMI-1640 medium supplemented with 10% fetal calf serum and L-glutamine (2 mM), 5% CO2 at 37° C.) for 24 h. The confluent cell monolayers were scratched in a straight line using a sterile plastic pipette tip. The de-attached cells were then carefully rinsed with RPMI-1640 medium to remove debris and free-floating cells. Media was removed and cells were treated with nucleases.

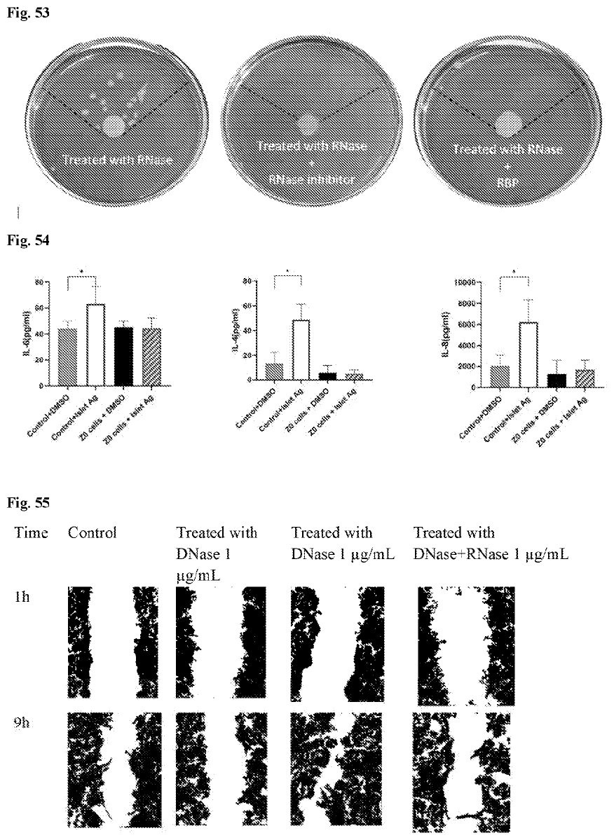

Then fresh media was added and cells. Scratch zones were photographed hourly by Zeiss Axiovert 40C (Carl Zeiss AG, Germany). Results are shown in FIG. 55, table 99.

TABLE 99

| | Acceleration | | Acceleration | | Acceleration |
|---|---|---|---|---|---|
| | of cell | | of cell | | of cell |
| | migration | | migration | | migration |
| Tested | compared | Tested | compared | Tested | compared |
| compound | to control | compound | to control | compound | to control |
| ExoR1 + RNase If | +56 | nucleophosmin | +31% | NF-κB | +45% |
| SMAD4 | +51% | DNA | +44% | Propidium | +36% |
| | | Methyltransferases | | iodine | |
| NONO protein | +64% | Modified | +66% | Modified | +42% |
| | | RNase + DNase | | Propidium | |
| | | | | iodine | |

As it seen, the use of tested products when they were used for the cell treatment significantly affected actin cytoskeleton, increased migration of cells that is essential for many physiological processes including wound repair, embryonic development, wound repair, tumor invasion, neoangiogenesis and metastasis.

### Example 100: Products and Methods to Regulate Sensitivity of Cells to Opioids

Subconfluent cultures of T98G human glioblastoma cells, highly expressing opioid receptor, were collected, washed twice with DMEM without FBS, and resuspended in DMEM supplemented with FBS. Cells were treated with nucleases at a final concentration of 100 μg/ml for 30 min as previously described. After the removal of nucleases, the cells were seeded in 96-well plates at a density of 4.0 log 10 cells per well and exposed to the freshly prepared tramadol (Sigma-Aldrich) at a concentration of 200 μM for 3 h at 37° C. with 5% CO2.

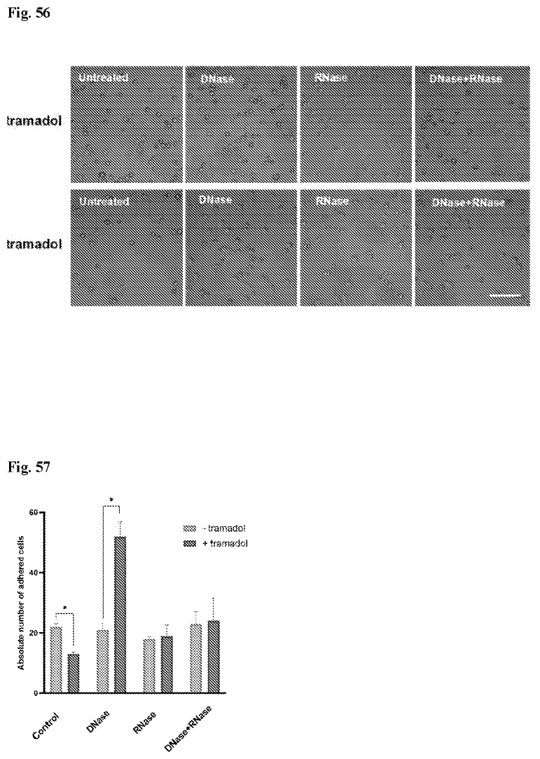

Resulted T98G cells were plated on media supplemented with tramadol and growth was compared to that of the same cells in media without tramadol. Tramadol treatment showed an inhibitory effect on cell attachment of control cells but not on that treated with nucleases (FIG. 54a,b). These results suggest that cells following the treatment with tested products do not react to tramadol-induced inhibition of cell adhesion, indicating that the use of products that destroy or inactivate TezRs can be used to supervises the work of protein receptors, including responses through PI3K/AKT pathway and to opioid compounds (Xia et al., 2016). Data are shows in FIGS. 56 and 57.

### Example 101: Products and Methods for the Increase of the Lifespan

Vaccines from genomic DNA and/or NAMACS and NAMACS-ANA of P. aeruginosa or autovaccine against mix of microorganisms isolated from the feces of mammal (mice) were obtained as described earlier. Mice (c57bl/6, #6 per group) were treated with different regimes of these vaccines and their longevity was monitored. Some mice were vaccinated at the young age (˜200 days) and some were old (˜400 days). Data are presented in table 100 (rounded to 1).

TABLE 100
Effect of vaccines on the lifespan of animals

| | | Median |
|---|---|---|
| | Age of animals at | lifespan |
| Type of vaccine | the first infection | (days) |
| Control | 200 | 534 |
| Vaccine from genomic DNA of | 200 | 795 |
| P. aeruginosa 1 time a month | | |
| Vaccine from genomic DNA of | 400 | 694 |
| P. aeruginosa 1 time a month | | |
| Vaccine from genomic DNA of | 200 | 856 |
| P. aeruginosa 1 time a week | | |
| Vaccine from genomic DNA of | 400 | 730 |
| P. aeruginosa 1 time a week | | |
| Vaccine from cell-surface associated DNA | 200 | 712 |
| of P. aeruginosa 1 time a month | | |
| Vaccine from cell-surface associated DNA | 400 | 677 |
| P. aeruginosa 1 time a month | | |
| Vaccine from cell-surface associated DNA | 200 | 763 |
| P. aeruginosa 1 time a week | | |
| Vaccine from cell-surface associated DNA | 400 | 709 |
| of P. aeruginosa 1 time a week | | |
| Vaccine from DNA from feces 1 time a | 200 | 788 |
| month | | |
| Vaccine from DNA from feces 1 time a | 400 | 710 |
| month | | |
| Vaccine from DNA from feces 1 time a | 200 | 862 |
| week | | |
| Vaccine from DNA from feces 1 time a | 400 | 804 |
| week | | |

Data clearly shows that vaccines having in their components bacterial DNA or bacterial TezRs significantly increase the lifespan.

### Example 102: Products and Methods for Managing the Resistance to Antibiotics

We studied how the treatment with tested products could affect sensitivity of microorganisms against antimicrobial agents. Zero-D, Zero-R and Zero-DR cells were obtained as described above following the use of tested products in a range of concentrations from 1 μg/ml up to 1 mg/ml for 30 sec-2 h.

Sensitivity to antibiotic was estimated by a standard disk-diffusion method with the SIR (susceptible, intermediate or resistant) according to Clinical and Laboratory Standards Institute (CLSI) recommendations (Tables 101, 102).

TABLE 97
Effect of putting cells to a zero state to a sensitivity to antibiotics

| | Sensitivity to antibiotics | | | | | |
|---|---|---|---|---|---|---|
| Cells/products | penicillin | oxacillin | erythromycin | rifamycin | cefoperazone | roxithromycin |
| Control | S | S | S | R | S | S |
| DNase I | S | R | S | R | S | S |
| Polymerase (T4) | S | R | S | R | S | S |
| Cre recombinase | S | R | S | R | S | S |
| RNase A | S | R | S | R | S | S |
| Antibodies | S | R | S | R | S | S |
| against cell- | | | | | | |
| surface bound | | | | | | |
| RNA | | | | | | |
| Argonaute protein | S | R | S | R | S | S |
| Endonuclease G + | S | R | S | R | S | S |
| modified Cas9 | | | | | | |
| Nuclease P1 + | S | R | S | R | S | S |
| Leucine zipper | | | | | | |
| N2G-trimethylene- | S | R | S | R | S | S |
| N2G + TetR- | | | | | | |
| binding RNA | | | | | | |
| aptamer | | | | | | |
| Zero-D | R | S | I | S | I | R |
| (treated with | | | | | | |
| DNase I) | | | | | | |
| Zero-D | R | S | I | S | I | R |
| (treated with | | | | | | |
| Polymerase (T4) | | | | | | |
| Zero-D | R | S | I | S | I | R |
| (treated with Cre | | | | | | |
| recombinase) | | | | | | |
| Zero-R | S | R | S | S | S | S |
| (treated with | | | | | | |
| RNase A) | | | | | | |
| Zero-R | S | R | S | S | S | S |
| (treated with | | | | | | |
| Antibodies | | | | | | |
| against TezR_R1) | | | | | | |
| Zero-R | S | R | S | S | S | S |
| (treated with | | | | | | |
| Argonaute protein) | | | | | | |
| Zero-DR | S | S | S | R | S | R |
| (treated with | | | | | | |
| endonuclease | | | | | | |
| G + modified) | | | | | | |
| Zero-DR | S | S | S | R | S | R |
| (treated with | | | | | | |
| nuclease P1 + | | | | | | |
| Leucine zipper) | | | | | | |
| Zero-DR | S | S | S | R | S | R |
| (treated with | | | | | | |
| N2G-trimethylene- | | | | | | |
| N2G + TetR- | | | | | | |
| binding RNA | | | | | | |
| aptamer) | | | | | | |

S—sensitive,
I—intermediate,
R—resistant

TABLE 102
Effect of zero-state cells in sensitivity to antibiotics

| Bacteria | Antibiotic | Intact cell | Zero DR cells |
|---|---|---|---|
| S. aureus VT 85 | Erythromycin | I | R |
| S. aureus VT 5588 | Erythromycin | R | S |
| S. aureus VT 85 | Co-trimoxasole | I | S |
| S. aureus VT 5588 | Co-trimoxasole | R | S |
| E. faecalis VT 67 | Doxyciclin | I | R |
| S. aureus VT 5588 | Doxyciclin | I | S |
| E. faecalis VT 67 | Tobramycin | I | R |

These data clearly show that products can alter sensitivity of bacteria to antimicrobial agents and making antibiotic resistant cells to become sensitive to antibiotics.

### Example 103: Products and Method for Managing Genome Rearrangement

B. pumilus 1278 and C. albicans VT-9 were treated once with products that inactivate cell-surface bound nucleic acids or with multiple cycles to generate zero cells as previously described. Next, B. pumilus 1278 were plated to Sabouraud Dextrose Broth (DB) and Potato Dextrose Broth (PDB) which are commonly used for fungi, but are not used for bacteria and are not “remembered” by bacteria. For bacteria in order to grow on these media, significant genomic rearrangement should be completed. (Table 103).

TABLE 103
Control of prokaryotic genome rearrangements
with tested products and methods

| | Start of | | CFU (7 h | | OD (7 h | |
|---|---|---|---|---|---|---|
| Cells | growth (h) | | growth) | | growth) | |
| (1278) | SDB | PDB | SDB | PDB | SDB | PDB |
| Control | 6 | 9 | 1.30E+05 | 3.50E+04 | 0.099 | 0.048 |
| Cut-D | 7 | >9 | 2.7E+04 | 4.2E+03 | 0.056 | 0.024 |
| Cut-R | 8 | >9 | 3.1E+04 | 5.1E+03 | 0.032 | 0.030 |
| Cut-DR | 8 | >9 | 2.6E+04 | 2.3E+03 | 0.027 | 0.033 |
| Zero-D | 6 | 9 | 8.90E+04 | 6.30E+04 | 0.103 | 0.061 |
| Zero-R | 4 | 6 | 5.70E+05 | 7.30E+05 | 0.113 | 0.081 |
| Zero-DR | 4 | 6 | 1.30E+06 | 9.20E+05 | 0.134 | 0.101 |

Next, C. albicans VT-9 were plated to Columbia Broth (CB) and Mueller Hinton Broth (MHB) which are commonly used for bacteria, but are not used for fungi and are not “remembered” by them. For C. albicans in order to grow on these media, significant genomic rearrangement should be completed. (Table 104).

TABLE 104
Control of eukaryotic genome rearrangements
with tested products and methods

| | Start of | | CFU (7 h | | OD (7 h | |
|---|---|---|---|---|---|---|
| Cells | growth (h) | | growth) | | growth) | |
| (C. albicans) | CB | MHB | CB | MHB | CB | MHB |
| Control | 4 | 7 | 5.7E+06 | 1.2E+04 | 0.238 | 0.042 |
| Cut-D | 6 | >9 | 7.4E+04 | 2.3E+03 | 0.098 | 0.021 |
| Cut-R | 5 | 7 | 4.2E+05 | 2.9E+03 | 0.124 | 0.051 |
| Cut-DR | 7 | >9 | 3.2E+03 | 3.1E+03 | 0.047 | 0.019 |
| Zero-D | 4 | 7 | 4.8E+06 | 2.6E+04 | 0.259 | 0.049 |
| Zero-R | 4 | 7 | 2.1E+06 | 2.2E+04 | 0.284 | 0.058 |
| Zero-DR | 2 | 4 | 7.2E+07 | 6.1E+06 | 0.643 | 0.194 |

These data clearly show that tested products and regimens of their use can be used to control pro- and eukaryotic genome rearrangements.

### Example 104: Products and Method to Control Cell Synthetic Activity and Aging

Primary human fibroblast cells derived from mice were obtained as previously described and used at passage 5 (http://www.jove.com/video/53565). Confluent skin fibroblasts cultured in 24-well plates were maintained in a standard DMEM supplemented with 0.1% fetal bovine serum, washed from extracellular matrix, then treated once or several times with tested products at the range of concentrations varied from 0.1 to 100 μg/mL as described above after which tested products were washed out with nutrient medium. Collagen production was determined after the cells being pulsed with 3 μCi/ml [3H]proline with subsequent measuring [3H]proline incorporation into collagenous proteins. Three aliquots of 250-μL each of a conditioned medium were mixed with 2 mM CaCl2), 1 mM phenylmethylsulfonylfluoride, 4 mM N-ethylmaleimide, and 25 μg BSA with 100 U/ml collagenase (or sterile water), with subsequent incubation for 4 h at 37 C. Remaining proteins were precipitated with 10% trichloroacetic acid for 45 min at +4 C, centrifuged and obtained pellets were again washed with 10% trichloroacetic acid after which solubilized in 0.3 N NaOH/1% sodium dodecyl sulfate. We next measured the radioactivity in obtained protein pellets and subtracting the collagenase-resistant uptake from the total uptake. Data (rounded to 5) are presented in table 105.

TABLE 105
Effect of tested products and methods to
control cell synthetic activity and aging

| | Collagen ([3 H]proline incorporation |
|---|---|
| Cells | (dpm/well)) |
| Control | 5770 |
| Cut-D | 7865* |
| Cut-R | 9560* |
| Cut-DR | 7240* |
| Zero-D | 10890* |
| Zero-R | 11435* |
| Zero-DR | 12820* |

*p < 0.05

Data received clearly show that tested products and methods can be used to modulate synthetic activity of cells, collagen production, aging and joint restoration.

Next, we injected these cells to the skin of mice and analyzed the expression of the collagen. For that 8 weeks old c57bl/6 mice were shaved on their back and ˜10,000 cells were injected to the derma with the 5 mm distance between the injection sites. To examine the increase in collagen levels, we quantified hydroxyproline content in the areas of the skin following the injection of different types of cells 8 weeks later. The level of hydroxyproline was elevated in all the sites of injections with cells following the treatment compared to the skin zones where control cells were injected. Data are presented at table 106.

TABLE 106
Effect of tested products, cells and methods
on regeneration and rejuvenation

| Cells | Hydroxyproline content μg/mg |
|---|---|
| Control | 0.33 ± 0.02 |
| Control untreated | 0.35 ± 0.04 |
| Cut-D | 0.43 ± 0.03* |
| Cut-R | 0.48 ± 0.05* |
| Cut-DR | 0.40 ± 0.03* |
| Zero-D | 0.66 ± 0.08* |
| Zero-R | 0.59 ± 0.09* |
| Zero-DR | 0.71 ± 0.11* |

*p < 0.05

Data received clearly show that the use of tested products or the cells obtained after the use of the tested products can significantly modulate the characteristics of the macroorganism following the transplantation to the macroorganism including its regeneration and rejuvenation.

### Example 105: Product and Methods for Managing of Wound Healing

We studied the use of cells following the treatment with tested products in wound healing. Mouse fibroblasts were obtained as described above, washed out form the extracellular matrix treated with tested products as described above at 50 μg/ml for 30 minutes, after which tested products were washed out with nutrient medium and applied to a full thickness 1 cm diameter skin defect of 8-week-old C57BL/6 as previously described. Data are shown in table 107.

TABLE 107
Effect of products on wound size

Wound size

| Group | Day 0 | Day 4 |
|---|---|---|
| Control | 100% | 86% |
| Control cells | 100% | 72% |
| Cut-D | 100% | 52%* |
| Cut-R | 100% | 47%* |
| Cut-DR | 100% | 43%* |
| Zero-D | 100% | 8%* |
| Zero-R | 100% | 5%* |
| Zero-DR | 100% | 0%* |

*p < 0.05

These data clearly show that the use of tested products or the cells following the treatment with tested products can be used for the treatment of different diseases including burns, ulcers, wounds.

### Example 106: Products and Methods for Managing Cells Memory

To isolate cancer associated fibroblasts (CAF), 5×10e5 4T1 cells were injected into mammary fat pads of BALB/c mouse (8 weeks old, female). Following 24 days of growth, the primary the primary tumor was resected and subsequently homogenized and digested in 1 mL of L-15 medium containing 0.25% trypsin and collagenase (2 mg/mL) and incubate at 37° C. for 60 min using Red Blood Cell Lysis Buffer. Immune cells were excluded with rat-anti-mouse CD45 and CD24 antibodies and superparamagnetic beads with affinity polyclonal sheep anti-rat IgG that bond to the bead surface. Isolation of CAF was conducted by Fluorescence Activated Cell Sorting, using FITC+/RFP−/DAPI− and excluding dead and cancer cells. Obtained CANs were washed from the extracellular matrix and treated with tested products as described previously (one- or multiple times) and after the treatment, tested products were washed out with nutrient medium. After that, modified CAFs were injected to the tumor site of 4T1-bearing tumors BALB/c mice (with 14 days post tumor cells implantation). Tumor volume (rounded to 5) was measured at day 28th (table 108).

TABLE 108
Effect of tested products, cells and
methods on tumor growth on 28th day

| | | Number |
|---|---|---|
| | Tumor size | of gross |
| Group | mm3 | metastasis |
| Control untreated | 1165 ± 230 | 100% |
| Control cells | 1165 ± 230 | 92% ± 7%* |
| Cut-D cells | 540 ± 90* | 42 ± 10%* |
| Cut-R cells | 690 ± 105* | 35 ± 5%* |
| Treatment with modified RNase | 675 ± 125* | 31 ± 8%* |
| Cut-DR cells | 445 ± 80* | 28 ± 5%* |
| Zero-D cells | 375 ± 65* | 21 ± 4%* |
| Zero-R cells | 290 ± 45* | 6 ± 4%* |
| Cells Zero-R (treated with modified | 275 ± 65* | 19 ± 7%* |
| RNase) | | |
| Zero-DR0 cells | 120 ± 40* | 3 ± 2%* |
| Cells following three rounds of treatment | 305 ± 55* | 18 ± 3%* |
| with Histone 5 + RNA polymerase | | |
| Cells following three rounds of treatment | 260 ± 50* | 15 ± 6%* |
| with antibodies against cell-surface | | |
| bound DNA and RNA | | |

*p < 0.05

These data clearly show that the use of listed products, or transfer of cells obtained following the treatment with tested products in different regimes can lead to anticancer effects.

### Example 107: Products and Methods for Erasing Cancer Cells Memory

PANC1 cells were maintained in recommended growth medium with 10% fetal bovine serum at 37° C., 5% CO2. Next, cells were separated from the extracellular matrix, treated with tested compounds, after which tested products were washed out with nutrient medium. The expression of KRAS was analyzed following RNA isolation as previously described with a subsequent RT-PCR with KRAS primers (FW 5-CAGGAAGCAAGTAGTAATTGATGG-3; REV 5-TTATGGCAAATACACAAAGAAAGC-3) and normalization to 18s rRNA. Data are presented in table 109.

TABLE 109
Effects of tested products and methods to trigger cell
reprogramming and erasure of oncology-focused memory

| | KRAS |
|---|---|
| | expression |
| Group | (%) |
| Control | 100 |
| Cut-D cells | 54* |
| Cut-R cells | 63* |
| Treatment with modified RNase | 61* |
| Cut-DR cells | 33* |
| Zero-D cells | 39* |
| Zero-R cells | 32* |
| Cells Zero-R (treated with modified RNase) | 27* |
| Zero-DR cells | 17* |
| Cells following three rounds of treatment with | 6* |
| antibodies against cell-surface bound DNA and RNA | |

*p < 0.05

Data received clearly show that proposed methods and products can be used for the reversal of prooncogenic state of cells, cell reprogramming and erasure of oncology-related memory.

### Example 108: Products and Methods for the Treatment of Traumas and Regrowth or Repair of Nervous Tissues and Cells

8 weeks old NOD-SCID mice were anesthetized as described above followed by laminectomy between 10th and 9th spinal vertebrae and triggering spinal cord injury with a special device at 70 kdyn (moderate injury). The wound was closed and mice were treated daily with gentamicin (6 mg/kg), with daily bladder evacuation. On the 6th day post spinal cord injury days after injury, animals were again anesthetized and neuronal stem cells (treated previously with tested compounds, after which tested products were washed out) were microinjected into the epicenter of cord injury from 1×10e2 to 5×10e9 cells. Motor function was analyzed weekly using a Basso Mouse Scale (BMS) soring system. Data are presented in table 110.

TABLE 110
Effects of tested products, methods and cells for
the recovery on 14th day post neuronal damage.

| Group | BMS |
|---|---|
| Control untreated | 1.8 ± 0.3 |
| Control cells 5 × 10e4 cells | 2.2 ± 0.3 |
| Cut-D cells 5 × 10e4 cells | 2.6 ± 0.1* |
| Cut-R cells 5 × 10e4 cells | 3.0 ± 0.4* |
| Treatment with modified RNase | 2.5 ± 0.4* |
| Cut-DR cells 5 × 10e4 cells | 3.7 ± 0.5* |
| Zero-D cells 5 × 10e4 cells | 3.2 ± 0.2* |
| Zero-R cells 5 × 10e4 cells | 4.4 ± 0.5* |
| Cells Zero-R (treated with modified RNase) 5 × 10e4 cells | 4.5 ± 0.3* |
| Zero-DR0 cells 5 × 10e4 cells | 4.9 ± 0.4* |
| Zero-DR cells 1 × 10e2 cells | 2.7 ± 0.2* |
| Zero-DR cells 5 × 10e9 cells | 5.3 ± 0.2* |
| Cells following three rounds of treatment with | 3.5 ± 0.3* |
| antibodies against cell-surface bound DNA and RNA | |
| Cells treated with Histone 5 | 2.8 ± 0.3* |
| Cells treated with TLR9 | 2.9 ± 0.3* |
| Cells treated with modified mitotane | 2.5 ± 0.2* |

*p < 0.05

Data receive indicate that modified cells may be used for treatment nervous tissues and cells regrowth or repair including those caused by traumas.

### Example 109: Products and Method Managing of Cartilage Regeneration

Destabilization of the medial meniscus of right knee was done in fully anesthetized 16-weeks old C57BL/6 mice as previously described (Christiansen et al., 2015) and treated daily with gentamicin (8 mg/kg) for three days.

Mesenchymal cells were treated with tested products as described earlier and were intra-articularly injected day 7 post injury in the same knee of anesthetized animals. Some animals received intraarticular injection of 10 μl of DNase (2000 Kunitz units/mg) and/or 10 μl of RNase (100 units/mg) one a week. OARSI score was assessed on week

TABLE 111
Quantification of Cartilage Injury Repair

| Group | OARSI score |
|---|---|
| Untreated Control | 100% |
| Control treated with unaltered cells | 93% |
| Cut-D cells | 56%* |
| Cut-R cells | 49%* |
| Treatment with modified RNase | 53%* |
| Cut-DR cells | 39%* |
| Zero-D cells | 34%* |
| Zero-R cells | 25% |
| Cells Zero-R (treated with modified RNase) | 24%* |
| Zero-DR cells | 17%* |
| Cells following three rounds of treatment with | 15%* |
| antibodies against cell-surface bound DNA and RNA | |
| Intra-articularly injected with DNase | 45%* |
| Intra-articularly injected with RNase | 39%* |
| Intra-articularly injected with DNase + RNase | 37%* |

*p < 0.05

Data received indicate that cartilage repair was significantly higher in animals treated with experimental cells and therapies.

### Example 111: Products and Method Managing of Pain

Primary keratinocytes were isolated from humans, from foreskins as described previously and were cultured in a serum-free medium supplemented with 4 ng/ml recombinant epidermal growth factor and 40 μg/ml bovine pituitary extract (all Invitrogen Life Technologies). After that cells were washed from extracellular matrix, then treated once or several times with tested products at the range of concentrations varied from 0.1 to 100 μg/mL as described above after which tested products were washed out with nutrient medium. Expression of IL-1α was quantified by real-time RT-PCR with the following primers: (forward) 5′-ATCAGTACCTCACGGCTGCT-3′, and (reverse) 5′-TGGGTATCTCAGGCATCTCC-3′. Data are shown in table 112.

TABLE 112
Effect of tested products on managing pain

| | Expression |
|---|---|
| Cells | level |
| Control | 100% |
| Cut-D cells | 77%* |
| Cut-R cells | 68%* |
| Cut-DR cells | 35%* |
| Zero-D cells | 52%* |
| Zero-R cells | 60%* |
| Zero-DR cells | 26%* |
| One-time treatment with histone H2B | 71%* |
| One-time treatment with histone Ribosomal protein bL12 | 54%* |
| One-time treatment with RNA recognition motif | 79%* |
| One-time treatment with Modified bleomycin | 63%* |
| Three-time treatment with Modified bleomycin | 41%* |
| One-time treatment with Antibodies against TezR_R1 | 52%* |

*p < 0.05

Data received indicate that the use of tested compounds decreases the expression of IL-α and IL-1α-NF-κB-CCL2 signaling pathway which is known to induce activation and migration of monocytes that contribute to pain-like sensitivity (Paish et al., 2018). Therefore tested products can be used for the pain management.

### Example 112: Products and Method for Autologous Mesotherapy

Forty patients with a progressive hair loss (grade III-IV) participated in the study. We excluded patients with one any of the following: any known cause of alopecia (anemia, malignancies, malnutrition, connective tissue disease, therapy from oncological disease, SARS-CoV-2 infection), use of medications that influence hair growth for the last 6 months, pregnancy, mental disorders. Patients were randomized to different groups and treated with mesotherapy once a months for 6 months. PRP was prepared using the Plateletex Kit (DCare, Chicago, Illinois, USA). 10 ml of whole blood was withdrawn using from a vein in EDTA covered tube, transferred to siliconized glass tube and spined 3500 rpm×10 min. This step resulted in separation of the whole blood into three layers. The blood got separated and two upper layers that contain platelets and white blood cell were taken and centrifuged at 1500 rpm for 15 min. The lower platelet rich part was taken and treated or not treated with nucleases. Nucleases were either washed out with another set of centrifugation or left within PRP suspension. PRP suspensions were injected using Mesotherapy Hypodermic Needles 30 g×4 mm. Data are shown in table 113.

TABLE 113
Effect of tested products on the efficacy of mesotherapy

| | Hair count | |
|---|---|---|
| Probe | Baseline | 28 days |
| Control no treatment | 100% | 98% |
| Control PRP suspension | 100% | 119% |
| PRP suspension treated with DNase, DNase | 100% | 126%* |
| left | | |
| PRP suspension treated with RNase, RNase | 100% | 145%* |
| left | | |
| PRP suspension treated with DNase + RNase, | 100% | 164%* |
| DNase and RNase left | | |
| PRP suspension treated with DNase, DNase | 100% | 132%* |
| removed | | |
| PRP suspension treated with RNase, RNase | 100% | 141%* |
| removed | | |
| PRP suspension treated with DNase + RNase, | 100% | 178%* |
| DNase and RNase removed | | |

*p < 0.05 compared with PRP suspension

Data received indicate that the use of tested products significantly potentiates the efficacy of mesotherapy products.

### Example 113: Products and Methods for Overcoming Antibiotic Resistance that Depends on Efflux Systems

Achromobacter xylosoxidans harboring multidrug resistant genes encoding efflux pumps (Bador et al., 2011, Berra et al., 2014, Adewoye et al., 2016, Isler et al., 2020) were treated with reverse-transcriptase and integrase inhibitors to overcoming bacterial resistance to fluoroquinol ones macrolides, rifamycin, tetracycline, chloramphenicol, sulfanilamide, trimethoprim. Nevirapine and etravirine were used at concentrations from 0.1 to 100 μg/mL us effectors of intracellular part of Tetz-receptor system. Minimal inhibitory concentration was evaluated according to CLSI guidelines. Data are presented in tables 114 and 115.

TABLE 114
Effects of tested products on modulation of antibiotic resistance

| | | MIC μg/mL against A. xylosoxidans | | | | | |
|---|---|---|---|---|---|---|---|
| | | | | | | | Integrase |
| | | | Reverse-transcriptase inhibitors | | | | inhibitor |
| Agent | | Control | Nevirapine | Etravirine | Lamivudine | Tenofovir | Raltegravir |
| Fluoroquinolones | Ciprofloxacin | 200 | 3.1 | 100 | 200 | 6.2 | 50 |
| | Levofloxacin | 12.5 | 1.5 | 12.5 | 12.5 | 6.2 | 25 |
| Aminoglycosides | Tobramycin | 400 | 100 | 300 | 300 | 100 | 100 |
| | Amikacin | 250 | 60 | 250 | 250 | 60 | 30 |
| Macrolides | Azithromycin | 500 | 16 | 31 | 31 | 31 | 16 |
| Beta-lactam | Ampicillin | 500 | 125 | 500 | 500 | 500 | 500 |
| antibiotics | | | | | | | |
| Dihydropteroate | Sulfamethoxazole | 500 | 500 | 500 | 500 | 500 | 500 |
| synthase | | | | | | | |
| inhibitor | | | | | | | |
| Dihydrofolate | Trimethoprim | 500 | 125 | 500 | 500 | 125 | 125 |
| reductase | | | | | | | |
| inhibitor | | | | | | | |
| Tetracyclines | Doxycycline | 250 | 62 | 250 | 250 | 250 | 62 |
| Rifamycins | Rifampicin | 500 | 500 | 500 | 500 | 500 | 500 |
| Amphenicols | Chloramphenicol | 12.5 | 3.1 | 12.5 | 12.5 | 12.5 | 12.5 |
| Glycopeptide- | Bleomycin | 250 | 60 | 250 | 250 | 250 | 60 |
| derived | | | | | | | |
| antibiotics | | | | | | | |

TABLE 115
Effects of tested products on modulation of antibiotic resistance

| | Increase of sensitivity | | | | |
|---|---|---|---|---|---|
| | | | Beta- | | Glycopeptide- |
| | | | lactam | | derived |
| Treatment group | Fluoroquinolones | Aminoglycosides | antibiotics | Tetracyclines | antibiotics |
| azidothymidine | Yes | Yes | Yes | Yes | Yes |
| abacavir | Yes | Yes | Yes | Yes | Yes |
| Cabotegravir | Yes | Yes | Yes | Yes | Yes |
| Censavudine | Yes | Yes | Yes | Yes | Yes |
| Elsulfavirine | Yes | Yes | Yes | Yes | Yes |
| Islatravir | Yes | Yes | Yes | Yes | Yes |
| Alafenamide | Yes | Yes | Yes | Yes | Yes |
| 3-(Hexadecyloxy)propyl | Yes | Yes | Yes | Yes | Yes |
| hydrogen ((((R)-1-(6- | | | | | |
| amino-9H-purin-9- | | | | | |
| yl)propan-2- | | | | | |
| yl)oxy)methyl)phosphonate) | | | | | |
| Festinavir | Yes | Yes | Yes | Yes | Yes |
| 4′-ethynyl stavudine, or | Yes | Yes | Yes | Yes | Yes |
| 4′-ethynyl-d4T); 3-[3- | | | | | |
| ethyl-5-isopropyl-2,6- | | | | | |
| dioxo-1,2,3,6- | | | | | |
| tetrahydro-pyrimidine- | | | | | |
| 4-carbonyl]-5-methyl | | | | | |
| benzonitrile | | | | | |
| Lersivirine | v | Yes | Yes | Yes | Yes |
| Didanosine | Yes | Yes | Yes | Yes | Yes |
| Rilpivirine | Yes | Yes | Yes | Yes | Yes |
| Efavirenz | Yes | Yes | Yes | Yes | Yes |
| Emtricitabine | Yes | Yes | Yes | Yes | Yes |
| Zidovudine | Yes | Yes | Yes | Yes | Yes |
| Disoproxil | Yes | Yes | Yes | Yes | Yes |
| Fumarate | Yes | Yes | Yes | Yes | Yes |
| Olaparib | Yes | Yes | Yes | Yes | Yes |
| Rucaparib | Yes | Yes | Yes | Yes | Yes |
| Veliparib | Yes | Yes | Yes | Yes | Yes |
| Talazoparib | Yes | Yes | Yes | Yes | Yes |
| Niraparib | Yes | Yes | Yes | Yes | Yes |
| Asunaprevir | Yes | Yes | Yes | Yes | Yes |
| Boceprevir | Yes | Yes | Yes | Yes | Yes |
| Grazoprevir | Yes | Yes | Yes | Yes | Yes |
| Glecaprevir | Yes | Yes | Yes | Yes | Yes |
| Paritaprevir | Yes | Yes | Yes | Yes | Yes |
| Simeprevir | Yes | Yes | Yes | Yes | Yes |
| Telaprevi | Yes | Yes | Yes | Yes | Yes |
| Amprenavir | Yes | Yes | Yes | Yes | Yes |
| Atazanavir | Yes | Yes | Yes | Yes | Yes |
| Darunavir | Yes | Yes | Yes | Yes | Yes |
| Fosamprenavir | Yes | Yes | Yes | Yes | Yes |
| Indinavir | Yes | Yes | Yes | Yes | Yes |
| Lopinavir | Yes | Yes | Yes | Yes | Yes |
| Nelfinavir | Yes | Yes | Yes | Yes | Yes |
| Ritonavir | Yes | Yes | Yes | Yes | Yes |
| Saquinavir | Yes | Yes | Yes | Yes | Yes |
| Tipranavir | Yes | Yes | Yes | Yes | Yes |
| raltegravir | Yes | Yes | Yes | Yes | Yes |
| Dolutegravir | Yes | Yes | Yes | Yes | Yes |
| Bictegravir | Yes | Yes | Yes | Yes | Yes |
| Cabotegravir | Yes | Yes | Yes | Yes | Yes |
| C27H26N2O4 | Yes | Yes | Yes | Yes | Yes |
| C21H21ClFN5O4 | Yes | Yes | Yes | Yes | Yes |
| Magnesium orotate | Yes | Yes | Yes | Yes | Yes |
| Calcium orotate | | | | | |

Surprisingly, tested products increased sensitivity of cells to chemotherapeutic agents.

### Example 114: Products and Methods for the Erasure of Cell Memory

Primary cancer cells with confirmed EGFR expression were either washed with PBS, centrifuged at 200 g×5 min to eliminate extracellular matrix or proceeded to the follow-up treatments without removal of the extracellular matrix. Next, all cells were treated one or three times with nucleases 50 μg/mL for 20 minutes (followed by the passage in the medium without fetal serum) with subsequent growth in DMVEM medium (Sigma), supplemented with 10% fetal bovine serum (Gibco) and 1% streptomycin (Sigma) at 37° C. in a humidified atmosphere containing 5% CO2.

Some Zero-D, Zero-R, Zero-DR0 cells between cycles of DNase use were additionally treated with integrase inhibitors (raltegravir, 2.5 μg/ml)

The expression of EGFR was assessed after that or following 5 passages in a regular media without any additional treatments. Data are shown in table 116.

TABLE 116
Effect of tested products on cell memory

| | | Expression of EGFR | Expression of EGFR after |
|---|---|---|---|
| | | after the 1^{st} passage | the 5th passage post |
| | | after treatment with | treatment with tested |
| Group | | tested products | products |
| Extracellular | Control | 100% | 100% |
| matrix | Cut-D cells | 67%* | 89%* |
| removed | Cut-R cells | 54%* | 85%* |
| | Cut-DR cells | 42%* | 67%* |
| | Zero-D cells | 44%* | 49%* |
| | Zero-R cells | 32%* | 21%* |
| | Zero-DR cells | 14%* | 21%* |
| | Zero-D cells + additionally | 4%*^{,} *** | 9%*^{,} *** |
| | treated with raltegravir | | |
| | Zero-R cells + additionally | 6%*^{,} *** | 3%*^{,} *** |
| | treated with raltegravir | | |
| | Zero-DR cells + additionally | 0%*^{,} *** | 0%*^{,} *** |
| | treated with raltegravir | | |
| | Three-time treatment netropsin + | 12%* | 0%* |
| | RNA helicase (Zero-DR cells) | | |
| | Three-time treatment Histone | 10%* | 0%* |
| | H3 + Ribosomal protein L3 | | |
| | (Zero-DR cells) | | |
| Extracellular | Control | 100% | 100% |
| matrix | Cut-D cells | 75%* | 100% |
| not | Cut-R cells | 74%* | 98% |
| removed | Cut-DR cells | 89%** | 94% |
| | Zero-D cells | 54%* | 69%*^{,} ** |
| | Zero-R cells | 18%** | 33%*^{,} ** |
| | Zero-DR cells | 24%* | 20%*^{,} ** |
| | Three-time treatment netropsin + | 36%*^{,} ** | 48%*^{,} ** |
| | RNA helicase (Zero-DR cells) | | |
| | Three-time treatment Histone | 25%*^{,} ** | 43%*^{,} ** |
| | H3 + Ribosomal protein L3 | | |
| | (Zero-DR cells) | | |

*p < 0.05 comparing with control;
**p < 0.05 between probes in which extracellular matrix was removed vs extracellular matrix was left;
***p < 0.05 between Zero-D, Zero-R, Zero-DR and between Zero-D, Zero-R, Zero-DR additionally treated with raltegravir.

Surprisingly, the data show that multiple cycles of treatment with tested products results in forgetting cells of their pro-oncogenic phenotype.

### Example 115. Products and Methods to Regulate Immune Cells Activity

Neutrophils were isolated from EDTA anticoagulated whole blood of two healthy volunteers by Ficoll density gradient centrifugation using Lymphoprep™ (Stemcell Technologies). Following the centrifugation for 30 min at 750×g, the lower cellular fraction containing neutrophils was collected, and remaining erythrocytes were lysed. Neutrophils were adjusted to 1×10e6 cells/ml in DMEM (serum-free). Cells were treated with tested compounds as described earlier. Next we induced NETosis by seeding purified neutrophils (5×10e5 cells/cm2) and stimulated with the mix of bacterial LPS isolated from P. aeruginosa and E. coli for 3 h at 37° C. After this neutrophils and NETs were washed twice with PBS. Extracellular DNA in supernatants was stained with 100 nM Sytox Orange and quantified by fluorometry (530/640 nm). Data are presented in table 117.

TABLE 117
Effect of tested products on disease-associate pathways

| Group | Extracellular DNA (au) |
|---|---|
| Control | 1 |
| LPS stimulated | 3 ± 1.2 |
| DNase I, LPS stimulated | 2.1 ± 0.9* |
| RNase I, LPS stimulated | 1.7 ± 0.7* |
| DNase I + RNase, LPS stimulated | 2.3 ± 1.4* |
| Zidovudine (AZT), Tenofovir (TNF), | 2.9 ± 1.9 |
| Nevirapine (NVP) and etravirine (ETR) at | |
| 5 μg/mL, LPS stimulated | |
| DNase I + Zidovudine (AZT), Tenofovir | 1.7 ± 1.0* |
| (TNF), Nevirapine (NVP) and etravirine | |
| (ETR) at 5 μg/mL, LPS stimulated | |
| RNase + Zidovudine (AZT), Tenofovir | 1.2 ± 0.4* |
| (TNF), Nevirapine (NVP) and etravirine | |
| (ETR) at 5 μg/mL, LPS stimulated | |
| Zero-D cells, LPS stimulated | 1.7 ± 0.3* |
| Zero-R cells, LPS stimulated | 1.5 ± 0.5* |
| Zero-DR cells, LPS stimulated | 1.1 ± 0.3* |
| Antibodies against cell-surface bound | 2.0 ± 1.1* |
| DNA, LPS stimulated | |
| Antibodies against cell-surface bound | 1.8 ± 0.7* |
| RNA, LPS stimulated | |
| Histone H5, LPS stimulated | 1.9 ± 1.2* |
| Ribosomal protein, LPS stimulated | 1.8 ± 0.9* |

*p < 0.05 compared to stimulated cells

These data clearly show that the use of tested compounds including the formation of zero cells can be used to inhibit neutrophil activation and formation of neutrophil extracellular traps.

### Example 116: Products and Methods for Changing Cell Settings and Cells Memory Formation

For the formation of cells with a new memory we formed zero-state C. albicans as previously described by three rounds of treatment with RNase A with or without DNase (each 50 μg/mL, 30 min exposition time at 37 C) followed by a wash-out period in minimal media without nutrients (i.e. M9 media without maltose) or by putting cells to a “Y” state by three rounds of treatment with RNase A with or without DNase (each 50 μg/mL, 30 min exposition time at 37 C) followed by a wash-out period in regular nutrient rich media. For some cells in minimal media we added unusual composition of nucleic acids (1 μg/mL DNA and 1 μg/mL RNA) isolated from the human feces with QIAamp DNA Stool Mini Kit and QIAgen RNA mini kit. Next we measured the lag phase (minimal time of contact to trigger maltose utilization) of these cells as well of the next generation of these cells obtained by maintaining in M9 media with maltose for another 24 h to restore cell-surface bound nucleic acids (table 118)

TABLE 118
Effect of altered memory formation.

| | Minimal time of | |
|---|---|---|
| | contact to trigger | |
| Treatment regimen of C. albicans | maltose utilization | |
| Maltose-naive control | 3 | h |
| Maltose-sentient control | 2 | h |
| Y-D cells | 1 | h |
| Y-R cells | 0.5 | h |
| Y-DR cells | 0.25 | h |
| Zero-R cells | 3 | h |
| Zero-DR cells | 3 | h |
| Zero-R cells with the treatment with feces- | 8 | h |
| derived DNA and RNA between cycles of | | |
| nuclease treatment | | |
| Zero-DR cells with the treatment with feces- | 12 | h |
| derived DNA and RNA between cycles of | | |
| nuclease treatment | | |
| Zero-R cells with the treatment with feces- | 7 | h |
| derived DNA and RNA with restored cell- | | |
| surface-bound nucleic acids | | |
| Zero-DR0 cells c with the treatment with feces- | 12 | h |
| derived DNA and RNA between cycles of | | |
| nuclease treatment | | |

These data show that it is possible to use zero state to change cell settings, and reprogram cells. Placing cells to a “Y” state can increase cell responses to the outer environment.

### Example 117: Products and Methods for the Regulation of Resistance to UV

To determine whether tested products can participate in UV resistance S. aureus VT209 were treated with tested products. Control probes were left untreated. Bacteria at 8.5 log 10 CFU/mL in PBS were added to 9-cm Petri dishes, placed under a light holder equipped with a new 254-nm UV light tube (TUV 30W/G30T8; Philips, Amsterdam, The Netherlands), and irradiated for different times at a distance of 50 cm. After treatment, bacteria were serially diluted, plated on nutrition agar plates, incubated for 24 h, and CFU were determined. Notably, the use of tested products protected bacteria from UV-induced death, and resulted in significantly higher viable counts compared to control S. aureus following UV irradiation (p<0.05) (FIG. 58).

Data received surprisingly show that the use of tested products could significantly protect from UV induced damage and UV induced cytotoxicity.

### Example 118: Products and Methods for the Targeted Cell Delivery, Development of Antibodies Against NAMACS and/or NAMACS-ANA and/or TEZRs

Maltose-naïve and maltose-sentient C. albicans obtained as previously described. Rabbits were vaccinated by NAMACS and NAMACS-ANA of maltose-sentient C. albicans with Freund's adjuvant as previously described. Next, maltose-naïve C. albicans at concentration 10e12 cells/ml were added to 10 ml of rabbits serum obtained after immunization, probes were incubated for 3-6 h at 37 C and fungi were removed by centrifugation at 4200 g/ml. The procedure was repeated three times, serum were purified from any residual fungi by filtration through a 0.22 m filter. As a result, the serum was depleted and had only antibodies against NAMACS and NAMACS-ANA with them and/or TEZER s implicated in sensing maltose by maltose-sentient Candida.

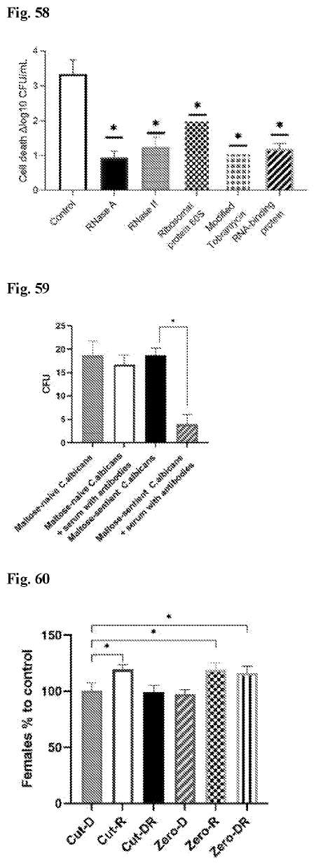

Next, we analyzed growth inhibitory activity of the serum against maltose-sentient C. albicans and maltose-naive C. albicans as previously discussed (Magliani et al., 1997). For that 3×10e2 cells of maltose-sentient C. albicans or maltose-naive C. albicans in 10 μl of PBS were incubated with 100 μl of serum for 22 h at 37° C. The inhibition was evaluated by the number of CFU that gave growth after the seeding the yeasts on Potato Dextrose Agar (Sigma-Aldrich) and incubation for 48 h at 37 C. Data are presented in FIG. 59.

These data clearly show that the proposed method enables the selection of highly specific antibodies to NAMACS and NAMACS-ANA and/or TEZRs.

### Example 119: Gender Control in Fish

Eggs from Salvelinus alpinus L. (7-10 eggs/mL) where treated with products (DNase, RNase, Histone 5, Ribosomal protein S40 taken at concentrations from 1 to 1000 μg/mL from 1 min to 24 h) to turn cells to “Cut” and “Zero” states. After each treatment products were removed by washing with water. Artificial insemination was done as previously described (Bellard et al., 1988). Next, a PCR for rapid sex identification was conducted as previously described (Rud et al., 2015), data are shown in FIG. 60.

Data received indicate on increasing female fishes by 15% after treatment fish eggs to states Cut-R and Zero-R and Zero-DR.

### Example 120: Effects of Products on Cell Surface Associated Electrophysiological Dysfunctions

To investigate the effects of tested products on electrophysiological properties of cardiomyocytes, we used hiPSC-CMs obtained as previously discussed and treated by 0.1 to 100 μg/ml of tested products for 30 minutes. Some probes were additionally treated with nuclease inhibitors (recombinant RNase inhibitor). Data are presented in table 119.

TABLE 119
Effect tested compounds on the development of fibrosis.

| Group | Vmax (V/s) | APD50 (ms) |
|---|---|---|
| Control | 34.3 ± 4.2 | 64.5 ± 9.5 |
| RNase 0.1 μg/ml | 42.1 ± 1.8* | 127.2 ± 19.4* |
| RNase 100 μg/ml | 47.5 ± 3.5* | 139.5 ± 20.2* |
| Ribosomal protein L22 100 μg/ml | 42.7 ± 2.0* | 114.5 ± 12.5* |
| Tobramycin (modified) 1 μg/ml | 43.3 ± 3.1* | 99.4 ± 6.0* |
| RNase 100 μg/ml + RNase inhibitor | 38.4 ± 2.6 | 69.7 ± 10.4 |

*p < 0.05

It is clearly seen that the use of tested compounds can be used for the modulation of cardiac cells repolarization and arrhythmias.

### Example. 121: Products Management of Natural Antibodies (NAb)

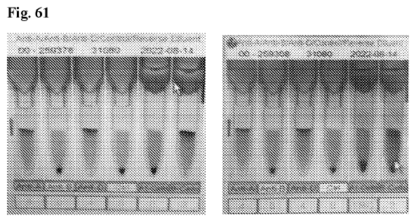

The blood of 32-year-old B group men was treated by DNase and RNase at concentrations from 0.1 μg/ml to 50 μg/ml from 1 min to 6 h. After the treatment blood was tested at the ORTHO AutoVue® Innova System. Treatment with DNase and RNase significantly decreased NAb against B cells in 2-3 times (FIG. 61).

### REFERENCES

Chen C, Okayama H. High-efficiency transformation of mammalian cells by plasmid DNA. Molecular and cellular biology. 1987 August; 7(8):2745-52.
Naldini L, Blömer U, Gallay P, Ory D, Mulligan R, Gage F H, Verma I M, Trono D. In vivo gene delivery and stable transduction of nondividing cells by a lentiviral vector. Science. 1996 Apr. 12; 272(5259):263-7.
Spicer A, Molnar A. Gene editing of microalgae: scientific progress and regulatory challenges in Europe. Biology. 2018 March; 7(1):21.
Tetz, G. and Tetz, V., 2021. Evaluation of a New Culture-Based AtbFinder Test-System Employing a Novel Nutrient Medium for the Selection of Optimal Antibiotics for Critically Ill Patients with Polymicrobial Infections within 4 h. Microorganisms, 9(5), p. 990.
Manukumar, H. M. & Umesha, S. MALDI-TOF-MS based identification and molecular characterization of food associated methicillin-resistant Staphylococcus aureus. Sci. Rep. 7, (2017).
Bennett, R. W. & Monday, S. R. S. aureus in International handbook of foodborne pathogens. in (ed. Miliotis M D, B. J.) 41-59 (2003).
Kwon O K, Mekapogu M, Kim K S. Effect of salinity stress on photosynthesis and related physiological responses in carnation (Dianthus caryophyllus). Horticulture, Environment, and Biotechnology. 2019 December; 60(6):831-9.
Zheng C, Sun Y H, Ye X L, Chen H Q, Ji H B. Establishment and characterization of primary lung cancer cell lines from Chinese population. Acta Pharmacologica Sinica. 2011 March; 32(3):385-92.
Loh C Y, Chai J Y, Tang T F, Wong W F, Sethi G, Shanmugam M K, Chong P P, Looi C Y. The E-cadherin and N-cadherin switch in epithelial-to-mesenchymal transition: signaling, therapeutic implications, and challenges. Cells. 2019 October; 8(10):1118.
Xia M, Tong J H, Ji N N, Duan M L, Tan Y H, Xu J G. Tramadol regulates proliferation, migration and invasion via PTEN/PI3K/AKT signaling in lung adenocarcinoma cells. Eur Rev Med Pharmacol Sci. 2016 Jun. 1; 20(12):2573-80.
Christiansen B A, Guilak F, Lockwood K A, Olson S A, Pitsillides A A, Sandell L J, Silva M J, van der Meulen M C, Haudenschild D R. Non-invasive mouse models of post-traumatic osteoarthritis.
Osteoarthritis and cartilage. 2015 Oct. 1; 23(10):1627-38.
Paish H L, Kalson N S, Smith G R, del Carpio Pons A, Baldock T E, Smith N, Swist-Szulik K, Weir D J, Bardgett M, Deehan D J, Mann D A. Fibroblasts promote inflammation and pain via IL-1α induction of the monocyte chemoattractant chemokine (CC Motif) ligand 2. The American journal of pathology. 2018 Mar. 1; 188(3):696-714.
Isler B, Kidd T J, Stewart A G, Harris P, Paterson D L. Achromobacter infections and treatment options. Antimicrobial Agents and Chemotherapy. 2020 Oct. 20; 64(11):e01025-20.
Bador J, Amoureux L, Duez J M, Drabowicz A, Siebor E, Llanes C, Neuwirth C. First description of an RND-type multidrug efflux pump in Achromobacter xylosoxidans, AxyABM.
Antimicrobial agents and chemotherapy. 2011 October; 55(10):4912-4.
Adewoye L, Topp E, Li X Z. Antimicrobial drug efflux genes and pumps in bacteria of animal and environmental origin. InEfflux-mediated antimicrobial resistance in bacteria 2016 (pp. 561-593). Adis, Cham.
Berra, S., Ayachi, S. and Ramotar, D., 2014. Upregulation of the Saccharomyces cerevisiae efflux pump Tpo1 rescues an Imp2 transcription factor-deficient mutant from bleomycin toxicity. Environmental and Molecular Mutagenesis, 55(6), pp. 518-524.
Magliani W, Conti S, Bernardis F D, Gerloni M, Bertolotti D, Mozzoni P, Cassone A, Polonelli L. Therapeutic potential of antiidiotypic single chain antibodies with yeast killer toxin activity.
Nature biotechnology. 1997 February; 15(2):155-8.
Bellard R. Artificial insemination and gamete management in fish. Marine & Freshwater Behaviour & Phy. 1988 Oct. 1; 14(1):3-21.
Rud Y P, Maistrenko M I, Buchatskii L P. [Sex identification of the rainbow trout Oncorhynchus mykiss by polymerase chain reaction]. Ontogenez. 2015 March-April; 46(2):87-93. PMID: 26021121.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1.-58. (canceled)

59. A method to increase the ability to increase production of a biomanufactured product, comprising:
(a) isolating one or more of a cell, a cell culture, an organoid, a tissue, an embryo, an organ, a single-cellular organism, a multicellular organism;
(b) treating the one or more of the cell, the cell culture, the organoid, the tissue, the embryo, the organ, the single-cellular organism, the multicellular organism with a DNAse and an RNAse;
(c) washing the one or more of the treated cell, the treated cell culture, the treated organoid, the treated tissue, the treated embryo, the treated organ, the treated single-cellular organism, the treated multicellular organism following treatment with the DNase and RNase;
(d) biomanufacturing a protein using the one or more of the treated cell, the treated cell culture, the treated organoid, the treated tissue, the treated embryo, the treated organ, the treated single-cellular organism, and the treated multicellular organism.

60. The method of claim 59, wherein the one or more of the treated cell, the treated cell culture, the treated organoid, the treated tissue, the treated embryo, the treated organ, the treated single-cellular organism and the treated multicellular organism are treated with two or more rounds with DNAse and RNAse.

61. The method of claim 59, wherein the treated cell is a CHO cell.

62. The method of claim 61, wherein the treatment of the CHO cell with a DNase and RNase increases amount of a protein produced by the CHO cell when compared to a CHO cell that is not treated with a DNase and an RNase.

63. The method of claim 59, wherein the treatment of the cell, the cell culture, the organoid, the tissue, the embryo, the organ, thea single-cellular organism and the multicellular organism increases the amount of antibodies, cytokines, growth factors, viral vectors, antigens, vaccines, complex engineered antibodies, trivalent T-cell engagers, checkpoint modulators, naive proteins, recombinant proteins, vitamins, hormones, vaccine, and antibody cytokine fusions produced when compared to a cell, a cell culture, an organoid, a tissue, an embryo, an organ, a single-cellular organism, a multicellular organism that is not treated with a DNase and an RNase.

64. The method of claim 59, wherein the cell is a fungal cell.

65. The method of claim 61, wherein the treatment of the fungal cell with a DNase and RNase increases amount of a protein produced by the CHO cell when compared to a fungal cell that is not treated with a DNase and an RNase.

66. The fungal cell of claim 65, the treatment of the fungal cell increases the yield of antibodies, cytokines, growth factors, viral vectors, antigens, vaccines, complex engineered antibodies, trivalent T-cell engagers, checkpoint modulators, naive proteins, recombinant proteins, vitamins, hormones, vaccine, and antibody cytokine fusions over a fungal cell that is not treated with a DNase and an RNase.

67. The method of claim 59, wherein the cell is a human cell.

68. The method of claim 61, wherein the treatment of the human cell with a DNase and RNase increases amount of a protein produced by the human cell when compared to a CHO cell that is not treated with a DNase and an RNase.

69. A method to increase the ability to increase production of a protein, comprising:
(a) isolating one or more of a cell, a cell culture, an organoid, a tissue, an embryo, an organ, a single-cellular organism, a multicellular organism;
(b) treating the one or more of the cell, the cell culture, the organoid, the tissue, the embryo, the organ, the single-cellular organism, the multicellular organism with an antibody that binds to an DNA and an antibody that binds to an RNA;
(c) washing the one or more of the treated cell, the treated cell culture, the treated organoid, the treated tissue, the treated embryo, the treated organ, the treated single-cellular organism, the treated multicellular organism following treatment with the antibody that binds to an DNA and the antibody that binds to an RNA;
(d) biomanufacturing a protein using the one or more of the treated cell, the treated cell culture, the treated organoid, the treated tissue, the treated embryo, the treated organ, the treated single-cellular organism, and the treated multicellular organism.

70. The method of claim 69, wherein the one or more of the treated cell, the treated cell culture, the treated organoid, the treated tissue, the treated embryo, the treated organ, the treated single-cellular organism and the treated multicellular organism are treated with two or more rounds with antibodies against a DNA and an RNA.

71. A method to increase the ability to increase production of a protein, comprising:
(a) isolating one or more of a cell, a cell culture, an organoid, a tissue, an embryo, an organ, a single-cellular organism, a multicellular organism;
(b) treating the one or more of the cell, the cell culture, the organoid, the tissue, the embryo, the organ, the single-cellular organism, the multicellular organism with a nuclease producing organism;
(c) washing the one or more of the treated cell, the treated cell culture, the treated organoid, the treated tissue, the treated embryo, the treated organ, the treated single-cellular organism, the treated multicellular organism following treatment with the nuclease producing organism;
(d) biomanufacturing a protein using the one or more of the treated cell, the treated cell culture, the treated organoid, the treated tissue, the treated embryo, the treated organ, the treated single-cellular organism, and the treated multicellular organism.

72. The method of claim 71, wherein the one or more of the treated cell, the treated cell culture, the treated organoid, the treated tissue, the treated embryo, the treated organ, the treated single-cellular organism, the treated multicellular organism is further treated prior to, during or following treatment with a nuclease-producing organism, with one or more of an RNase, a DNase, a DNase producing cell or an RNase producing cell.

73. The method of claim 71, wherein the treated cell is a CHO cell.

74. The method of claim 73, wherein the treatment of the CHO cell with a DNase and RNase increases amount of a protein produced by the CHO cell when compared to a CHO cell that is not treated with a DNase and an RNase.

75. The method of claim 71, wherein the treatment of the cell, the cell culture, the organoid, the tissue, the embryo, the organ, the single-cellular organism and the multicellular organism increases the amount of antibodies, cytokines, growth factors, viral vectors, antigens, vaccines, complex engineered antibodies, trivalent T-cell engagers, checkpoint modulators, naive proteins, recombinant proteins, vitamins, hormones, vaccine, and antibody cytokine fusions produced when compared to a cell, a cell culture, an organoid, a tissue, an embryo, an organ, a single-cellular organism, a multicellular organism that is not treated with a DNase and an RNase.

76. The method of claim 71, wherein the cell is a fungal cell.

77. The method of claim 76, wherein the treatment of the fungal cell with a DNase and RNase increases amount of a protein produced by the CHO cell when compared to a fungal cell that is not treated with a DNase and an RNase.

78. The fungal cells of claim 77, the treatment of the fungal cell increases the yield of antibodies, cytokines, growth factors, viral vectors, antigens, vaccines, complex engineered antibodies, trivalent T-cell engagers, checkpoint modulators, naive proteins, recombinant proteins, vitamins, hormones, vaccine, and antibody cytokine fusions over a fungal cell that is not treated with a DNase and an RNase.
